(19) United States
(12) Patent Application Publication
(10) Pub. No.: US 2025/0092067 A1
(43) Pub. Date: Mar. 20, 2025

(54) APT1 and APT2 Inhibitors and Uses Thereof

(71) Applicant: Cornell University, Ithaca, NY (US)

(72) Inventors: Hening Lin, Ithaca, NY (US); Kevin Wielenberg, Brooktondale, NY (US); Min Yang, Ithaca, NY (US)

(21) Appl. No.: 18/562,243

(22) PCT Filed: May 19, 2022
(86) PCT No.: PCT/US2022/030113

§ 371 Date: Nov. 17, 2023

Related U.S. Application Data
(60) Provisional application No. 63/190,447, filed on May 19, 2021.

(51) Int. Cl. C07F 5/02; A61K 31/496; A61K 31/675; A61K 31/69; C07D 409/06; C07D 495/04; C07F 9/6509; C07F 9/6558

(52) U.S. Cl. CPC C07F 5/025; A61K 31/496; A61K 31/675; A61K 31/69; C07D 409/06; C07D 495/04; C07F 9/650952; C07F 9/65586

(57) ABSTRACT

Compounds, compositions including one or more of the compound(s), and methods of using the compounds and the compositions. The compounds comprise an (I) group. In various examples, a compound is an Acyl Protein Thioesterase 1 inhibitor (APT1 inhibitor) and/or an Acyl Protein Thioesterase 2 inhibitor (APT2 inhibitor). Compound(s) or composition(s), which may be pharmaceutical composition(s), can be used in methods of treating diseases or disorders, such as, for example, autoimmune disorders, neurodegenerative disorders, inflammatory disorders, and immune-mediated cancer diseases.

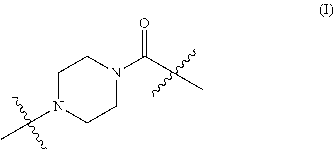 [SMILES: *C(=O)N1CCN(*)CC1]

### CROSS-REFERENCE TO RELATED APPLICATIONS

This application claims the benefit of U.S. Provisional Patent Application No. 63/190,447, filed May 19, 2021, the contents of the above-identified application are hereby fully incorporated herein by reference in their entirety.

### STATEMENT REGARDING FEDERALLY SPONSORED RESEARCH AND DEVELOPMENT

This invention was made with government support under grant number GM008500 awarded by the National Institutes of Health. The government has certain rights in this invention.

### BACKGROUND OF THE DISCLOSURE

Acyl Protein Thioesterase 2 (APT2; gene name LYPLA2) is a serine hydrolase enzyme that catalyzes the removal of palmitoylation on protein cysteine residues. Several substrate proteins are known, including CD95 (Blood. 2015, Vol125, P2948-2957), Scribble (Cell Chem Biol. 2017, Vol24, P87-97), ZDHHC6 (Elife. 2017, Vol6, Pe27826.), MC1R (Nat Commun, 2019, Vol10, P877), TEAD (Proc Natl Acad Sci USA, Vol116, P9877-9882), TNF-R1 (Cell Commun Signal, 2019, Vol17, P90), and STAT3 (Nature. 2020 Vol586, P434-439). However, the therapeutic value of targeting APT2 has been unknown until a recent study showed that APT2 inhibition could inhibit STAT3 activity and Th17 cell development (Nature. 2020, Vol586, P434-439). Th17 cells are proinflammatory cells that are involved in many human autoimmune diseases (Trends Pharmacol Sci. 2014, Vol35, P493-500; Semin Immunopathol. 2019, Vol41, P283-297.).

Cysteine palmitoylation (S-palmitoylation) is a post-translational modification wherein cysteine residues are acylated with palmitic acid or other long chain fatty acids. Since the acyl group is attached through a labile thioester bond, the modification is readily enzymatically reversible allowing for dynamic control of the modification. S-palmitoylation is installed by about two dozen zinc finger aspartate-histidine-histidine-cysteine containing proteins (DHHC) and removed by several serine hydrolyzes, including Acyl Protein Thioesterase 1 (APT1 or LYPLA1), APT2 (or LYPLA2), ABHD17A/B/C, and ABHD10. S-palmitoylation can affect protein membrane localization, trafficking, activity, stability, and protein-protein interactions.

While many reports point to the S-palmitoylation of immune signaling proteins, such as NOD2 and STING, the molecular understanding of the function of S-palmitoylation in immune signaling remains limited. Recently, a palmitoylation-depalmitoylation cycle catalyzed by DHHC7 and APT2 is reported to regulate STAT3, providing a new model for how dynamic palmitoylation could regulate immune signaling. The transcription activator STAT3 aids the differentiation of CD4⁺ T cells into T Helper 17 (TH17) cells by promoting the transcription of certain genes, such as RORC and IL17A. STAT3 is activated by the phosphorylation of Y705 by Janus kinase 2 (JAK2) in response to certain cytokine stimulation, such as IL-6. STAT3 C108 palmitoylation by DHHC7 promotes the membrane targeting of STAT3, where JAK2 is localized, thus promoting phosphorylation by JAK2. However, subsequent nuclear translocation requires the depalmitoylation by APT2. Thus, the palmitoylation-depalmitoylation cycle together promotes STAT3 activation. Therefore, breaking this palmitoylation cycle by either disrupting DHHC7 or APT2 could inhibit STAT3 and suppress T_{H}17 differentiation. Increased T_{H}17 cell differentiation contributes to inflammatory bowel disease (IBD). Hence, inhibiting DHHC7 or APT2 could help to treat IBD, as demonstrated in an IBD mouse model. Additionally, APT2 inhibitors have been reported to have potential to treat melanoma.

APT1 and APT2 share 65% sequence identity and several small molecule inhibitors have been developed for them. The β-lactone Palmostatin B was developed as a covalent APT inhibitor and blocks the catalytic serine by esterifying it. Though very potent, Palmostatin B is not APT1/2 selective and inhibits many serine hydrolases. More recently, aided by high-throughput screening studies, certain piperazine amides have been found to be effective inhibitors of APT1 and APT2. Specifically, ML348 and ML349 have been identified as selective, noncovalent inhibitors of APT1 and APT2, respectively. However, neither is as potent in vitro as Palmostatin B. Together Palmostatin B, ML348 and ML349 have proven to be highly useful for examining numerous cellular functions of APT1 and APT2, including regulating Scribble localization and pro-tumor signaling, regulating Ras signaling, and regulating MC1R palmitoylation in melanoma. Similarly, the APT1-selective inhibitor ML348 has been reported to rescue behavior and neuropathology in Huntington disease mice. Given these promising reports, developing more and improved APT1 and APT2 inhibitors will be critical to further test the therapeutic potential of inhibiting APT1 and APT2.

### SUMMARY OF THE DISCLOSURE

In various examples, a compound has the following structure:

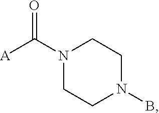

where the A group is an aryl group or heterocyclic group, or the like, or a substituted derivative or analog thereof, and the B group is chosen from alkyl groups, phenyl groups, alkoxylphenyl groups, N-aryl alkylamide groups, N-alkyl alkylamide groups, and the like, and substituted derivatives and analogs thereof, or a pharmaceutically acceptable salt, a salt, a partial salt, a solvate, a polymorph, a prodrug, or the like thereof, or a stereoisomer or a mixture of stereoisomers, an isotopic variant, a tautomer, or the like thereof, with the proviso the compound does not have the following structure:

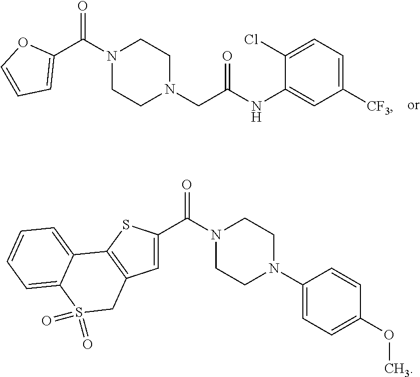 [SMILES: O=C(CN1CCN(C(=O)c2ccco2)CC1)Nc1cc(C(F)(F)F)ccc1Cl ; COc1ccc(N2CCN(C(=O)c3cc4c(s3)-c3ccccc3S(=O)(=O)C4)CC2)cc1]

In various examples, the A group is chosen from furanyl groups, thienyl groups, naphtho thiophen-2-yl groups, oxido/dioxido thieno thiochromeneyl groups, naphthalenyl groups, phenyl groups, and the like, and substituted derivatives and analogs thereof. In various examples, the A group and/or the B group comprises one or more

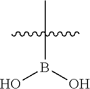 [SMILES: *B(O)O]

group(s) or ester(s) or cyclic ester(s) thereof, one or more

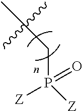

group(s), where n is 0, 1, or 2, and each Z is independently chosen from —OH group, halide groups, and alkoxyl groups, one or more

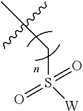

group(s), where n is 0, 1, or 2, and W is a halide group, or the like, or the like, or any combination thereof. In various examples, the A group is chosen from

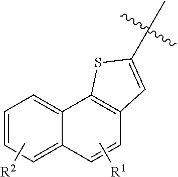

and the like, and positional isomers thereof, where R¹ and R² are independently chosen from —H group, —OH group, halide groups, alkyl groups, alkoxyl groups, aryl groups, halogenated hydrocarbon groups, acyl groups, alkylamino groups, cyano group, nitro group, thioether groups, a

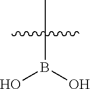 [SMILES: *B(O)O]

group and esters thereof and cyclic esters thereof,

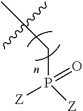

groups, where n is 0, 1, or 2, and each Z is independently chosen from —OH group, halide groups, and alkoxyl groups,

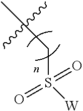

groups, where n is 0, 1, or 2, and W is a halide group, or the like, and the like, and any combination thereof. In various examples, the A group is chosen from

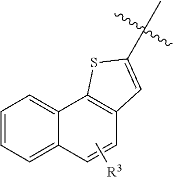

and the like, and positional isomers thereof, where R³ is chosen from —H group, —OH group, halide groups, alkyl groups, alkoxyl groups, aryl groups, halogenated hydrocarbon groups, acyl groups, alkylamino groups, cyano group, nitro group, thioether groups,

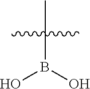 [SMILES: *B(O)O]

group and esters thereof and cyclic esters thereof,

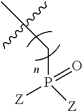

groups, where n is 0, 1, or 2, and each Z is independently chosen from —OH group, halide groups, and alkoxyl groups,

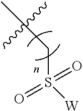

groups, where n is 0, 1, or 2, and W is a halide group, or the like, and the like. In various examples, the A group has the following structure:

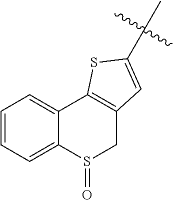 [SMILES: *c1cc2c(s1)-c1ccccc1S(=O)C2]

or the like, or a positional isomer thereof. In various examples, the A group is chosen from

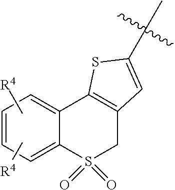

and the like, and positional isomers thereof, where each R⁴ is independently chosen from —H group, —OH group, halide groups, alkyl groups, alkoxyl groups, aryl groups, halogenated hydrocarbon groups, acyl groups, alkylamino groups, cyano group, nitro group, thioether groups,

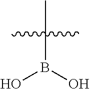 [SMILES: *B(O)O]

group and esters thereof and cyclic esters thereof,

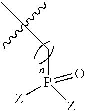

groups, where n is 0, 1, or 2, and each Z is independently chosen from —OH group, halide groups, and alkoxyl groups,

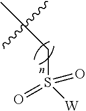

groups, where n is 0, 1, or 2, and W is a halide group, or the like, and the like. In various examples, the A group has the following structure:

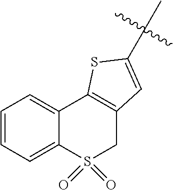 [SMILES: *c1cc2c(s1)-c1ccccc1S(=O)(=O)C2]

or the like, or a positional isomer thereof. In various examples, the A group has the following structure:

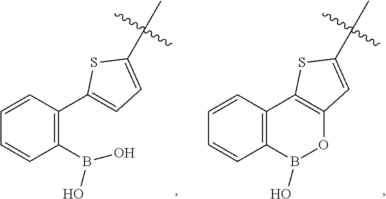 [SMILES: *c1ccc(-c2ccccc2B(O)O)s1 ; *c1cc2c(s1)-c1ccccc1B(O)O2]

or the like, or a positional isomer thereof. In various examples, the A group has the following structure:

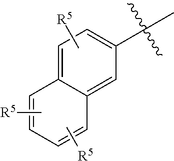

or the like, or a positional isomer thereof, where each R⁵ is independently chosen from —H group, —OH group, halide groups, alkyl groups, alkoxyl groups, aryl groups, halogenated hydrocarbon groups, acyl groups, alkylamino groups, cyano group, nitro group, thioether groups, a

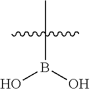 [SMILES: *B(O)O]

group and esters thereof and cyclic esters thereof,

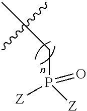

groups, where n is 0, 1, or 2, and each Z is independently chosen from —OH group, halide groups, and alkoxyl groups, and

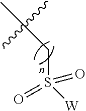

groups, where n is 0, 1, or 2, and W is a halide group, or the like, and the like. In various examples, the B group is chosen from

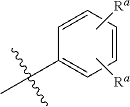

and the like, where each R^{a} is independently chosen from —H group, —OH group, halide groups, alkyl groups, alkoxyl groups, halogenated hydrocarbon groups, acyl groups, alkylamino groups, cyano group, nitro group, thioether groups,

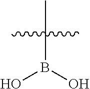 [SMILES: *B(O)O]

group and esters thereof and cyclic esters thereof,

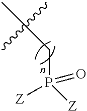

groups, where n is 0, 1, or 2, and each Z is independently chosen from —OH group, halide groups, and alkoxyl groups,

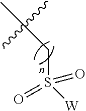

groups, where n is 0, 1, or 2, and W is a halide group, or the like, and the like.

In various examples, the B group is chosen from

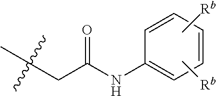

or the like, where each R^{b} is independently chosen from —H group, —OH group, halide groups, alkyl groups, alkoxyl groups, halogenated hydrocarbon groups, acyl groups, alkylamino groups, cyano group, nitro group, thioether groups,

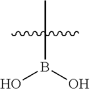 [SMILES: *B(O)O]

group and esters thereof and cyclic esters thereof,

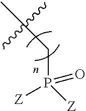

groups, where n is 0, 1, or 2, and each Z is independently chosen from —OH group, halide groups, and alkoxyl groups,

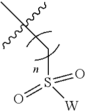

groups, where n is 0, 1, or 2, and W is a halide group, or the like, and the like. In various examples, the B group has the following structure:

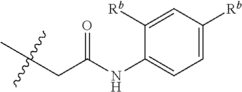

or the like. In various examples, the compound comprises any one of the aforementioned A groups and any one of the aforementioned B groups. In various examples, the compound has the following structure:

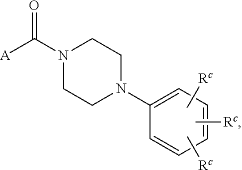

or the like, or a positional isomer thereof, where each R^{c} is independently chosen from —H group, —OH group, halide groups, alkyl groups, alkoxyl groups, halogenated hydrocarbon groups, acyl groups, alkylamino groups, cyano group, nitro group, thioether groups,

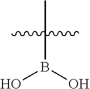 [SMILES: *B(O)O]

group and esters thereof and cyclic esters thereof,

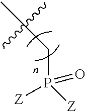

groups, where n is 0, 1, or 2, and each Z is independently chosen from —OH group, halide groups, and alkoxyl groups, and

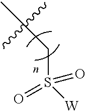

groups, where n is 0, 1, or 2, and W is a halide group, or the like, and the like. In various examples, the compound has the following structure:

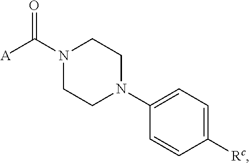

or the like. In various examples, the compound is chosen from compounds having the following structure:

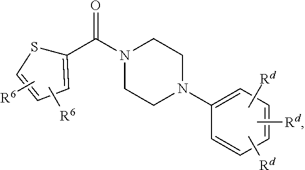

or the like, or a positional isomer thereof, where each R^{d} is independently chosen from —H group, —OH group, halide groups, alkyl groups, alkoxyl groups, halogenated hydrocarbon groups, acyl groups, alkylamino groups, cyano group, nitro group, thioether groups, and the like, and each R⁶ is independently chosen from —H group, —OH group, halide groups, alkyl groups, alkoxyl groups, halogenated hydrocarbon groups, acyl groups, alkylamino groups, cyano group, nitro group, thioether groups,

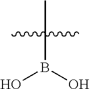 [SMILES: *B(O)O]

group and esters thereof and cyclic esters thereof,

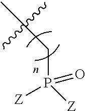

groups, where n is 0, 1, or 2, and each Z is independently chosen from —OH group, halide groups, and alkoxyl groups,

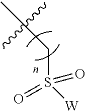

groups, where n is 0, 1, or 2, and W is a halide group, or the like, and the like. In various example, only one R⁶ is a

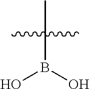 [SMILES: *B(O)O]

group or an ester thereof or a cyclic ester thereof, a

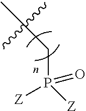

group, where n is 0, 1, or 2, and each Z is independently chosen from —OH group, halide groups, and alkoxyl groups, a

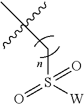

group, where n is 0, 1, or 2, and W is a halide group, or the like, or the like. In various examples, the compound has the following structure:

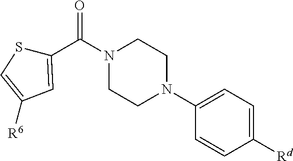

or the like. In various examples, the compound is chosen from compounds having the following structure:

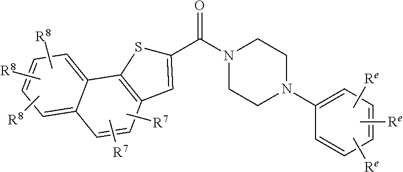

or the like, or a positional isomer thereof, where each R^{e} is independently chosen from —H group, —OH group, halide groups, alkyl groups, alkoxyl groups, halogenated hydrocarbon groups, acyl groups, alkylamino groups, cyano group, nitro group, thioether groups, and the like, and each R⁸ is independently chosen from —H group, —OH group, halide groups, alkyl groups, alkoxyl groups, halogenated hydrocarbon groups, acyl groups, alkylamino groups, cyano group, nitro group, thioether groups,

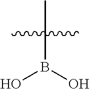 [SMILES: *B(O)O]

group and esters thereof and cyclic esters thereof,

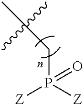

groups, where n is 0, 1, or 2, and each Z is independently chosen from —OH group, halide groups, and alkoxyl groups,

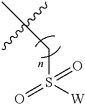

groups, where n is 0, 1, or 2, and W is a halide group, or the like, and the like. In various examples, only one R⁷ and/or only one R⁸ are present and R⁷ and/or R⁸ is/are independently a

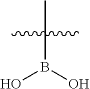 [SMILES: *B(O)O]

group or an ester thereof or a cyclic ester thereof, a

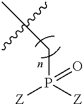

group, where n is 0, 1, or 2, and each Z is independently chosen from —OH group, halide groups, and alkoxyl groups, a

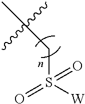

group, where n is 0, 1, or 2, and W is a halide group, or the like, or the like. In various examples, the compound has the following structure:

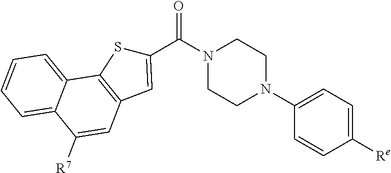

or the like. In various examples, the compound is chosen from compounds having the following structure:

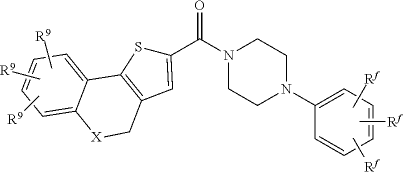

or the like, or a positional isomer thereof, where each R^{f} is independently chosen from —H group, —OH group, halide groups, alkyl groups, alkoxyl groups, halogenated hydrocarbon groups, acyl groups, alkylamino groups, cyano group, nitro group, thioether groups, and the like, each R⁹ is independently chosen from —H group, —OH group, halide groups, alkyl groups, alkoxyl groups, halogenated hydrocarbon groups, acyl groups, alkylamino groups, cyano group, nitro group, thioether groups, and the like, and X is —S(O)— or —S(O)₂—, or the like. In various examples, the compound has the following structure:

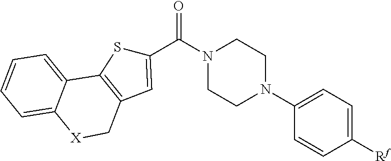

or the like. In various examples, the compound has the following structure:

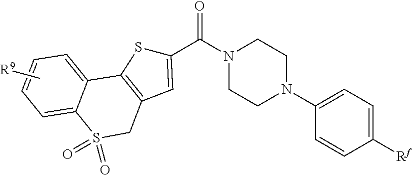

or the like, or a positional isomer thereof, where R^{f} is chosen from —H group, halide groups, alkyl groups, alkoxyl groups, alkylamino groups, cyano group, nitro group, and the like and R⁹ is chosen from —H group, halide groups, alkoxyl groups, alkylamino groups, cyano group, nitro group, and the like. In various examples, the compound is chosen from compounds having the following structure:

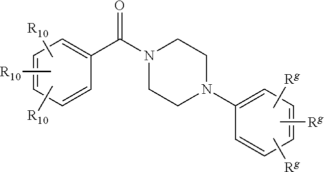

or the like, or a positional isomer thereof, where each R^{g} is independently chosen from —H group, —OH group, halide groups, alkyl groups, alkoxyl groups, halogenated hydrocarbon groups, acyl groups, alkylamino groups, cyano group, nitro group, thioether groups,

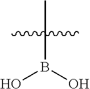 [SMILES: *B(O)O]

group and esters thereof and cyclic esters thereof,

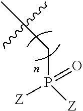

groups, where n is 0, 1, or 2, and each Z is independently chosen from —OH group, halide groups, and alkoxyl groups,

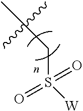

groups, where n is 0, 1, or 2, and W is a halide group, or the like, and the like and each R¹⁰ is independently chosen from chosen from —H group, —OH group, halide groups, alkyl groups, alkoxyl groups, halogenated hydrocarbon groups, acyl groups, alkylamino groups, cyano group, nitro group, thioether groups,

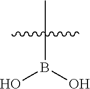 [SMILES: *B(O)O]

group and esters thereof and cyclic esters thereof,

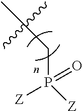

groups, where n is 0, 1, or 2, and each Z is independently chosen from halide groups and alkoxyl groups,

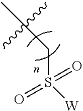

groups, where n is 0, 1, or 2, and W is a halide group, or the like, and the like. In various examples, only one R^{g} and/or only one R¹⁰ group is/are independently a

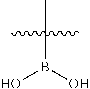 [SMILES: *B(O)O]

group or an ester thereof or a cyclic ester thereof, a

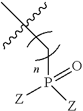

group, where n is 0, 1, or 2, and each Z is independently chosen from —OH group, halide groups, and alkoxyl groups, a

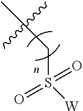

group, where n is 0, 1, or 2, and W is a halide group, or the like. In various examples, the compound has the following structure:

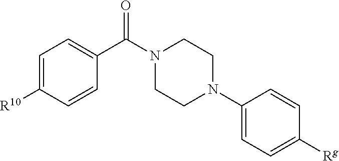

or the like. In various examples, the compound has the following structure:

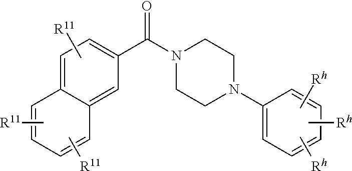

or the like, or a positional isomer thereof, where each R^{h} is independently chosen from —H group, —OH group, halide groups, alkyl groups, alkoxyl groups, halogenated hydrocarbon groups, acyl groups, alkylamino groups, cyano group, nitro group, thioether groups,

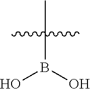 [SMILES: *B(O)O]

group and esters thereof and cyclic esters thereof,

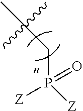

groups, where n is 0, 1, or 2, and each Z is independently chosen from —OH group, halide groups, and alkoxyl groups,

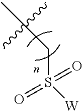

groups, where n is 0, 1, or 2, and W is a halide group, or the like, and the like and each R¹¹ is independently chosen from chosen from —H group, —OH group, halide groups, alkyl groups, alkoxyl groups, halogenated hydrocarbon groups, acyl groups, alkylamino groups, cyano group, nitro group, thioether groups,

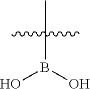 [SMILES: *B(O)O]

group and esters thereof and cyclic esters thereof,

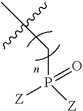

groups, where n is 0, 1, or 2, and each Z is independently chosen from —OH group, halide groups, and alkoxyl groups,

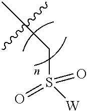

groups, where n is 0, 1, or 2, and W is a halide group, or the like, and the like. In various examples, only one R^{h} and/or only one R¹¹ group is/are present and R^{g} and/or R¹¹ is/are independently a

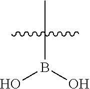 [SMILES: *B(O)O]

group or an ester thereof or a cyclic ester thereof, a

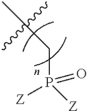

group, where n is 0, 1, or 2, and each Z is independently chosen from —OH group, halide groups, and alkoxyl groups, a

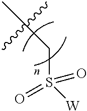

group, where n is 0, 1, or 2, and W is a halide group or the like, or the like. In various examples, the compound has the following structure:

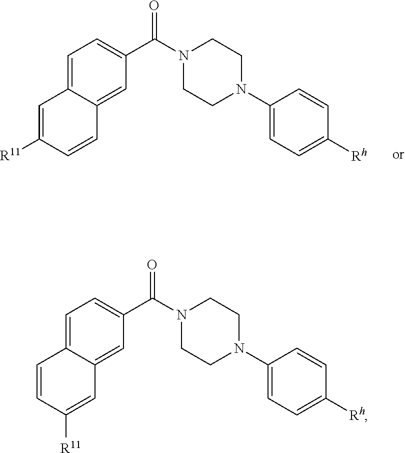

or the like, where R^{h} is chosen from alkyl groups, alkoxyl groups, alkyl amino groups, and the like and R¹¹ is a

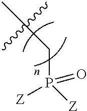

group, where n is 0, 1, or 2 and each Z is independently chosen from —OH group, halide groups, alkoxyl groups, and the like. In various examples, the compound is APT-MY1, APT-MY2, APT-MY4, APT-MY8, APT-MY9, APT-MY11, APT-MY12, KW05129, KW05175, KW05191, KW05192, KW06034, KW0526pp, KW05151, KW05153, KW05169, KW05177, KW05179, KW05200, KW05201, KW05202, KW05203, KW05204, KW05205, KW05206, KW05207, KW05208, KW05209, KW05210, KW05211, KW05212, KW05213, KW05214, KW05215, KW05216, KW05217, KW05218, KW05219, KW05220, KW05221, KW05222, KW05223, KW05224, KW05225, KW05226, KW05227, KW05228, KW05229, KW05230, KW05231, KW05232, KW05130, KW05108, or KW05116. In various examples, the compound has the following structure:

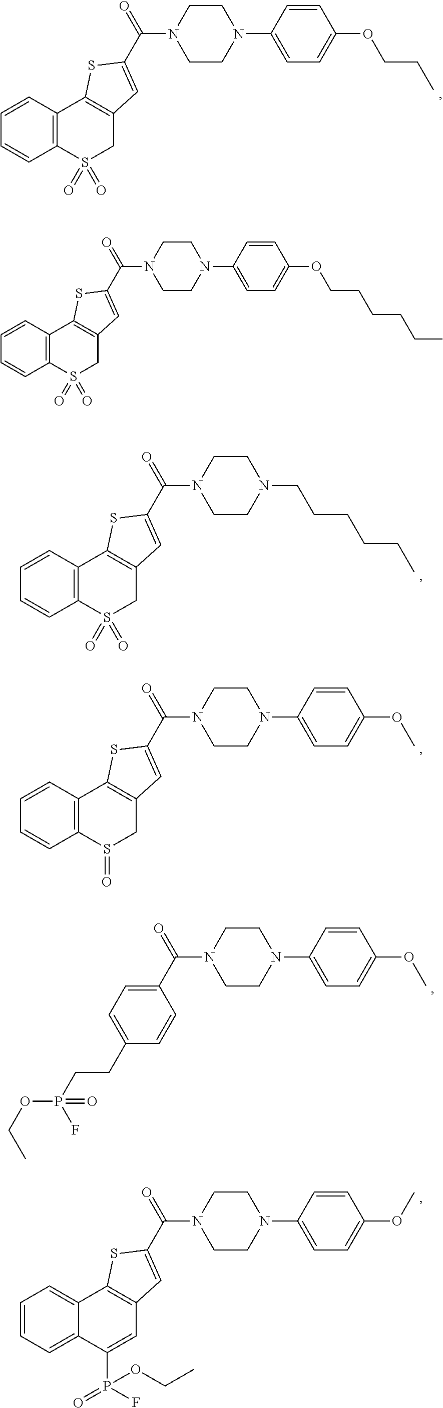 [SMILES: CCCOc1ccc(N2CCN(C(=O)c3cc4c(s3)-c3ccccc3S(=O)(=O)C4)CC2)cc1 ; CCCCCCOc1ccc(N2CCN(C(=O)c3cc4c(s3)-c3ccccc3S(=O)(=O)C4)CC2)cc1 ; CCCCCCN1CCN(C(=O)c2cc3c(s2)-c2ccccc2S(=O)(=O)C3)CC1 ; COc1ccc(N2CCN(C(=O)c3cc4c(s3)-c3ccccc3S(=O)C4)CC2)cc1 ; CCOP(=O)(F)CCc1ccc(C(=O)N2CCN(c3ccc(OC)cc3)CC2)cc1 ; CCOP(=O)(F)c1cc2cc(C(=O)N3CCN(c4ccc(OC)cc4)CC3)sc2c2ccccc12]

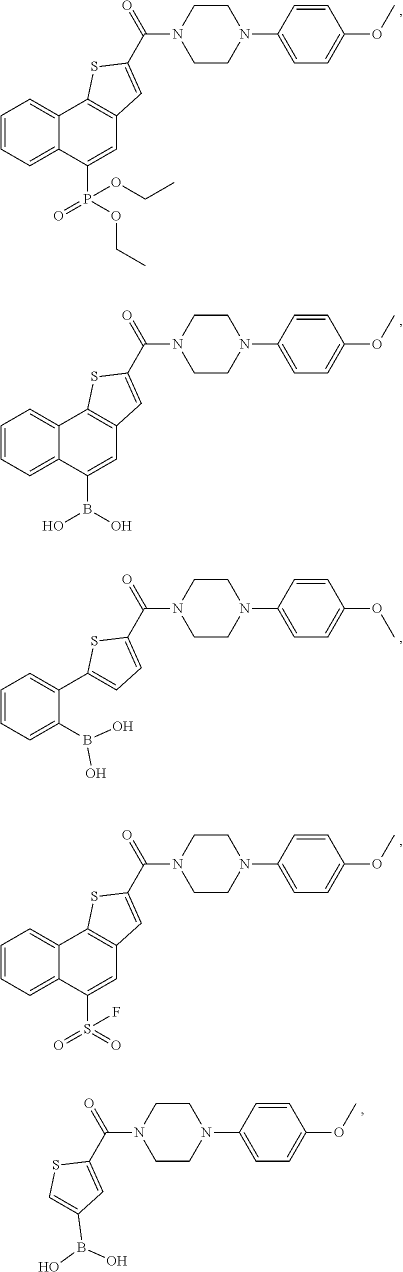 [SMILES: CCOP(=O)(OCC)c1cc2cc(C(=O)N3CCN(c4ccc(OC)cc4)CC3)sc2c2ccccc12 ; COc1ccc(N2CCN(C(=O)c3cc4cc(B(O)O)c5ccccc5c4s3)CC2)cc1 ; COc1ccc(N2CCN(C(=O)c3ccc(-c4ccccc4B(O)O)s3)CC2)cc1 ; COc1ccc(N2CCN(C(=O)c3cc4cc(S(=O)(=O)F)c5ccccc5c4s3)CC2)cc1 ; COc1ccc(N2CCN(C(=O)c3cc(B(O)O)cs3)CC2)cc1]

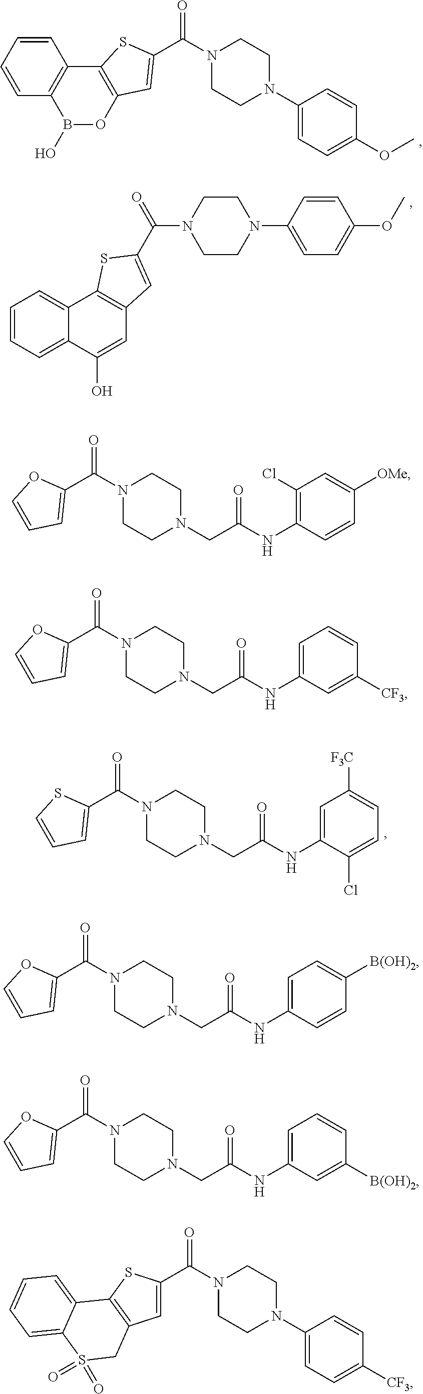

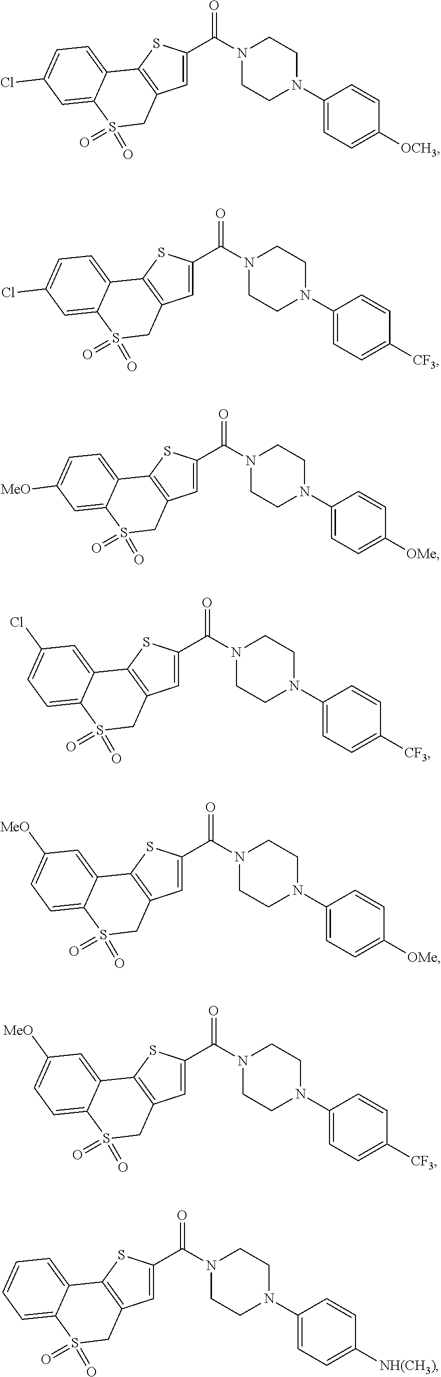

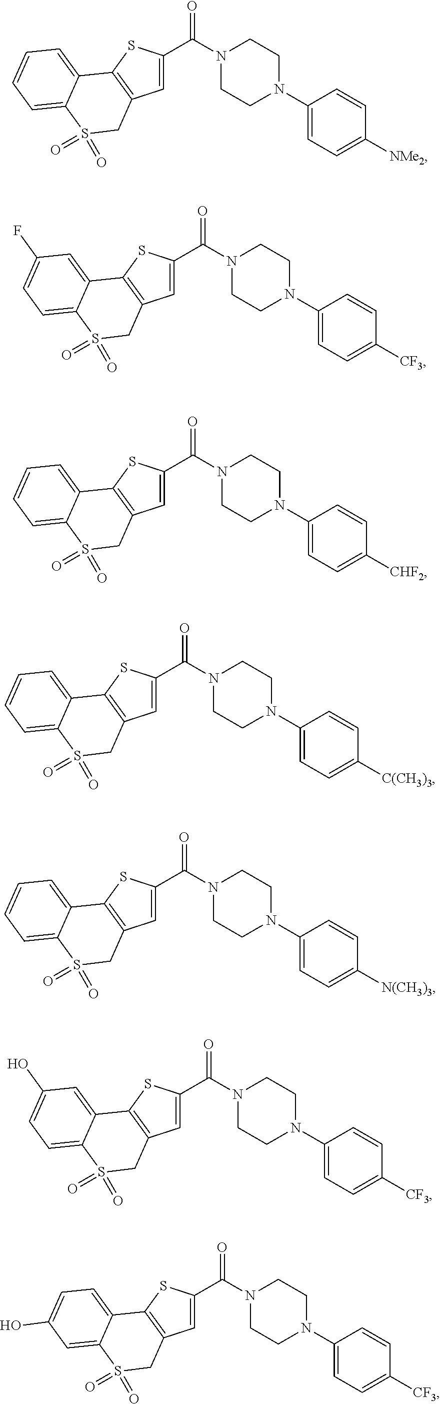

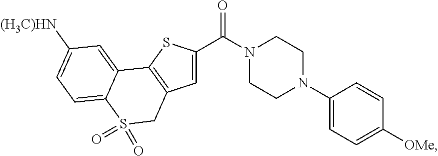

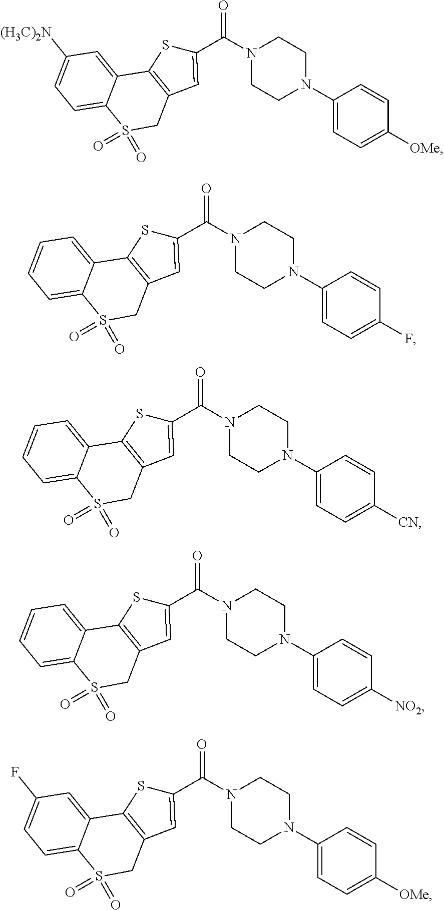

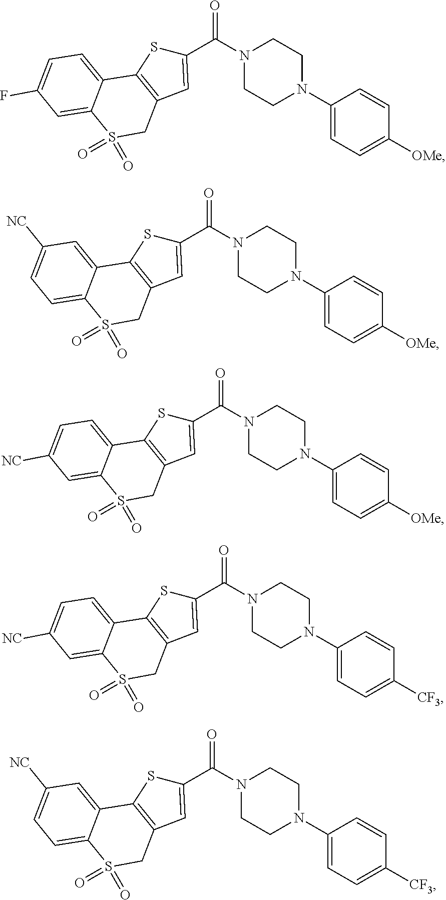 [SMILES: COc1ccc(N2CCN(C(=O)c3cc4c(s3)-c3ccc(F)cc3S(=O)(=O)C4)CC2)cc1 ; COc1ccc(N2CCN(C(=O)c3cc4c(s3)-c3cc(C#N)ccc3S(=O)(=O)C4)CC2)cc1 ; COc1ccc(N2CCN(C(=O)c3cc4c(s3)-c3ccc(C#N)cc3S(=O)(=O)C4)CC2)cc1 ; N#Cc1ccc2c(c1)S(=O)(=O)Cc1cc(C(=O)N3CCN(c4ccc(C(F)(F)F)cc4)CC3)sc1-2 ; N#Cc1ccc2c(c1)-c1sc(C(=O)N3CCN(c4ccc(C(F)(F)F)cc4)CC3)cc1CS2(=O)=O]

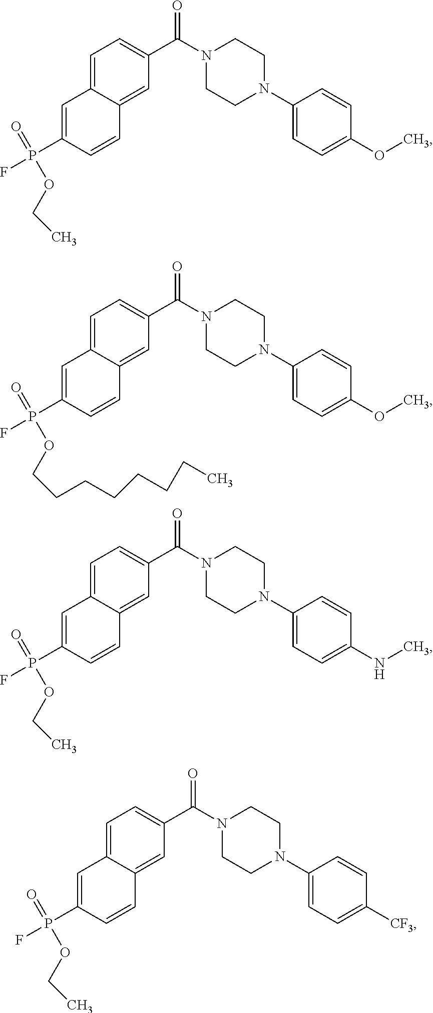

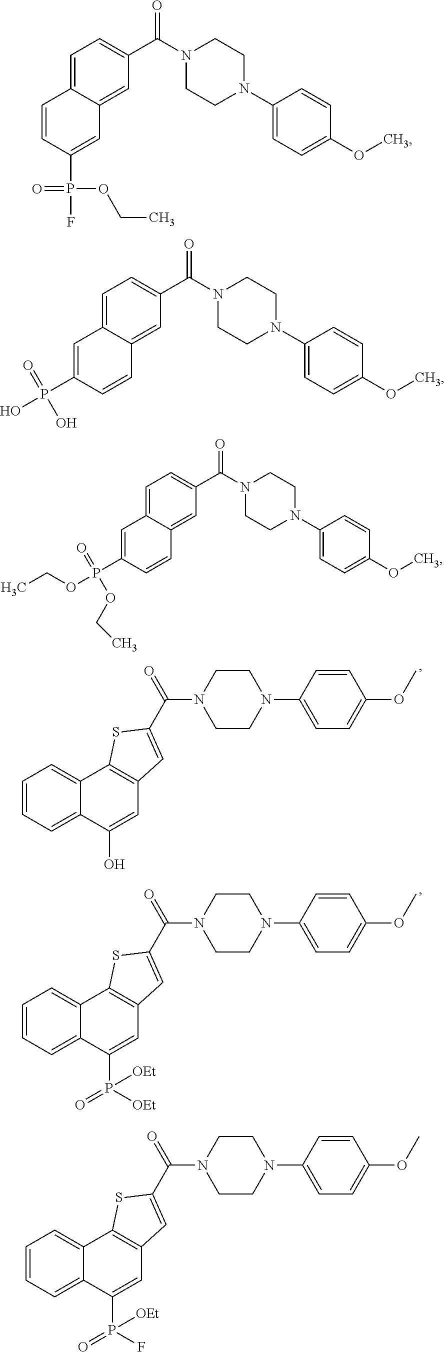

or the like.

In various examples, a pharmaceutical composition comprises one or compound(s) of the present disclosure, such as, for example, one or more of the compound(s) above. In various examples, the pharmaceutical composition further comprises one or more pharmaceutically acceptable excipient(s).

In various examples, a method of at least partially or completely inhibiting Acyl Protein Thioesterase 1 (APT1) and/or Acyl Protein Thioesterase 2 (APT2) comprises: administering to a subject an effective amount of one or compound(s) of the present disclosure, such as, for example, one or more of the compound(s) above, where the APT1 and/or the APT2 in the subject is at least partially or completely inhibited. In various examples, the APT1 is at least partially or completely inhibited and the APT2 is not at least partially or completely inhibited, or the APT2 is at least partially or completely inhibited and the APT1 is not at least partially or completely inhibited.

In various examples, a method for treating a disorder characterized by accelerated differentiation of T helper 17 (Th17) cells comprises: administering to subject diagnosed with or suffering from the disorder an effective amount of one or more inhibitor(s) of an enzyme or enzymes that regulates the S-palmitoylation of STAT3. In various examples, the enzyme is Acyl Protein Thioesterase (APT1) and/or Acyl Protein Thioesterase (APT2). In various examples, the inhibitor(s) is/are a compound or compounds of the present disclosure, such as, for example, one or more of the compound(s) above. In various examples, the inhibitor(s), individually, are a non-covalent inhibitor or non-covalent inhibitors and/or a covalent inhibitor or covalent inhibitors of APT1 and/or APT2. In various examples, the disorder is an autoimmune disorder, a neurodegenerative disorder, an inflammatory disorder, an immune-mediated disease, or the like. In various examples, the autoimmune disorder is inflammatory bowel disease, multiple sclerosis, rheumatoid arthritis, lupus, graft versus host disease, type I diabetes, psoriasis, atopic dermatitis, eczema, alopecia areata, myeloproliferative neoplasms, dermatomyositis, Guillain-Barre Syndrome or the like; the neurodegenerative disorder is Parkinson's disease, Alzheimer's disease, Huntington's disease, or the like; or the immune mediated disease is lymphoma leukemia, or the like.

In various examples, a method of treating a subject diagnosed with or is in need of treatment for an autoimmune disorder, a neurodegenerative disorder, an inflammatory disorder, or the like, comprises administering to a subject an effective amount of one or more compound(s) of one or more of the compound(s) above, where one or more symptom(s) and/or indication(s) of the subject is at least partially alleviated. In various examples, the subject has APT1 and/or APT2 and the APT1 and/or the APT2 is at least partially or completely inhibited.

### BRIEF DESCRIPTION OF THE FIGURES

For a fuller understanding of the nature and objects of the disclosure, reference should be made to the following detailed description taken in conjunction with the accompanying figures.

FIG. 1 shows that MY11 and KW05129 can increase SMAD2 palmitoylation in cells compared to ML349 as a control.

FIG. 2 shows that MY11 and KW05219 can increase STAT3 palmitoylation in cells compared to ML349 as a control.

FIGS. 3A-3B show crystal structure for APT2 crystallized with ML349. The para methoxy group of ML349 points toward an unoccupied channel in APT2 (FIG. 3A). The sulfone oxygens of ML349 interact with two structural water molecules located near the APT2 active site and are positioned equally near to the catalytic S122 (FIG. 3B). The indicated distances are measured in angstroms.

FIG. 4 shows designs of new inhibitors derived from ML349. Warheads and long alkyl chains were incorporated to engage the APT2 binding pocket and the catalytic residue S122. In vitro IC50 values for APT1 (shown above) and APT2 (shown below) are listed with standard deviations. Inhibition of APT2 with KW5116 increased when the pre-incubation (PI) time was increased to 30 min.

FIG. 5 shows a synthesis of KW5108, KW5116, KW5129, KW5130 and KW5191 (Step a) POCl₃, DMF, (Step b) Sodium, EtOH, ethyl thioglycolate, (Step c) NBS, benzoyl peroxide, (Step d) Br₂, acetic acid, (Step e) NaOH, (Step f) EDC, DMAP, 1-(4-methoxyphenyl)piperazine, (Step g) chlorosulfonic acid, (Step h) NaOH, POCl₃, (Step i) KHF₂, (Step j) bis boronic acid, Xphos, Xphos-Pd-G2, KOAc. (Step k) Pd(OAc)₂ PPh₃, DIPEA, diethyl phosphonate, (Step 1) LiBr, (Step m) Diethylamino sulfur trifluoride. KW5130 was isolated as a side product of the reaction of Step j.

FIGS. 6A-6C show activity-based protein profiling (ABPP) using ActivX™ TAMRA-FP serine hydrolase probe (TAMRA-FP) to determine the mode of action and potency of ML349, KW5116, and KW5129. The fluorescent intensities were normalized using the loading intensity of each lane. (FIG. 6A) Lysates of HEK 293T cells expressing APT2 was treated with different inhibitors or control. The protein solution was passed over a size exclusion column multiple times then treated with TAMRA-FP to label APT2 that is not covalently inhibited. The protein solution was then resolved by sodium dodecyl sulfate-polyacrylamide gel electrophoresis (SDS-PAGE) and the fluorescence labeling determined by in-gel fluorescence. (FIGS. 6B and 6C) A cell lysate solution spiked with APT2 (FIG. 6B) or APT1 (FIG. 6C) was treated with inhibitor for 15 min then labeled with TAMRA-FP for 30 min. The protein solution was then resolved by SDS-PAGE and the fluorescence labeling determined by in-gel fluorescence.

FIG. 7 shows that KW5116 and KW5129 increased STAT3 palmitoylation in cells. HEK 293T cells expressing flag-tagged STAT3 and HA-tagged DHHC7 were treated with APT2 inhibitors, and metabolically labeled with Alk14 for 6 hours. After obtaining the cell lysate, STAT3 was immunoprecipitated and fluorescently labeled via click chemistry to detect its acylation level. The acylation level was normalized by the protein level as measured by coomassie brilliant blue (CBB) staining.

FIG. 8 shows TAMRA-FP ABPP with ML349, KW5129, and KW5116 in APT2 spiked cell lysate. An increase in fluorescent intensity is observed with decreasing inhibitor concentration in multiple bands with KW5116. This indicates that KW5116 reacts with off target proteins in addition to APT2. Change in fluorescent intensities with ML349 and KW5129 is only seen in the APT2 band.

FIGS. 9A-9B show that KW5129 protects mice in the dextran sodium sulfate (DSS) induced colitis model. Groups of female (A) (** P value=0.0022), and male (B) (* P value=0.166) mice were treated with 2.5% DSS in their drinking water and injected with ML349 or KW5129 starting on the same day as DSS treatment started. Body weights were tracked throughout the experiment. After sacrificing the mice, their colons were removed and measured.

FIG. 10 shows prior art compounds (Prior Art).

FIG. 11 shows prior art compounds (Prior Art).

FIG. 12 shows prior art compounds (Prior Art).

FIG. 13 shows prior art compounds (Prior Art).

FIG. 14 shows prior art compounds (Prior Art).

FIG. 15 shows a reaction scheme for KW05200.

FIG. 16 shows a reaction scheme for KW05201.

FIG. 17 shows a reaction scheme for KW05202.

FIG. 18 shows a reaction scheme for KW05203.

FIG. 19 shows a reaction scheme for KW05204.

FIG. 20 shows a reaction scheme for KW05205.

FIG. 21 shows a reaction scheme for KW05206.

FIG. 22 shows a reaction scheme for KW05207.

FIG. 23 shows a reaction scheme for KW05208.

FIG. 24 shows a reaction scheme for KW05209.

FIG. 25 shows a reaction scheme for KW05210.

FIG. 26 shows a reaction scheme for KW05211.

FIG. 27 shows a reaction scheme for KW05212.

FIG. 28 shows a reaction scheme for KW05213.

FIG. 29 shows a reaction scheme for KW05214.

FIG. 30 shows a reaction scheme for KW05215.

FIG. 31 shows a reaction scheme for KW05216.

FIG. 32 shows a reaction scheme for KW05217.

FIG. 33 shows a reaction scheme for KW05218.

FIG. 34 shows a reaction scheme for KW05219.

FIG. 35 shows a reaction scheme for KW05220.

FIG. 36 shows a reaction scheme for KW05221.

FIG. 37 shows a reaction scheme for KW05222.

FIG. 38 shows a reaction scheme for KW05223

FIG. 39 shows a reaction scheme for KW05224.

FIG. 40 shows a reaction scheme for KW05225.

FIG. 41 shows a reaction scheme for each of KW05232 and KW-5226.

FIG. 42 shows a reaction scheme for KW05227.

FIG. 43 shows a reaction scheme for KW05228.

FIG. 44 shows a reaction scheme for KW05229.

FIG. 45 shows a reaction scheme for KW05230.

FIG. 46 shows a reaction scheme for KW05231.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative, non-limiting examples of groups include:

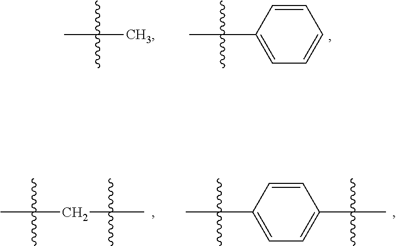

and the like.

As used herein, unless otherwise indicated, the term “alkyl group” refers to branched or unbranched hydrocarbon groups that include only single bonds between carbon atom). In various examples, an alkyl group is a C₁ to C₁₀ (e.g., C₁, C₂, C₃, C₄, C₅, C₆, C₇, C₈, C₉, or C₁₀ alkyl group), including all integer numbers of carbons and ranges of numbers of carbons therebetween, alkyl group. In various examples, an alkyl group is a saturated group. In various examples, an alkyl group is a cyclic alkyl group. Examples of alkyl groups include, but are not limited to, methyl groups, ethyl groups, propyl groups, butyl groups, isopropyl groups, tert-butyl groups, and the like. In various examples, an alkyl group is unsubstituted or substituted with one or more substituent(s). Examples of substituents include, but are not limited to, various substituents such as, for example, hydroxyl group, halide groups (—F, —Cl, —Br, and —I), halogenated alkyl groups (e.g., trifluoromethyl group and the like), aryl groups, halogenated aryl groups, alkoxide groups, amine groups, ether groups, carboxylate groups, carboxylic acid, ester groups, amide groups, cyano groups, nitro groups, thioether groups, silyl ether groups, isocyanate groups, and the like, and any combination thereof.

As used herein, unless otherwise indicated, the term “aryl group” refers to C₅ to C₃₀ (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀) aromatic or partially aromatic carbocyclic groups, including all integer numbers of carbons and ranges of numbers of carbons therebetween. In various examples, an aryl group is also referred to as an aromatic group. In various examples, aryl groups comprise polyaryl groups such as, for example, fused ring groups, biaryl groups, or a combination thereof. In various examples, the aryl group is unsubstituted or substituted with one or more substituent(s). Examples of substituents include, but are not limited to, various substituents such as, for example, hydroxyl group, halide groups (—F, —Cl, —Br, and —I), aliphatic groups (e.g., additional alkyl groups, alkenyl groups, alkynyl groups, and the like), halogenated aliphatic groups (e.g., trifluoromethyl group and the like), cycloaliphatic groups, aryl groups, halogenated aryl groups, alkoxide groups, amine groups, ether groups, carboxylate groups, carboxylic acid, ester groups, amide groups, cyano groups, nitro groups, thioether groups, silyl ether groups, isocyanate groups, and the like, and any combination thereof. In various examples, aryl groups contain one or more hetero atom(s), such as, for example, oxygen, nitrogen (e.g., pyridinyl groups and the like), sulfur, and the like, and any combination thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, and the like.

As used herein, unless otherwise indicated, a halogenated hydrocarbon group is a hydrocarbon group (e.g., an alkyl group, a cyclic alkyl group, an aryl group, or the like) in which one or more hydrogen substituent(s) is/are replaced with halide atom substituent(s) (e.g., fluorine atom(s) (e.g., a fluorinated hydrocarbon group, such as, for example, a fluorinated alkyl group, a fluorinated cyclic alkyl group, a fluorinated aryl group, or the like, or the like), bromine atom(s), chlorine atom(s), iodine atom(s), or the like, and any combination thereof). As used herein, unless otherwise indicated, a perhalogenated hydrocarbon group is a hydrocarbon group (e.g., an alkyl group, a cyclic alkyl group, an aryl group, or the like) in which all hydrogen substituent(s) is/are replaced with halogen atom substituent(s) (e.g., fluorine atom(s) (e.g., a perfluorinated hydrocarbon group, such as, for example, a perfluorinated alkyl group, a perfluorinated cyclic alkyl group, a perfluorinated aryl group, or the like, or the like), bromine atom(s), chlorine atom(s), iodine atom(s), and the like, and any combination thereof).

As used herein, unless otherwise indicated, the term “analog” refers to a compound or group that can be envisioned to arise from another compound or group, respectively, if one atom or group of atoms, functional groups, or substructures is replaced with another atom or group of atoms, functional groups, or substructures.

As used herein, unless otherwise indicated, the term “derivative” refers to a compound or group that is envisioned to or is derived from a similar compound or group, respectively, by a chemical reaction, where the compound or group is modified or partially substituted such that at least one structural feature of the original compound or group is retained.

The present disclosure describes compounds and compositions. The present disclosure also describes uses of the compounds and compositions.

In an aspect, the present disclosure provides compounds. In various examples, a compound is an Acyl Protein Thioesterase inhibitor (APT1 inhibitor) (which may be referred to as Lysophospholipase 1 (LYPLA1) or the like) and/or Acyl Protein Thioesterase inhibitor (APT2 inhibitor) (which may be referred to as Lysophospholipase 2 (LYPLA2), or the like). The compounds may be made by a method (or modified version thereof) of the present disclosure. Non-limiting examples of the compounds are described herein.

In various examples, a compound comprises a substituted piperazine-N-carbonyl group (such as, for example, a substituted piperazine-1-carbonyl group or the like) having the following structure:

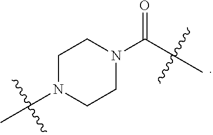 [SMILES: *C(=O)N1CCN(*)CC1]

In various examples, a compound comprises the following structure:

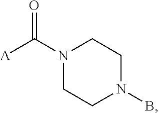

or a pharmaceutically acceptable salt, a salt, a partial salt, a solvate, a polymorph, or a prodrug thereof, or a stereoisomer or a mixture of stereoisomers, an isotopic variant, or a tautomer thereof. An A group may or may not be conjugated. In various examples, an A group comprises (or is) a substituted or unsubstituted aryl group, a substituted or unsubstituted heterocyclic group (which may or may not be aromatic), or the like, or any combination thereof. In various examples, an A group is chosen from furanyl groups, thienyl groups (e.g., 2-thienyl groups, 2-(thiophen-2-yl)phenyl groups, and the like, and positional isomers thereof), naphtho thiophen-2-yl groups (e.g., naphtho[1,2-b]thiophen-2-yl groups, and the like, and positional isomers thereof), oxido/dioxido thieno thiochromeneyl groups (e.g., 5,5-dioxido-4H-thieno[3,2-c]thiochromen-2-yl groups, and the like, and positional isomers thereof), phenyl groups, naphthalenyl groups, and the like, any of which may be substituted or unsubstituted. In various examples, a B group is chosen from alkyl groups (e.g., C₁-C₁₀ alkyl groups) (which may be fluorinated alkyl groups, such as, for example, perfluorinated alkyl groups and the like) (which may be fluorinated alkyl groups, such as, for example, perfluorinated alkyl groups and the like), phenyl groups, alkoxylphenyl groups (e.g., C₁-C₁₀ alkoxyl phenyl groups), alkylamide groups (e.g., C₁ alkylamide groups, such as, for example, acetamide groups and the like), N-aryl alkylamide groups, N-alkyl alkylamide groups, and the like, any of which may be substituted or unsubstituted. In various examples, the compound is not

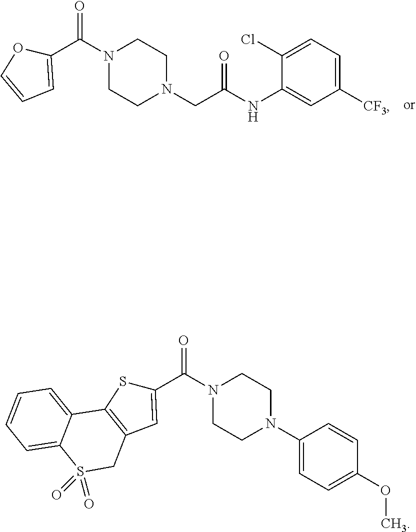 [SMILES: O=C(CN1CCN(C(=O)c2ccco2)CC1)Nc1cc(C(F)(F)F)ccc1Cl ; "or" ; COc1ccc(N2CCN(C(=O)c3cc4c(s3)-c3ccccc3S(=O)(=O)C4)CC2)cc1]

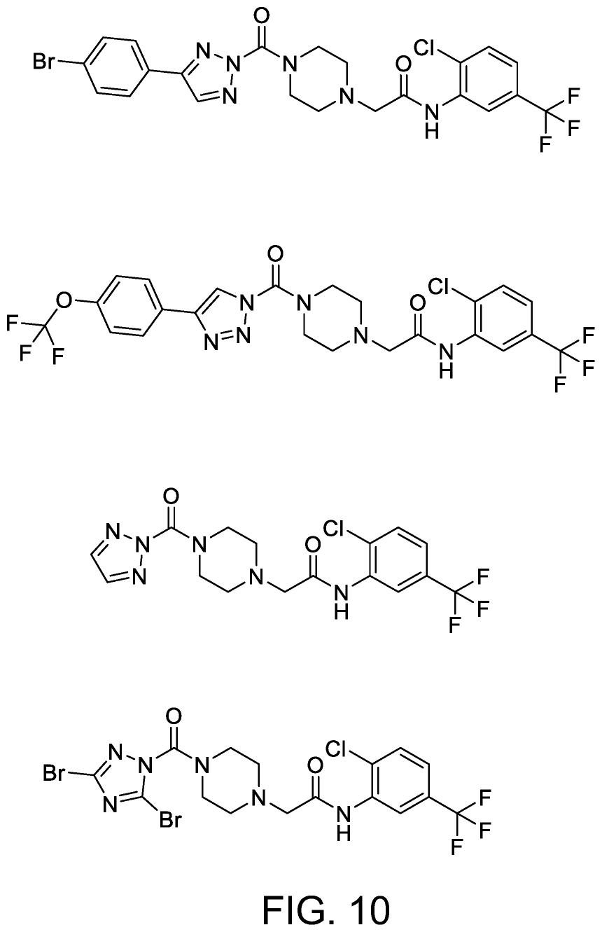

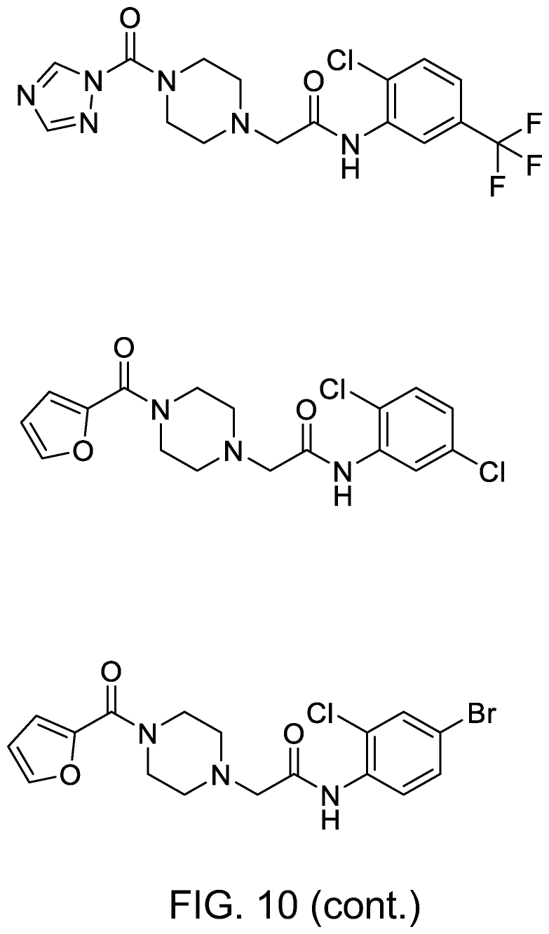

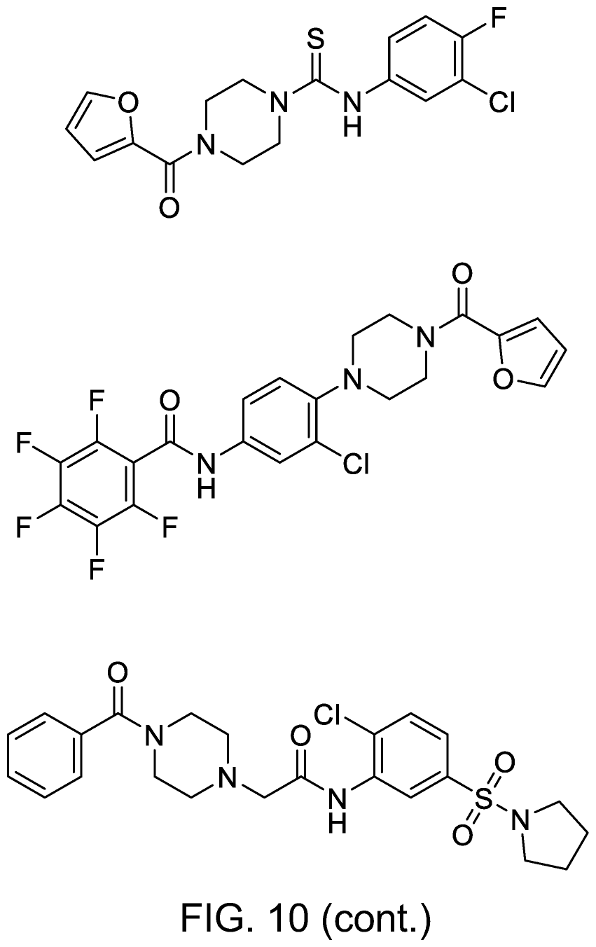

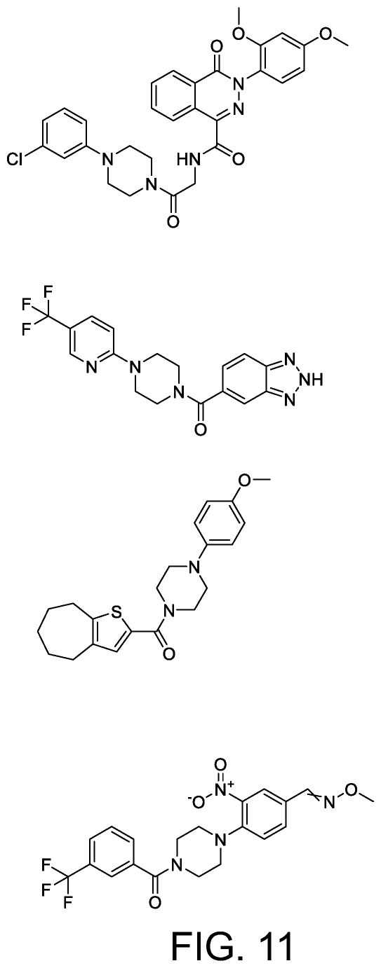

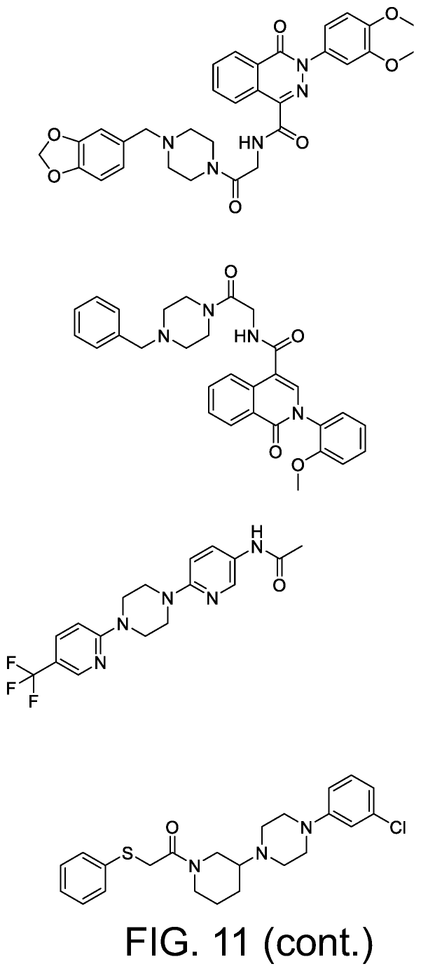

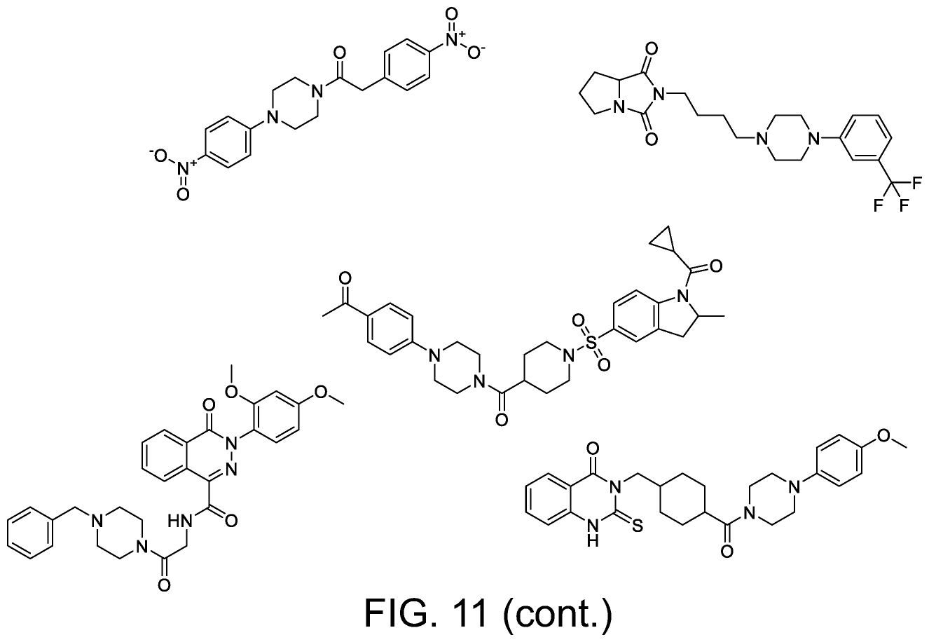

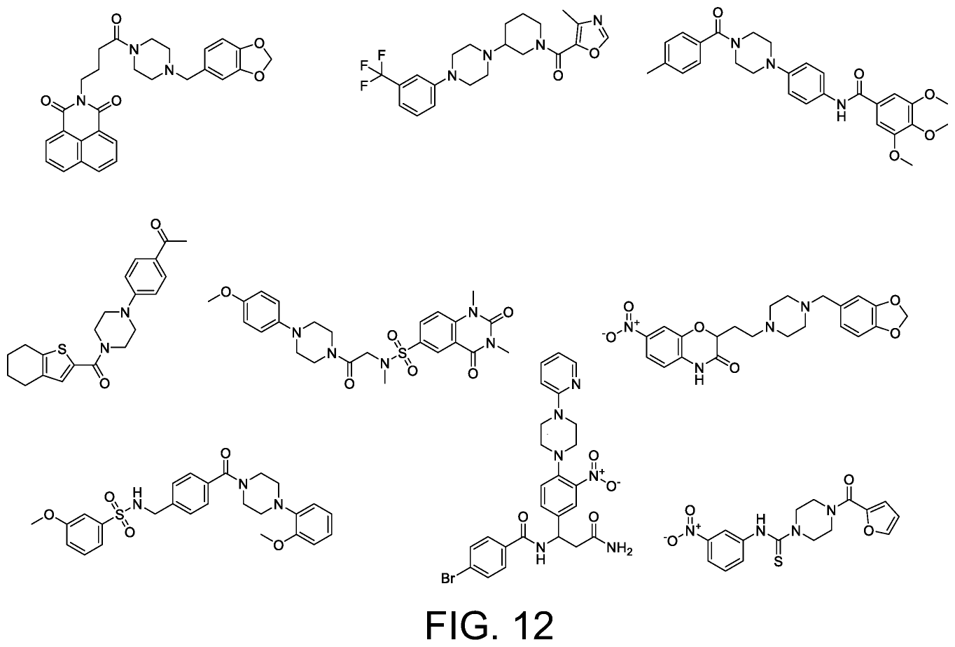

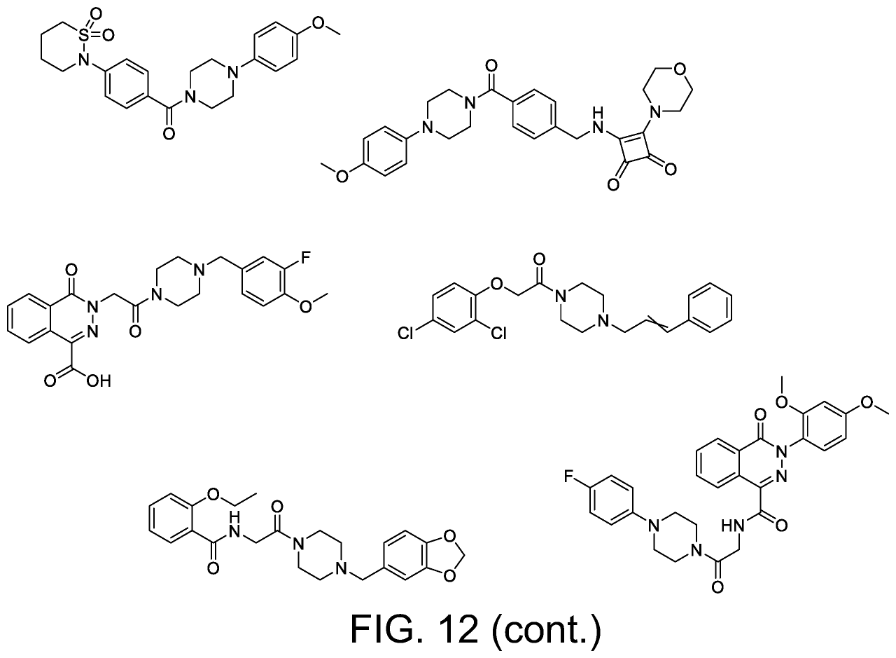

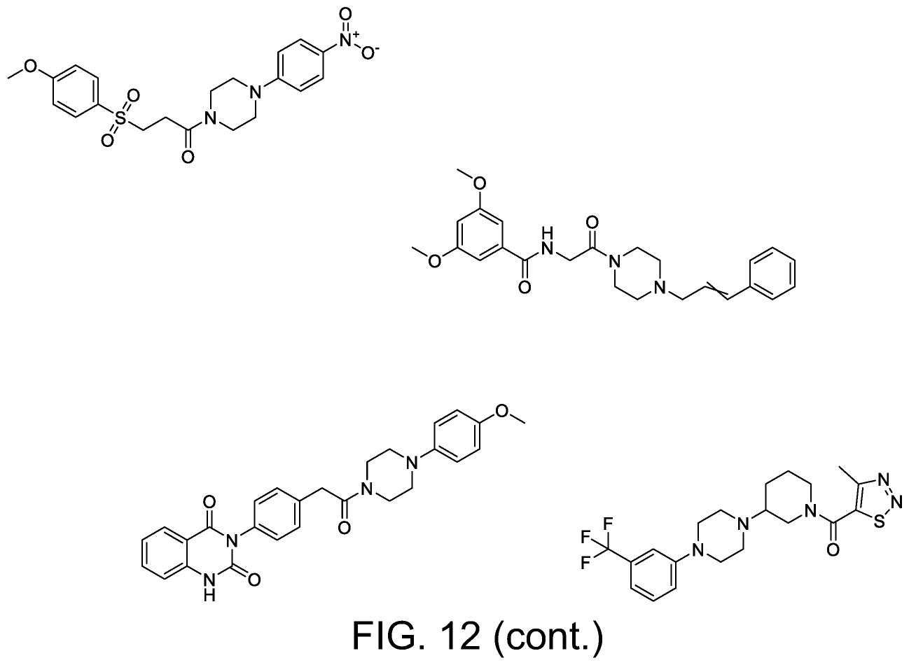

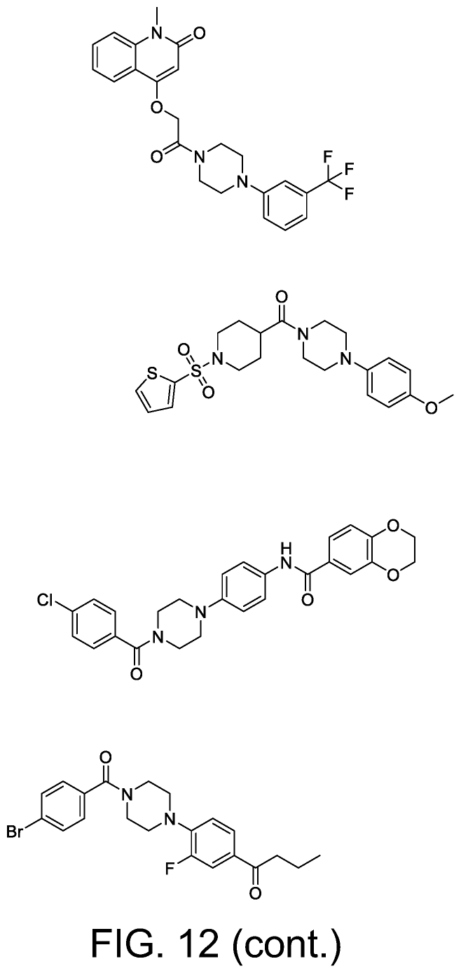

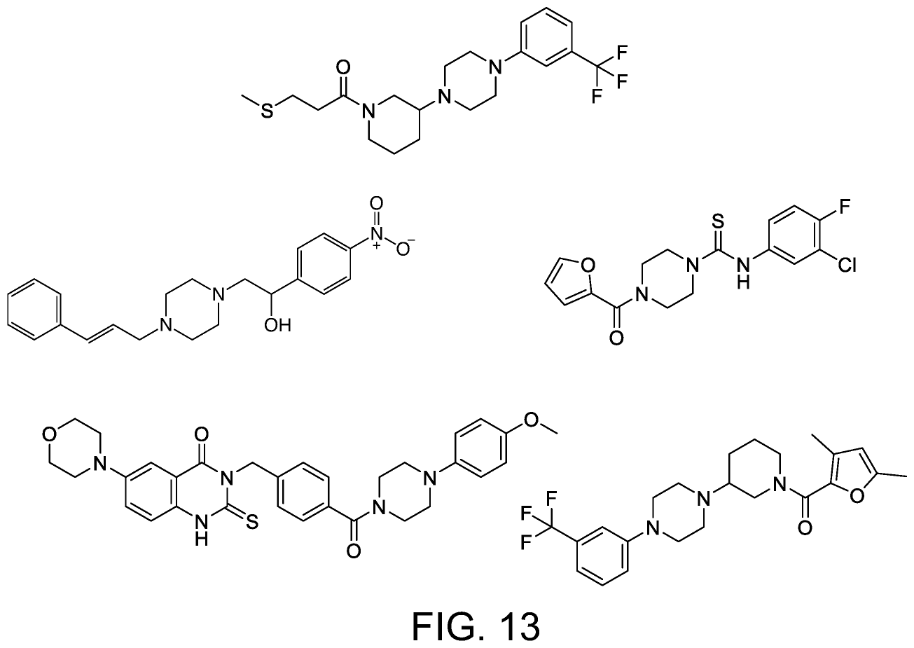

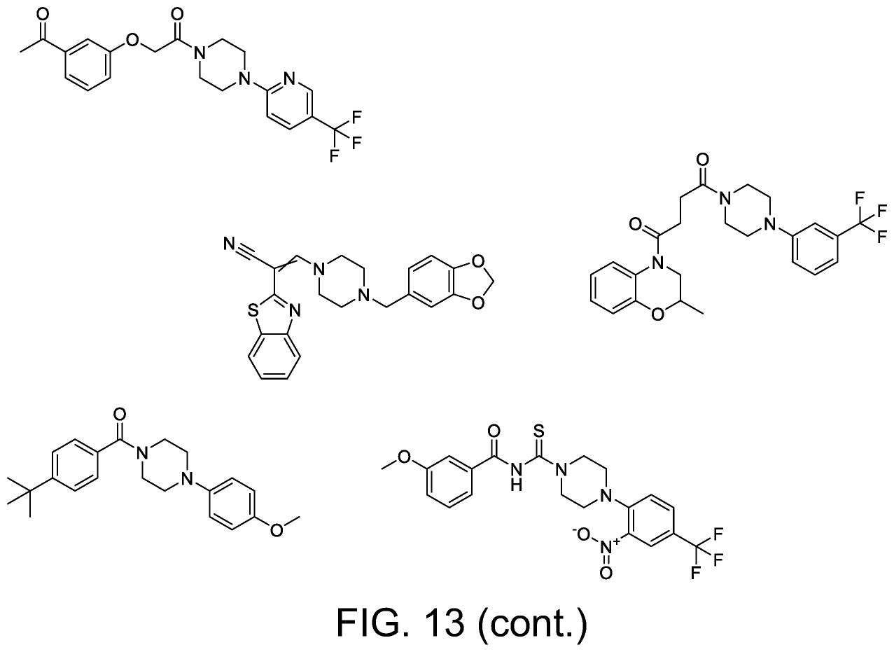

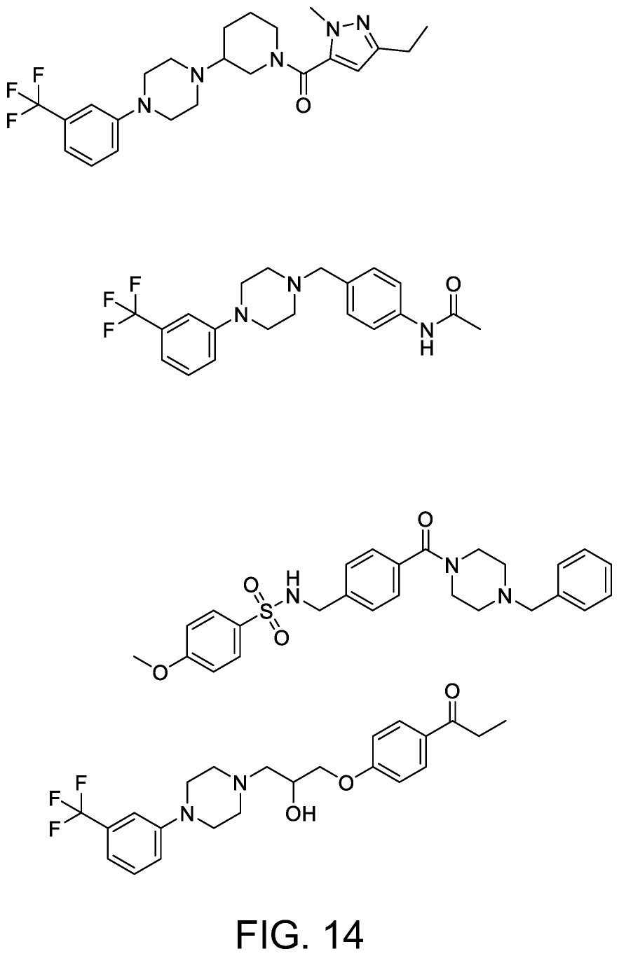

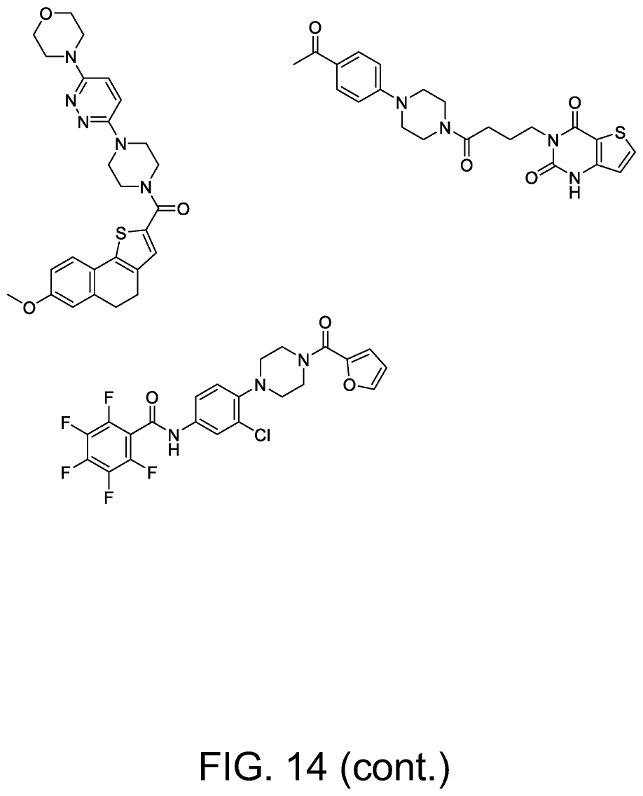

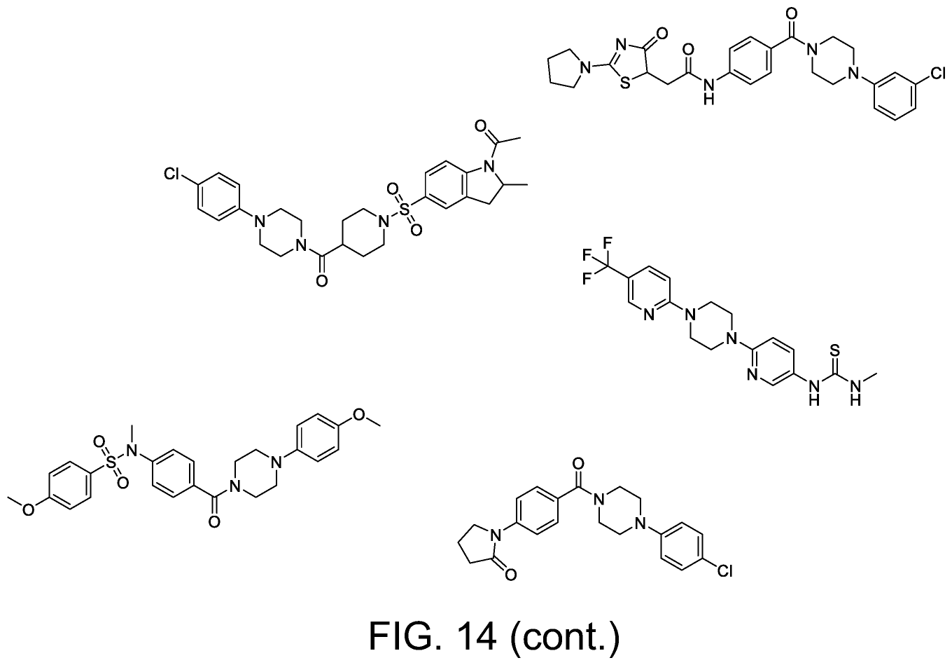

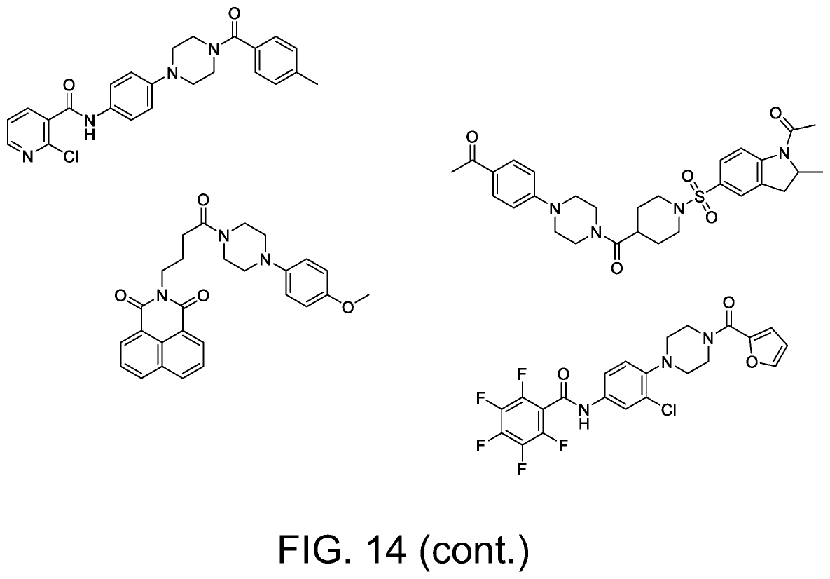

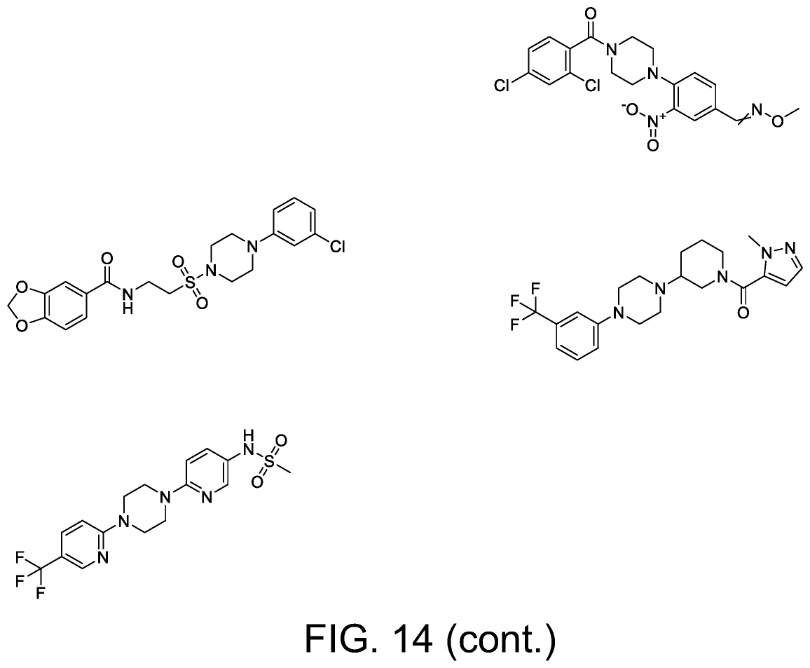

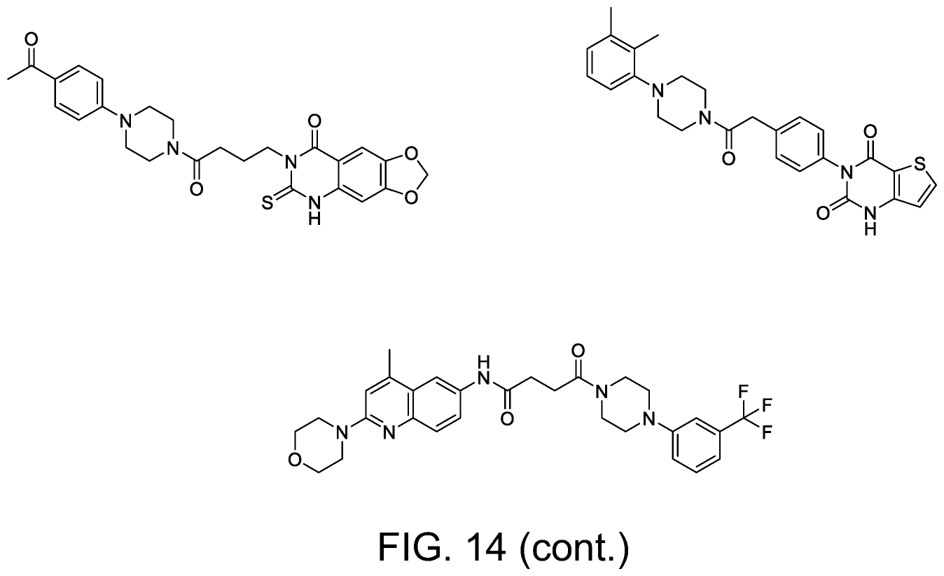

In various other examples, a compound is not a compound shown any of FIGS. 10-14, or any combination thereof. In various examples, a furanyl group or a triazolyl group.

An A group and/or B group may be substituted with one or more substituent(s). Non-limiting examples of substituents include-H group, —OH group, halide groups (e.g., —F, —Cl, —Br, and —I groups), alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group or the like), aryl groups, acyl groups (e.g., —C(O) R^{h}, where R^{h} is a C₁-C₆ alkyl group or the like (which may be a halogenated alkyl group, such as, for example, a chloroalkyl group (e.g., a chloromethyl group and the like), a bromoalkyl group (e.g., a bromomethyl group and the like), an iodoalkyl group (e.g., an iodomethyl group and the like), a fluoroalkyl group (e.g., a fluoromethyl group and the like) (which may be a perfluorinated group, such as, for example, a trifluoromethyl group, and the like), and the like)), halogenated hydrocarbon groups, alkylamino groups (e.g., —NHRⁱ groups or —NRⁱRⁱ groups where each Rⁱ is independently a C₁-C₆ alkyl group or the like), cyano group, nitro group, thioether groups (e.g., —SR^{j} groups, where R^{j} is a C₁-C₆ alkyl group or the like), and the like, substituted derivatives or analogs thereof, and any combination thereof. In various examples, a substituent comprises a one or more

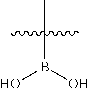 [SMILES: *B(O)O]

group(s) or ester(s) or cyclic esters(s) thereof,

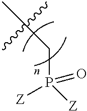

group(s), where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group or the like), and the like,

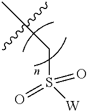

group(s), where n is 0, 1, or 2, and W is a halide group (e.g., a —F, —Cl, —Br, or —I group) or the like, or the like, or any combination thereof.

A compound can comprise various A and B groups. In various examples, an A group (e.g., a furanyl group, a thienyl group, a naphtho thiophen-2-yl group, a phenyl group, a naphthalenyl group, or the like) and/or a B group (e.g., an alkyl group, a phenyl group, an alkoxylphenyl group, an N-aryl alkylamide groups, an N-alkyl alkylamide group, or the like) comprises one or more

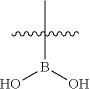 [SMILES: *B(O)O]

group(s) or ester(s) or cyclic esters(s) thereof,

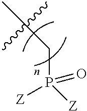

group(s), where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group or the like), and the like,

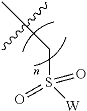

group(s), where n is 0, 1, or 2, and W is a halide group (e.g., a —F, —Cl, —Br, or —I group) or the like, or the like, or any combination thereof. In various examples, one or more of the one or more

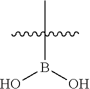 [SMILES: *B(O)O]

group(s) or ester(s) or cyclic esters(s) thereof,

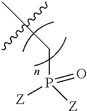

group(s), where n is 0, 1, or 2, and Z is independently at each occurrence chosen from halide groups (e.g., —F, —Cl, —Br, and —I groups), alkoxyl groups (e.g., —OR groups, where R^{g} is a C₁-C₁₀ alkyl group or the like), and the like,

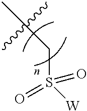

group(s), where n is 0, 1, or 2, and W is a halide group (e.g., a —F, —Cl, —Br, or —I group) or the like, or the like, or any combination thereof is present on one or more substituent(s) of an A group and/or a B group.

In various examples, an A group is chosen from unsubstituted and substituted

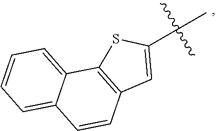 [SMILES: *c1cc2ccc3ccccc3c2s1]

and the like, and positional isomers thereof. In various examples, A is chosen from

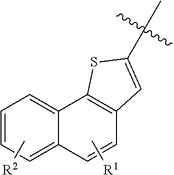

and the like, and positional isomers thereof (e.g.,

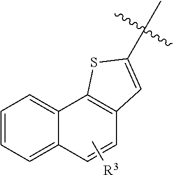

and the like, and positional isomers thereof). In various examples, each R¹ and R² are (or R³ is) a substituent (a substituent as described herein). In various examples, each R¹ and R² (or R³) are independently chosen from —H group, —OH group, halide groups, alkyl groups, alkoxyl groups, aryl groups, halogenated hydrocarbon groups, acyl groups, alkylamino groups, cyano group, nitro group, thioether groups,

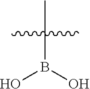 [SMILES: *B(O)O]

group and esters and cyclic esters thereof,

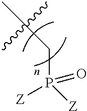

groups, where n is 0, 1, or 2, and Z is independently at each occurrence chosen from halide groups, alkoxyl groups, and the like,

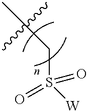

groups, where n is 0, 1, or 2, and W is a halide group (e.g., a —F, —Cl, —Br, or —I group) or the like, and the like.

In various examples, an A group is chosen from unsubstituted and substituted

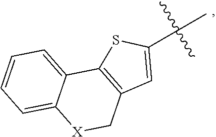

where X is —S(O)— or —S(O)₂—, and the like, and positional isomers thereof. In various examples, an A group is chosen from

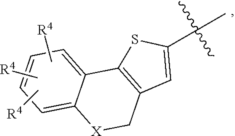

where X is —S(O)— or —S(O)₂— (e.g.,

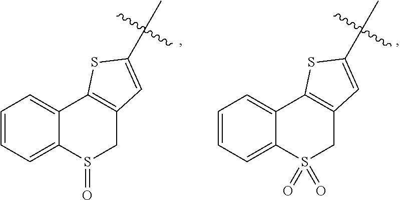 [SMILES: *c1cc2c(s1)-c1ccccc1S(=O)C2 ; *c1cc2c(s1)-c1ccccc1S(=O)(=O)C2]

and the like, and positional isomers thereof), and the like, and positional isomers thereof. In various examples, each R⁴ is a substituent (a substituent as described herein). In various examples, each R⁴ is independently chosen from —H group, —OH group, halide groups, alkyl groups, alkoxyl groups, aryl groups, halogenated hydrocarbon groups, acyl groups, alkylamino groups, cyano group, nitro group, thioether groups,

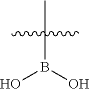 [SMILES: *B(O)O]

group and esters and cyclic esters thereof,

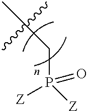

groups, where n is 0, 1, or 2, and Z is independently at each occurrence chosen from halide groups, alkoxyl groups, and the like,

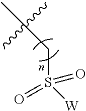

groups, where n is 0, 1, or 2, and W is a halide group (e.g., a —F, —Cl, —Br, or —I group) or the like, and the like.

In various examples, an A group is chosen from unsubstituted and substituted

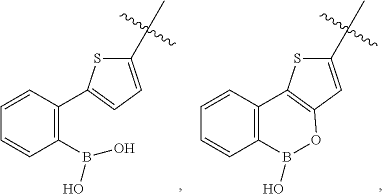 [SMILES: *c1ccc(-c2ccccc2B(O)O)s1 ; *c1cc2c(s1)-c1ccccc1B(O)O2]

and the like, and positional isomers thereof.

In various examples, an A group is chosen from unsubstituted and substituted

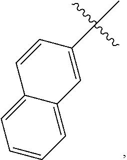 [SMILES: *c1ccc2ccccc2c1]

and the like, and positional isomers thereof. In various examples, an A group is chosen from

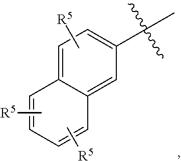

positional isomers thereof, and the like, where each R⁵ is independently chosen from —H group, —OH group, halide groups, alkyl groups, alkoxyl groups, aryl groups, halogenated hydrocarbon groups, acyl groups, alkylamino groups, cyano group, nitro group, thioether groups,

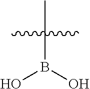 [SMILES: *B(O)O]

group and esters and cyclic esters thereof,

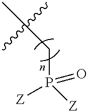

groups, where n is 0, 1, or 2, and Z is independently at each occurrence chosen from halide groups, alkoxyl groups, and the like,

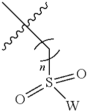

groups, where n is 0, 1, or 2, and W is a halide group (e.g., a —F, —Cl, —Br, or —I group) or the like, and the like.

In various examples, an A group is chosen from the preceding A group examples and is substituted with one or more substituent(s). In various examples, the A group substituent(s) is/are independently at each occurrence chosen from —H group, —OH group, halide groups (e.g., —F, —Cl, —Br, and —I groups), alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group or the like), acyl groups (e.g., —C(O) R^{h} groups, where R^{h} is a C₁-C₆ alkyl group or the like (which may be a halogenated alkyl group, such as, for example, a chloroalkyl group (e.g., a chloromethyl group and the like), a bromoalkyl group (e.g., a bromomethyl group and the like), an iodoalkyl group (e.g., an iodomethyl group and the like), a fluoroalkyl group (e.g., a fluoromethyl group and the like) (which may be a perfluorinated group, such as, for example, a trifluoromethyl group, and the like), and the like)), halogenated hydrocarbon groups, alkylamino groups (e.g., —NHRⁱ groups or —NRⁱRⁱ groups, where each Rⁱ is independently a C₁-C₆ alkyl group or the like), cyano group, nitro group, thioether groups (e.g., —SR^{j} groups, where R^{j} is a C₁-C₆ alkyl group or the like),

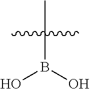 [SMILES: *B(O)O]

group or esters or cyclic esters thereof,

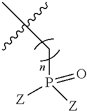

group(s), where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group or the like), and the like,

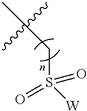

group(s), where n is 0, 1, or 2, and W is a halide group (e.g., a —F, —Cl, —Br, or —I group) and the like, and any combination thereof.

In various examples, a B group is chosen from unsubstituted and substituted

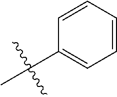 [SMILES: *c1ccccc1]

and the like. In various examples, a B group is chosen from

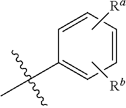

and the like, such as, for example,

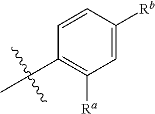

and the like, and positional isomers thereof, where each R^{a} and R^{b} are independently chosen from —H group, —OH group, halide groups, alkyl groups, alkoxyl groups, aryl groups, halogenated hydrocarbon groups, acyl groups, alkylamino groups, cyano group, nitro group, thioether groups,

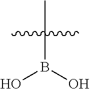 [SMILES: *B(O)O]

group and esters and cyclic esters thereof,

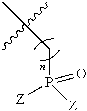

groups, where n is 0, 1, or 2, and Z is independently at each occurrence chosen from halide groups, alkoxyl groups, and the like,

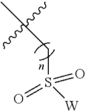

groups, where n is 0, 1, or 2, and W is a halide group (e.g., a —F, —Cl, —Br, or —I group) or the like, and the like.

In certain examples, only one of R^{a} and/or only one of R^{b} is/are present and a

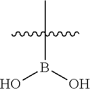 [SMILES: *B(O)O]

group or an ester or a cyclic ester thereof, a

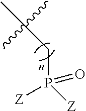

group, where n is 0, 1, or 2, and Z is independently at each occurrence chosen from halide groups (e.g., —F, —Cl, —Br, and —I groups), alkoxyl groups (e.g., —OR groups, where R^{g} is a C₁-C₁₀ alkyl group), and the like, a

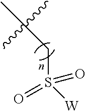

group, where n is 0, 1, or 2, and W is a halide group (e.g., —F, —Cl, —Br, or —I group) or the like, or the like.

In various examples, a B group is chosen from unsubstituted and substituted

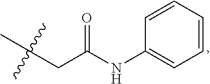 [SMILES: *CC(=O)Nc1ccccc1]

and the like. In various examples, a B group is chosen from

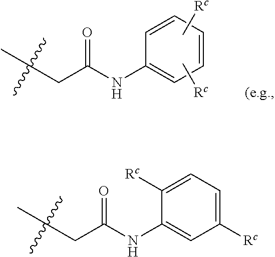

and the like, and positional isomers thereof), and the like, and positional isomers thereof, where each R^{c} is independently chosen from —H group, —OH group, halide groups, alkyl groups, alkoxyl groups, aryl groups, halogenated hydrocarbon groups, acyl groups, alkylamino groups, cyano group, nitro group, thioether groups,

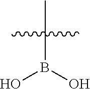 [SMILES: *B(O)O]

group and esters and cyclic esters thereof,

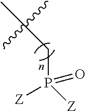

groups, where n is 0, 1, or 2, and Z is independently at each occurrence chosen from halide groups, alkoxyl groups, and the like,

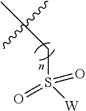

groups, where n is 0, 1, or 2, and W is a halide group (e.g., a —F, —Cl, —Br, or —I group) or the like, and the like.

In various examples, a B group is chosen from the preceding B group examples and is substituted with one or more substituent(s). In various examples, the B group substituent(s) is/are independently at each occurrence chosen from —H group, —OH group, halide groups (e.g., —F, —Cl, —Br, and —I groups), alkyl groups (e.g., C₁-C₁₀ alkyl groups and the like) (which may be fluorinated alkyl groups, such as, for example, perfluorinated alkyl groups and the like), alkoxyl groups (e.g., —OR^{g} groups, where R^{g} is a C₁-C₁₀ alkyl group or the like), acyl groups (e.g., —C(O) R^{h} groups, where R^{h} is a C₁-C₆ alkyl group or the like (which may be a halogenated alkyl group, such as, for example, a chloroalkyl group (e.g., a chloromethyl group and the like), a bromoalkyl group (e.g., a bromomethyl group and the like), an iodoalkyl group (e.g., an iodomethyl group and the like), a fluoroalkyl group (e.g., a fluoromethyl group and the like) (which may be a perfluorinated group, such as, for example, a trifluoromethyl group, and the like), and the like)), halogenated hydrocarbon groups, alkylamino groups (e.g., —NHRⁱ groups or —NRⁱRⁱ groups, where each Rⁱ is independently a C₁-C₆ alkyl group or the like), cyano group, nitro group, thioether groups (e.g., —SR^{j} groups, where R^{j} is a C₁-C₆ alkyl group or the like),

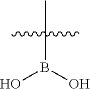 [SMILES: *B(O)O]

group or esters or cyclic esters thereof,

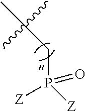

group(s), where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group or the like), and the like,

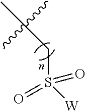

group(s), where n is 0, 1, or 2, and W is a halide group (e.g., a —F, —Cl, —Br, or —I group), and the like, and any combination thereof.

In various examples, a compound has the following structure:

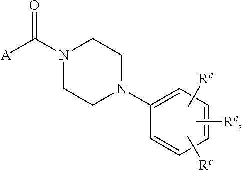

or the like, or a positional isomer thereof (e.g.,

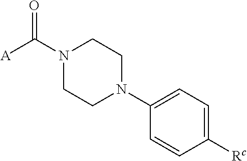

and the like, and positional isomers thereof), where R^{c} is independently at each occurrence chosen from —H group, —OH group, halide groups, alkyl groups, alkoxyl groups, aryl groups, halogenated hydrocarbon groups, acyl groups, alkylamino groups, cyano group, nitro group, thioether groups,

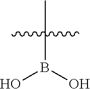 [SMILES: *B(O)O]

group and esters and cyclic esters thereof,

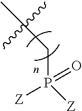

groups, where n is 0, 1, or 2, and Z is independently at each occurrence chosen from halide groups, alkoxyl groups, and the like,

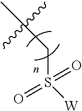

groups, where n is 0, 1, or 2, and W is a halide group (e.g., a —F, —Cl, —Br, or —I group) or the like, and the like.

In various examples, a compound is chosen from the following:

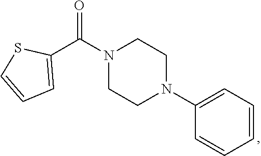 [SMILES: O=C(c1cccs1)N1CCN(c2ccccc2)CC1]

and the like, and positional isomers thereof, where the compound is unsubstituted or substituted. In various examples, the compound is chosen from the following:

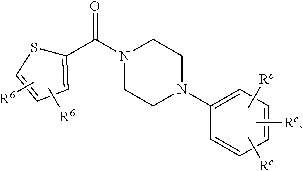

and the like, and positional isomers thereof (e.g.,

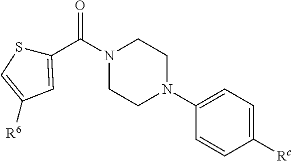

and the like, and positional isomers thereof), where R^{c} is independently at each occurrence chosen from —H group, —OH group, alkyl groups (e.g., C₁-C₁₀ alkyl groups and the like) (which may be fluorinated alkyl groups, such as, for example, perfluorinated alkyl groups and the like), alkoxyl groups (e.g., —OR^{g} groups, where R^{g} is a C₁-C₁₀ alkyl group or the like) acyl groups (e.g., —C(O) R^{h} groups, where R^{h} is a C₁-C₆ alkyl group or the like (which may be a halogenated alkyl group, such as, for example, a chloroalkyl group (e.g., a chloromethyl group and the like), a bromoalkyl group (e.g., a bromomethyl group and the like), an iodoalkyl group (e.g., an iodomethyl group and the like), a fluoroalkyl group (e.g., a fluoromethyl group and the like) (which may be a perfluorinated group, such as, for example, a trifluoromethyl group, and the like), and the like)), halogenated hydrocarbon groups, alkylamino groups (e.g., —NHRⁱ groups or —NRⁱRⁱ groups, where each Rⁱ is independently a C₁-C₆ alkyl group or the like), cyano group, nitro group, thioether groups (e.g., —SRⁱ groups, where Rⁱ is a C₁-C₆ alkyl group, or the like), and the like, and where R⁶ is independently at each occurrence chosen from —H group, —OH group, alkyl groups (e.g., C₁-C₁₀ alkyl groups) (which may be fluorinated alkyl groups, such as, for example, perfluorinated alkyl groups and the like), acyl groups (e.g., —C(O) R^{h} groups, where R^{h} is a C₁-C₆ alkyl group or the like (which may be a halogenated alkyl group, such as, for example, a chloroalkyl group (e.g., a chloromethyl group and the like), a bromoalkyl group (e.g., a bromomethyl group and the like), an iodoalkyl group (e.g., an iodomethyl group and the like), a fluoroalkyl group (e.g., a fluoromethyl group and the like) (which may be a perfluorinated group, such as, for example, a trifluoromethyl group, and the like), and the like)), halogenated hydrocarbon groups, alkylamino groups (e.g., —NHRⁱ groups or —NRⁱRⁱ groups, where each Rⁱ is independently a C₁-C₆ alkyl group or the like), cyano group, nitro group, thioether groups (e.g., —SR^{j} groups, where Rⁱ is a C₁-C₆ alkyl group or the like),

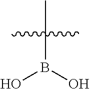 [SMILES: *B(O)O]

group and esters and cyclic esters thereof,

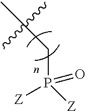

groups, where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group), and the like,

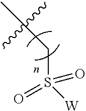

groups, where n is 0, 1, or 2, and W is a halide group (e.g., —F, —Cl, —Br, or —I group) or the like, and the like.

In various examples, a compound is chosen from the following:

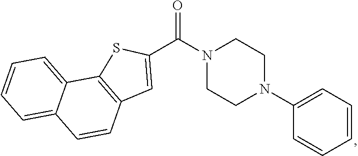 [SMILES: O=C(c1cc2ccc3ccccc3c2s1)N1CCN(c2ccccc2)CC1]

and the like, and positional isomers thereof, where the structure is unsubstituted or substituted. In various examples, the compound is chosen from the following:

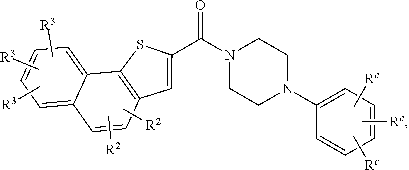

and the like, and positional isomers thereof (e.g.,

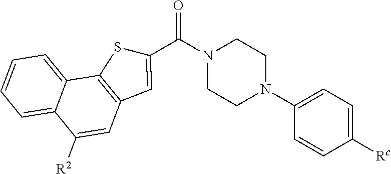

and the like, and positional isomers thereof), where R^{c} is independently at each occurrence chosen from —H group, —OH group, alkyl groups (e.g., C₁-C₁₀ alkyl groups and the like) (which may be fluorinated alkyl groups, such as, for example, perfluorinated alkyl groups and the like), alkoxyl groups (e.g., —OR^{g} groups, where R^{g} is a C₁-C₁₀ alkyl group or the like), acyl groups (e.g., —C(O) R^{h} groups, where R^{h} is a C₁-C₆ alkyl group or the like (which may be a halogenated alkyl group, such as, for example, a chloroalkyl group (e.g., a chloromethyl group and the like), a bromoalkyl group (e.g., a bromomethyl group and the like), an iodoalkyl group (e.g., an iodomethyl group and the like), a fluoroalkyl group (e.g., a fluoromethyl group and the like) (which may be a perfluorinated group, such as, for example, a trifluoromethyl group, and the like), and the like)), halogenated hydrocarbon groups, alkylamino groups (e.g., —NHRⁱ groups or —NRⁱRⁱ groups, where each Rⁱ is independently a C₁-C₆ alkyl group or the like), cyano group, nitro group, thioether groups (e.g., —SR^{j} groups, where R^{j} is a C₁-C₆ alkyl group or the like), and the like, and where R² and R³ are independently at each occurrence chosen from —H group, —OH group, halide groups (e.g., —F, —Cl, —Br, and —I groups), alkyl groups (e.g., C₁-C₁₀ alkyl groups and the like) (which may be fluorinated alkyl groups, such as, for example, perfluorinated alkyl groups and the like), alkoxyl groups (e.g., —OR^{g} groups, where R^{g} is a C₁-C₁₀ alkyl group or the like), acyl groups (e.g., —C(O) R^{h} groups, where R^{h} is a C₁-C₆ alkyl group or the like (which may be a halogenated alkyl group, such as, for example, a chloroalkyl group (e.g., a chloromethyl group and the like), a bromoalkyl group (e.g., a bromomethyl group and the like), an iodoalkyl group (e.g., an iodomethyl group and the like), a fluoroalkyl group (e.g., a fluoromethyl group and the like) (which may be a perfluorinated group, such as, for example, a trifluoromethyl group, and the like), and the like)), halogenated hydrocarbon groups, alkylamino groups (e.g., —NHRⁱ groups or —NRⁱRⁱ groups, where each Rⁱ is independently a C₁-C₆ alkyl group or the like), cyano group, nitro group, thioether groups (e.g., —SR^{j} groups, where R^{j} is a C₁-C₆ alkyl group or the like),

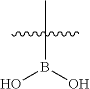 [SMILES: *B(O)O]

group and esters and cyclic esters thereof,

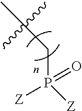

groups, where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group or the like), and the like,

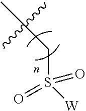

groups, where n is 0, 1, or 2, and W is a halide group (e.g., —F, —Cl, —Br, or —I group) or the like, and the like. In certain examples, only one of R² and/or only R³ are present and R³ and/or R^{c} is/are a

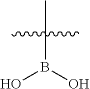 [SMILES: *B(O)O]

group or ester or cyclic ester thereof, a

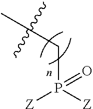

group, where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group or the like), and the like, a

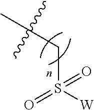

group, where n is 0, 1, or 2, and W is a halide group (e.g., —F, —Cl, —Br, or —I group) or the like, or the like.

In various examples, a compound is chosen from the following:

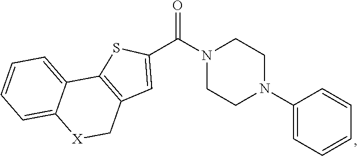

where X is —S(O)— or —S(O)₂—, and the like, and positional isomers thereof, where the structure is unsubstituted or substituted. In various examples, the compound is chosen from the following:

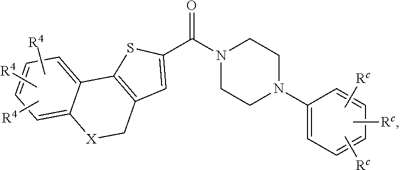

and the like, and positional isomers thereof, (e.g.,

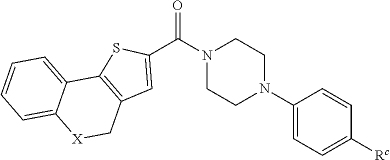

and the like, and positional isomers thereof), where R^{c} is independently at each occurrence chosen from —H group, —OH group, alkyl groups (e.g., C₁-C₁₀ alkyl groups and the like) (which may be fluorinated alkyl groups, such as, for example, perfluorinated alkyl groups and the like), alkoxyl groups (e.g., —OR^{g} groups, where R^{g} is a C₁-C₁₀ alkyl group or the like), acyl groups (e.g., —C(O) R^{h} groups, where R^{h} is a C₁-C₆ alkyl group or the like (which may be a halogenated alkyl group, such as, for example, a chloroalkyl group (e.g., a chloromethyl group and the like), a bromoalkyl group (e.g., a bromomethyl group and the like), an iodoalkyl group (e.g., an iodomethyl group and the like), a fluoroalkyl group (e.g., a fluoromethyl group and the like) (which may be a perfluorinated group, such as, for example, a trifluoromethyl group, and the like), and the like)), thioether groups (e.g., —SRⁱ groups, where Rⁱ is a C₁-C₆ alkyl group, or the like), and the like, and where R⁴ is independently at each occurrence chosen from —H group, —OH group, alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group or the like), acyl groups (e.g., —C(O) R^{h} groups, where R^{h} is a C₁-C₆ alkyl group or the like (which may be a halogenated alkyl group, such as, for example, a chloroalkyl group (e.g., a chloromethyl group and the like), a bromoalkyl group (e.g., a bromomethyl group and the like), an iodoalkyl group (e.g., an iodomethyl group and the like), a fluoroalkyl group (e.g., a fluoromethyl group and the like) (which may be a perfluorinated group, such as, for example, a trifluoromethyl group, and the like), and the like)), a trifluoromethyl group, and the like), and the like)), halogenated hydrocarbon groups, alkylamino groups (e.g., —NHRⁱ groups or —NRⁱRⁱ groups, where each Rⁱ is independently a C₁-C₆ alkyl group or the like), cyano group, nitro group, thioether groups (e.g., —SR^{j} groups, where R^{j} is a C₁-C₆ alkyl group or the like), and the like, and where X is —S(O)— or —S(O)₂—,

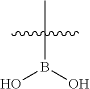 [SMILES: *B(O)O]

group and esters and cyclic esters thereof,

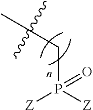

groups, where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group or the like), and the like,

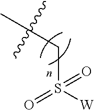

groups, where n is 0, 1, or 2, and W is a halide group (e.g., —F, —Cl, —Br, or —I group) or the like, and the like. In an example, the compound is not ML349.

In various examples, a compound is chosen from the following:

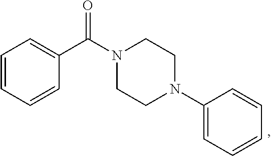 [SMILES: O=C(c1ccccc1)N1CCN(c2ccccc2)CC1]

and the like, where the structure is unsubstituted or substituted. In various examples, the compound is chosen from the following:

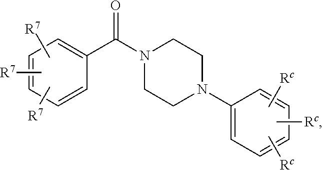

and the like, and positional isomers thereof (e.g.,

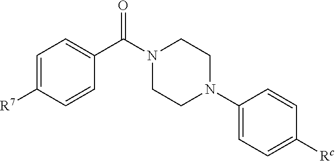

and the like, and positional isomers thereof), where R^{c} is independently at each occurrence chosen from —H group, —OH group, alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group), acyl groups (e.g., —C(O) R^{h} groups, where R^{h} is a C₁-C₆ alkyl group or the like (which may be a halogenated alkyl group, such as, for example, a chloroalkyl group (e.g., a chloromethyl group or the like), a bromoalkyl group (e.g., a bromomethyl group or the like), an iodoalkyl group (e.g., an iodomethyl group or the like), a fluoroalkyl group (e.g., a fluoromethyl group or the like) (which may be a perfluorinated group, such as, for example, a trifluoromethyl group, or the like), and the like)), halogenated hydrocarbon groups, alkylamino groups (e.g., —NHRⁱ groups or —NRⁱRⁱ groups, where each Rⁱ is independently a C₁-C₆ alkyl group or the like), cyano group, nitro group, thioether groups (e.g., —SR^{j} groups, where Rⁱ is a C₁-C₆ alkyl group or the like),

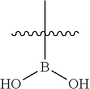 [SMILES: *B(O)O]

group and esters and cyclic esters thereof,

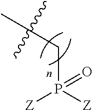

groups, where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group) and the like,

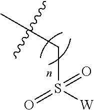

groups, where n is 0, 1, or 2, and W is a halide group (e.g., —F, —Cl, —Br, or —I group) or the like, and the like, and where R⁷ is independently at each occurrence chosen from chosen from —H group, —OH group, alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group),

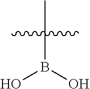 [SMILES: *B(O)O]

groups and esters and cyclic esters thereof,

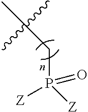

groups, where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group) and the like,

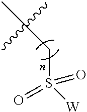

group(s), where n is 0, 1, or 2, and W is a halide group (e.g., —F, —Cl, —Br, or —I group) or the like, and the like. In various examples, only one of R^{c} and/or one of R⁷ group is present and a

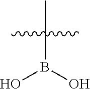 [SMILES: *B(O)O]

group or an ester or a cyclic ester thereof, a

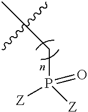

group, where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group), and the like, a

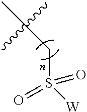

group, where n is 0, 1, or 2, and W is a halide group (e.g., —F, —Cl, —Br, or —I group), or the like, or the like.

In various examples, a compound is chosen from the following:

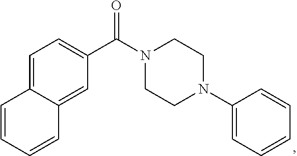 [SMILES: O=C(c1ccc2ccccc2c1)N1CCN(c2ccccc2)CC1]

and the like, and positional isomers thereof, where the structure is unsubstituted or substituted. In various examples, the compound is chosen from:

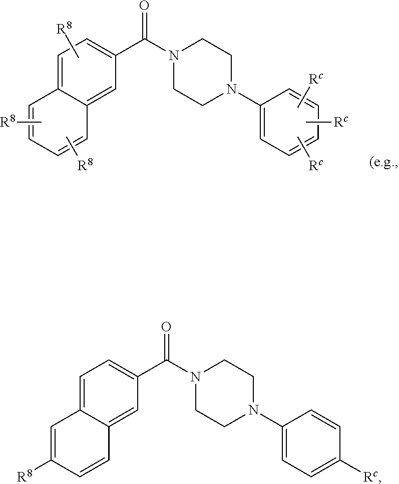

and the like and positional isomers thereof), and the like, and positional isomers thereof, where each R^{c} is independently chosen from —H group, —OH group, halide groups, alkyl groups, alkoxyl groups, halogenated hydrocarbon groups, acyl groups, alkylamino groups, cyano group, nitro group, thioether groups,

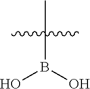 [SMILES: *B(O)O]

group and esters thereof and cyclic esters thereof,

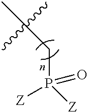

group(s), wherein n is 0, 1, or 2, and each Z is independently chosen from —OH group, halide groups, and alkoxyl groups,

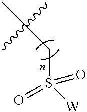

group(s), wherein n is 0, 1, or 2, and W is a halide group, and each R⁸ is independently chosen from chosen from —H group, —OH group, halide groups, alkyl groups, alkoxyl groups, halogenated hydrocarbon groups, acyl groups, alkylamino groups, cyano group, nitro group, thioether groups,

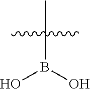 [SMILES: *B(O)O]

group and esters thereof and cyclic esters thereof,

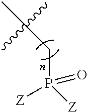

group(s), wherein n is 0, 1, or 2, and each Z is independently chosen from —OH group, halide groups, alkoxyl groups,

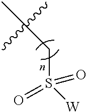

group(s), wherein n is 0, 1, or 2, and W is a halide group, and the like, and any combination thereof. In various examples, R^{c} is chosen from alkyl groups, hydrocarbon hydrocarbon groups and the like, and R⁸ is a

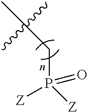

group, where n is 0, 1, or 2, and each Z is independently chosen from —OH group, halide groups, alkoxyl groups,

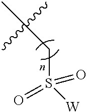

group(s), wherein n is 0, 1, or 2, and W is a halide group, and the like.

In various examples, a compound is chosen from the preceding compound examples and is substituted with one or more substituent(s). In various examples, the substituent(s) is/are independently at each occurrence chosen from —H group, —OH group, halide groups (e.g., —F, —Cl, —Br, and —I groups), alkyl groups (e.g., C₁-C₁₀ alkyl groups and the like) (which may be fluorinated alkyl groups, such as, for example, perfluorinated alkyl groups and the like), alkoxyl groups (e.g., —OR^{g} groups, where R^{g} is a C₁-C₁₀ alkyl group or the like), acyl groups (e.g., —C(O) R^{h} groups, where R^{h} is a C₁-C₆ alkyl group or the like (which may be a halogenated alkyl group, such as, for example, a chloroalkyl group (e.g., a chloromethyl group and the like), a bromoalkyl group (e.g., a bromomethyl group and the like), an iodoalkyl group (e.g., an iodomethyl group and the like), a fluoroalkyl group (e.g., a fluoromethyl group and the like) (which may be a perfluorinated group, such as, for example, a trifluoromethyl group, and the like), and the like)), halogenated hydrocarbon groups, alkylamino groups (e.g., —NHRⁱ groups or —NRⁱRⁱ groups, where each Rⁱ is independently a C₁-C₆ alkyl group or the like), cyano group, nitro group, thioether groups (e.g., —SR^{j} groups, where R^{j} is a C₁-C₆ alkyl group or the like),

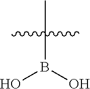 [SMILES: *B(O)O]

group or esters or cyclic esters thereof,

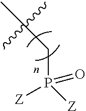

group(s), where n is 0, 1, or 2, and Z is independently at each occurrence chosen from halide groups (e.g., —F, —Cl, —Br, and —I groups), alkoxyl groups (e.g., —OR^{g} groups, where R^{g} is a C₁-C₁₀ alkyl group or the like), and the like,

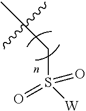

group(s), where n is 0, 1, or 2, and W is a halide group (e.g., a —F, —Cl, —Br, or —I group) and the like, and any combination thereof.

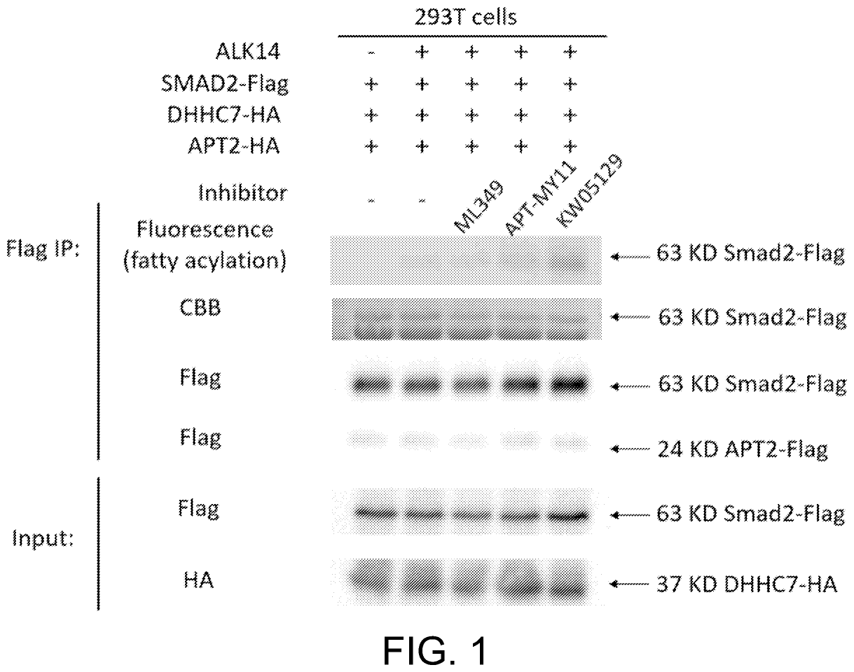

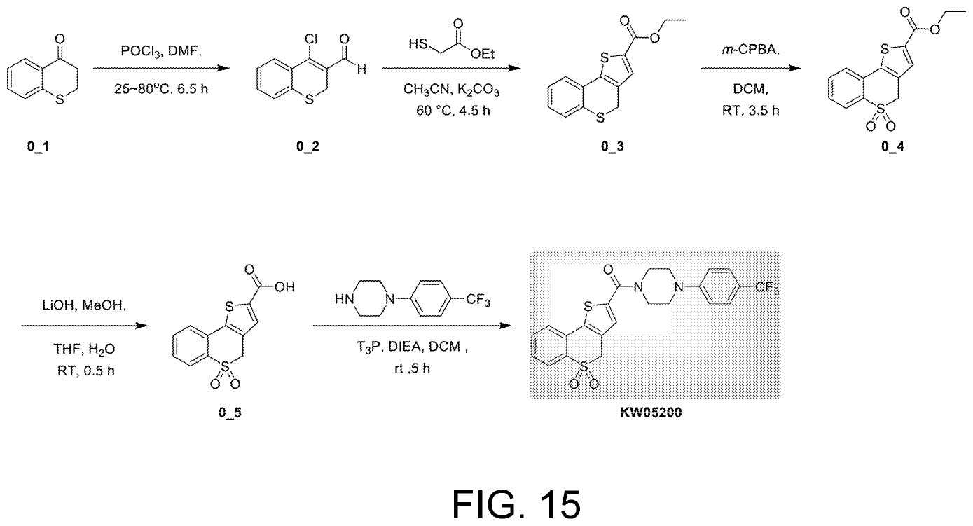

In various examples, the compound has a structure of a compound of FIGS. 1-9, or FIGS. 15-46. In various examples, the compound has a structure of a compound of Examples 1-6. In various examples, the compound has a structure of a compound of Table 1 of Example 2 or of Table 3 of Example 5. In various examples, the compound is APT-MY1, APT-MY2, APT-MY4, APT-MY8, APT-MY9, APT-MY11, APT-MY12, KW05129, KW05175, KW05191, KW05192, KW06034, KW0526pp, KW05151, KW05153, KW05169, KW05177, KW05179, KW05200, KW05201, KW05202, KW05203, KW05204, KW05205, KW05206, KW05207, KW05208, KW05209, KW05210, KW05211, KW05212, KW05213, KW05214, KW05215, KW05216, KW05217, KW05218, KW05219, KW05220, KW05221, KW05222, KW05223, KW05224, KW05225, KW05226, KW05227, KW05228, KW05229, KW05230, KW05231, KW05232, KW05130, KW05108, or KW05116.

In an aspect, the present disclosure provides compositions. The compositions may be pharmaceutical compositions. Non-limiting examples of the compositions are described herein.

A composition may comprise (or consist essentially of or consist of) one or more one or more compound(s) of the present disclosure, which may be APT1 and/or APT2 inhibitor(s). A composition may also comprise one or more additional component(s), one or more or all of which may be pharmaceutically acceptable components.

As used herein, unless otherwise indicated, the term “pharmaceutically acceptable” refers to those components and dosage forms that are, within the scope of sound medical judgment, suitable for use in contact with the tissues of humans or animals without excessive toxicity, irritation, or other problem or complication, commensurate with a reasonable benefit/risk ratio.

Some non-limiting examples of materials which can be used as additional component(s) in a composition include sugars, such as, for example, lactose, glucose, sucrose, and the like; starches, such as, for example, corn starch, potato starch, and the like; cellulose, and its derivatives, such as, for example, sodium carboxymethyl cellulose, ethyl cellulose, cellulose acetate, and the like; powdered tragacanth; malt; gelatin; talc; excipients, such as, for example, cocoa butter, suppository waxes, and the like; oils, such as, for example, peanut oil, cottonseed oil, safflower oil, sesame oil, olive oil, corn oil, soybean oil, and the like; glycols, such as, for example, propylene glycol and the like; polyols, such as, for example, glycerin, sorbitol, mannitol, polyethylene glycol, and the like; esters, such as, for example, ethyl oleate, ethyl laurate, and the like; agar; buffering agents, such as, for example, magnesium hydroxide, aluminum hydroxide, and the like; alginic acid; pyrogen-free water; isotonic saline; Ringer's solution; ethyl alcohol; phosphate buffer solutions; and other non-toxic compatible substances employed in pharmaceutical formulations. (See, e.g., REMINGTON'S PHARM. SCI., 15th Ed. (Mack Publ. Co., Easton (1975)).

In an aspect, the present disclosure provides uses of one or more compound(s) and/or composition(s) of the present disclosure. Non-limiting examples of uses of the one or more compound(s) and/or composition(s) of the present disclosure are described herein.

In various examples, one or more compound(s) and/or composition(s) of the present disclosure used in treatment methods. A method of treatment may comprise (or consist essentially of or consist of) administration of one or more APT1 and APT2 inhibitor(s) to a subject.

A method may at least partially or completely inhibit APT1, APT2, or both. In various examples, a method of at least partially or completely inhibiting APT1, APT2, or both, comprises administering to a subject an amount of one or more compound(s) of the present disclosure (one or more or all of which may be present as pharmaceutical compositions), where APT1, APT2, or both, is at least partially or completely inhibited. In various examples, APT1 is at least partially or completely inhibited and APT2 is not at least partially or completely inhibited, APT2 is at least partially or completely inhibited and APT1 is not at least partially or completely inhibited.

In various examples, the present disclosure provides a means for at least partially or completely inhibiting APT1, APT2, or both. In various examples, the present disclosure provides a means for at least partially or completely inhibiting APT1, APT2, or both, in a subject at an IC₅₀ of 0.100 μM or less, less than 0.100 μM, 0.5 μM or less, less than 0.5 μM, 1 μM or less, less than 1 μM, 5 μM or less, less than 5 μM, 10 μM or less, less than 10 μM, 25 μM or less, less than 25 μM, 40 μM or less, less than 40 μM.

A method may treat a subject diagnosed with or in need of treatment for a disease, a disorder, or the like, or any combination thereof. In various examples, a method for treating a disease, disorder, or the like, or any combination thereof, comprises administering to a subject an amount of one or more compound(s) of the present disclosure (one or more or all of which may be present as pharmaceutical composition), where one or more symptom(s), indication(s), or the like, or any combination thereof, of the subject is at least partially alleviated. In various examples, the subject has APT1 and/or APT2 and APT1 and/or APT2 is at least partially or completely inhibited by the method.

A method may treat a disease or a disorder, or the like, or any combination thereof, characterized by accelerated differentiation of T helper 17 (Th17) cells. In various examples, a method for treating a disease or a disorder, or the like, or any combination thereof, characterized by accelerated differentiation of T helper17 (Th17) cells comprises administering to subject diagnosed with, suffering from, or the like, or any combination thereof, the disease or the disorder, or the like, or any combination thereof, an effective amount of one or more inhibitor(s) of an enzyme or enzymes (e.g., Acyl Protein Thioesterase (APT1) (which may be referred to as Lysophospholipase 1 (LYPLA1)), Acyl Protein Thioesterase (APT2) (which may be referred to as Lysophospholipase 2 (LYPLA2)), or the like, or both) that regulate(s) the S-palmitoylation of STAT3. Non-limiting examples of inhibitor(s) of an enzyme or enzymes that regulate(s) the S-palmitoylation of STAT3 include compound(s) of the present disclosure. The inhibitor(s) of an enzyme or enzymes that regulate(s) the S-palmitoylation of STAT3, may individually be a non-covalent inhibitor and/or a covalent inhibitor of APT1 and/or APT2.

A method may treat an autoimmune disorder, a neurodegenerative disorder, an inflammatory disorder, or the like, or any combination thereof. In various examples, a method of treating a subject diagnosed with or is in need of treatment, or the like, or any combination thereof, for an autoimmune disorder, a neurodegenerative disorder, an inflammatory disorder, or the like, or any combination thereof, comprises administering to a subject an amount of one or more compound(s) of the present disclosure (one or more or all of which may be present as pharmaceutical compositions), where one or more symptom(s), indication(s), or the like, or any combination thereof, of the subject is at least partially alleviated.

Non-limiting examples of disorders include autoimmune disorders, neurodegenerative disorders, inflammatory disorders, immune-mediated cancer diseases, and the like. Non-limiting examples of autoimmune disorders include inflammatory bowel disease, multiple sclerosis, rheumatoid arthritis, lupus (such as, for example, cutaneous lupus, systemic lupus, and the like), graft versus host disease, type I diabetes, psoriasis, atopic dermatitis, eczema, alopecia areata, myeloproliferative neoplasms, dermatomyositis, Guillain-Barre Syndrome, and the like. Non-limiting examples of neurodegenerative disorders include Parkinson's disease, Alzheimer's disease, Huntington's disease, and the like. Non-limiting examples of immune-mediated cancer diseases include lymphoma, leukemia, and the like.

A subject (e.g., a subject in need of treatment or the like) may be a human or other animal (which may be a non-human mammal). Non-limiting examples of non-human animals (which may be mammals) include cows, pigs, mice, rats, rabbits, cats, dogs, and other agricultural animals, pets (such as, for example, dogs, cats, and the like), service animals, and the like.

“Treating” or “treatment” of any disease or disorder refers, in various examples, to ameliorating (e.g., arresting, reversing, alleviating, or the like) the disease, disease state, condition, disorder, side effect, potential disease, potential disease state, potential condition, potential disorder, potential side effect, or the like, or a combination thereof, or reducing the manifestation, extent or severity of one or more clinical symptom(s) thereof, or the like. In various other examples, “treating” or “treatment” refers to ameliorating one or more physical parameter(s), which, independently, may or may not be discernible by the subject. In yet other examples, “treating” or “treatment” refers to modulating disease, disease state, condition, disorder, side effect, or the like, or a combination thereof, either physically, (e.g., stabilization of one or more discernible symptom(s), or the like), physiologically, (e.g., stabilization of one or more physical parameter, or the like), or both. In yet other examples, treating” or “treatment” relates to slowing the progression of the disease, disease state, condition, disorder, side effect, or the like, or a combination thereof. Treating may include administration of an effective amount of the composition(s).

As used herein, unless otherwise indicated, the term “effective amount” means that amount of the compound(s) and/or composition(s) that will elicit the biological or medical response of subject (or a tissue, system, or the like, thereof) that is being sought, for instance, by a researcher, clinician, or the like. An effective amount may be a therapeutically effective amount. The term “therapeutically effective amount” includes any amount which, as compared to a corresponding subject who has not received such amount, results in improved treatment, healing, prevention, or amelioration of a disease, disease state, condition, disorder, side effect, potential disease, potential disease state, potential condition, potential disorder, potential side effect, or the like, or a combination thereof, or a decrease in the rate of advancement of a disease, disease state, condition, disorder, potential disease, potential disease state, potential condition, potential disorder, potential side effect, or the like, or the like. The term also includes within its scope amounts effective to enhance normal physiological function.

An effective amount may result in prophylaxis. The term “prophylaxis” includes prevention and refers to a measure or procedure which is to prevent rather than cure or treat a disease. Preventing may refer to a reduction in risk of acquiring or developing a disease causing at least one clinical symptom of the disease not to develop in a subject that may be exposed to a disease causing agent or a subject predisposed to the disease in advance of disease outset.

A physician or veterinarian having ordinary skill in the art can readily determine and prescribe the effective amount of the compound(s) and/or composition(s) required. The selected dosage level can depend upon a variety of factors including, but not limited to, the activity of the particular composition employed, the time of administration, the rate of excretion or metabolism of the particular composition being employed, the rate and extent of absorption, the duration of the treatment, other drugs, compounds and/or materials used in combination with the particular composition employed, the age, sex, weight, condition, general health and prior medical history of the patient being treated, and like factors well known in the medical arts. For example, the physician or veterinarian could start doses of the composition employed at levels lower than that required in order to achieve the desired therapeutic effect and gradually increase the dosage until the desired effect is achieved.

In an aspect, the present disclosure provides kits. A kit comprises (or consist essentially of or consist of) one or more one or more compound(s) and/or composition(s) of the present disclosure.

In various examples, a kit comprises one or more compound(s) and/or composition(s) (e.g., one or more pharmaceutical composition(s)) of the present disclosure). In various examples, a kit includes a closed or sealed package that contains the one or more one or more compound(s) and/or composition(s). In various examples, the package comprises one or more closed or sealed vials, bottles, blister (bubble) packs, or any other suitable packaging for the sale, distribution, or use of the one or more compound(s) and/or composition(s). The printed material may include printed information. The printed information may be provided on a label, on a paper insert, printed on a packaging material, or the like. The printed information may include information that identifies the compound(s) in the package, the amounts and types of other active and/or inactive ingredients in the composition, and instructions for taking the compound(s) and/or composition(s). The instructions may include information, such as, for example, the number of doses to take over a given period of time, and/or information directed to a pharmacist and/or another health care provider, such as, for example, a physician or the like, or a patient. The printed material may include an indication or indications that the one or more compound(s) and/or composition(s) and/or any other agent provided therein is for treatment of a subject. In various examples, the kit includes a label describing the contents of the kit and providing indications and/or instructions regarding use of the contents of the kit to treat a subject.

The steps of the method described in the various examples disclosed herein are sufficient to carry out the methods of the present disclosure. Thus, in an example, a method consists essentially of a combination of the steps of the methods disclosed herein. In another example, a method consists of such steps.

The following Statements describe various examples of methods, products and systems of the present disclosure and are not intended to be in any way limiting:

Statement 1. A compound having the following structure:

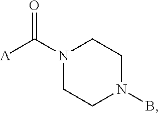

wherein A is a substituted or unsubstituted aryl group, heterocyclic group (which may or may not be aromatic), or the like, or a combination thereof, which may be chosen from furanyl groups, thienyl groups (e.g., 2-thienyl groups, 2-thiophen-2-yl)phenyl groups, and the like, and positional isomers thereof), naphtho thiophen-2-yl groups (e.g., naphtho[1,2-b]thiophen-2-yl groups, and the like, and positional isomers thereof), oxido/dioxido thieno thiochromeneyl groups (e.g., 5,5-dioxido-4H-thieno[3,2-c]thiochromen-2-yl groups, and the like, and positional isomers thereof), phenyl groups, and the like, any of which may be substituted or unsubstituted, and B is chosen from alkyl groups (e.g., C₁-C₁₀ alkyl groups) (which may be fluorinated alkyl groups, such as, for example, perfluorinated alkyl groups and the like) (which may be fluorinated alkyl groups, such as, for example, perfluorinated alkyl groups and the like), phenyl groups, alkoxylphenyl groups (e.g., C₁-C₁₀ alkyoxy phenyl groups), and alkylamide groups (e.g., C₁ alkylamide groups, such as, for example, acetamide groups and the like), or the like, any of which may be substituted or unsubstituted, or a pharmaceutically acceptable salt, a salt, a partial salt, a solvate, a polymorph, or a prodrug thereof, or a stereoisomer or a mixture of stereoisomers, an isotopic variant, or tautomer thereof.
Statement 2. A compound according to Statement 1, the compound having the following structure:

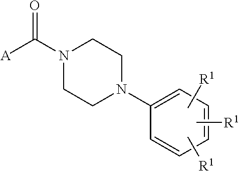

or the like,
wherein R¹ is independently at each occurrence chosen from —H, halide groups, (e.g., —F, —Cl, —Br, and —I) alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group), acyl groups (e.g., —C(O) R^{h}, where R^{h} is a C₁-C₆ alkyl group (which may be a halogenated alkyl group, such as, for example, a chloroalkyl group (e.g., a chloromethyl group or the like), a bromoalkyl group (e.g., a bromomethyl group or the like), an iodoalkyl group (e.g., an iodomethyl group or the like), a fluoroalkyl group (e.g., a fluoromethyl group or the like) (which may be a perfluorinated group, such as, for example, a trifluoromethyl group, or the like), and the like), thioether groups (e.g., —SRⁱ, where Rⁱ is a C₁-C₆ alkyl group, or the like),

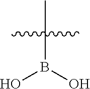 [SMILES: *B(O)O]

groups and esters and cyclic esters thereof,

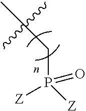

group(s), where n is 0, 1, or 2, and each Z is independently chosen from halide groups (e.g., —F, —Cl, —Br, and —I groups), alkoxyl groups (e.g., —OR^{g} groups, where R^{g} is a C₁-C₁₀ alkyl group), and the like,

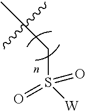

group(s), where n is 0, 1, or 2, and W is a halide group (e.g., —F, —Cl, —Br, or —I group), and the like.
Statement 3. A compound according to Statement 2, wherein the compound having the following structure:

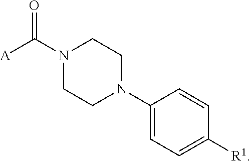

Statement 4. A compound according to Statement 1 or 2, wherein the compound having the following structure:

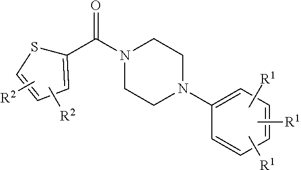

or the like or a positional isomer thereof, wherein R¹ is independently at each occurrence chosen from —H, —OH, alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group) acyl groups (e.g., —C(O) R^{h}, where R^{h} is a C₁-C₆ alkyl group (which may be a halogenated alkyl group, such as, for example, a chloroalkyl group (e.g., a chloromethyl group or the like), a bromoalkyl group (e.g., a bromomethyl group or the like), an iodoalkyl group (e.g., an iodomethyl group or the like), a fluoroalkyl group (e.g., a fluoromethyl group or the like) (which may be a perfluorinated group, such as, for example, a trifluoromethyl group, or the like), and the like), thioether groups (e.g., —SRⁱ, where Riis a C₁-C₆ alkyl group, or the like), and the like, and R² is independently at each occurrence chosen from —H, —OH, alkyl groups (e.g., C₁-C₁₀ alkyl groups) (which may be fluorinated alkyl groups, such as, for example, perfluorinated alkyl groups and the like), acyl groups (e.g., —C(O) R^{h}, where R^{h} is a C₁-C₆ alkyl group (which may be a halogenated alkyl group, such as, for example, a chloroalkyl group (e.g., a chloromethyl group or the like), a bromoalkyl group (e.g., a bromomethyl group or the like), an iodoalkyl group (e.g., an iodomethyl group or the like), a fluoroalkyl group (e.g., a fluoromethyl group or the like) (which may be a perfluorinated group, such as, for example, a trifluoromethyl group, or the like), and the like), thioether groups (e.g., —SRⁱ, where Rⁱ is a C₁-C₆ alkyl group, or the like),

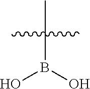 [SMILES: *B(O)O]

groups and esters and cyclic esters thereof,

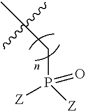

group(s), where n is 0, 1, or 2, and each Z is independently chosen from halide groups (e.g., —F, —Cl, —Br, and —I groups), alkoxyl groups (e.g., —OR^{g} groups, where R⁸ is a C₁-C₁₀ alkyl group), and the like,

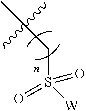

group(s), where n is 0, 1, or 2, and W is a halide group (e.g., —F, —Cl, —Br, or —I group), and the like, and positional isomers thereof.
Statement 5. A compound according to Statement 4, the compound having the following structure:

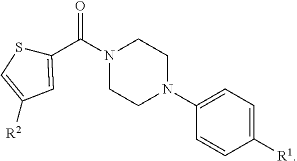

Statement 6. A compound according to Statement 1 or 2, the compound having the following structure:

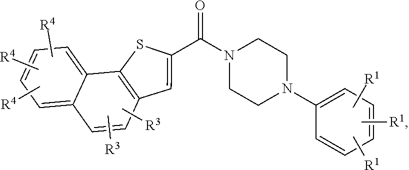

or the like, or a positional isomer thereof, wherein R¹ is independently at each occurrence chosen from —H, —OH, alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group), acyl groups (e.g., —C(O) R^{h}, where R^{h} is a C₁-C₆ alkyl group (which may be a halogenated alkyl group, such as, for example, a chloroalkyl group (e.g., a chloromethyl group or the like), a bromoalkyl group (e.g., a bromomethyl group or the like), an iodoalkyl group (e.g., an iodomethyl group or the like), a fluoroalkyl group (e.g., a fluoromethyl group or the like) (which may be a perfluorinated group, such as, for example, a trifluoromethyl group, or the like), and the like), thioether groups (e.g., —SRⁱ, where Rⁱ is a C₁-C₆ alkyl group, or the like), and the like, R³ and R⁴ are independently at each occurrence chosen from —H, —OH, halide groups (e.g., —F, —Cl, —Br, and —I), alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group), acyl groups (e.g., —C(O) R^{h}, where R^{h} is a C₁-C₆ alkyl group (which may be a halogenated alkyl group, such as, for example, a chloroalkyl group (e.g., a chloromethyl group or the like), a bromoalkyl group (e.g., a bromomethyl group or the like), an iodoalkyl group (e.g., an iodomethyl group or the like), a fluoroalkyl group (e.g., a fluoromethyl group or the like) (which may be a perfluorinated group, such as, for example, a trifluoromethyl group, or the like), and the like), thioether groups (e.g., —SRⁱ, where Rⁱ is a C₁-C₆ alkyl group, or the like),

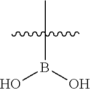 [SMILES: *B(O)O]

groups and esters and cyclic esters thereof,

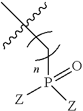

group(s), where n is 0, 1, or 2, and each Z is independently chosen from halide groups (e.g., —F, —Cl, —Br, and —I groups), alkoxyl groups (e.g., —OR^{g} groups, where R^{g} is a C₁-C₁₀ alkyl group), and the like,

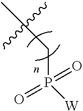

group(s), where n is 0, 1, or 2, and W is a halide group (e.g., —F, —Cl, —Br, or —I group), and the like.
Statement 7. A compound according to Statement 6, wherein the compound has the following structure:

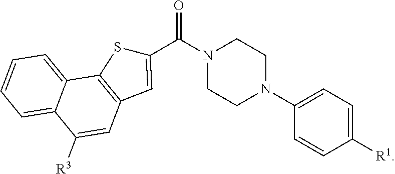

Statement 8. A compound according to Statement 1 or 2, the compound having one of the following structures:

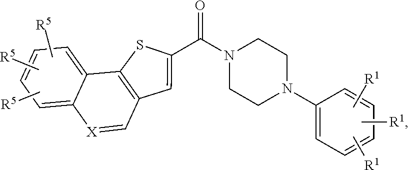

or the like, or a positional isomer thereof, wherein R¹ is independently at each occurrence chosen from —H, —OH, alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group), acyl groups (e.g., —C(O) R^{h}, where R^{h} is a C₁-C₆ alkyl group (which may be a halogenated alkyl group, such as, for example, a chloroalkyl group (e.g., a chloromethyl group or the like), a bromoalkyl group (e.g., a bromomethyl group or the like), an iodoalkyl group (e.g., an iodomethyl group or the like), a fluoroalkyl group (e.g., a fluoromethyl group or the like) (which may be a perfluorinated group, such as, for example, a trifluoromethyl group, or the like), and the like), thioether groups (e.g., —SRⁱ, where Rⁱ is a C₁-C₆ alkyl group, or the like), and the like, wherein R⁵ is independently at each occurrence chosen from —H, —OH, alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group), acyl groups (e.g., —C(O) R^{h}, where R^{h} is a C₁-C₆ alkyl group (which may be a halogenated alkyl group, such as, for example, a chloroalkyl group (e.g., a chloromethyl group or the like), a bromoalkyl group (e.g., a bromomethyl group or the like), an iodoalkyl group (e.g., an iodomethyl group or the like), a fluoroalkyl group (e.g., a fluoromethyl group or the like) (which may be a perfluorinated group, such as, for example, a trifluoromethyl group, or the like), and the like), thioether groups (e.g., —SRⁱ, where Rⁱ is a C₁-C₆ alkyl group, or the like), and the like, and X is —S(O)— or —S(O)₂—.
Statement 9. A compound according to Statement 8, the compound having one of the following structures:

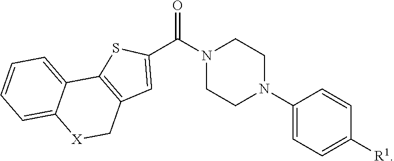

Statement 10. A compound according to Statement 1 or 2, the compound having one of the following structures:

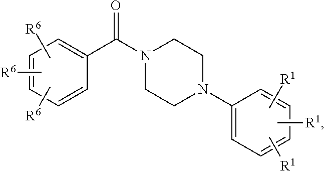

or the like,
wherein R¹ is independently at each occurrence chosen from —H, alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group), acyl groups (e.g., —C(O) R^{h}, where R^{h} is a C₁-C₆ alkyl group (which may be a halogenated alkyl group, such as, for example, a chloroalkyl group (e.g., a chloromethyl group or the like), a bromoalkyl group (e.g., a bromomethyl group or the like), an iodoalkyl group (e.g., an iodomethyl group or the like), a fluoroalkyl group (e.g., a fluoromethyl group or the like) (which may be a perfluorinated group, such as, for example, a trifluoromethyl group, or the like), and the like), thioether groups (e.g., —SRⁱ, where Riis a C₁-C₆ alkyl group, or the like),

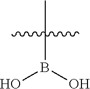 [SMILES: *B(O)O]

groups and esters and cyclic esters thereof,

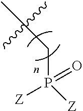

group(s), where n is 0, 1, or 2, and each Z is independently chosen from halide groups (e.g., —F, —Cl, —Br, and —I groups), alkoxyl groups (e.g., —OR^{g} groups, where R^{c} is a C₁-C₁₀ alkyl group) and the like,

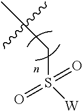

group(s), where n is 0, 1, or 2, and W is a halide group (e.g., —F, —Cl, —Br, or —I group), and the like, and
R⁶ is independently at each occurrence chosen from chosen from —H, alkyl groups (e.g., C₁-C₁₀ alkyl groups) (which may be fluorinated alkyl groups, such as, for example, perfluorinated alkyl groups and the like), alkoxyl groups (e.g., —OR^{g} groups, where R^{g} is a C₁-C₁₀ alkyl group),

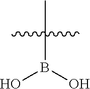 [SMILES: *B(O)O]

groups and esters and cyclic esters thereof,

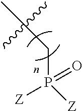

group(s), where n is 0, 1, or 2, and each Z is independently chosen from halide groups (e.g., —F, —Cl, —Br, and —I groups), alkoxyl groups (e.g., —OR^{g} groups, where R^{g} is a C₁-C₁₀ alkyl group) and the like,

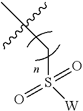

group(s), where n is 0, 1, or 2, and W is a halide group (e.g., —F, —Cl, —Br, or —I group), and the like.
Statement 11. A compound according to Statement 10, the compound having one of the following structures:

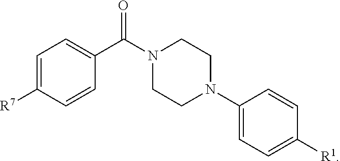

Statement 12. A compound according to any one of the previous Statements, wherein the compound has a structure of a compound of Table 1 of Example 2.
Statement 13. A pharmaceutical composition comprising one or compound(s) of the present disclosure (one or more or all of which may be a compound or compounds of any one of Statements 1-12).
Statement 14. A pharmaceutical composition according to Statement 13, wherein the pharmaceutical composition further comprises one or more pharmaceutically acceptable excipient(s).
Statement 15. A method of at least partially or completely inhibiting APT1 and/or APT2, the method comprising: administering to a subject an amount of one or more compound(s) (one or more or all of which may be present as pharmaceutical compositions) (e.g., compound(s) of any one of Statements 1-12, pharmaceutical composition(s) of Statement 13 or 14, or a combination thereof), wherein APT1 and/or APT2 is at least partially or completely inhibited.
Statement 16. A method for treating a disorder characterized by accelerated differentiation of T helper 17 (Th17) cells, the method comprising administering to subject diagnosed with, suffering from, or the like, the disorder an effective amount of one or more inhibitor(s) of an enzyme or enzymes that regulates the S-palmitoylation of STAT3.
Statement 17. A method according to Statement 16, wherein the enzyme is Acyl Protein Thioesterase (APT1) (which may be referred to as Lysophospholipase 1 (LYPLA1)) and/or Acyl Protein Thioesterase (APT2) (which may be referred to as Lysophospholipase 2 (LYPLA2)).
Statement 18. A method according to Statement 16 or 17, wherein the inhibitor(s) are chosen from compounds of the present disclosure (one or more or all of which may be present as pharmaceutical compositions).
Statement 19. A method according to any one of Statements 16-18, wherein the disorder is an autoimmune disorder, a neurodegenerative disorder, an inflammatory disorder, or the like. Non-limiting examples of autoimmune disorders include inflammatory bowel disease, multiple sclerosis, rheumatoid arthritis, lupus, graft versus host disease, type I diabetes, psoriasis, and the like.
Statement 20. A method of treating a subject diagnosed with or in need of treatment for an autoimmune disorder, a neurodegenerative disorder, an inflammatory disorder, or the like, the method comprising administering to a subject an amount of one or more compound(s) of the present disclosure (one or more or all of which may be present as pharmaceutical compositions) (e.g., compound(s) of any one of Statements 1-12, pharmaceutical composition(s) of Statement 13 or 14, or a combination thereof), wherein one or more symptom(s), indication(s), or the like, of the subject is at least partially alleviated.
Statement 21. A method according to Statement 20, wherein the subject has APT1 and/or APT2 and APT1 and/or APT2 is at least partially or completely inhibited.

The following examples are presented to illustrate the present disclosure. The examples are not intended to be limiting in any manner.

### Example 1

This example describes synthesis and characterization of examples of compounds of the present disclosure.

General Procedure 1: A carboxylic acid starting material (1 eq.) was dissolved in dimethyl formamide (DMF). Hexafluorophosphate Azabenzotriazole Tetramethyl Uronium (HATU) (1.25 eq.) was added portion wise followed by 1-(4-methoxyphenyl)piperazine (1.1 eq.). The solution was stirred at room temperature for 18 h then concentrated under reduced pressure. The crude reaction mixture was mixed with water and extracted with an equal volume of dichloromethane (DCM) three times. The organic layers were collected, pooled, and dried over anhydrous sodium sulfate. The solvent was removed under reduced pressure to give the second crude material which was purified by flash chromatography to give the final product.

General Procedure 2: An aryl halide (1 eq.), 2-bromophenyl boronic acid (1 eq.), and Pd(PPh₃)₄ (0.04 eq) were combined in a in a screw cap vial, degassed and charged with N2. 1,4-Dioxane was added to the vial. In a separate vial, sodium carbonate (3 eq.) was dissolved in water (0.25 volume eq. of the dioxane). The water solution was degassed, charged with N2, and added to the first vial. The mixture was then stirred at 100° C. When the aryl halide starting material was completely consumed as determined by liquid chromatography-mass spectrometry (LC-MS), the reaction mixture was cooled to room temperature. Dioxane was removed under reduced pressure. The resulting crude was diluted with water and extracted three times with an equal volume of DCM. The organic layers were pooled and dried over sodium sulfate. Solvent was removed under reduced pressure to give the second crude material which was purified by flash chromatography to give the final product.

General Procedure 3: An aryl halide (1 eq.), bis boronic acid (3 eq.), XPhos-Pd-G2 (0.01 eq.), XPhos (0.02 eq.) and KOAc (3 eq.) were combined in a screw cap vial, degassed, and charged with N2. Ethanol was added to the vial. The mixture was heated to 80° C. LC-MS was used to monitor the reaction progress. When the aryl halide had been completely consumed, the reaction mixture was cooled to room temperature and the solvent was removed under reduced pressure. The crude product was taken up in water then extracted three times with an equal volume of DCM. The organic layers were collected, pooled, and dried over sodium sulfate. The solvent was removed under reduced pressure to yield the second crude which was purified by flash chromatography to afford the final product.

General Procedure 4: Chloroacetyl chloride (1 eq.) and potassium carbonate (0.75 eq.) were combined in DCM and chilled to 0° C. A phenyl amine (0.5 eq.) was added portion wise. The reaction mixture warmed to room temperature and stirred for 16 h. Solvent was removed under reduced pressure. Ice cold water was added to the resulting crude which was warmed to room temperature and stirred for 30 min. Undissolved material was collected by vacuum filtration and taken to the next step without further purification.

General Procedure 5:1,1-Dimethylethyl 4-(2-furanylcarbonyl)-1-piperazinecarboxylate was dissolved in DCM and trifluoroacetic acid (TFA, 0.1 eq. by volume to DCM) was added dropwise. The solution was stirred at room temperature for 4 h. Solvent was removed under reduced pressure. The resulting product was re-dissolved in DMF to which was added potassium carbonate (3 eq.) and one of compounds 32-35 (1 eq.). This mixture was heated to 80° C. and kept for 4 h. Then the solvent was removed under reduced pressure. The crude product was diluted with water and extracted three times with an equal volume of DCM. The organic layers were pooled and dried over sodium sulfate. Solvent was removed under reduced pressure and the resulting second crude product was purified by flash chromatography to give the final products 38-41.

General Procedure 6: The carboxylic acid starting material (1 eq.) and an amine (1 eq.) were dissolved in DCM. 1-ethyl-3-(3-dimethylaminopropyl) carbodiimide (EDC, 3 eq.) was added to the solution followed by 4-dimethylaminopyridine (DMAP, 0.11 eq.). The solution was stirred at room temperature for 16 h then washed with HCl (0.1 M), water, and brine. The organic layer was then dried over anhydrous sodium sulfate and concentrated under reduced pressure. The resulting crude product was purified by flash chromatography to give the final product.

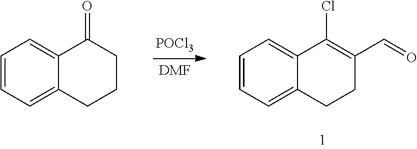

1-chloro-3,4-dihydro-2-naphthaldehyde (1): POCl₃ (0.67 mL, 7.3 mmol) was added dropwise to DMF (6 mL). The solution was chilled to 0° C. and alpha tetralone (0.91 mL, 6.8 mmol) was added drop wise. The solution was warmed to room temperature then heated to 80° C. for 1.5 h. Heating was removed and the solution was cooled to room temperature then poured slowly into 1 M aqueous sodium acetate (50 mL) to quench the reaction. This solution was extracted 3 times with DCM (50 mL each). The organic layers were pooled and dried over sodium sulfate. The solvent was removed under reduced pressure and the resulting product was taken to the next step without further purification.

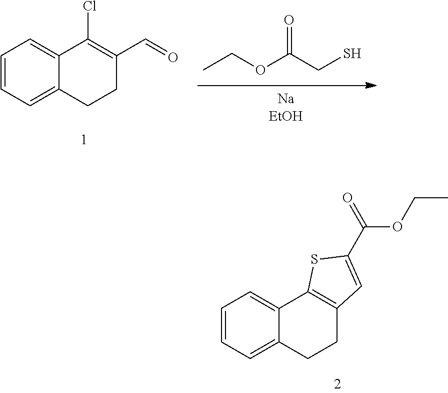

Ethyl 4,5-dihydronaphtho[1,2-b]thiophene-2-carboxylate (2): Sodium metal (0.20 g, 8.7 mmol) was slowly added to ethanol (20 mL) and dissolved completely before chilling the solution to 0° C. Ethyl thioglycolate (0.83 mL, 7.5 mmol) was added dropwise followed by compound 1 from the previous reaction. The solution was warmed to room temperature while stirring for 16 h. The solution was then heated to 70° C. for 2 h. The solvent was then removed under reduced pressure and the resulting crude was dissolved in DCM (15 mL), washed with water and brine (15 mL each), and dried over sodium sulfate. The second crude product was concentrated under reduced pressure and purified by flash chromatography (hexane→3:2 hexane: DCM) to give compound 2 (1.0 g, 3.9 mmol, 57% yield over two steps).

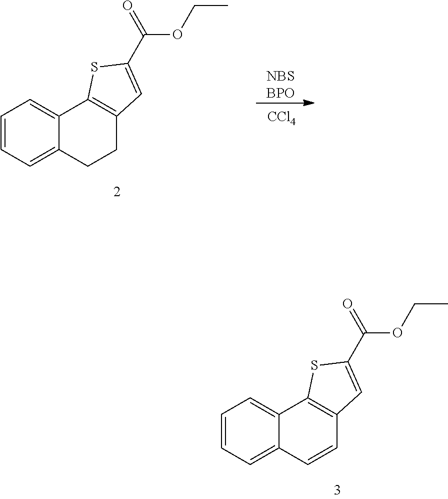

Ethyl naphtho[1,2-b]thiophene-2-carboxylate (3): Compound 2 (1.0 g, 3.9 mmol), N-bromosuccinimide (NBS, 0.69 g, 3.9 mmol) and benzoyl peroxide (BPO, 1.0 mg, 0.041 mmol) were dissolved in CCl₄ (30 mL) and heated to reflux for 2 h. Additional NBS and BPO (0.69 g, 3.9 mmol and 1.0 mg, 0.041 mmol respectively) were added at this time and the mixture refluxed again for 2 h. The mixture was cooled and vacuum filtered to remove the solid. The filtrate was concentrated under reduced pressure and the crude material was purified by flash chromatography (hexane→3:17 ethylacetate (EtOAc):hexane) to give compound 3 (0.88 g, 3.4 mmol, 89% yield).

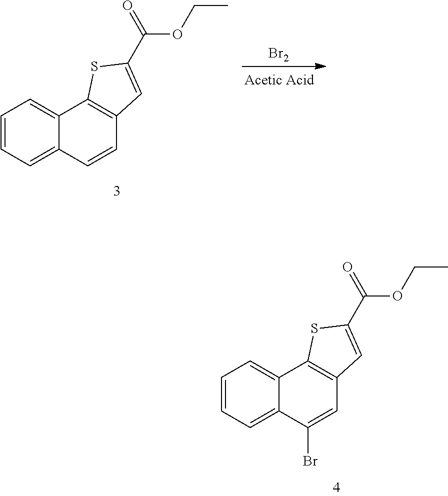 [SMILES: CCOC(=O)c1cc2ccc3ccccc3c2s1 ; "Br2" ; "Acetic Acid" ; CCOC(=O)c1cc2cc(Br)c3ccccc3c2s1]

Ethyl 5-bromonaphtho[1,2-b]thiophene-2-carboxylate (4): Compound 3 (0.88 g, 3.4 mmol) was dissolved in the acetic acid (17 mL) and bromine was added (0.2 mL, 3.9 mmol). The solution was heated to reflux for 4 h. After cooling to room temperature, the liquid was poured into water (100 mL) and extracted three times with EtOAc (100 mL each). The organic fractions were pooled, dried over anhydrous sodium sulfate, and concentrated under reduced pressure. The crude product was purified by flash chromatography (hexane→2:3 DCM:hexane) to give compound 4 (0.52 g, 1.6 mmol, 47% yield).

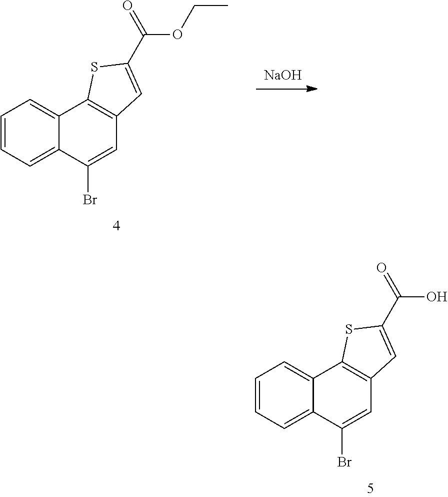 [SMILES: CCOC(=O)c1cc2cc(Br)c3ccccc3c2s1 ; "NaOH" ; O=C(O)c1cc2cc(Br)c3ccccc3c2s1]

5-Bromonaphtho[1,2-b]thiophene-2-carboxylic acid (5): Compound 4 (0.52 g, 1.6 mmol) was dissolved in tetrahydrofuran (THF, 7 mL) to which was added a 2 M aqueous NaOH solution (7 mL). The mixture was heated to reflux for 16 h. After cooling, THF was removed under reduced pressure. The remaining aqueous solution was acidified with 6 M HCl (5 mL). The precipitated compound 5 was collected by vacuum filtration and taken to the next step without further purification (0.46 g, 1.5 mmol, 94% yield).

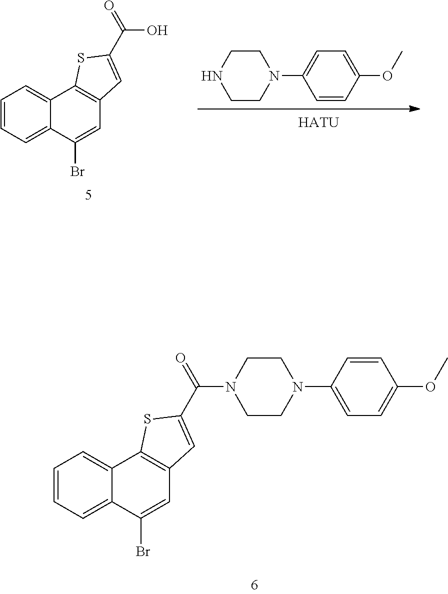

(5-Bromonaphtho[1,2-b]thiophen-2-yl) (4-(4-methoxyphenyl)piperazin-1-yl) methanone (6): Compound 6 was synthesized from compound 5 by procedure 1 (47% yield).

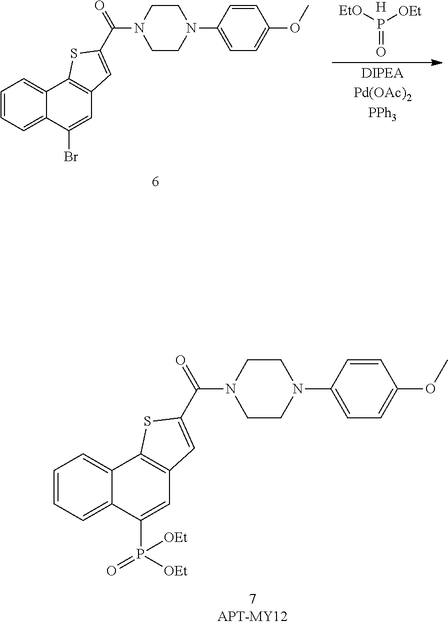

Diethyl (2-(4-(4-methoxyphenyl)piperazine-1-carbonyl)naphtho[1,2-b]thiophen-5-yl)phosphonate (7): Compound 6 (0.51 g, 1.1 mmol), diethyl phosphonate (0.20 mL, 1.5 mmol), N,N-diisopropylethylamine (DIPEA, 0.36 mL, 2 mmol), Pd(OAc)₂ (11 mg, 0.049 mmol) and triphenyl phosphine (33 mg, 0.13 mmol) were combined in ethanol (44 mL). The mixture was heated to reflux for 16 h. The reaction was cooled, and the solvent was removed under reduced pressure. The crude material was purified by flash chromatography (1:1 EtOAC: hexane, then 3:97 methanol: DCM) to give compound 7 (0.534 g, 0.99 mmol, 93% yield). ¹H NMR (500 MHZ, CDCl₃) δ 8.66 (d, J=17.1 Hz, 1H), 8.62-8.58 (m, 1H), 8.21 (dt, J=7.1, 2.4 Hz, 1H), 7.71 (s, 1H), 7.70-7.66 (m, 2H), 6.99-6.93 (m, 2H), 6.92-6.86 (m, 2H), 4.26 (dp, J=10.2, 7.2 Hz, 2H), 4.12 (ddq, J=10.1, 8.3, 7.1 Hz, 2H), 4.04-3.97 (m, 4H), 3.80 (d, J=0.9 Hz, 3H), 3.18 (t, J=5.0 Hz, 4H), 1.34 (t, J=7.0 Hz, 6H). ¹³C NMR (126 MHz, CDCl₃) δ 163.18, 154.56, 145.11, 143.48, 143.45, 136.71, 134.93, 134.78, 132.02, 131.94, 129.81, 129.72, 128.98, 128.88, 127.99, 127.96, 127.63, 127.51, 126.80, 126.78, 124.40, 124.39, 123.65, 122.19, 119.06, 114.59, 62.37, 62.33, 55.57, 51.27, 16.42, 16.37. ³¹P NMR (202 MHZ, CDCl₃) δ 19.20. HRMS (ESI) calc. for [M+H]⁺ C₂₈H₃₂N₂O₅PS 539.17641, obsd. 539.17735.

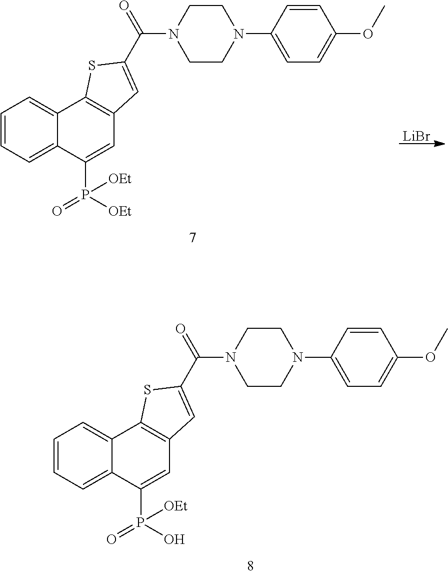 [SMILES: CCOP(=O)(OCC)c1cc2cc(C(=O)N3CCN(c4ccc(OC)cc4)CC3)sc2c2ccccc12 ; "LiBr" ; CCOP(=O)(O)c1cc2cc(C(=O)N3CCN(c4ccc(OC)cc4)CC3)sc2c2ccccc12]

Ethyl hydrogen (2-(4-(4-methoxyphenyl)piperazine-1-carbonyl)naphtho[1,2-b]thiophen-5-yl)phosphonate (8): Compound 7 (0.50 g, 0.92 mmol) and LiBr (0.951 g, 11 mmol) were combined in acetone (9 mL) and heated to reflux for 3 days. Solvent was removed under reduced pressure. The crude material was purified by flash chromatography (DCM→1:9 MeOH:DCM) to give compound 8 (0.168 g, 0.33 mmol, 36% yield).

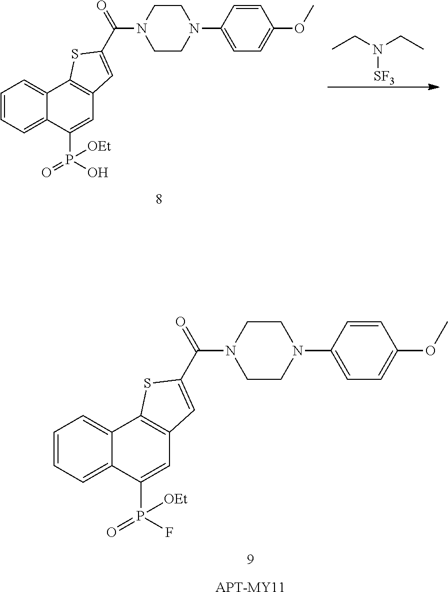

Ethyl (2-(4-(4-methoxyphenyl)piperazine-1-carbonyl)naphtho[1,2-b]thiophen-5-yl)phosphonofluoridate (9): Compound 8 (42 mg, 0.082 mmol) was dissolved in DCM (25 mL) to which (diethylamino) sulfur trifluoride (22 μL, 0.17 mmol) was added drop wise. The solution was stirred at room temperature for 3 h. Solvent was removed under reduced pressure. The resulting material was purified by flash chromatography (1:49 methanol: DCM) to give compound 9 (16 mg, 0.031 mmol, 38% yield). ¹H NMR (500 MHZ, CDCl₃) δ 8.61 (d, J=18.5 Hz, 1H), 8.45 (dq, J=7.9, 2.8 Hz, 1H), 8.22 (dt, J=7.2, 2.4 Hz, 1H), 7.74-7.68 (m, 3H), 6.97-6.91 (m, 2H), 6.90-6.85 (m, 2H), 4.50-4.36 (m, 2H), 3.98 (t, J=5.1 Hz, 4H), 3.78 (s, 3H), 3.17 (t, J=5.0 Hz, 4H), 1.45 (t, J=7.1 Hz, 3H). ¹³C NMR (126 MHZ, CDCl₃) δ 162.94, 154.60, 145.08, 144.43, 144.40, 137.23, 134.63, 134.47, 131.91, 131.84, 131.82, 129.24, 129.14, 128.94, 128.83, 128.19, 127.95, 127.34, 127.31, 126.62, 124.61, 124.60, 119.09, 114.60, 77.28, 77.03, 76.84, 76.78, 64.36, 64.31, 55.58, 51.30, 29.72, 16.43, 16.38. ¹⁹F NMR (470 MHz, CDCl₃) δ −60.34, −62.56. ³¹P NMR (202 MHZ, CDCl₃) δ 20.24, 15.08. HRMS (ESI) calc. for [M+H]⁺ C₂₆H₂₇FN₂O₄PS 513.14077, obsd. 513.14041.

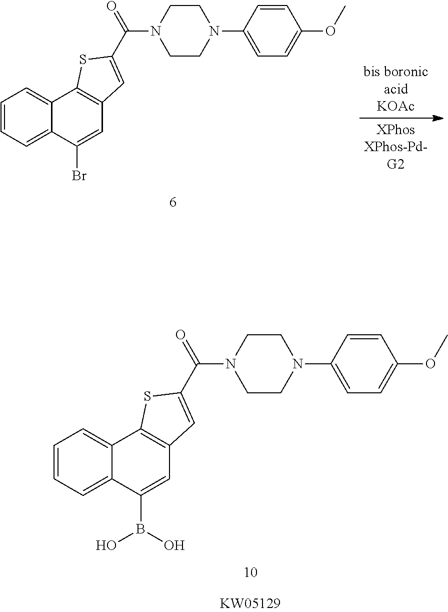 [SMILES: COc1ccc(N2CCN(C(=O)c3cc4cc(Br)c5ccccc5c4s3)CC2)cc1 ; COc1ccc(N2CCN(C(=O)c3cc4cc(B(O)O)c5ccccc5c4s3)CC2)cc1]

(2-(4-(4-Methoxyphenyl)piperazine-1-carbonyl)naphtho[1,2-b]thiophen-5-yl)boronic acid (10): Compound 10 was synthesized from compound 3 by procedure 3 (34% yield). Compound 10 was converted to the corresponding aryl trifluoroborate salt to obtain NMR spectra due compound 10 having poor solubility in MeOH. ¹H NMR (500 MHZ, DMSO) δ 8.53 (d, J=8.0 Hz, 1H), 8.03-7.99 (m, 1H), 7.95 (d, J=3.7 Hz, 1H), 7.83 (s, 1H), 7.51-7.41 (m, 1H), 6.97 (d, J=6.6 Hz, 2H), 6.86 (d, J=8.6 Hz, 2H), 3.89 (s, 4H), 3.70 (s, 3H), 3.14 (t, J=5.0 Hz, 4H). ¹³C NMR (126 MHz, DMSO) δ 163.31, 137.22, 136.20, 135.66, 134.84, 131.90, 128.33, 127.99, 125.79, 125.40, 125.37, 125.23, 123.51, 118.63, 114.81, 55.67, 50.70, 46.20. ¹⁹F NMR (470 MHz, DMSO) δ −135.56. ¹¹B NMR (160 MHZ, DMSO) δ 3.30. HRMS data was obtained from compound 10 directly. HRMS (ESI) calc. for [M+H]⁺ C₂₄H₂₄N₂O₄BS 447.15499, obsd. 447.15580.

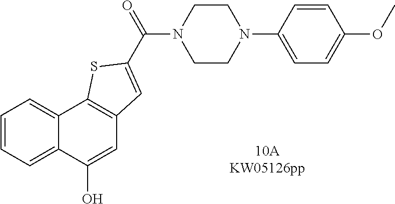 [SMILES: COc1ccc(N2CCN(C(=O)c3cc4cc(O)c5ccccc5c4s3)CC2)cc1]

(5-Hydroxynaphtho[1,2-b]thiophen-2-yl) (4-(4-methoxyphenyl)piperazin-1-yl) methanone (10A): Compound 10A was isolated as a side product of the synthesis of compound 10 from compound 6 (9% yield). ¹H NMR (500 MHZ, DMSO) § 10.34 (s, 1H), 8.27 (dd, J=8.2, 1.4 Hz, 1H), 8.07 (d, J=8.0 Hz, 1H), 7.81 (s, 1H), 7.66 (ddd, J=8.2, 7.0, 1.4 Hz, 1H), 7.60 (ddd, J=8.3, 6.9, 1.3 Hz, 1H), 7.27 (s, 1H), 6.96 (d, J=8.8 Hz, 2H), 6.91-6.80 (m, 2H), 3.87 (t, J=5.0 Hz, 4H), 3.70 (s, 3H), 3.12 (t, J=5.0 Hz, 4H). ¹³C NMR (126 MHz, DMSO) δ 162.94, 153.88, 152.06, 145.50, 137.50, 136.42, 129.27, 128.94, 128.04, 127.08, 126.26, 124.99, 124.00, 123.97, 118.58, 114.80, 103.62, 55.67, 55.39, 50.65. HRMS (ESI) calc. for [M+H]⁺ C₂₄H₂₃N₂O₃S 419.14239, obsd. 419.14238.

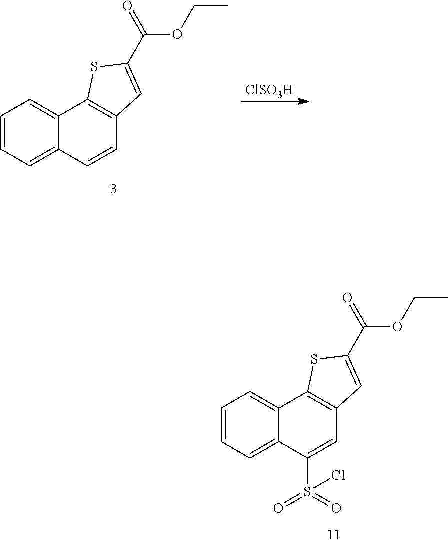 [SMILES: CCOC(=O)c1cc2ccc3ccccc3c2s1 ; "ClSO3H" ; CCOC(=O)c1cc2cc(S(=O)(=O)Cl)c3ccccc3c2s1]

Ethyl 5-(chlorosulfonyl)naphtho[1,2-b]thiophene-2-carboxylate (11): Chlorosulfonic acid (78 μL, 1.2 mmol) was added to chloroform (6 mL) and chilled to 0° C. In a separate vessel, compound 3 (0.10 g, 0.38 mmol) was dissolved in chloroform (3 mL) and added dropwise to the solution on ice. The solution was warmed gradually to room temperature and stirred for 4 h. The mixture was poured into ice cold water (50 ml) and stirred for 30 min. The water was saturated with NaCl and the precipitate that formed was decanted into a vacuum filter apparatus to collect the solid. This solid was purified by flash chromatography (acetone→1:9 MeOH:acetone) to give compound 11 (30 mg, 0.83 mmol, 22% yield).

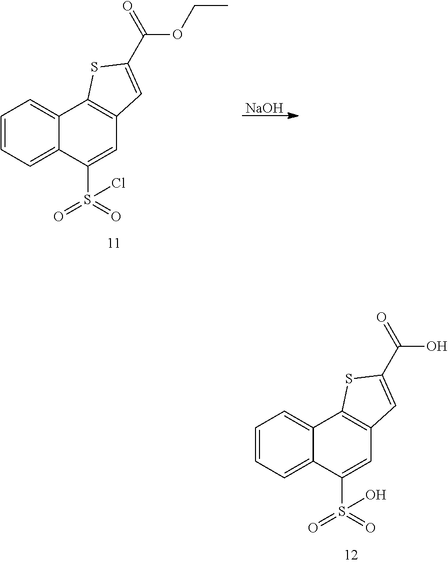 [SMILES: CCOC(=O)c1cc2cc(S(=O)(=O)Cl)c3ccccc3c2s1 ; "NaOH" ; O=C(O)c1cc2cc(S(=O)(=O)O)c3ccccc3c2s1]

5-sulfonaphtho[1,2-b]thiophene-2-carboxylic acid (12): Compound 11 (0.16 g, 0.44 mmol) was added to a 2M aqueous NaOH solution (8 mL) and stirred and room temperature for 16 h. The solution was acidified with 6 M HCl (5 mL) and saturated with NaCl to precipitate compound 12. The solid was collected by vacuum filtration and taken to the next step without further purification (0.053 g, 0.17 mmol, 39% yield).

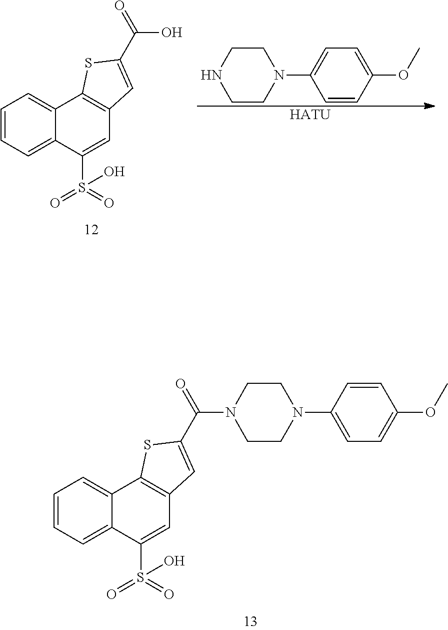

2-(4-(4-methoxyphenyl)piperazine-1-carbonyl)naphtho[1,2-b]thiophene-5-sulfonic acid (13): Compound 13 was synthesized from compound 12 by procedure 1 (25% yield).

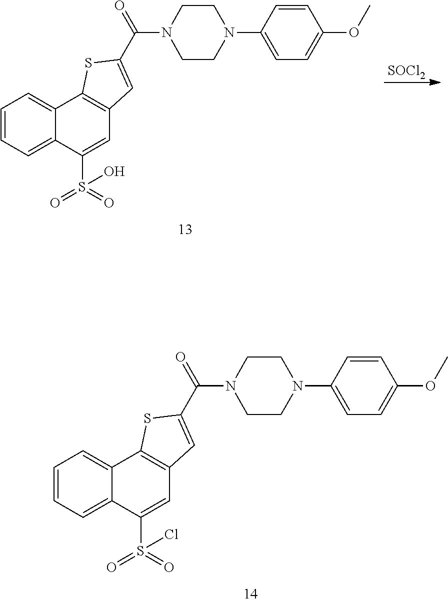

2-(4-(4-Methoxyphenyl)piperazine-1-carbonyl)naphtho[1,2-b]thiophene-5-sulfonyl chloride (14): Compound 13 (0.15 g, 0.30 mmol) was dissolved in DMF (1.2 mL) and chilled to 0° C. SOCl₂ (66 μL, 0.91 mmol) was added drop wise. The solution was warmed to room temperature and stirred for 5 h. The reaction was quenched by pouring into ice cold water (15 mL) and stirring for 30 min. The aqueous mixture was extracted three times with EtOAc (15 mL each). The organic fractions were pooled, dried over sodium sulfate, and concentrated under reduced pressure. The crude product was purified by flash chromatography (EtOAc->1:9 MeOH: EtOAc) to give compound 14 (0.11 g, 0.23 mmol, 75% yield).

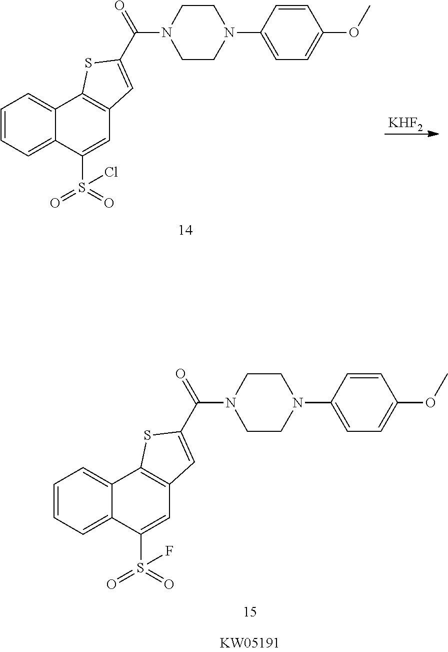 [SMILES: COc1ccc(N2CCN(C(=O)c3cc4cc(S(=O)(=O)Cl)c5ccccc5c4s3)CC2)cc1 ; "KHF2" ; COc1ccc(N2CCN(C(=O)c3cc4cc(S(=O)(=O)F)c5ccccc5c4s3)CC2)cc1]

2-(4-(4-Methoxyphenyl)piperazine-1-carbonyl)naphtho[1,2-b]thiophene-5-sulfonyl fluoride (15): Compound 14 (30 mg, 0.06 mmol) was dissolved in a 1:1 solution of acetonitrile and water (0.6 mL). In a separated vessel, KHF₂ (52 mg, 0.67 mmol) was dissolved in water (0.6 mL) and added to the first solution drop wise. The resulting solution was stirred at room temperature. The progress of the reaction was monitored by LC-MS. When all the starting material had been consumed, the solution was diluted with water (10 mL) and extracted with EtOAc 3 times (10 mL each). The organic fractions were pooled, dried over sodium sulfate, and concentrated under reduced pressure to give compound 15 (11 mg, 0.023 mmol, 38% yield). ¹H NMR (500 MHZ, CDCl₃) δ 8.74 (d, J=1.6 Hz, 1H), 8.62 (dt, J=8.1, 2.2 Hz, 1H), 8.28 (ddd, J=8.5, 5.3, 1.7 Hz, 1H), 7.88-7.77 (m, 2H), 7.76 (d, J=2.3 Hz, 1H), 6.97-6.91 (m, 2H), 6.90-6.85 (m, 2H), 4.02-3.96 (m, 4H), 3.79 (s, 3H), 3.17 (t, J=5.0 Hz, 4H). ¹³C NMR (126 MHz, CDCl₃) δ 162.41, 154.66, 145.96, 145.00, 138.47, 133.46, 133.18, 129.21, 128.84, 128.34, 126.49, 125.66, 124.75, 119.13, 114.61, 55.58, 51.30. ¹⁹F NMR (470 MHZ, CDCl₃) δ 62.81. HRMS (ESI) calc. for [M+H]⁺ C₂₄H₂₂N₂O₄S₂F 485.09995, obsd. 485.09997.

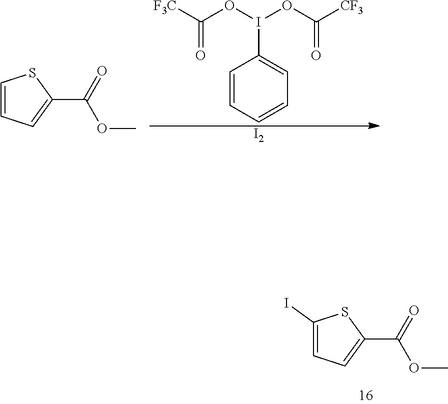

Methyl 5-iodo-2-thiophenecarboxylate (16): Methyl-2-thiophenecarboxylate (0.18 g, 1.3 mmol), [bis(trifluoroacetoxy) iodo]benzene (0.32 g, 0.73 mmol), and iodine (0.17 g, 0.67 mmol) were dissolved in CCl₄ (1.8 mL) and stirred at room temperature for 2 h. The solvent was removed under reduced pressure. The crude product was purified by flash chromatography (hexane→1:3 DCM:hexane) to give compound 16 (0.20 g, 0.76 mmol, 58% yield).

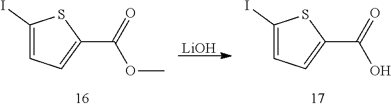

5-Iodo-2-thiophenecarboxylic acid (17): LiOH (0.152 g, 3.6 mmol) was dissolved in water (5.7 mL) to which was added a solution of compound 16 (0.20 g, 0.76 mmol) in THF (0.76 mL). The mixture was stirred at room temperature for 4 h. The solution was acidified with 6 M HCl to pH 4 and then extracted with EtOAc three times (10 mL each). The organic fractions were pooled, dried over sodium sulfate, and concentrated under reduced pressure to give compound 17, which was taken to the next step without further purification (0.131 g, 0.52 mmol, 68% yield).

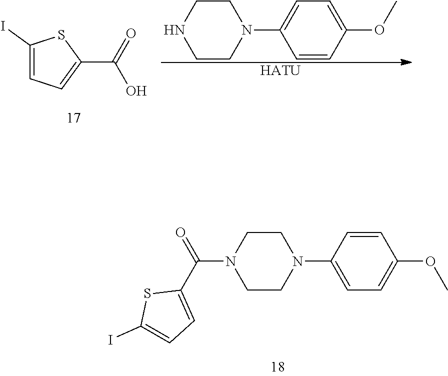

(5-Iodothiophen-2-yl) (4-(4-methoxyphenyl)piperazin-1-yl) methanone (18): Compound 18 was synthesized from compound 17 by procedure 1 (60% yield).

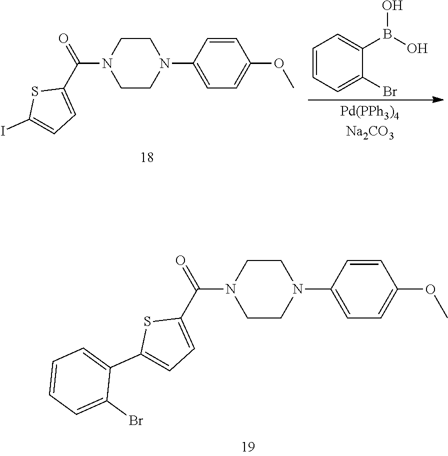

(5-(2-Bromophenyl) thiophen-2-yl) (4-(4-methoxyphenyl)piperazin-1-yl) methanone (19): Compound 19 was synthesized from compound 18 by procedure 2 (92% yield).

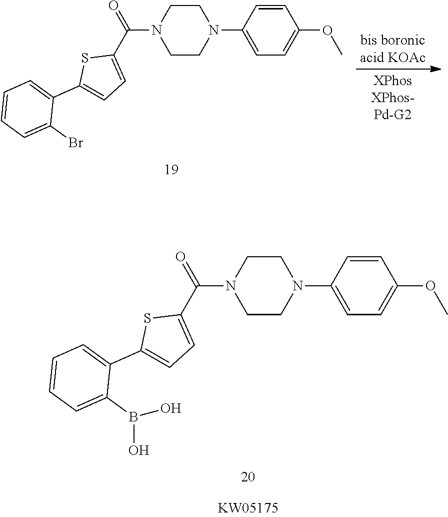 [SMILES: COc1ccc(N2CCN(C(=O)c3ccc(-c4ccccc4Br)s3)CC2)cc1 ; COc1ccc(N2CCN(C(=O)c3ccc(-c4ccccc4B(O)O)s3)CC2)cc1]

(2-(5-(4-(4-methoxyphenyl)piperazine-1-carbonyl) thiophen-2-yl)phenyl)boronic acid (20): Compound 20 was synthesized from compound 19 by procedure 3 (45% yield). 1H NMR (500 MHZ, MeOD) δ 7.58 (dt, J=7.6, 1.0 Hz, 1H), 7.48-7.38 (m, 4H), 7.14 (d, J=3.8 Hz, 1H), 7.01-6.96 (m, 2H), 6.89-6.84 (m, 2H), 3.98-3.89 (m, 4H), 3.75 (s, 3H), 3.17-3.08 (m, 4H). ¹³C NMR (126 MHZ, MeOD) δ 163.66, 154.67, 149.50, 145.18, 136.08, 135.88, 132.02, 130.34, 129.08, 127.82, 127.70, 124.19, 118.81, 114.09, 54.54, 50.99, 48.47. ¹¹B NMR (160 MHz, MeOD) δ 31.22. HRMS (ESI) calc. for [M+H]⁺ C₂₂H₂₄N₂O₄BS 423.1550 obsd. 423.1150.

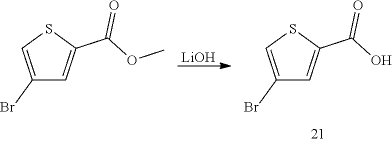

Synthesis of 4-bromo-2-thiophenecarboxylic acid (21): LiOH (0.48 g, 11 mmol) was dissolved in water (18 mL) to which was added a solution of methyl 4-bromo-2-thiophenecarboxylate (0.55 g, 2.5 mmol) in THF (2.5 mL). The mixture was stirred at room temperature for 4 h. The liquid was acidified to pH 4 with 6 M HCl and extracted three times with EtOAc (20 mL each). The organic fractions were pooled, dried over sodium sulfate, and concentrated under reduced pressure to give compound 21 which was taken to the next step without further purification (0.44 g, 2.1 mmol, 84% yield).

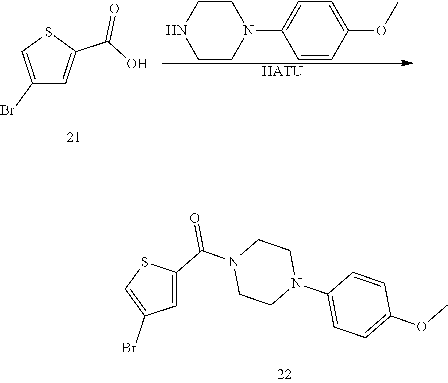

(4-Bromothiophen-2-yl) (4-(4-methoxyphenyl)piperazin-1-yl) methanone (22): Compound 22 was synthesized from compound 21 by procedure 1 (73% yield).

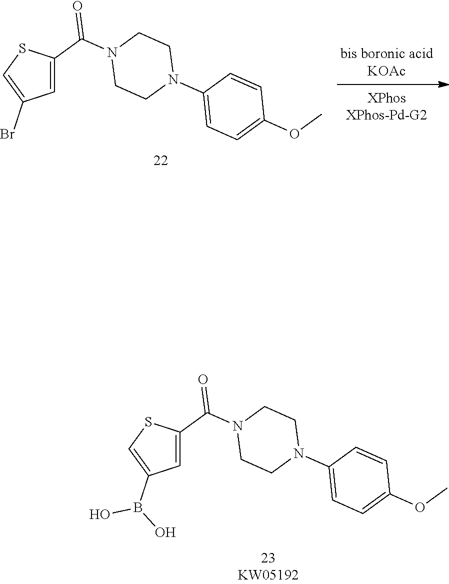 [SMILES: COc1ccc(N2CCN(C(=O)c3cc(Br)cs3)CC2)cc1 ; COc1ccc(N2CCN(C(=O)c3cc(B(O)O)cs3)CC2)cc1]

(5-(4-(4-Methoxyphenyl)piperazine-1-carbonyl) thiophen-3-yl)boronic acid (23): Compound 23 was synthesized by procedure 3 from compound 22 (59% yield). ¹H NMR (500 MHZ, MeOD) δ 8.05 (s, 1H), 7.64 (s, 1H), 7.01-6.91 (m, 2H), 6.90-6.81 (m, 2H), 3.89 (t, J=5.1 Hz, 4H), 3.74 (s, 3H), 3.08 (t, J=5.1 Hz, 4H). ¹³C NMR (126 MHZ, MeOD) δ 164.20, 154.65, 145.14, 137.71, 136.07, 134.12, 118.80, 114.09, 54.54, 50.99, 48.50. ¹¹B NMR (160 MHz, MeOD) δ 26.25. HRMS (ESI) calc. for [M+H]⁺ C₁₆H₂₀N₂O₄BS 347.1237, obsd. 347.1235.

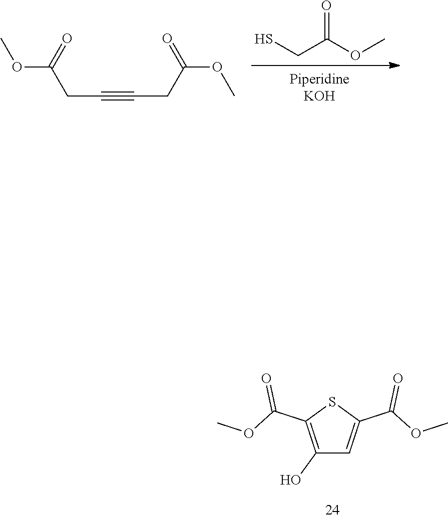

Dimethyl 3-hydroxy-2,5-thiophenedicarboxylate (24): Dimethyl acetylenedicarboxylate (5.0 g, 35 mmol) was dissolved in MeOH (11 mL) and chilled to 0° C. Piperidine (0.35 mL, 3.5 mmol) was added drop wise followed by methyl thioglycolate (3.2 mL, 35 mmol). In a separate vessel, KOH (2.53 g, 45 mmol) was dissolved in MeOH (45 mL) then added dropwise to the chilled solution. The solution was warmed to room temperature and stirred for 30 min. This was then poured into ice cold water (500 mL) and acidified with 6 M HCl to precipitate compound 24. The solid was collected by vacuum filtration and washed with 0.1 M HCl then taken to the next step without further purification (6.4 g, 29 mmol, 84%).

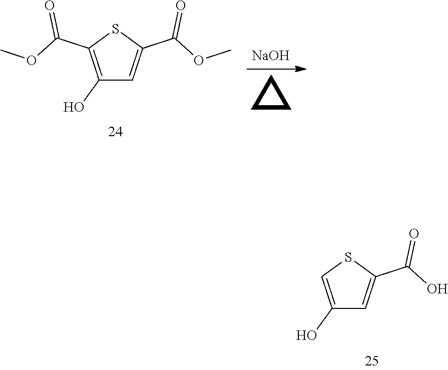

4-Hydroxy-2-thiophenecarboxylic acid (25): Compound 24 (6.4 g, 29 mmol), was dissolved in 2 M aqueous NaOH (90 mL) and heated to reflux for 1 h. While the liquid was still hot, the solution was neutralized with 6 M HCl (30 mL) then acidified by slow addition of concentrated HCl (6 mL). The solution was then heated to reflux for an additional 30 min. When the solution had cooled to room temperature, the liquid was saturated with NaCl and extracted three times with EtOAc (150 mL each). The organic fractions were pooled, dried over sodium sulfate, and concentrated under reduced pressure. This yielded compound 25 which was taken to the next step without further purification (4.1 g, 28 mmol, 98% yield).

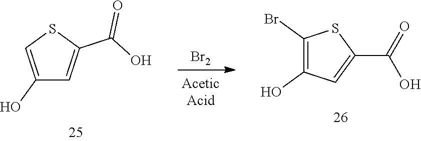 [SMILES: O=C(O)c1cc(O)cs1 ; "Br2" ; "AceticAcid" ; O=C(O)c1cc(O)c(Br)s1]

5-Bromo-4-hydroxy-2-thiophenecarboxylic acid (26): Compound 25 (4.2 g, 29 mmol) and bromine (1.8 mL, 35 mmol) were dissolved in acetic acid (150 mL). The solution was heated to reflux for 16 h. After cooling the solution was poured into water (1 L) and extracted 3 times with EtOAc (500 mL each). The organic fractions were pooled, dried over sodium sulfate, and concentrated under reduced pressure. The crude material was purified by flash chromatography (DCM→3:47 MeOH:DCM) to give compound 26 (5.2 g, 22 mmol, 77% yield).

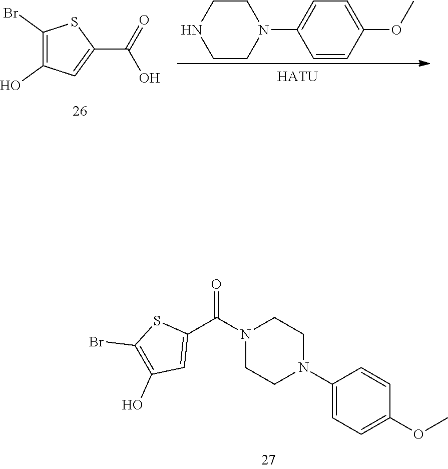

(5-Bromo-4-hydroxythiophen-2-yl) (4-(4-methoxyphenyl)piperazin-1-yl) methanone (27): Compound 27 was synthesized from compound 26 by procedure 1 (32% yield).

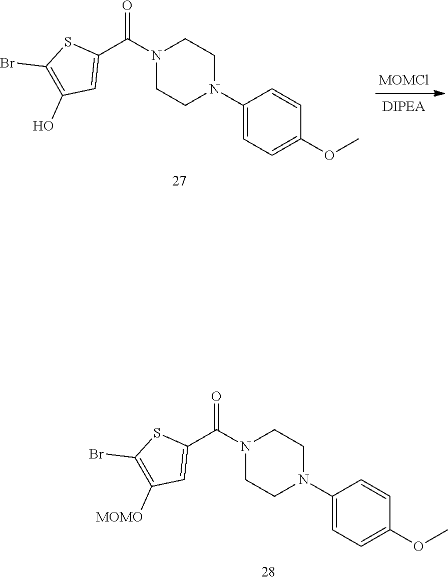

(5-Bromo-4-(methoxymethoxy) thiophen-2-yl) (4-(4-methoxyphenyl)piperazin-1-yl) methanone (28): Compound 27 (0.24 g, 0.60 mmol) and N,N-diisopropylethylamine (DIPEA, 16 mL, 0.90 mmol) were dissolved in DMF (6 mL) and chilled to 0° C. Chloromethyl methyl ether (MOMCl, 68 μL, 0.9 mmol) was added drop wise. The solution was warmed to room temperature and stirred for 16 h. The solution was poured into water (50 mL) and extracted three times with EtOAc (50 mL each). The organic fractions were pooled, dried over sodium sulfate, and concentrated under reduced pressure. The crude was purified by flash chromatography (DCM→1:49 MeOH:DCM) to give compound 28 (0.13 g, 0.30 mmol, 50% yield).

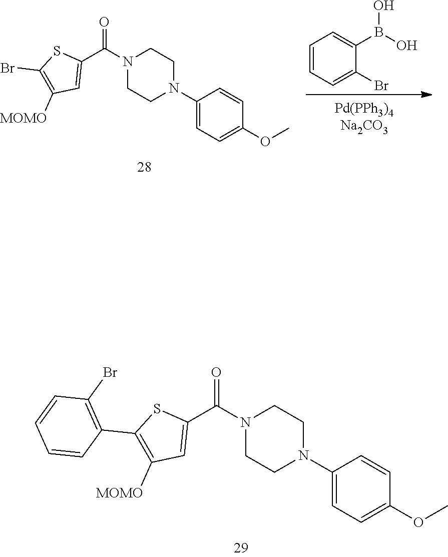

(5-(2-Bromophenyl)-4-(methoxymethoxy) thiophen-2-yl) (4-(4-methoxyphenyl)piperazin-1-yl) methanone (29): Compound 29 was synthesized from compound 28 by procedure 2. Flash chromatography purification resulted in a mixture of compound 29 and dehalogenated compound 28. This mixture was taken to the next step without further purification.

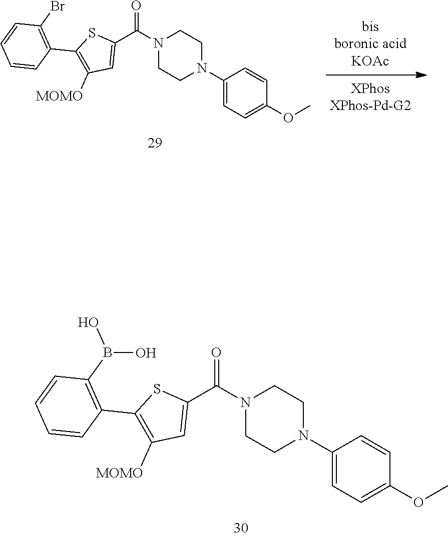

(2-(3-(Methoxymethoxy)-5-(4-(4-methoxyphenyl)piperazine-1-carbonyl) thiophen-2-yl)phenyl)boronic acid (30): Compound 30 was synthesized from compound 29 by procedure 3 (24% yield).

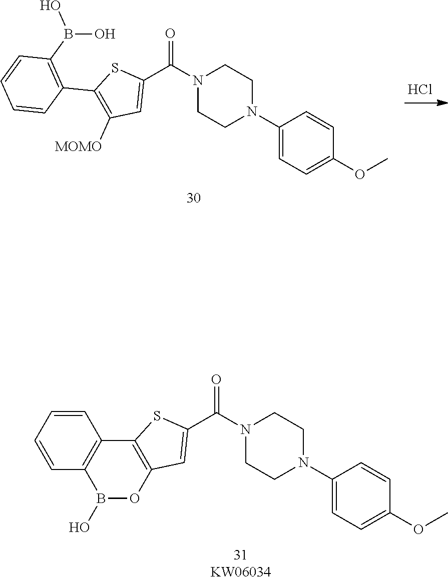

(5-Hydroxy-5H-benzo[c]thieno[2,3-e][1,2]oxaborinin-2-yl) (4-(4-methoxyphenyl)piperazin-1-yl) methanone (31): Compound 30 (12 mg, 0.025 mmol) was dissolved in a 1:1 solution of water and methanol. 6 M HCl was added (43 μL, 0.26 mmol) and the solution was stirred at room temperature for 6 h. Solvent was removed under reduced pressure. The crude product was purified by flash chromatography (DCM→1:49 MeOH:DCM) to give compound 31 (9 mg, 0.021 mmol, 86% yield). ¹H NMR (500 MHZ, DMSO) δ 8.11 (d, J=7.5, 1H), 7.72-7.64 (m, 2H), 7.49-7.42 (m, 2H), 6.98-6.92 (m, 2H), 6.87-6.82 (m, 2H), 3.89-3.76 (m, 4H), 3.70 (s, 3H), 3.10 (dd, J=6.3, 3.9 Hz, 4H), 2.09 (s, 1H). ¹³C NMR (126 MHz, DMSO) δ 162.39, 153.84, 149.54, 145.51, 138.01, 134.53, 133.71, 133.36, 127.45, 122.34, 122.23, 122.13, 118.54, 114.79, 55.66, 50.52. ¹¹B NMR (160 MHz, DMSO) δ 28.41. HRMS (ESI) calc. for [M+H]⁺ C₂₂H₂₂N₂O₄BS 421.1393, obsd. 421.1394.

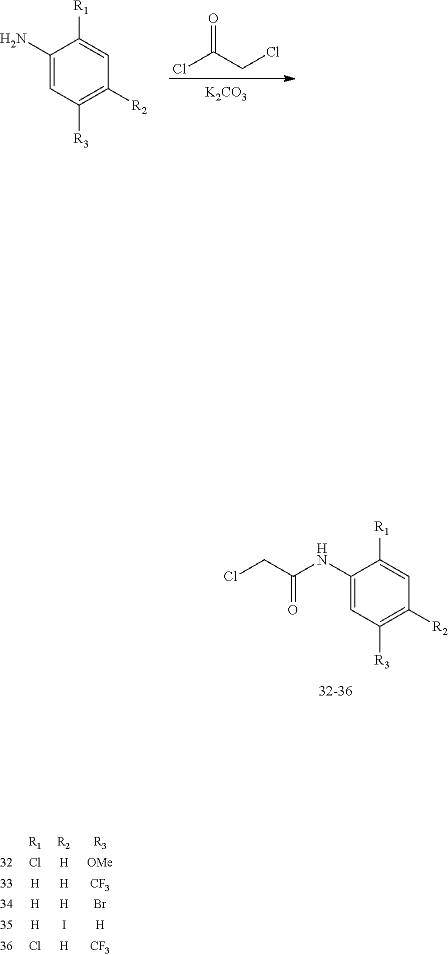

2-Chloro-N-(2-chloro-5-methoxyphenyl)acetamide (32), 2-chloro-N-(3-(trifluoromethyl)phenyl)acetamide (33), N-(3-bromophenyl)-2-chloroacetamide (34), 2-chloro-N-(4-iodophenyl)acetamide (35), & 2-chloro-N-(2-chloro-5-(trifluoromethyl)phenyl)acetamide (36): Compounds 32-36 were synthesized by procedure 4 (83%, 85%, 93%, 95%, and 74% yield, respectively).

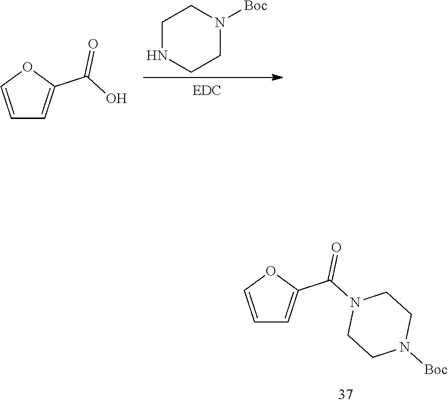

1,1-Dimethylethyl 4-(2-furanylcarbonyl)-1-piperazinecarboxylate (37): 2-furoic acid (0.10 g, 0.93 mmol) was dissolved in DCM (1 mL) and EDC (0.22 g, 1.1 mmol) was added followed by N-Boc-piperazine (0.18 g, 0.99 mmol). The solution was stirred at room temperature for 4 h. DCM (4 mL) was added to dilute the solution which was washed with water and brine (5 mL each). The organic layer was dried over sodium sulfate and concentrated under reduced pressure. The crude product was purified by flash chromatography (1:3→9:11 EtOAc:hexane) to give compound 37 (0.12 g, 0.43 mmol, 46% yield).

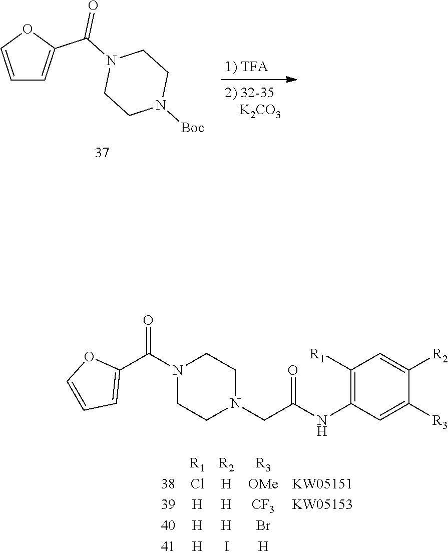

N-(2-chloro-5-methoxyphenyl)-2-(4-(furan-2-carbonyl)piperazin-1-yl)acetamide (38), 2-(4-(furan-2-carbonyl)piperazin-1-yl)-N-(3-(trifluoromethyl)phenyl)acetamide (39), N-(3-bromophenyl)-2-(4-(furan-2-carbonyl)piperazin-1-yl)acetamide (40), & 2-(4-(furan-2-carbonyl)piperazin-1-yl)-N-(4-iodophenyl)acetamide (41): Compounds 38-41 were synthesized from compound 37 and compounds 32-35 respectively by procedure 5 (48%, 51%, 66%, and 33% yield, respectively). (38) ¹H NMR (500 MHZ, CDCl₃) δ 9.91 (s, 1H), 8.17 (d, J=3.0 Hz, 1H), 7.50 (dd, J=1.8, 0.9 Hz, 1H), 7.26 (d, J=8.8 Hz, 1H), 7.04 (dd, J=3.5, 0.8 Hz, 1H), 6.63 (dd, J=8.9, 3.0 Hz, 1H), 6.50 (dd, J=3.5, 1.8 Hz, 1H), 3.93 (s, 4H), 3.82 (s, 4H), 3.24 (s, 2H), 2.73 (t, J=5.0 Hz, 4H). ¹³C NMR (126 MHz, CDCl₃) δ 167.98, 159.15, 159.07, 147.76, 143.83, 134.96, 129.26, 116.81, 113.89, 111.41, 111.04, 111.02, 105.94, 62.01, 55.65, 53.43. HRMS (ESI) calc. for [M+H]⁺ C₁₈H₂₁N₃O₄Cl 378.12151, obsd. 378.12107. (39) ¹H NMR (500 MHz, CDCl₃) δ 9.21 (s, 1H), 7.85 (t, J=2.0 Hz, 1H), 7.78 (dd, J=8.5, 2.1 Hz, 1H), 7.47 (dd, J=1.8, 0.8 Hz, 1H), 7.43 (t, J=8.0 Hz, 1H), 7.36-7.32 (m, 1H), 7.00 (dd, J=3.5, 0.8 Hz, 1H), 6.47 (dd, J=3.5, 1.8 Hz, 1H), 3.88 (s, 4H), 3.19 (s, 2H), 2.67 (t, J=5.1 Hz, 4H). ¹³C NMR (126 MHz, CDCl₃) δ 168.13, 159.11, 147.59, 143.93, [138.01, 131.69, 131.43, 131.17,] (¹⁹F coupling, (q, J=32.4 Hz)), 130.91, 129.62, 124.95, 122.68, [120.89, 120.86, 120.83, 120.80,] (¹⁹F coupling, (q, J=3.8 Hz)), 116.80, [116.27, 116.24, 116.21, 116.18,] (¹⁹F coupling, (q, J=4.0 Hz)), 111.41, 61.90, 53.52, 44.56. ¹⁹F NMR (470 MHz, CDCl₃) δ −62.62. HRMS (ESI) calc. for [M+H]⁺ C₁₈H₁₉N₃O₃F₃ 382.13730 obsd. 382.13699.

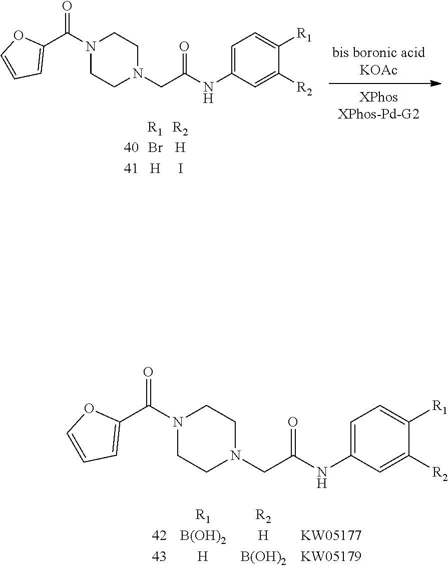

(3-(2-(4-(Furan-2-carbonyl)piperazin-1-yl) acetamido)phenyl)boronic acid (42) & (4-(2-(4-(furan-2-carbonyl)piperazin-1-yl) acetamido)phenyl)boronic acid (43): Compounds 42 and 43 were synthesized from compounds 40 and 41 respectively by procedure 3 (28% and 17% yield respectively). (42) ¹H NMR (500 MHZ, MeOD) δ 7.74 (s, 1H), 7.69 (d, J=2.4 Hz, 1H), 7.65-7.56 (m, 3H), 7.05 (dd, J=3.5, 0.8 Hz, 1H), 6.59 (dd, J=3.5, 1.8 Hz, 1H), 3.90 (s, 4H), 3.24 (s, 2H), 2.71-2.65 (m, 4H). ¹³C NMR (126 MHZ, MeOD) δ 169.30, 159.61, 146.94, 144.58, 139.12, 134.21, 118.88, 116.29, 111.05, 61.28, 52.96. ¹¹B NMR (160 MHz, MeOD) δ 28.66. HRMS (ESI) calc. for [M+H]⁺ C₁₇H₂₁N₃O₅B 358.1574, obds. 358.1576, (43) ¹H NMR (500 MHZ, MeOD) δ 7.79 (s, 1H), 7.68 (s, 1H), 7.64 (d, J=2.0 Hz, 1H), 7.41-7.30 (m, 2H), 7.04 (dd, J=3.5, 0.8 Hz, 1H), 6.57 (dd, J=3.5, 1.8 Hz, 1H), 3.92 (s, 4H), 3.24 (s, 2H), 2.71 (t, J=5.1 Hz, 4H), 1.28 (s, 2H). ¹³C NMR (126 MHZ, MeOD) δ 169.11, 159.64, 146.96, 144.50 136.96, 129.91, 127.99, 125.01, 121.51, 116.62, 111.19, 61.39, 53.10. ¹¹B NMR (160 MHz, MeOD) δ 28.61. HRMS (ESI) calc. for [M+H]⁺ C₁₇H₂₁N₃O₅B 358.1574, obds. 358.1574.

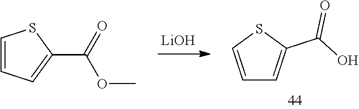

2-Thiophenecarboxylic acid (44): LiOH (0.74 g, 18 mmol) was dissolved in water (28 mL) to which was added a solution of methyl-2-thiophenecarboxylate (0.52 g, 3.7 mmol) in THF (4 mL) The solution was stirred are room temperature for 6 h. The liquid was acidified to pH 4 with 6 M HCl and extracted three times with EtOAc (20 mL each). The organic fractions were pooled, dried over sodium sulfate, and concentrated under reduced pressure to give compound 44 which was taken to the next step without further purification (0.40 g, 3.0 mmol, 86% yield).

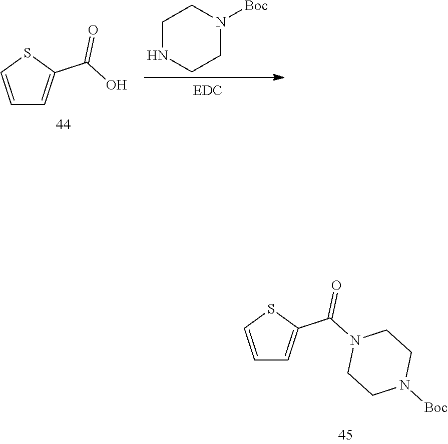

1,1-Dimethylethyl 4-(2-thienylcarbonyl)-1-piperazinecarboxylate (45): Compound 44 (0.30 g, 2.3 mmol) was dissolved in DCM (8 mL) and EDC (0.56 g, 2.9 mmol) was added followed by N-Boc-piperazine (0.43 g, 2.3 mmol). The solution was stirred at room temperature for 4 h. DCM (30 mL) was added to dilute the solution which was washed with water and brine (40 mL each). The organic layer was dried over sodium sulfate and concentrated under reduced pressure. Crude product was purified by flash chromatography (1:3→9:11 EtOAc:hexane) to give compound 45 (0.51 g, 1.7 mmol, 74% yield).

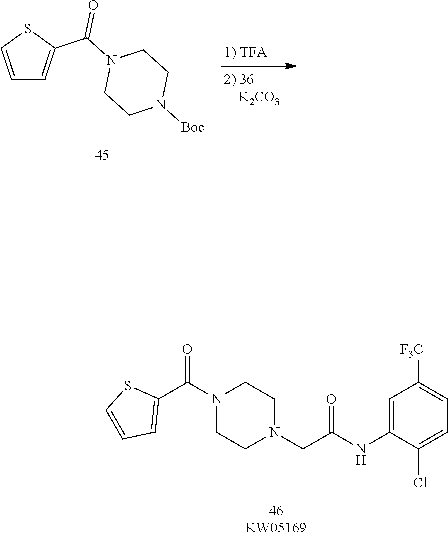 [SMILES: CC(C)(C)OC(=O)N1CCN(C(=O)c2cccs2)CC1 ; O=C(CN1CCN(C(=O)c2cccs2)CC1)Nc1cc(C(F)(F)F)ccc1Cl]

N-(2-chloro-5-(trifluoromethyl)phenyl)-2-(4-(thiophene-2-carbonyl)piperazin-1-yl)acetamide (46): Compound 46 was synthesized from compounds 45 and 36 by procedure 4 by substituting 1,1-Dimethylethyl 4-(2-furanylcarbonyl)-1-piperazinecarboxylate (37) for 1,1-Dimethylethyl 4-(2-thienylcarbonyl)-1-piperazinecarboxylate (45) (34% yield). ¹H NMR (500 MHz, CDCl₃) δ 10.05 (s, 1H), 8.86 (d, J=2.1 Hz, 1H), 7.54 (dd, J=8.4, 1.0 Hz, 1H), 7.50 (dd, J=5.0, 1.1 Hz, 1H), 7.36-7.32 (m, 2H), 7.09 (dd, J=5.0, 3.6 Hz, 1H), 3.90 (t, J=4.6 Hz, 4H), 3.30 (s, 2H), 2.75 (t, J=5.0 Hz, 4H). ¹³C NMR (126 MHz, CDCl₃) δ 168.12, 163.81, 136.57, 134.92, [130.81, 130.55, 130.29, 130.02,] (¹⁹F coupling, (q, J=33.1 Hz,)), 129.53, 129.09, 128.97, 126.82, 125.82, 124.58, 122.41, [121.19, 121.16, 121.13, 121.09,] (¹⁹F coupling, (q, J=3.8 Hz,)), [117.72, 117.69, 117.66, 117.62,] (¹⁹F coupling (q, J=4.0 Hz,)), 61.89, 53.39, 45.69. ¹⁹F NMR (470 MHz, CDCl₃) δ −62.70. HRMS (ESI) calc. for [M+H]⁺ C₁₈H₁₈N₃O₂ClF₃S 423.07549, obsd. 423.07515.

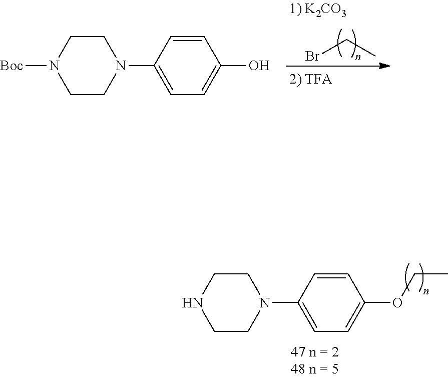

1-(4-Propoxyphenyl)piperazine (47) δ 1-(4-(hexyloxy)phenyl)piperazine (48): Tert-butyl 4-(4-hydroxyphenyl)piperazine-1-carboxylate (1 eq.) and the corresponding bromoalkane (3 eq.) were dissolved in acetone. Potassium carbonate (1.5 eq.) was then added, and the mixture was heated to reflux and stirred for 16 h. Solvent was removed under reduced pressure and the residue was taken up into DCM and washed with 1 M aqueous NaOH, water, and brine. The organic layer was dried over sodium sulfate and concentrated under reduced pressure. The resulting solid was dissolved in DCM to which was added trifluoracetic acid (TFA, 0.4 eq. by volume to DCM). This solution was stirred at room temperature for 2 h. Solvent was removed under reduced pressure and the crude product was dissolved in DCM, washed with 1 M sodium carbonate, water, and brine. The organic layer was dried over sodium sulfate and concentrated under reduced pressure. The crude product was purified by flash chromatography to give compounds 47 and 48 (58% and 50% yield, respectively).

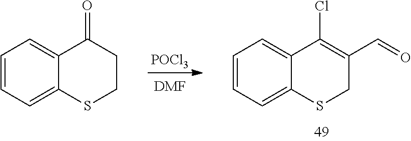

4-Chloro-2H-thiochromene-3-carbaldehyde (49): POCl₃ (0.57 mL, 6.1 mmol) was added dropwise to DMF (6 mL). The solution was chilled to 0° C. and thiochroman-4-one (1 g, 6.1 mmol) was added drop wise. The solution was warmed to room temperature and stirred for 16 h. The solution was poured into ice cold water (100 mL) to quench the reaction. This solution was extracted 3 times with DCM (100 mL each). The organic layers were pooled and dried over sodium sulfate. The solvent was removed under reduced pressure and the resulting product was taken to the next step without further purification (1.1 g, 82% yield).

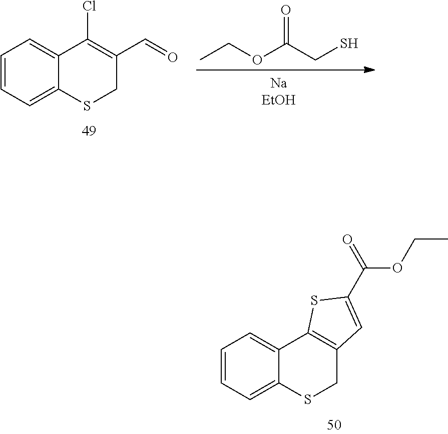

Ethyl 4H-thieno[3,2-c]thiochromene-2-carboxylate (50): Sodium metal (0.16 g, 6.9 mmol) was slowly added to EtOH (20 mL) and dissolved completely before chilling the solution to 0° C. Ethyl thioglycolate (0.28 mL, 6.6 mmol) was added dropwise followed by compound 49. The solution was warmed to room temperature while stirring for 16 h. The solution was then heated to 70° C. for 2 h. The solvent was removed under reduced pressure and the resulting crude was dissolved in EtOAc (50 mL), washed with water and brine (50 mL each), and dried over sodium sulfate. The second crude product was concentrated under reduced pressure and purified by flash chromatography (6:1 hexane: EtOAc) to obtain compound 50 (0.85 g, 60% yield)

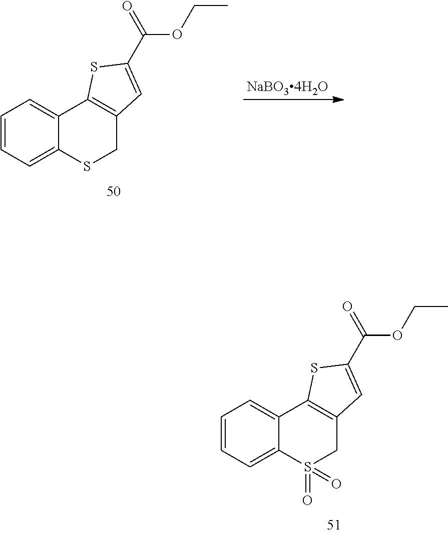 [SMILES: CCOC(=O)c1cc2c(s1)-c1ccccc1SC2 ; "NaBO3·4H2O" ; CCOC(=O)c1cc2c(s1)-c1ccccc1S(=O)(=O)C2]

Ethyl 4H-thieno[3,2-c]thiochromene-2-carboxylate 5,5-dioxide (51): Compound 50 (2.4 g, 8.8 mmol) was dissolved in acetic acid (24 mL) and heated to 60° C. NaBO₃·4H₂O (6.8 g, 44 mmol) was added slowly. The solution was stirred at 60° C. for 5 h. The solution was cooled, and solvent was removed under reduced pressure. The residue was washed with water and dried to obtain compound 51 which was taken to the next step without further purification (1.8 g, 65% yield).

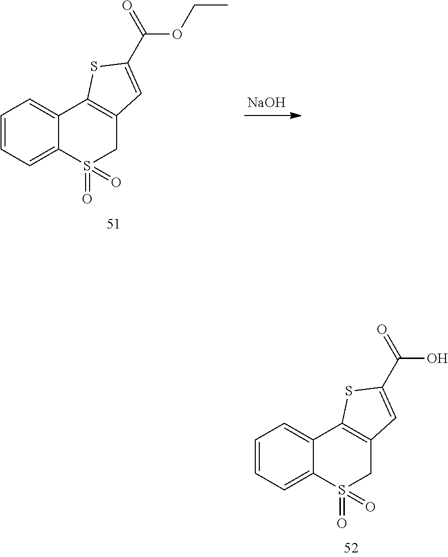 [SMILES: CCOC(=O)c1cc2c(s1)-c1ccccc1S(=O)(=O)C2 ; "NaOH" ; O=C(O)c1cc2c(s1)-c1ccccc1S(=O)(=O)C2]

4H-Thieno [3,2-c]thiochromene-2-carboxylic acid 5,5-dioxide (52): To a solution of compound 51 (1.2 g, 3.9 mmol) in THF (30 mL) was added water (10 mL) and NaOH (1.2 g, 29 mmol). The resulting solution was heated to 80° C. and stirred for 3 h. THF was removed under reduced pressure and the aqueous solution was acidified with 6 M HCl (7 mL). The precipitated solid was collected by vacuum filtration and air dried to obtain compound 52 (1.1 g, 99% yield).

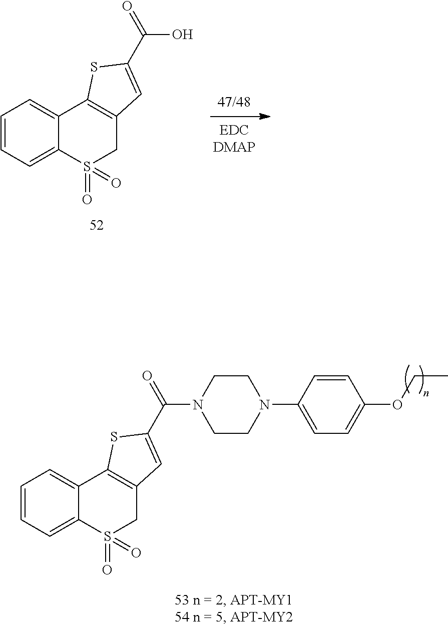

(5,5-Dioxido-4H-thieno[3,2-c]thiochromen-2-yl) (4-(4-propoxyphenyl)piperazin-1-yl) methanone (53) & (5,5-dioxido-4H-thieno[3,2-c]thiochromen-2-yl) (4-(4-(hexyloxy)phenyl)piperazin-1-yl) methanone (54): Compounds 53 and 54 was synthesized from compound 52 and compounds 47 and 48 respectively by procedure 6 (52% and 86% yield respectively). (53) ¹H NMR (500 MHz, CDCl₃) δ 8.05 (dd, J=7.8, 1.3 Hz, 1H), 7.71-7.60 (m, 2H), 7.54 (td, J=7.5, 1.3 Hz, 1H), 7.21 (s, 1H), 6.94-6.88 (m, 2H), 6.88-6.84 (m, 2H), 4.44 (s, 2H), 3.95-3.91 (m, 4H), 3.88 (t, J=6.6 Hz, 2H), 3.18-3.04 (m, 4H), 1.87-1.71 (m, 2H), 1.03 (t, J=7.4 Hz, 3H). ¹³C NMR (126 MHZ, CDCl₃) δ 162.16, 154.17, 144.89, 137.41, 136.23, 133.98, 133.62, 130.10, 129.82, 129.27, 127.85, 125.87, 124.29, 119.06, 115.30, 69.93, 51.27, 51.22, 22.67, 10.55. HRMS (ESI) calc. for [M+H]⁺ C₂₅H₂₇N₂O₄S₂ 483.14068, obsd. 483.41061. (54) ¹H NMR (500 MHZ, CDCl₃) δ 8.06 (dd, J=7.8, 1.2 Hz, 1H), 7.66 (ddd, J=18.3, 7.7, 1.3 Hz, 2H), 7.55 (td, J=7.5, 1.4 Hz, 1H), 7.24 (s, 1H), 6.96-6.90 (m, 2H), 6.90-6.85 (m, 2H), 4.46 (s, 2H), 3.94 (td, J=6.7, 5.9, 2.7 Hz, 6H), 3.19-3.09 (m, 4H), 1.84-1.71 (m, 2H), 1.47 (ddd, J=8.2, 5.3, 2.7 Hz, 2H), 1.35 (ddd, J=7.2, 4.6, 3.3 Hz, 4H), 0.96-0.87 (m, 3H). ¹³C NMR (126 MHZ, CDCl₃) δ 162.14, 154.15, 144.89, 137.42, 136.19, 133.98, 133.62, 130.10, 129.89, 129.26, 127.91, 125.87, 124.25, 119.02, 115.28, 68.42, 51.23, 51.21, 31.62, 29.34, 25.75, 22.63, 14.08. HRMS (ESI) calc. for [M+H]⁺ C₂₈H₃₃N₂O₄S2 525.18763, obds. 525.18730.

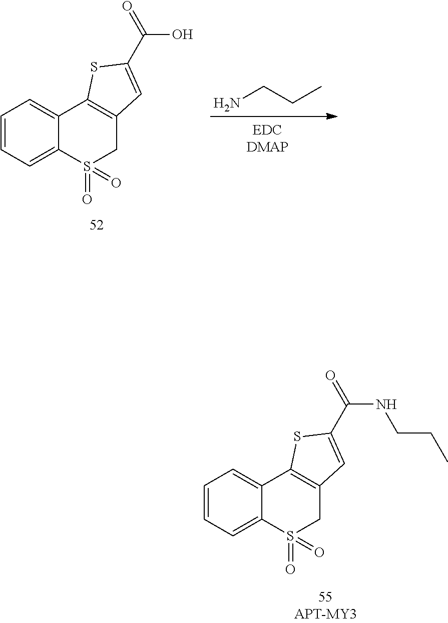

N-Propyl-4H-thieno[3,2-c]thiochromene-2-carboxamide 5,5-dioxide (55): Compound 55 was synthesized from propylamine and compound 52 by procedure 6 (70% yield). ¹H NMR (500 MHZ, DMSO) δ 8.72 (t, J=5.7 Hz, 1H), 7.96 (dd, J=7.6, 1.1 Hz, 1H), 7.83-7.78 (m, 2H), 7.77 (s, 1H), 7.65 (ddd, J=8.5, 6.8, 2.0 Hz, 1H), 4.94 (s, 2H), 3.27-3.19 (m, 2H), 1.54 (q, J=7.2 Hz, 2H), 0.90 (t, J=7.4 Hz, 3H). ¹³C NMR (126 MHz, DMSO) δ 160.69, 140.63, 135.91, 134.66, 134.21, 130.83, 130.28, 129.91, 129.66, 126.46, 123.80, 50.88, 41.48, 22.78, 11.88. HRMS (ESI) calc. for [M+H]⁺ C₁₅H₂₆NO₃S₂ 322.05661, obds. 322.05640.

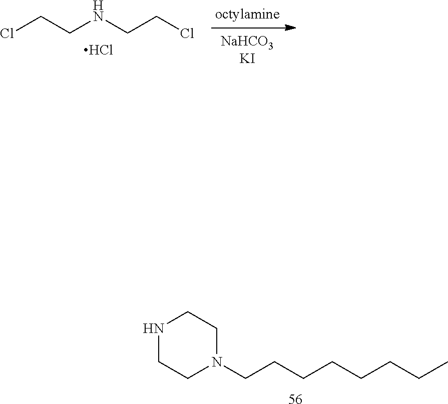

1-Octylpiperazine (56): To a solution of bis(2-chloroethyl amine) hydrochloride (1 g, 5.6 mmol) and octylamine (0.94 mL, 5.7 mmol) in EtOH (30 mL) was added sodium bicarbonate (1.4 g, 17 mmol) and KI (92 mg, 0.56 mmol). The mixture was heated to reflux for 16 h. Solvent was removed under reduced pressure and the residue was taken up into water (50 mL) and extracted three times with DCM (50 mL each). Organic fractions were pooled, washed with water and brine, dried over sodium sulfate, and concentrated under reduced pressure. The crude product was purified by flash chromatography (60:1 DCM: MeOH) to give compound 56 (1.1 g, 95% yield).

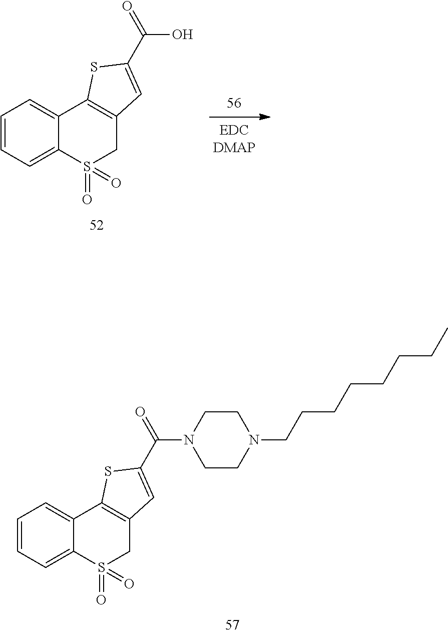 [SMILES: O=C(O)c1cc2c(s1)-c1ccccc1S(=O)(=O)C2 ; CCCCCCCCN1CCN(C(=O)c2cc3c(s2)-c2ccccc2S(=O)(=O)C3)CC1 ; "57"]

(5,5-Dioxido-4H-thieno[3,2-c]thiochromen-2-yl) (4-octylpiperazin-1-yl) methanone (57): Compound 57 was synthesized from compounds 56 and 52 by procedure 6 (86% yield).

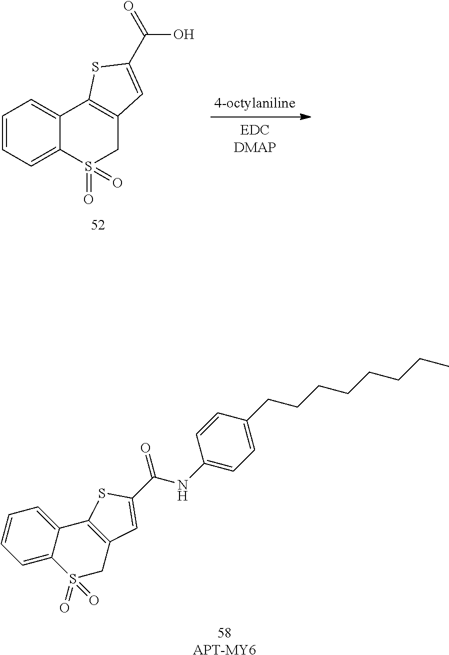 [SMILES: O=C(O)c1cc2c(s1)-c1ccccc1S(=O)(=O)C2 ; CCCCCCCCc1ccc(NC(=O)c2cc3c(s2)-c2ccccc2S(=O)(=O)C3)cc1 ; "58APT-MY6"]

N-(4-octylphenyl)-4H-thieno[3,2-c]thiochromene-2-carboxamide 5,5-dioxide (58): Compound 58 was synthesized from 4-octylaniline and compound 52 by procedure 6 (85% yield). ¹H NMR (500 MHZ, CDCl₃) δ 8.23 (s, 1H), 7.97 (dd, J=7.7, 1.2 Hz, 1H), 7.53-7.45 (m, 2H), 7.45-7.37 (m, 4H), 7.08-7.02 (m, 2H), 4.34 (s, 2H), 2.54 (dd, J=8.8, 6.7 Hz, 2H), 1.56 (d, J=7.2 Hz, 2H), 1.36-1.20 (m, 10H), 0.89 (t, J=6.9 Hz, 3H). ¹³C NMR (126 MHz, CDCl₃) δ 158.81, 140.23, 139.46, 137.86, 134.93, 134.14, 133.04, 130.04, 129.26, 128.84, 128.61, 128.22, 126.06, 124.07, 120.12, 51.22, 35.41, 31.92, 31.60, 29.51, 29.35, 29.32, 22.70, 14.15. HRMS (ESI) calc. for [M+H]⁺ C₂₆H₃₀NO₃S₂ 468.16616, obds. 468.16606.

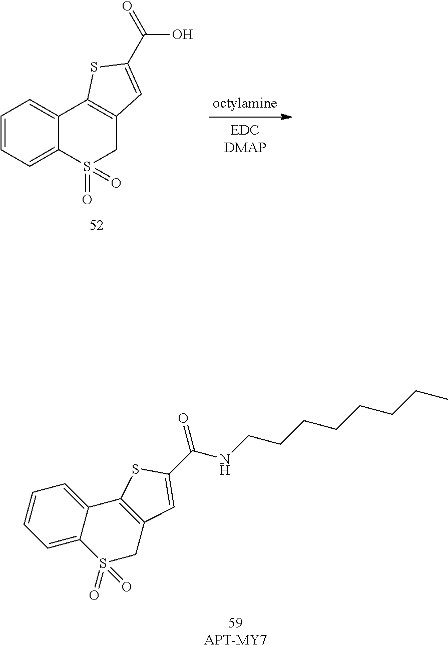 [SMILES: O=C(O)c1cc2c(s1)-c1ccccc1S(=O)(=O)C2 ; CCCCCCCCNC(=O)c1cc2c(s1)-c1ccccc1S(=O)(=O)C2 ; "59APT-MY7"]

N-octyl-4H-thieno[3,2-c]thiochromene-2-carboxamide 5,5-dioxide (59): Compound 59 was synthesized from octylamine and compound 52 by procedure 6 (77% yield). ¹H NMR (500 MHZ, CDCl₃) δ 8.04 (dd, J=7.8, 1.3 Hz, 1H), 7.70-7.61 (m, 2H), 7.54 (td, J=7.5, 1.4 Hz, 1H), 7.34 (s, 1H), 6.09 (dt, J=11.5, 6.0 Hz, 1H), 4.42 (s, 2H), 3.40 (td, J=7.2, 5.8 Hz, 2H), 1.64-1.51 (m, 3H), 1.40-1.19 (m, 12H), 0.88 (t, J=6.8 Hz, 3H). ¹³C NMR (126 MHz, CDCl₃) δ 160.72, 139.55, 137.10, 134.02, 133.66, 130.26, 129.34, 128.44, 128.30, 125.90, 124.26, 51.27, 40.25, 31.80, 29.58, 29.26, 29.21, 26.95, 22.65, 14.11. HRMS (ESI) calc. for [M+H]⁺ C₂₀H₂₆NO₃S₂ 392.13486, obds. 392.13462.

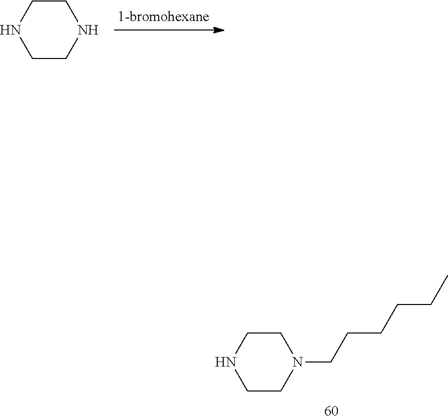

1-hexylpiperazine (60): A solution of piperazine (1.2 g, 14 mmol) and 1-bromohexane (0.50 mL, 3.6 mmol) in EtOH (30 mL) was heated to reflux for 16 h. Solvent was removed under reduced pressure. The residue was re-dissolved in DCM (50 mL) and washed with saturated aqueous sodium bicarbonate and brine (50 mL each). The organic layer was dried over sodium sulfate and concentrated under reduced pressure. Crude material was purified by flash chromatography to give compound 60 (0.51 g, 85% yield).

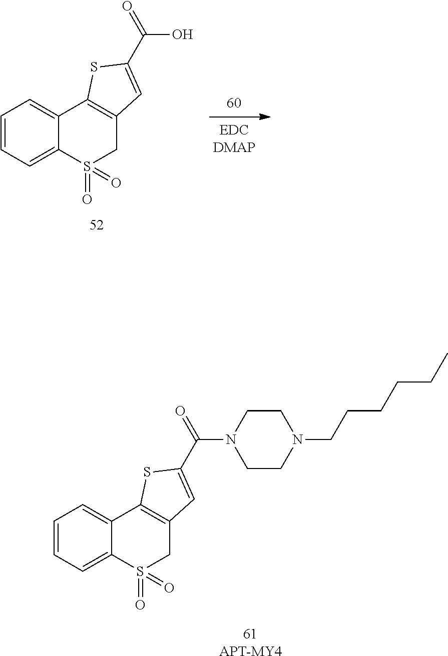 [SMILES: O=C(O)c1cc2c(s1)-c1ccccc1S(=O)(=O)C2 ; CCCCCCN1CCN(C(=O)c2cc3c(s2)-c2ccccc2S(=O)(=O)C3)CC1 ; "61APT-MY4"]

(5,5-dioxido-4H-thieno[3,2-c]thiochromen-2-yl) (4-hexylpiperazin-1-yl) methanone (61): Compound 61 was synthesized from compounds 60 and 52 by procedure 6 (82% yield). ¹H NMR (500 MHZ, CDCl₃) δ 8.04 (dd, J=7.9, 1.3 Hz, 1H), 7.66 (dd, J=7.4, 1.3 Hz, 1H), 7.62 (dd, J=7.9, 1.3 Hz, 1H), 7.54 (td, J=7.5, 1.3 Hz, 1H), 7.18 (s, 1H), 4.44 (s, 2H), 3.79 (t, J=5.0 Hz, 4H), 2.50 (t, J=4.9 Hz, 4H), 2.44-2.31 (m, 2H), 1.51 (t, J=7.5 Hz, 2H), 1.40-1.21 (m, 6H), 0.96-0.83 (m, 3H). ¹³C NMR (126 MHz, CDCl₃) δ 161.96, 137.63, 135.98, 133.94, 133.58, 130.14, 129.71, 129.17, 127.81, 125.84, 124.21, 58.56, 53.17, 51.20, 31.75, 27.15, 26.74, 22.61, 14.07. HRMS (ESI) calc. for [M+H]⁺ C₂₂H₂₉N₂O₃S₂ 433.16141, obsd. 433.16133.

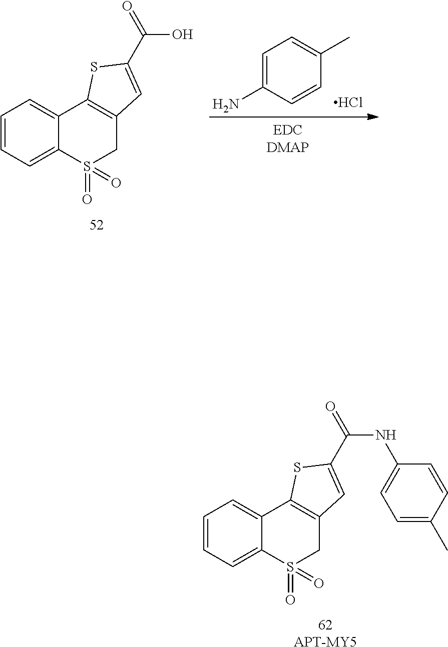

N-(p-tolyl)-4H-thieno[3,2-c]thiochromene-2-carboxamide 5,5-dioxide (62): Compound 62 was synthesized from p-toluidine hydrochloride and compound 52 by procedure 6 (91% yield). ¹H NMR (500 MHz, DMSO) § 10.41 (s, 1H), 8.03 (s, 1H), 7.98 (dd, J=7.9, 1.2 Hz, 1H), 7.86 (dd, J=7.9, 1.2 Hz, 1H), 7.81 (td, J=7.6, 1.3 Hz, 1H), 7.67 (td, J=7.6, 1.3 Hz, 1H), 7.64-7.60 (m, 2H), 7.18 (d, J=8.2 Hz, 2H), 5.00 (s, 2H), 2.29 (s, 3H). ¹³C NMR (126 MHz, DMSO) δ 159.39, 140.48, 136.86, 136.42, 134.69, 134.31, 133.56, 130.94, 130.71, 130.13, 129.63, 126.63, 123.84, 120.84, 50.89, 20.98. HRMS (ESI) calc. for [M+H]⁺ C₁₉H₁₅NO₃S₂ 370.05661, obsd. 370.05646.

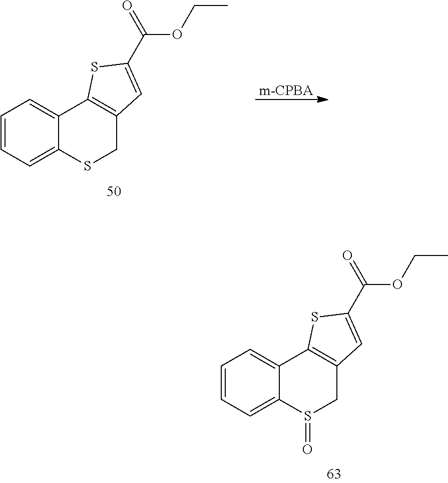 [SMILES: CCOC(=O)c1cc2c(s1)-c1ccccc1SC2 ; "m-CPBA" ; CCOC(=O)c1cc2c(s1)-c1ccccc1S(=O)C2]

Ethyl 4H-thieno[3,2-c]thiochromene-2-carboxylate 5-oxide (63): To a solution of compound 50 (1.0 g, 3.6 mmol) in DCM (40 mL) was added meta-chloroperoxybenzoic acid (m-CPBA, 0.81 g, 3.6 mmol). The reaction mixture was stirred at room temperature for 1 h then washed twice with saturated aqueous sodium bicarbonate and brine (15 mL each). The organic layer was dried over sodium sulfate and concentrated under reduced pressure. Crude product was purified by flash chromatography (1:1→5:6 hexane: EtOAc) to give compound 63 (78% yield).

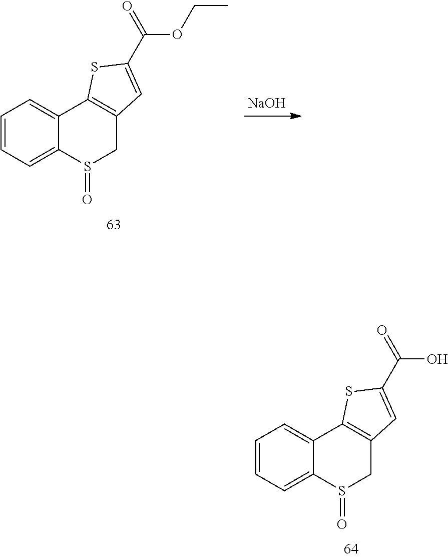 [SMILES: CCOC(=O)c1cc2c(s1)-c1ccccc1S(=O)C2 ; "NaOH" ; O=C(O)c1cc2c(s1)-c1ccccc1S(=O)C2]

4H-Thieno [3,2-c]thiochromene-2-carboxylic acid 5-oxide (64): Compound 63 (0.20 g, 0.68 mmol) was dissolved in MeOH (7 mL) and 1 M aqueous NaOH (3 mL) was added. The mixture was stirred at room temperature for 2 h then heated to 60° C. for 1 h. MeOH was removed under reduced pressure and the crude material was diluted with water (5 mL). The solution was acidified with 6 M HCl (1 mL) and the resulting precipitate was collected by vacuum filtration to give compound 64 (0.18 g, 99% yield).

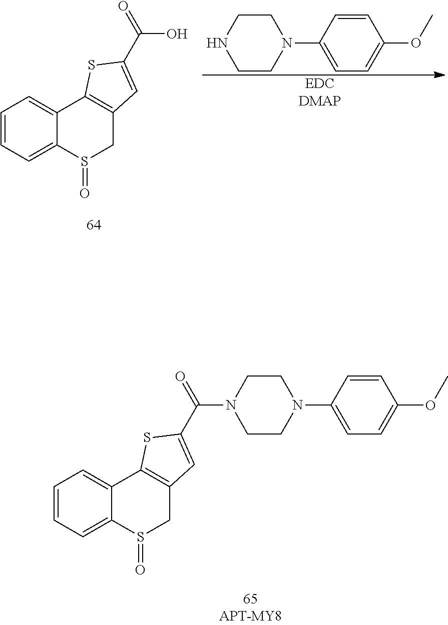

(4-(4-Methoxyphenyl)piperazin-1-yl) (5-oxido-4H-thieno[3,2-c]thiochromen-2-yl) methanone (65): Compound 65 was synthesized from 1-(4-methoxyphenyl)piperazine and compound 64 by procedure 6 (43% yield). ¹H NMR (500 MHz, CDCl₃) δ 7.88 (dd, J=7.6, 1.3 Hz, 1H), 7.67-7.59 (m, 2H), 7.52 (td, J=7.4, 1.6 Hz, 1H), 7.28 (s, 1H), 6.97-6.92 (m, 2H), 6.91-6.86 (m, 2H), 4.49 (d, J=15.3 Hz, 1H), 4.00-3.92 (m, 5H), 3.80 (s, 3H), 3.14 (t, J=5.1 Hz, 4H). ¹³C NMR (126 MHz, CDCl₃) δ 162.54, 154.55, 145.09, 137.89, 136.91, 135.34, 133.06, 130.77, 129.37, 129.30, 128.12, 125.59, 125.54, 119.04, 114.59, 55.58, 51.24, 47.44. HRMS (ESI) calc. for [M+H]⁺ C₂₃H₂₃N₂O₃S₂ 439.11446, obsd. 439.11415.

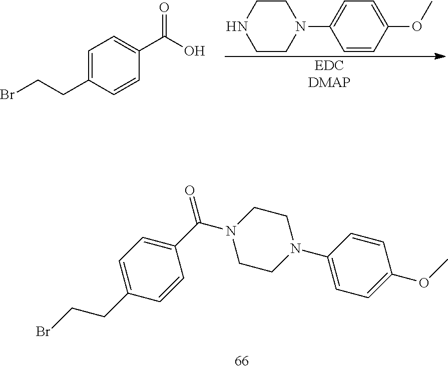

(4-(2-Bromoethyl)phenyl) (4-(4-methoxyphenyl)piperazin-1-yl) methanone (66): Compound 66 was synthesized from 1-(4-methoxyphenyl)piperazine and 4-(2-bromoethyl)benzoic acid by procedure 6 (72% yield).

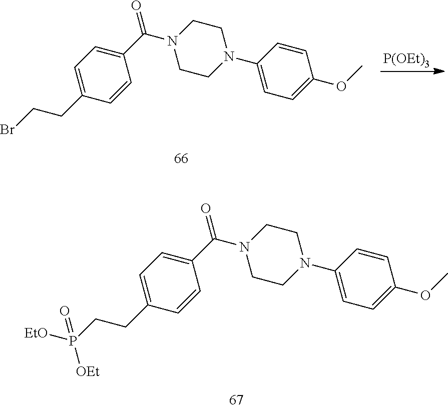 [SMILES: COc1ccc(N2CCN(C(=O)c3ccc(CCBr)cc3)CC2)cc1 ; CCOP(=O)(CCc1ccc(C(=O)N2CCN(c3ccc(OC)cc3)CC2)cc1)OCC ; "P(OEt)3"]

diethyl (4-(4-(4-methoxyphenyl)piperazine-1-carbonyl)phenethyl)phosphonate (67): A mixture of compound 66 (0.30 g, 0.74 mmol) and triethyl phosphine (2 mL) was heated to 170° C. and stirred for 3 h. The mixture was cooled to room temperature and concentrated under reduced pressure. Residue was purified by flash chromatography (30:1 DCM: MeOH) to give compound 67 (0.21 g, 61% yield).

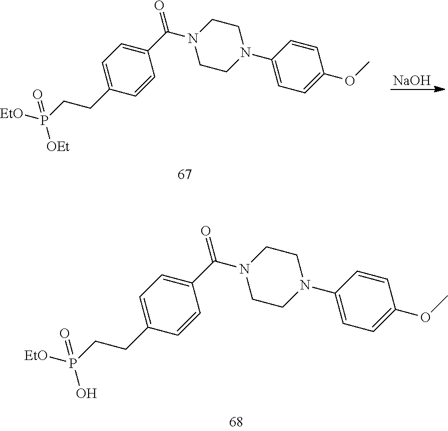

Ethyl hydrogen (4-(4-(4-methoxyphenyl)piperazine-1-carbonyl)phenethyl)phosphonate (68): To a solution of compound 67 (0.50 g, 1.1 mmol) in EtOH (10 mL) was added 1M NaOH (2.5 mL). The resulting mixture was heated to 60° C. and stirred for 3 h. The solution was then cooled and EtOH was removed under reduced pressure. Water (10 mL) was added, and the solution was extracted twice with EtOAc (3 mL each) then acidified with 6 M HCl (0.5 mL) and extracted additional three times with EtOAc (10 mL each). Organic layers were pooled, dried over sodium sulfate, and concentrated under reduced pressure. Crude product was purified by flash chromatography (10:1 DCM: MeOH) to give compound 68 (0.28 g, 59% yield).

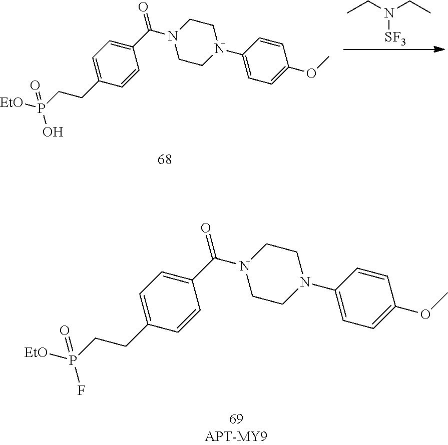

Ethyl (4-(4-(4-methoxyphenyl)piperazine-1-carbonyl)phenethyl)phosphonofluoridate (69): To a solution of compound 68 (50 mg, 0.12 mmol) in DCM (5 mL) under N₂ was added (diethylamino) sulfur trifluoride (30 μL, 0.23 mmol). The reaction mixture was stirred at room temperature for 1 h. Water (0.5 mL) was added to quench the reaction and the solvent was removed under reduced pressure. Residue was purified by flash chromatography to give compound 69 (17 mg, 32% yield). ¹H NMR (500 MHZ, CDCl₃) δ 7.39 (d, J=7.8 Hz, 2H), 7.28 (d, J=7.8 Hz, 2H), 6.95 (d, J=8.6 Hz, 2H), 6.89-6.84 (m, 2H), 4.16-4.08 (m, 2H), 3.79 (s, 3H), 3.49 (s, 4H), 3.21-2.91 (m, 6H), 2.14-2.01 (m, 2H), 1.38-1.32 (m, 3H). ¹³C NMR (126 MHz, CDCl₃) δ 170.34, 143.07, 133.62, 129.93, 128.24, 127.56, 119.13, 114.57, 77.32, 77.27, 77.06, 76.81, 61.32, 61.27, 55.57, 52.08, 50.71, 28.40, 27.12, 16.4. ¹⁹F NMR (470 MHz, CDCl₃) δ −62.95, −65.23. ³¹P NMR (202 MHZ, CDCl₃) δ 32.33, 30.32. HRMS (ESI) calc. for [M+H]⁺ C₂₂H₂₈N₂O₄PF 435.18435, obsd. 435.18432.

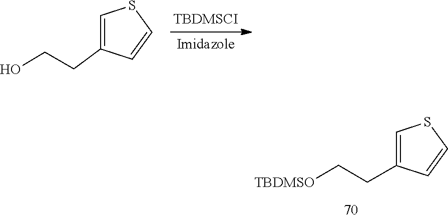 [SMILES: OCCc1ccsc1 ; CC(C)(C)[Si](C)(C)OCCc1ccsc1]

Tert-butyldimethyl (2-(thiophen-3-yl) ethoxy) silane (70): To a solution of 2-(thiophen-3-yl) ethan-1-ol (0.50 g, 3.9 mmol) in DMF (10 mL) was added tert-butyldimethylsilyl chloride (TBDMSCl, 0.65 g, 4.3 mmol) and imidazole (0.40 g, 5.9 mmol). The solution was stirred at room temperature for 12 h. Water (50 mL) was added to quench the reaction and the solution was extracted three times with DCM (50 mL each). The organic fractions were pooled, washed with water and brine, dried over sodium sulfate, and concentrated under reduced pressure. The crude product was purified by flash chromatography (10:1 hexane: EtOAc) to give compound 70 (0.91 g, 99%).

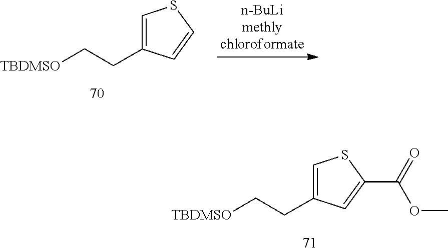 [SMILES: CC(C)(C)[Si](C)(C)OCCc1ccsc1 ; COC(=O)c1cc(CCO[Si](C)(C)C(C)(C)C)cs1]

Methyl 4-(2-((tert-butyldimethylsilyl)oxy) ethyl) thiophene-2-carboxylate (71): At −78° C., n-butyllithium (2.5 M in hexane, 1.4 mL, 3.4 mmol) was added to a solution of compound 70 (0.68 g, 2.8 mmol) in THF (10 mL). The solution was stirred at −78° C. and allowed to warm to room temperature over 5 h. The solution was re-cooled to −78° C. and methyl chloroformate (0.26 mL, 3.4 mmol) was added drop wise. The reaction was warmed to room temperature and stirred for 16 h. Water (10 mL) was added to quench the reaction which was then extracted twice with diethyl ether (50 mL each). Organic fractions were pooled, dried over sodium sulfate, and concentrated under reduced pressure. Crude product was purified by flash chromatography (45:1 hexane: EtOAc) to give compound 71 (0.15 g, 18% yield).

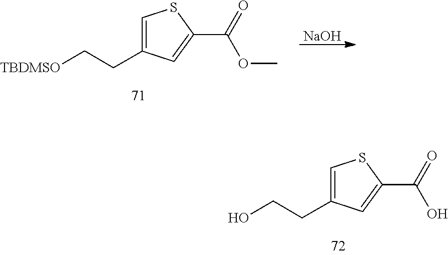

4-(2-Hydroxyethyl) thiophene-2-carboxylic acid (72): Compound 71 (0.28 g, 0.95 mmol) was dissolved in MeOH (3 mL) and then 1 M NaOH (3 mL) was added. The resulting solution was stirred at room temperature for 16 h and then 6 M HCl (0.5 mL) was added. The solvents were removed under reduced pressure. The residue was purified by flash chromatography (1:1 hexane: EtOAc) to give compound 72 (0.14 g, 87% yield

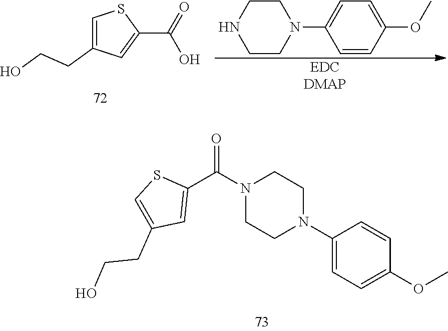

(4-(2-Hydroxyethyl) thiophen-2-yl) (4-(4-methoxyphenyl)piperazin-1-yl) methanone (73): Compound 73 was synthesized from 1-(4-methoxyphenyl)piperazine and compound 72 by procedure 6 (58% yield).

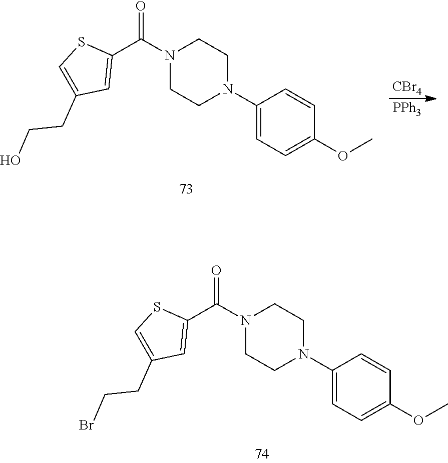 [SMILES: COc1ccc(N2CCN(C(=O)c3cc(CCO)cs3)CC2)cc1 ; "CBr4" ; COc1ccc(N2CCN(C(=O)c3cc(CCBr)cs3)CC2)cc1 ; "PPh3"]

(4-(2-Bromoethyl) thiophen-2-yl) (4-(4-methoxyphenyl)piperazin-1-yl) methanone (74): To dry compound 73 (0.17 g, 0.48 mmol) was added CBr₄ (0.19 g, 0.57 mmol) and PPh₃ (0.15 g, 0.57 mmol). The reaction solution was stirred at room temperature for 5 h. The solvent was removed under reduced pressure and the residue was purified by flash chromatography (3:2 hexane: EtOAc) to give compound 74 (0.16 g, 82% yield).

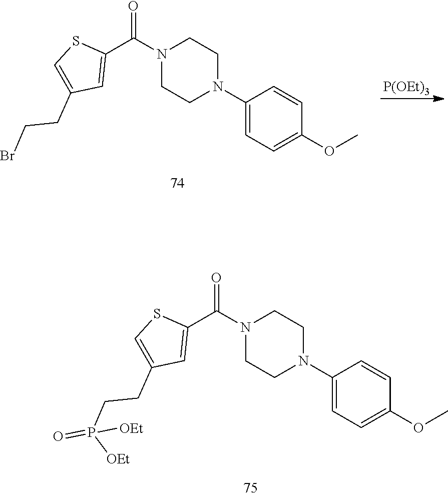 [SMILES: COc1ccc(N2CCN(C(=O)c3cc(CCBr)cs3)CC2)cc1 ; CCOP(=O)(CCc1csc(C(=O)N2CCN(c3ccc(OC)cc3)CC2)c1)OCC ; "P(OEt)3"]

Diethyl (2-(5-(4-(4-methoxyphenyl)piperazine-1-carbonyl) thiophen-3-yl) ethyl)phosphonate (75): A mixture of compound 74 (0.16 g, 0.39 mmol) and triethyl phosphine (2 mL) was heated to 165° C. and stirred for 2.5 h. The mixture was cooled to room temperature and concentrated under reduced pressure. The residue was purified by flash chromatography (30:1 DCM: MeOH) to give compound 75 (0.11 g, 62% yield).

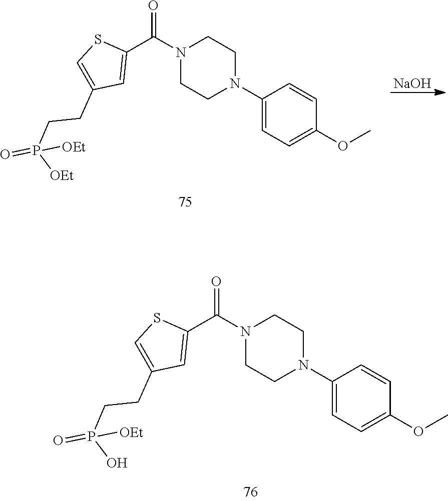 [SMILES: CCOP(=O)(CCc1csc(C(=O)N2CCN(c3ccc(OC)cc3)CC2)c1)OCC ; "NaOH" ; CCOP(=O)(O)CCc1csc(C(=O)N2CCN(c3ccc(OC)cc3)CC2)c1]

Ethyl hydrogen (2-(5-(4-(4-methoxyphenyl)piperazine-1-carbonyl) thiophen-3-yl) ethyl)phosphonate (76): To a solution of compound 75 (0.11 g, 23 mmol) in MeOH (3 mL) was added 1M NaOH (0.69 mL). The solution was stirred at room temperature for 3 h. Then 6M HCl (0.2 mL) was added. The solvents were removed under reduced pressure. The crude product was purified by flash chromatography (30:1 DCM: MeOH) to give compound 76 (91 mg, 91% yield).

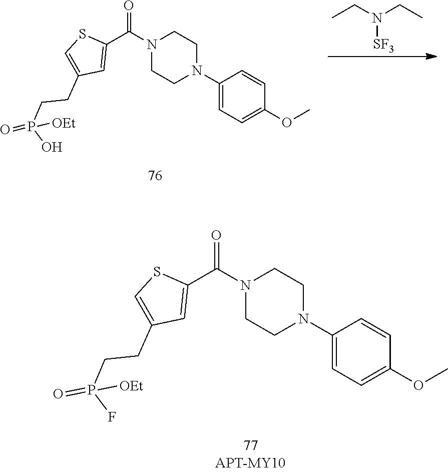

Ethyl (2-(5-(4-(4-methoxyphenyl)piperazine-1-carbonyl) thiophen-3-yl) ethyl)phosphonofluoridate (77): To a solution of compound 76 (50 mg, 0.11 mmol) in DCM (3 mL) under N₂ was added (diethylamino) sulfur trifluoride (31 μL, 0.23 mmol). The reaction mixture was stirred at room temperature for 1 h. Water (0.2 mL) was added to quench the reaction and solvent was removed under reduced pressure. Residue was purified by flash chromatography (50:1, DCM: MeOH) to give compound 77 (35 mg, 70% yield).

### Example 2

This example describes use of compounds of the present disclosure as APT1 and/or APT 2 inhibitors.

APT1 and/or APT2 inhibitors were developed, including some with novel functional groups that covalently target the catalytic serine residue of APT2 (Table 1). Because APT2 is highly homologous to APT1, the inhibition effects of these compounds on APT1 were also tested. Among these new inhibitors, several with comparable APT2 inhibition potency (e.g. APT-MY1, APT-MY11, KW05129, and KW05192) were found. In particular, KW05129 has an IC₅₀ value of 8.54-fold lower than that of ML349, which is the most potent APT2 inhibitor known.

TABLE 1
Structures and IC₅₀ values of inhibitors. The IC₅₀ values were measured
using resorufin acetate as the substrate as previously reported (J Am Chem Soc. 2012, Vol.
134, 10345-10348).

| | | IC₅₀ (μM) | |
|---|---|---|---|
| Name | Structure | APT1 | APT2 |
| ML348 | 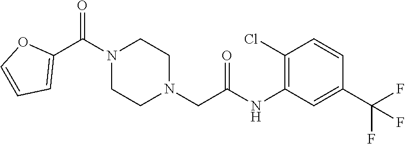 [SMILES: O=C(CN1CCN(C(=O)c2ccco2)CC1)Nc1cc(C(F)(F)F)ccc1Cl] | 0.816 | >200 |
| ML349 | 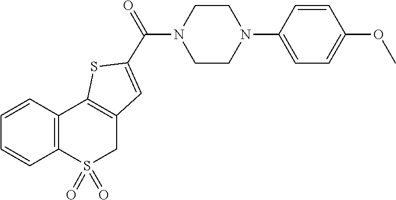 [SMILES: COc1ccc(N2CCN(C(=O)c3cc4c(s3)-c3ccccc3S(=O)(=O)C4)CC2)cc1] | >200 | 0.675 |
| APT-MY1 | 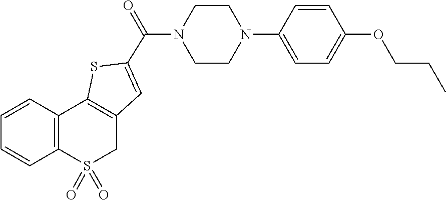 [SMILES: CCCOc1ccc(N2CCN(C(=O)c3cc4c(s3)-c3ccccc3S(=O)(=O)C4)CC2)cc1] | >200 | 0.836 |
| APT-MY2 | 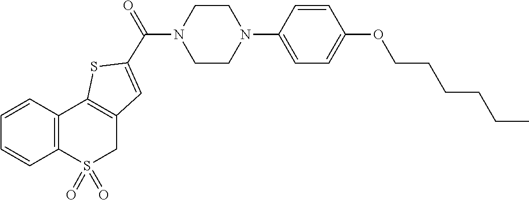 [SMILES: CCCCCCOc1ccc(N2CCN(C(=O)c3cc4c(s3)-c3ccccc3S(=O)(=O)C4)CC2)cc1] | >200 | 2.03 |
| APT-MY3 | 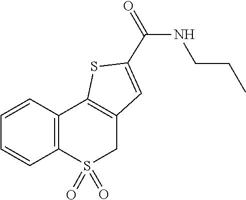 [SMILES: CCCNC(=O)c1cc2c(s1)-c1ccccc1S(=O)(=O)C2] | >200 | >200 |
| APT-MY4 | 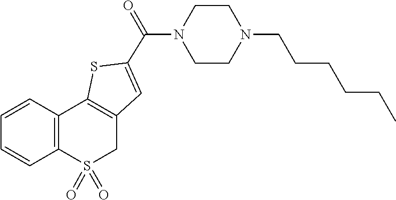 [SMILES: CCCCCCN1CCN(C(=O)c2cc3c(s2)-c2ccccc2S(=O)(=O)C3)CC1] | >200 | 3.99 |
| APT-MY5 | 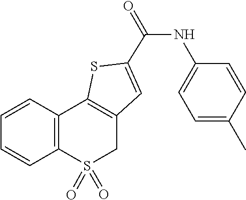 [SMILES: Cc1ccc(NC(=O)c2cc3c(s2)-c2ccccc2S(=O)(=O)C3)cc1] | >200 | >200 |
| APT-MY6 | 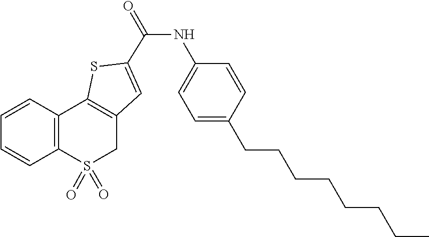 [SMILES: CCCCCCCCc1ccc(NC(=O)c2cc3c(s2)-c2ccccc2S(=O)(=O)C3)cc1] | >200 | >200 |
| APT-MY7 | 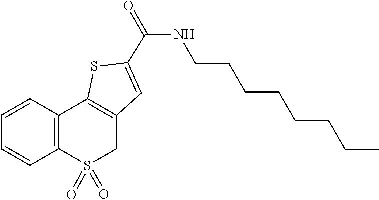 [SMILES: CCCCCCCCNC(=O)c1cc2c(s1)-c1ccccc1S(=O)(=O)C2] | >200 | >200 |
| APT-MY8 | 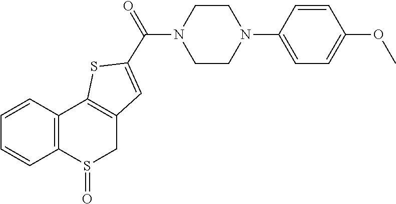 [SMILES: COc1ccc(N2CCN(C(=O)c3cc4c(s3)-c3ccccc3S(=O)C4)CC2)cc1] | >200 | 5.27 |
| APT-MY9 | 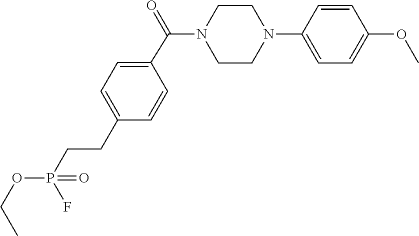 [SMILES: CCOP(=O)(F)CCc1ccc(C(=O)N2CCN(c3ccc(OC)cc3)CC2)cc1] | >200 | 4.07 |
| APT-MY10 | 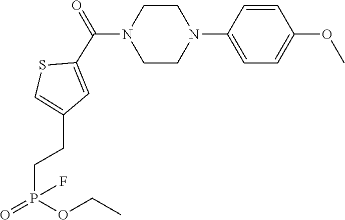 [SMILES: CCOP(=O)(F)CCc1csc(C(=O)N2CCN(c3ccc(OC)cc3)CC2)c1] | >200 | >200 |
| APT-MY11 | 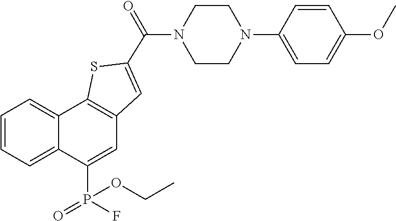 [SMILES: CCOP(=O)(F)c1cc2cc(C(=O)N3CCN(c4ccc(OC)cc4)CC3)sc2c2ccccc12] | 1.06 | 1.42 (0.6 with 30 min preincubation) |
| APT-MY12 | 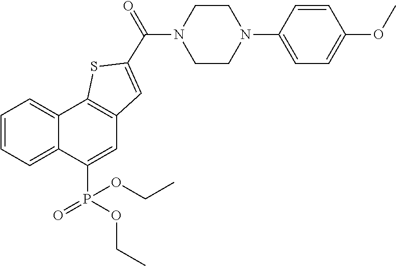 [SMILES: CCOP(=O)(OCC)c1cc2cc(C(=O)N3CCN(c4ccc(OC)cc4)CC3)sc2c2ccccc12] | 1.60 | 4.92 |
| KW05129 | 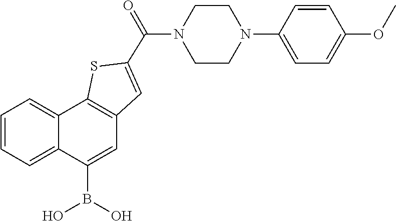 [SMILES: COc1ccc(N2CCN(C(=O)c3cc4cc(B(O)O)c5ccccc5c4s3)CC2)cc1] | .19 | .079 |
| KW05175 | 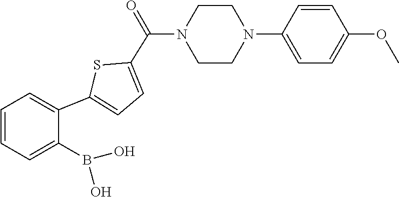 [SMILES: COc1ccc(N2CCN(C(=O)c3ccc(-c4ccccc4B(O)O)s3)CC2)cc1] | TBD | 16.1 |
| KW05191 | 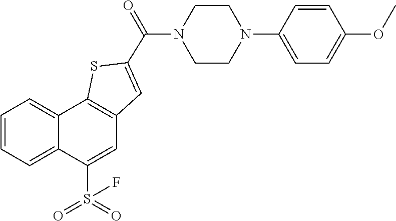 [SMILES: COc1ccc(N2CCN(C(=O)c3cc4cc(S(=O)(=O)F)c5ccccc5c4s3)CC2)cc1] | TBD | 1.77 |
| KW05192 | 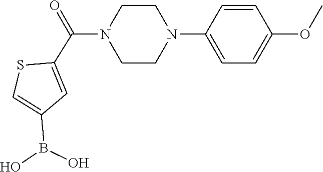 [SMILES: COc1ccc(N2CCN(C(=O)c3cc(B(O)O)cs3)CC2)cc1] | TBD | 1.06 |

Interestingly, it was found that APT-MY11 has a time-dependent inhibition. Pre-incubating APT-MY11 with APT2 for 30 min before adding the substrate resorufin acetate decreased the IC₅₀ to 0.60 μM. In contrast, a time dependent inhibition with the strongest inhibitor KW05129 was not observed. These results suggest that APT-MY11 could be slow binding but irreversible covalent inhibitor of APT2.

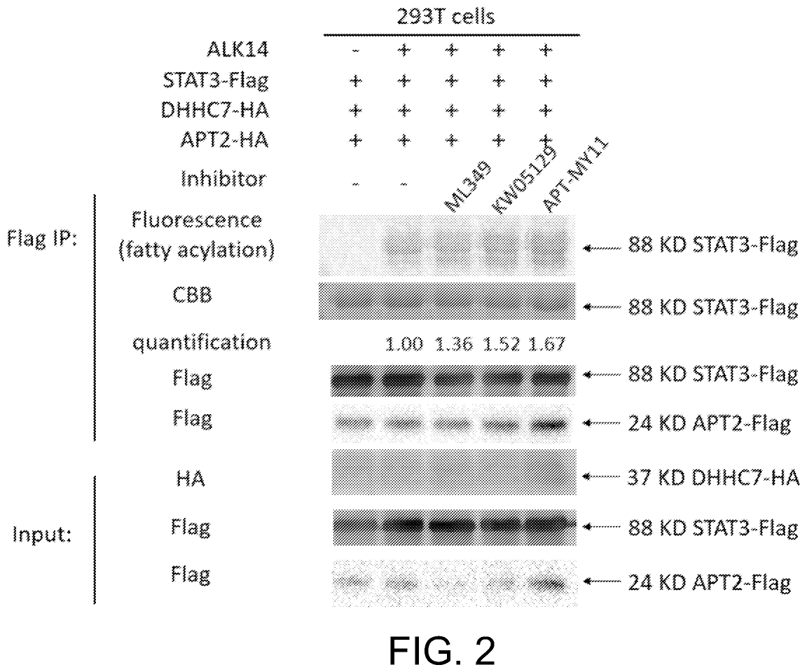

APT-MY11 and KW05129 were tested in HEK 293T cells using two substrate proteins of APT2, SMAD2 (FIG. 1) and STAT3 (FIG. 2). ML349, the known APT2 inhibitor, was used as a control. All three compounds have no effect on cell viability at 50 mM, suggesting that they have very little toxicity to the cells. The palmitoylation levels were measured using a previously established assay (Nature. 2020, Vol. 586, 434-439). Excitingly, both APT-MY11 and KW05129 have slightly better activity at increasing the palmitoylation of SMAD2 (FIG. 1) and STAT3 (FIG. 2). For the SMAD2 assay, the cells were treated with the inhibitors for a shorter time (6 hours), while for the STAT3 assay, the cells were treated with the inhibitors for longer (16 hours). Consistent with the time-dependent inhibition ability of APT-MY11, with shorter incubation time, APT-MY11 seems to be less effective than KW05129 (FIG. 1) but with longer incubation time APT-MY11 seems to be similar or even slightly better than KW05129 (FIG. 2).

FIG. 1 shows that MY11 and KW05129 can increase SMAD2 palmitoylation in cells. HEK 293T cells were transfected with SMAD2-Flag (2 ug), APT2-Flag 2 (ug), DHHC7-HA (1 μg). Cells were treated with the inhibitors at 25 μM and alkyne-tagged fatty acid (Alk14) at 50 μM for 6 h and then harvested to extract the protein for the analysis shown. The fatty acylation level of STAT3 was monitored using in-gel fluorescence after click chemistry mediate conjugation of Rhodamine-azide. Both KW05129 and MY-11 seem to be slightly better than ML349, with KW05129 being better than APT-MY11.

FIG. 2 shows that MY11 and KW05219 can increase STAT3 palmitoylation in cells. HEK 293T cells were transfected with STAT3-Flag (3 μg), APT2-Flag 2 (ug), DHHC7-HA (1 μg). Cells were treated with the inhibitors at 25 μM for 16 hours and with alkyne-tagged fatty acid (Alk14) at 50 M for 6 h and then harvested to extract the protein for the analysis shown. The fatty acylation level of STAT3 was monitored using in-gel fluorescence after click chemistry mediate conjugation of Rhodamine-azide. The quantification shows the acylation level normalized by the STAT3 protein levels, which indicates that all three inhibitors could increase STAT3 palmitoylation levels with KW05129 and MY-11 being slightly better than ML349.

### Example 3

This example describes use of compounds of the present disclosure as APT1 and/or APT 2 inhibitors.

Acyl Protein Thioesterase 2 (APT2) is a serine hydrolase that removes palmitoyl groups from acylated cysteine residues. Recently, the proinflammatory transcription factor STAT3 was reported to be depalmitoylated by APT2, which promotes the translocation of phosphorylated STAT3 to the nucleus where it can promote the expression of RORC and IL17A genes which induces the differentiation of CD4⁺ T cells into T helper 17 cells. The activation of STAT3 contributes to the development of inflammatory bowel diseases (IBD) and APT2 is considered a therapeutic target for IBD treatment. To this end, a series of new APT2 inhibitors were synthesized based on the APT2 selective inhibitor ML349. One of these compounds, KW5129 (also referred to herein as KW05129), which contains a boronic acid moiety, is about nine times more potent than ML349 in vitro but inhibits both APT1 and APT2. KW5129 increases the level of STAT3 palmitoylation in cells and alleviates the symptoms of colitis in mice. This example describes insights for the development of improved APT1/2 inhibitors and supports APT2 as a potential target for inflammatory and autoimmune diseases that involve STAT3 activation.

New covalent and noncovalent APT1 and APT2 inhibitors are presented that are derived from ML349. It is shown that these compounds are more potent APT1 and APT2 inhibitors in vitro and in cells, more effectively prevent STAT3 depalmitoylation, and alleviate the inflammation of dextran sodium sulfate (DSS) induced colitis in mice.

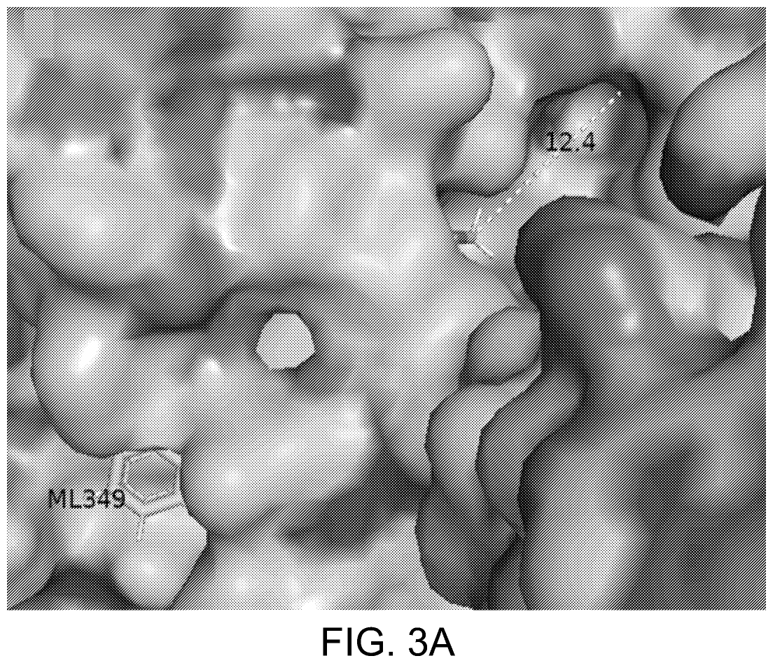

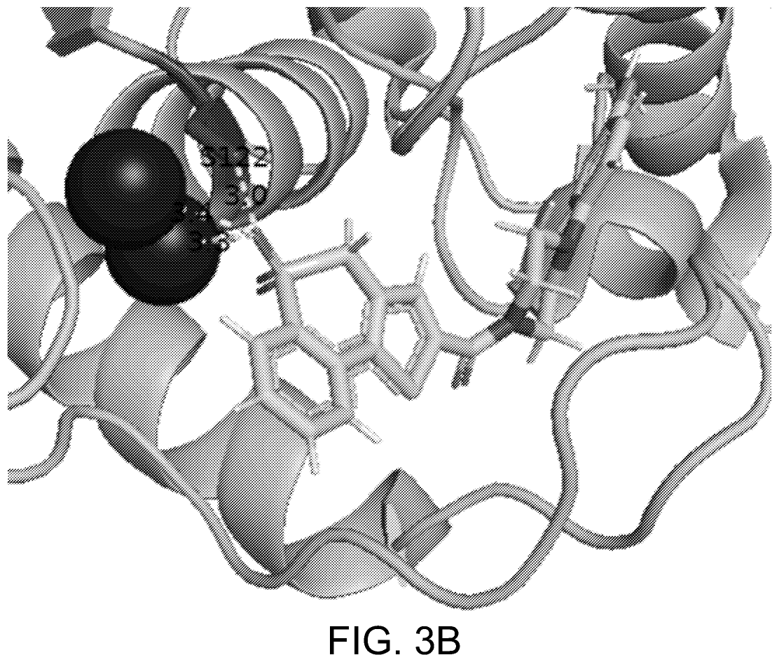

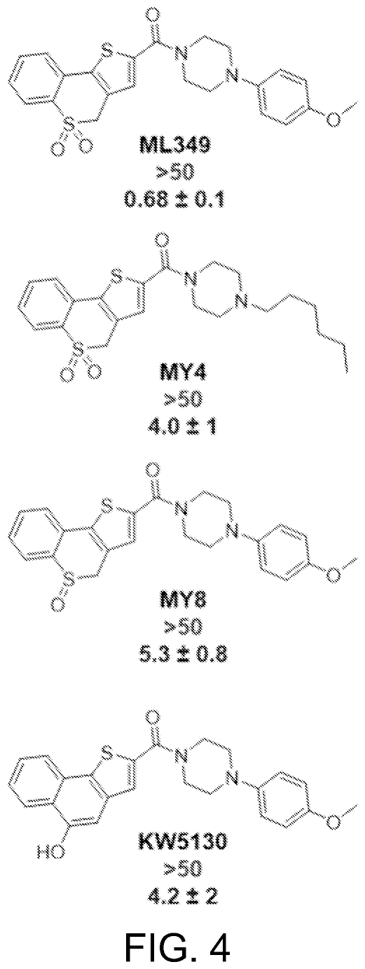

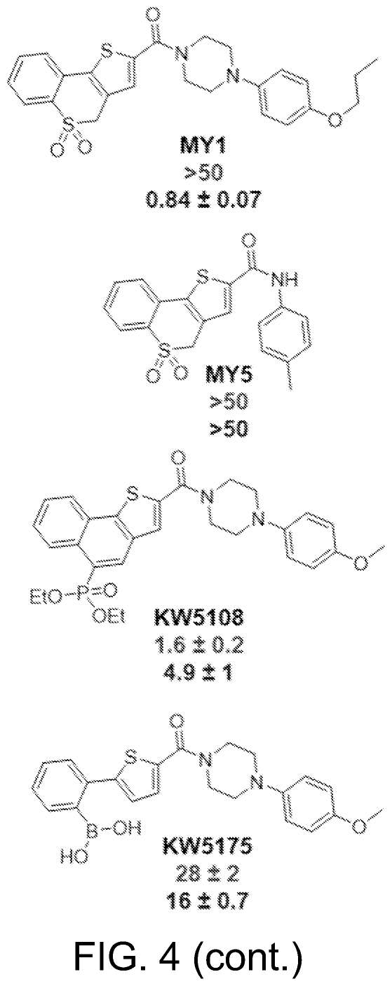

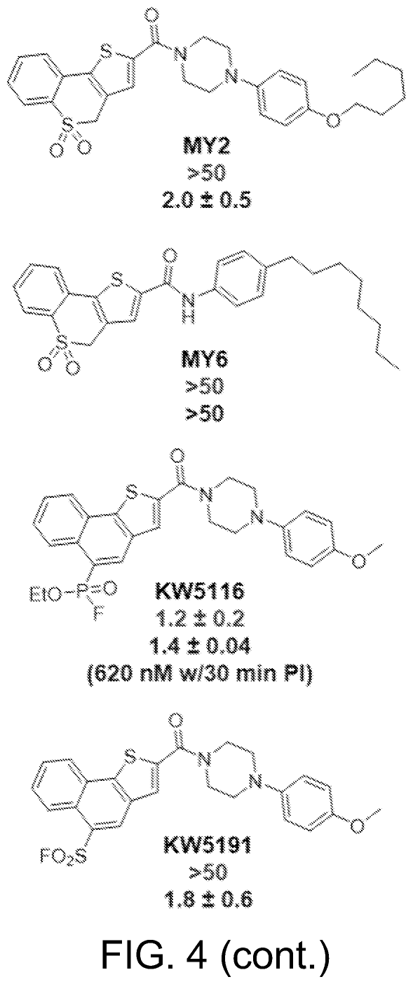

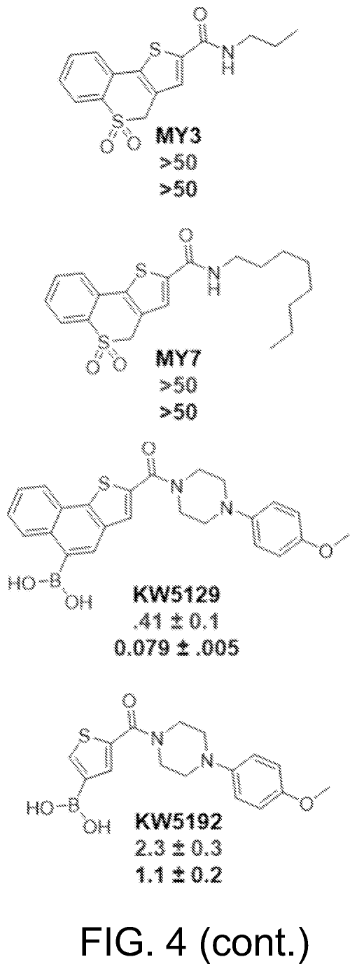

Results and Discussion. New APT2 inhibitor design based on ML349. Currently the most potent APT2-selective inhibitor is ML349. Thus, ML349 was used as the starting point to design and synthesize new APT2 inhibitors. An X-ray crystal structure of APT2 in complex with ML349 showed that the methoxy group of ML349 is located near a relatively hydrophobic channel (FIGS. 3A-3B). Thus, MY1 and MY2 (FIG. 4) were designed to find out whether elongating the methoxy group could enhance APT2 inhibition. MY3-MY7 (FIG. 4) were designed to replace the benzene ring or the piperazine ring to see whether they are critical for APT2 inhibition.

The structure of APT2 in complex with ML349 also showed that the sulfone moiety is positioned near the catalytic serine (S122) and hydrogen bonds to two water molecules near the oxyanion hole (FIG. 3). It was explored whether changing the sulfone moiety could affect APT2 inhibition (FIG. 4). In MY8, the sulfone in ML349 was changed to a sulfoxide. In KW5108 and KW5116, a diethylphosphonate and an ethyl fluorophosphonate were used, respectively. With the ethyl fluorophosphonate, it was hypothesized that it may become a covalent inhibitor by reacting with the catalytic serine residue. In KW5129, a boronic acid was used to mimic the sulfone moiety of ML349. In KW5175 and KW5192, the boronic acid group was kept but the ML349 tricyclic ring was modified. In KW5130 and KW5191, a hydroxyl group and a sulfonyl fluoride were used to mimic the sulfone in ML349.

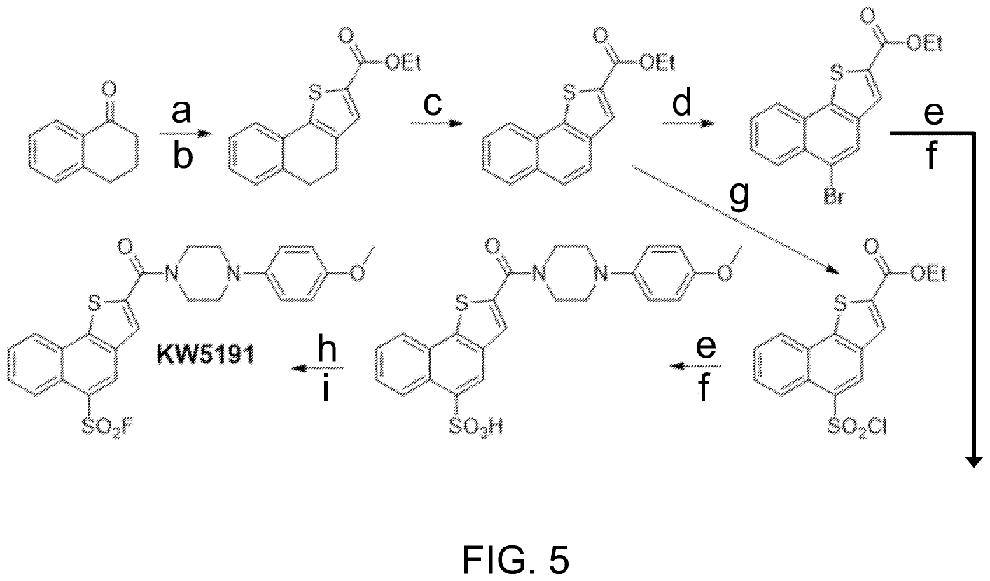

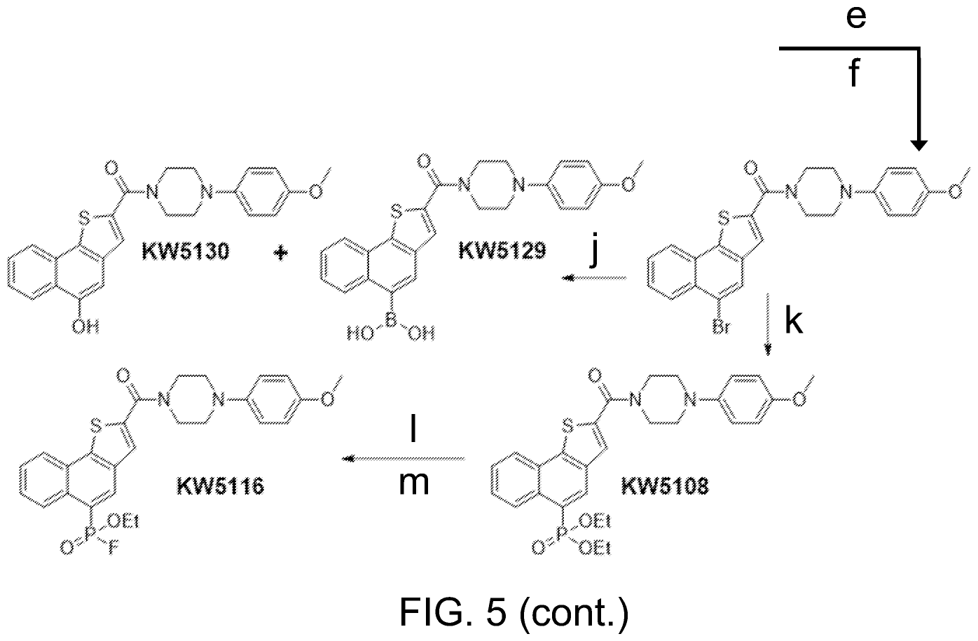

The MY series of compounds were synthesized based on the established synthesis of ML349. KW5108, KW5116, KW5129, KW5130, and KW5191 followed similar synthetic routes except the initial starting material used was «-tetralone (FIG. 5). After constructing the thiophene ring, the middle ring was oxidized to make a fully conjugated system by NBS bromination followed by HBr elimination in the same pot. The naphtho[1,2-b]thiophene product readily underwent electrophilic aromatic substitution with bromine or chlorosulfonic acid selectively at the 5 position. The aryl bromide product was cross-coupled with palladium to attach a boronic acid (KW5129), or diethyl phosphonate which was hydrolyzed and fluorinated to produce KW5116. The remaining KW series compounds required their own unique synthetic routes, as shown herein.

KW5129 and KW5116 are more potent APT2 inhibitors than ML349. The potency of the new compounds against APT1 and APT2 was measured in vitro with a fluorogenic assay. Resorufin acetate was used as a fluorogenic substrate to monitor reaction progress. Deacetylation of resorufin acetate by APT1/APT2 produces resorufin which is strongly fluorescent at 584 nm, allowing the reaction to be easily monitored over time to calculate the IC₅₀ values of each compound (FIG. 4).

Extending the para methoxy group with longer alkyl chains (MY1 and MY2) did not improve the potency. Even a short extension as in MY1 was detrimental. Replacing the benzene or piperazine ring (MY3-MY7) demonstrated that the piperazine amide moiety is critical for APT2 inhibition as its removal abolishes APT2 inhibition while the benzene ring is not as critical.

Modification of the sulfone group in the KW compounds provided better results. The boronic acid-containing KW5129 is ˜9 times more potent at inhibiting APT2 compared with ML349 (FIG. 4). The other two boronic acid compounds (KW5175 and KW5192) without the tricyclic ring in ML349, were still able to inhibit APT2. KW5192 has an IC₅₀ (1.1 μM) that is only slightly worse than that of ML349, while KW5175 is much less potent.

The ethyl fluorophosphonate compound KW5116 gave an IC₅₀ value (1.4 μM) slightly worse than that of ML349. However, when the pre-incubation period was doubled from 15 minutes to 30 minutes, its IC₅₀ dropped to 620 nM, which is slightly better than that of ML349, suggesting that KW5116 is a covalent inhibitor. Similarly, the sulfonyl fluoride compound KW5191 inhibits APT2 with an IC₅₀ value of 1.8 μM, which is slightly worse than KW5116. Additionally, other compounds that remove the sulfone moiety of ML349 (MY8, KW5108, and KW5130) can inhibit APT2 with IC₅₀ of ˜5 μM.

The compounds' inhibition effects on the close homolog, APT1, were also tested. Although ML349 is selective for APT2, many of the new inhibitors lost APT2 selectivity and started to inhibit APT1 (FIG. 4). These include the phosphonate compounds KW5108 and KW5116, as well as the boronic acid compound KW5129. KW5129 inhibits APT1 with an IC₅₀ value of 0.41 μM, which is about 5.2-fold higher than that (0.079 μM) for APT2 and similar to the reported APT1 selective inhibitor ML348, which we found to have an APT1 IC₅₀ value of 0.82 μM. Being dual APT1/APT2 inhibitors, some of these compounds could allow us to test whether inhibiting both APT1/APT2 could be problematic or beneficial in later cellular and animal studies.

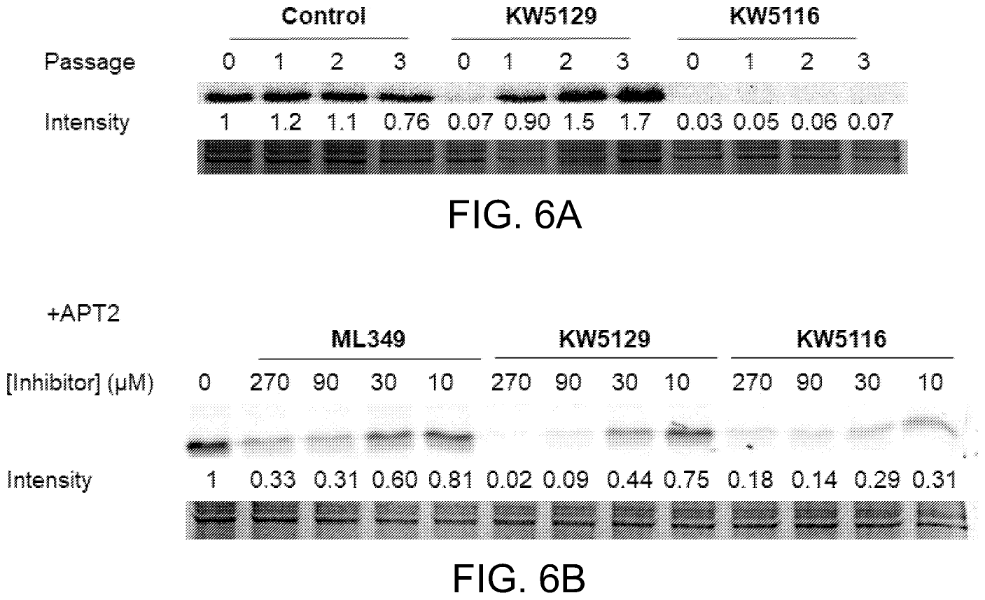

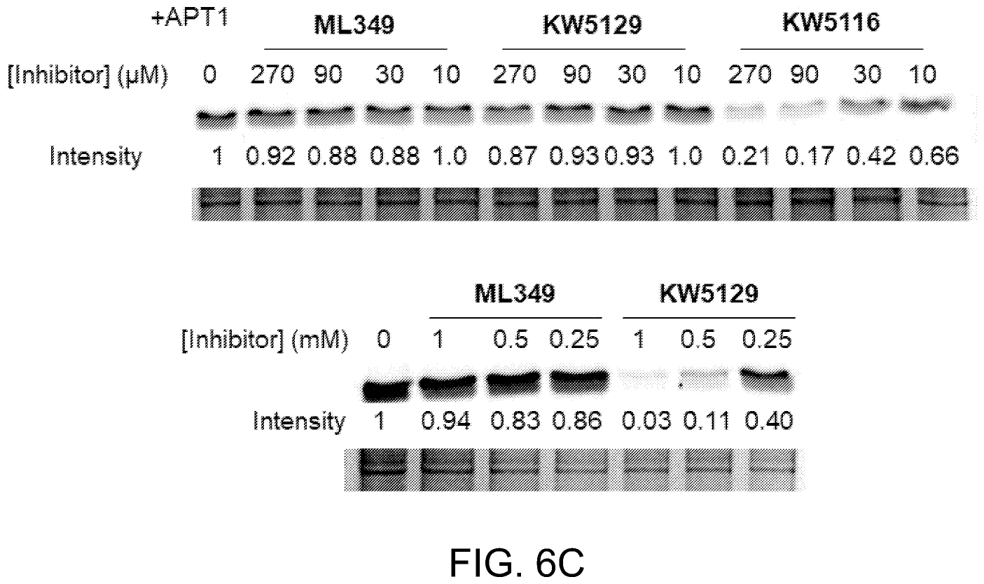

For further characterization, two compounds were focused on, KW5116 and KW5129, which showed the best APT2 inhibition potency, with KW5116 being potentially a covalent inhibitor. It was first determined if KW5116 and KW5129 were covalent or noncovalent inhibitors of APT2 using a gel filtration assay disclosed herein. A cell lysate containing APT2 was treated with the APT2 inhibitors and then passed over a size exclusion column multiple times to remove unbound small molecules. The samples were then incubated with tetramethyl rhodamine fluorophosphonate (TAMRA-FP), which can covalently label the active site serine residue of APT2. In the presence of an inhibitor, APT2 will not be fluorescently labeled with TAMRA-FP. If the inhibitor is a non-covalent inhibitor, after passing over the column, the inhibitor will be lost and APT2 will be fluorescently labeled with TAMRA-FP. In contrast, with a covalent inhibitor, APT2 will remain unlabeled by TAMRA-FP even after multiple gel filtrations. KW5129 showed almost complete fluorescent labeling after just one gel filtration while KW5116 showed none after three passes, indicating the fluorophosphonate moiety allowed it to become a covalent inhibitor (FIG. 6A). In contrast, the boronic acid-containing compound KW5129 is a non-covalent inhibitor of APT2.

Next an in vitro competitive activity-based protein profiling (ABPP) was used to determine the relative potency of KW5116 and KW5129 to that of ML349. A cell lysate containing APT2 was treated with different concentrations of ML349, KW5116, or KW5129 and labeled with TAMRA-FP. In this case, compounds with higher binding affinities would better out compete TAMRA-FP for the active site and lower intensity bands would be observed with more potent compounds (FIG. 6B). Quantification of the fluorescent intensities of the bands showed that addition of ML349 at 90 μM or 270 μM was only able to inhibit ˜70% of fluorescent labeling intensity. KW5129 almost completely abolished TAMRA-FP fluorescence labeling of APT2 at 270 μM. At 90 μM, KW5129 inhibited APT2 labeling by about 90%. The covalent inhibitor KW5116 produced 70% signal reduction even at 10 μM and thus was the strongest among the three. In addition, consistent with the IC₅₀ values measured with purified proteins (FIG. 4), KW5116 and KW5129 could also inhibit APT1 in a similar competitive ABBP assay (FIG. 6C). All the results indicate that KW5129 and KW5116 are more potent APT2 inhibitor than ML349 but can also inhibit APT1.

KW5116 and KW5129 increase STAT3 palmitoylation. To test whether KW5116 and KW5129 can inhibit APT2 in cells, the S-palmitoylation of STAT3 in HEK 293T cells was detected. Based on previous studies, STAT3 is palmitoylated mainly by DHHC7 on C108 and is depalmitoylated by APT2 especially for phosphorylated STAT3 (pSTAT3). To visualize the palmitoylation level of STAT3, HEK 293T cells were co-transfected with Flag-tagged STAT3 and HA-tagged DHHC7 to increase STAT3 palmitoylation. The cells were treated with different APT2 inhibitors and incubated with alkyne-14 (Alk14), which allowed for later fluorescent detection of STAT3 palmitoylation with click chemistry.

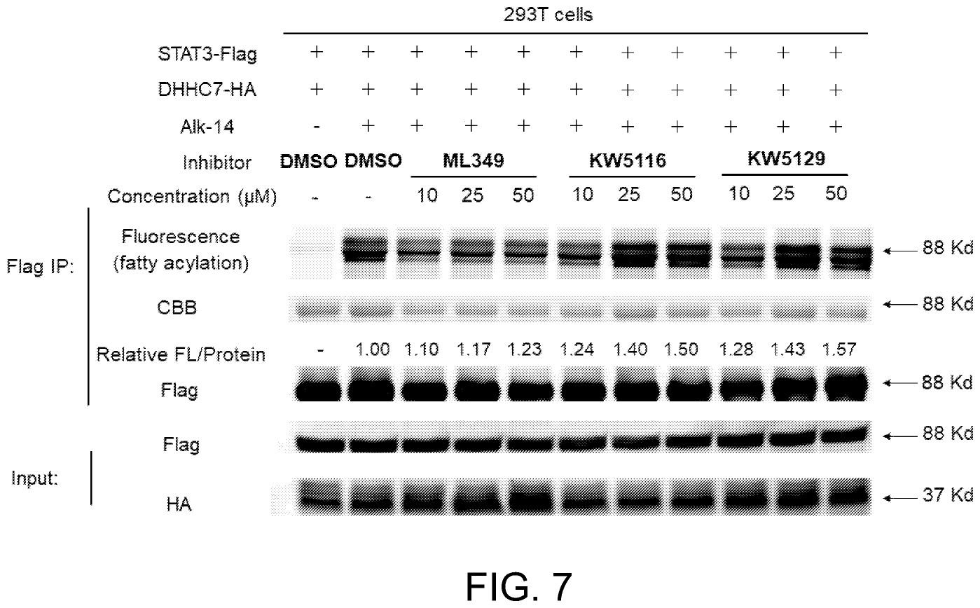

Since only STAT3 and DHHC7 were expressed, this method allowed the inhibition of the endogenous APT2. The overall increase of the palmitoylated STAT3 signal was not dramatic, partially because APT2 prefers to depalmitoylate pSTAT3. With a fixed 16-hour treatment of the inhibitors, KW5116 and KW5129 were compared to ML349. By quantification, 50 μM of ML349 slightly increased the palmitoylation signal of STAT3. At the same concentration, KW5116 and KW5129 were more potent (FIG. 7). In addition, a significant increase of palmitoylated STAT3 with only 25 μM of KW5116 or KW5129 was observed. Overall, KW5129 and KW5116 showed similar effects, and both appeared to be stronger than ML349 at increasing STAT3 palmitoylation. KW5129 reduces the symptoms of colitis in mice. Then the ability of KW5116

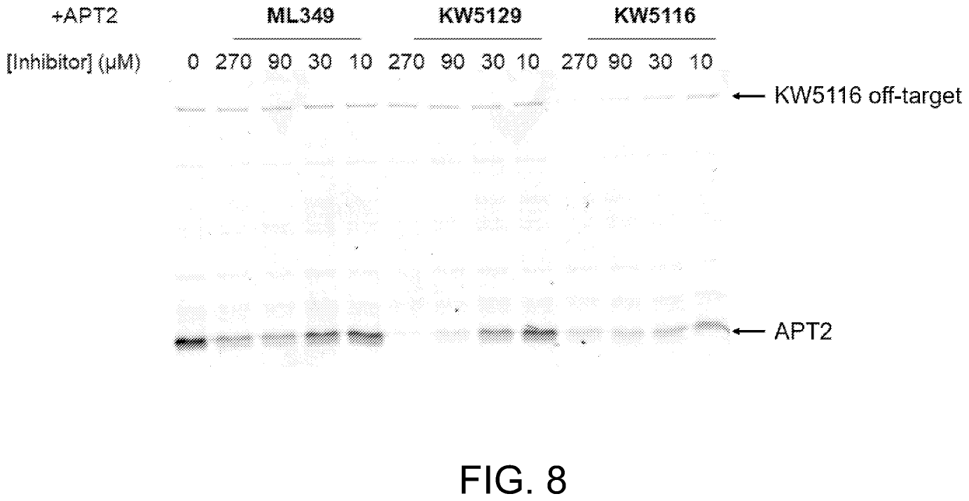

and KW5129 to alleviate the symptoms of IBD in mice was tested. The mice were grouped by sex and supplied with dextran sodium sulfate (DSS) in their drinking water to induce colitis. Simultaneously, they were treated with APT2 inhibitors (50 mg/kg) by intraperitoneal injection for 11 days after which the colons were removed and measured. Before testing the compounds in the DSS model, an initial toxicity assessment of KW5116 and KW5129 was first carried out. While KW5129 is well tolerated at 50 mg/kg daily injection, KW5116 appears to be toxic. This could be due to KW5116 reacting with off target proteins as was seen with the ABPP experiments (FIG. 8). Thus, in the DSS model, we only compared KW5129 to ML349.

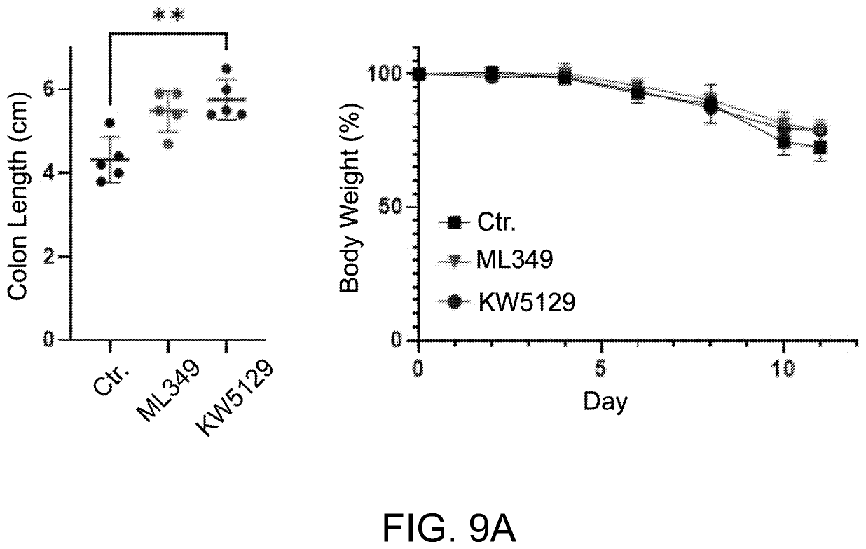

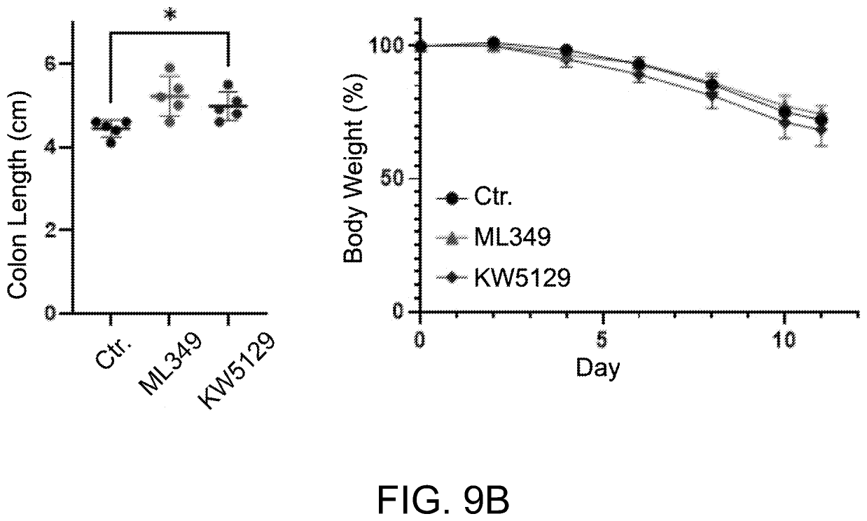

In all cases, treatment with ML349 or KW5129 showed a significant increase in the lengths of the colons compared to the control groups (FIG. 9A). However, while KW5129 appeared to be slightly better than ML349, there was no statistically significant difference between the groups treated with ML349 and KW5129. Female groups responded better to treatment than did the male groups. Body weight loss was less severe in females treated with ML349 and KW5129 and the colons were an average of 1.4 cm longer than the control group. In contrast, the colon length difference was only ˜0.5 cm and there was no statistically significant difference in body weight in the male mice (FIG. 9B). The results indicate that KW5129 is at least as effective at attenuating the symptoms of colitis in mice as ML349 which has been previously shown to protect mice in DSS induced colitis.

In summary, this study further confirmed the previous finding that APT2 inhibitors have the potential to function as IBD therapeutics. In total, 15 new compounds were synthesized based on the APT2 inhibitor ML349 and their potency was tested in vitro. From these, KW5116 and KW5129 were selected as our lead compounds based on their IC₅₀ values. Competitive ABPP showed KW5116 and KW5129 to be a covalent and a noncovalent inhibitor, respectively, and can inhibit APT2 more potently than ML349. Experiments in HEK 293T cells showed a better increase of STAT3 palmitoylation level compared to ML349. In mouse studies, the covalent inhibitor KW5116 appears to be toxic, likely because it targets proteins other than APT1/APT2, while KW5219 is well tolerated. In the DSS-induced colitis mouse model, treatment with ML349 or KW5129 resulted in a reduction in colon shrinkage indicating a reduction in colitis symptoms. Taken together we have demonstrated that new APT2 inhibitor KW5129 is more potent in vitro than ML349 and that KW5129 also inhibits STAT3 depalmitoylation and alleviates symptoms of colitis in mice.

These results highlight several interesting issues. First, the selectivity between APT1 and APT2 can be affected by very small changes in the inhibitor structure. Even though this study started with the APT2-selective inhibitor ML349, many of the changes to the inhibitor structure led to dual inhibitors instead of APT2 selective inhibitors. Thus, future investigations are warranted to understand how the selectivity is determined. Second, these studies identified several warhead structures (boronic acid and phosphonate), which are different from the original sulfone group that interacts with the catalytic serine residue. On the therapeutic front, these studies suggest that a dual APT1/APT2 inhibitor KW5129 is well tolerated and did not cause obvious toxicity, which is similar to the APT2-selective inhibitor ML349. Furthermore, these studies here underscore the potential for the use of APT2 inhibitors as IBD therapeutics.

Materials and Methods. General methods. All solvents and reagents were purchased from commercial vendors as analytical grade or higher purity. Flash chromatography was done using SiliaFlash Irregular Silica Gel, P60, 40-63 μm, 60 Å. NMR spectra were collected at the Cornell NMR Facility using a Brucker 500 spectrometer. HRMS data was collected at the Cornell Chemistry Mass Spectrometry Facility using a Thermo Exactive Orbitrap ESI mass spectrometer. For in vitro IC₅₀ assays, a Cytation 5 cell imaging multi-mode reader was used to monitor the fluorescent intensities with excitation and emission wavelengths set to 535 nm and 590 nm respectively. ActivX TAMRA-FP serine hydrolase probe was purchased from Thermo Fisher Scientific (catalog number 88318). PD-10 Columns prepacked with Sephadex G-25 M for gel filtration assays were purchased from Cytiva.

Cell culture and transfections. HEK 293T cells were cultured in Dulbecco's Modified Eagle Medium (DMEM) supplemented with 10% fetal bovine serum (FBS) and kept in an incubator set to 37° C. with 5% CO₂. To express ZDHHC7 and STAT3, plasmids with the corresponding gene inserts were transfected into 293T cells using polyethylenimine (PEI) (24765, Polysciences) according to the manufacturer's protocol. Flag immunoprecipitation was done with Anti-Flag M2 affinity gel (Millipore Sigma) according to the manufacturer's protocol.

Expression and purification of APT1 and APT2. Human APT1 and APT2 containing a C-terminal 6×His tag in pQE60 vector were transformed into BL21 (DE3) chemically competent E. coli. Typically, 6-8 L of LB broth with 100 μg/mL Ampicillin was inoculated with an overnight starter grown at 30° C. Cultures were grown at 30° C. for ˜4 hours until the OD 600 reached 0.6-0.8, then induced with 1 mM IPTG and incubated at 18° C. overnight to allow protein expression. Cells were harvested by centrifugation at 6000 g. Cell pellets were frozen at −80° C. or immediately used for purification. Pellets were resuspended in lysis buffer (50 mM phosphate buffer pH 8.0, 100 mM NaCl, 10% glycerol, 0.5 mg/mL lysozyme, 100 μM leupeptin, and Pierce universal nuclease). Following a 30 min incubation, cells were sonicated on ice with an ultrasonic liquid processor (Fisherbrand Sonic Dismembrator, FB-505 equipped with a 0.5-inch diameter probe, FB4220) for 4 min total at 60% amplitude. Lysate was clarified by centrifugation at 4° C. and 30,000 g for 35 min. Clarified lysate was incubated with Ni-NTA resin, washed with 50 mL of wash buffer (50 mM phosphate buffer pH 8.0, 100 mM NaCl, 10% glycerol, 10 mM imidazole), and eluted in 2 mL of elution buffers containing a 30-500 mM imidazole. Fractions containing APT1 or APT2 were pooled, concentrated to 3 mL, and desalted using an Econo-Pac 10DG desalting column equilibrated in the final protein storage buffer (50 mM phosphate buffer pH 8.0, 100 mM NaCl, 10% glycerol). The protein was aliquoted, flash-frozen in liquid nitrogen, and stored at −80° C. for future use.

APT1 and APT2 enzymatic assays. Reaction buffer (1×PBS, 0.01% pluronic) was prepared and adjusted to pH 6.5 with HCl. Compounds were dissolved in DMSO to 10 different concentrations, with each being 48.4× of the final concentration to be used in the final enzymatic assay. Purified APT1 or APT2 was diluted in reaction buffer (450 μL) to give 10.8 nM solution. The inhibitor solution (10 μL) was added and incubated at room temperature for 15 min. During the incubation period, a resorufin acetate solution (5 μL, 1 mM in DMSO) was added to wells of a black, flat bottom 96 well plate. Then the enzyme solution (95 μL) was added to each well and the fluorescent intensity of each well was recorded (ex.=571 nm, em.=584 nm) at 70 sec. intervals for 30 min. Three biological replicates were performed for each concentration. Fluorescent intensities were plotted against time to calculate v₀ which was then plotted against concentration using GraphPad Prism to calculate IC₅₀ values for each compound.

Gel Filtration Assay to identify covalent inhibitors. A cell lysate was made from 293T cells transfected to express APT2. This solution was diluted in 1×PBS to give 2 mL of a 1 mg/mL solution. A 50× solution of the inhibitor in DMSO was made, added to the protein solution, and incubated at 37° C. for 30 min. An aliquot (60 μL) was taken, and the remaining sample was loaded onto a Sephadex G-25m column and eluted with PBS (2.5 mL). This process was repeated two more times. The protein concentration of each aliquot was calculated by a BCA assay and adjusted to equalize the protein concentration. Then 25 μL of each aliquot were taken, combined with a ActivX TAMRA-FP solution (0.5 μL, 250 μM in DMSO) and incubated at room temperature for 30 min. Samples were quenched with 6× loading dye then run on a 12% polyacrylamide gel. Gels was imaged with a Typhoon 7000 variable mode imager to determine the fluorescent intensity of the APT2 bands (˜25 kDa).

Competitive in vitro ABPP assay. A cell lysate (0.5 mg/ml) was spiked with purified APT1 or APT2 and 25 μL aliquots were taken. The indicated concentrations of the inhibitor (1 μL of a 25× stock) were added to each aliquot and incubated at room temperature for 15 min. ActivX TAMRA-FP (0.5 μL of a 50× stock) was added and the samples were incubated at room temperature for 30 min. The reaction was quenched with 6× loading dye and the resulting samples were run on a 12% polyacrylamide gel. The fluorescent intensities of the APT1 or APT2 bands were then examined (550 nm ex., 570 nm em. filter) with a Typhoon 7000 variable mode imager.

Detection of STAT3 palmitoylation in cells. HEK 293T cells were transfected with STAT3-Flag and ZDHHC7-HA plasmids for 36 h, and were treated with 25 μM ML349, KW5116, KW5129 for 16 h and 50 μM Alk14 (hexadec-15-ynoic acid) for 6 h before collecting. Cells were collected and lysed in the 1% NP-40 lysis buffer (25 mM Tris-HCl pH 8.0, 10% glycerol, 150 mM NaCl, 1% Nonidet P-40) with protease inhibitor cocktail (P8340, Sigma). STAT3-Flag was purified by anti-Flag agarose beads. Click chemistry was done by adding a mixture of 50 μL IP wash buffer (25 mM Tris-HCl pH 8.0, 150 mM NaCl, 0.2% Nonidet P-40), 3 μL of 2 mM TAMRA-azide (#47130, Lumiprobe), 3.6 μL of 10 mM tris [(1-benzyl-1H-1,2,3-triazol-4-yl)methyl]amine (TBTA), 3 μL of 40 mM CuSO₄, 3 μL of 40 mM tris(2-carboxyethyl) phosphine HCl (TCEP hydrochloride) to each sample. The mixtures were mixed by vortex and incubated in the dark at room temperature for 30 min. To each reaction mixture, 20 μL of 6× loading buffer was added and the mixture was boiled at 95° C. for 10 min to denature. The mixture was then separated by SDS-PAGE gel. The gel was scanned by the Typhoon 7000 Variable Mode Imager to visualize the fatty acylation of STAT3 and was stained with Coomassie Brilliant Blue (B7920, Sigma) to confirm protein loading. Relative fluorescent/protein quantification was done by ImageJ.

Mouse studies. Experiments using live mice were conducted with approval of the Cornell Institutional Animal Care and Use Committee (IACUC) and complied with the laws, policies, and animal care and use procedures that govern the use of live vertebrates in research. 10-week-old C57BL/6J mice (5 males or 5 females per group) were treated with 2.5% DSS in their drinking water for 8 days. On the same day as DSS treatment was started, the mice were injected interperitoneally with a 50 mg/kg does of compound. Injections continued every other day for 11 days. On day 12 the mice were sacrificed, and the colons were removed and measured. Body weights and the health of the mice were tracked throughout the experiment.

Experimental Procedures. Synthetic Methods. Procedure 1, Amide couplings. The carboxylic acid (1 eq.) and amine (1 eq.) starting materials were dissolved in dichloromethane (DCM). 1-Ethyl-3-(3-dimethylaminopropyl) carbodiimide (EDC, 3 eq.) was added to the solution followed by 4-dimethylaminopyridine (DMAP, 0.11 eq.). The solution was stirred at room temperature for 16 h then washed with HCl (0.1 M), water, and brine. The organic layer was then dried over anhydrous sodium sulfate and concentrated under reduced pressure. The resulting crude product was purified by flash chromatography to give the final product.

Procedure 2, Suzuki couplings. An aryl halide (1 eq.), 2-bromophenyl boronic acid (1 eq.), and Pd(PPh₃)₄ (0.04 eq) were combined in a screw cap vial, degassed and charged with N₂. 1,4-Dioxane (volume to make a solution 0.1M in aryl halide) was added to the vial. In a separate vial, sodium carbonate (3 eq.) was dissolved in water (0.25 volume eq. of the dioxane). The water solution was degassed, charged with N2, and added to the first vial. The mixture was then stirred at 100° C. When the aryl halide starting material was completely consumed as determined by liquid chromatography-mass spectrometry (LC-MS), the reaction mixture was cooled to room temperature. Dioxane was removed under reduced pressure. The resulting crude mixture was diluted with water and extracted three times with an equal volume of DCM. The organic layers were pooled and dried over sodium sulfate. Solvent was removed under reduced pressure to give the second crude material which was purified by flash chromatography to give the final product.

Procedure 3, Borylations. An aryl halide (1 eq.), bis boronic acid (3 eq.), XPhos-Pd-G2 (0.01 eq.), XPhos (0.02 eq.) and KOAc (3 eq.) were combined in a screw cap vial, degassed, and charged with N2. Ethanol (volume to make. 1M solution in aryl halide) was added to the vial. The mixture was heated to 80° C. LC-MS was used to monitor the reaction progress. When the aryl halide had been completely consumed, the reaction mixture was cooled to room temperature and the solvent was removed under reduced pressure. The crude product was taken up in water and extracted three times with an equal volume of DCM. The organic layers were collected, pooled, and dried over sodium sulfate. The solvent was removed under reduced pressure to yield the second crude which was purified by flash chromatography to afford the final product.

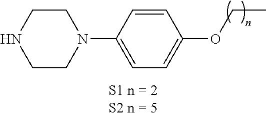

1-(4-Propoxyphenyl)piperazine (S1) δ 1-(4-(hexyloxy)phenyl)piperazine (S2). Tert-butyl 4-(4-hydroxyphenyl)piperazine-1-carboxylate (1 eq.) and the corresponding bromoalkane (3 eq.) were dissolved in acetone (0.3M). Potassium carbonate (1.5 eq.) was then added, and the mixture was stirred and heated to reflux for 16 h. Solvent was removed under reduced pressure and the residue was taken up into DCM and washed with 1 M aqueous NaOH, water, and brine. The organic layer was dried over sodium sulfate and concentrated under reduced pressure. The resulting solid was dissolved in DCM to which was added trifluoracetic acid (TFA, 0.4 eq. by volume to DCM). This solution was stirred at room temperature for 2 h. Solvent was removed under reduced pressure and the crude product was dissolved in DCM, washed with 1 M sodium carbonate, water, and brine. The organic layer was dried over sodium sulfate and concentrated under reduced pressure. The crude product was purified by flash chromatography to give compounds S1 and S2 (58% and 50% yield, respectively).

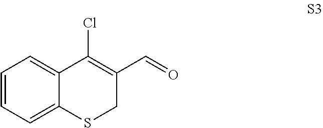 [SMILES: O=CC1=C(Cl)c2ccccc2SC1]

4-Chloro-2H-thiochromene-3-carbaldehyde (S3). POCl₃ (0.57 mL, 6.1 mmol) was added dropwise to DMF (6 mL). The solution was chilled to 0° C. and thiochroman-4-one (1.0 g, 6.1 mmol) was added drop wise. The solution was warmed to room temperature and stirred for 16 h. The solution was poured into ice cold water (100 mL) to quench the reaction. This solution was extracted 3 times with DCM (100 mL each). The organic layers were pooled and dried over sodium sulfate. The solvent was removed under reduced pressure and the resulting product was taken to the next step without further purification (1.1 g, 82% yield).

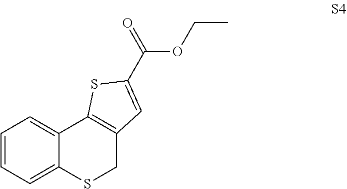 [SMILES: CCOC(=O)c1cc2c(s1)-c1ccccc1SC2]

Ethyl 4H-thieno[3,2-c]thiochromene-2-carboxylate (S4). Sodium metal (0.16 g, 6.9 mmol) was slowly added to EtOH (20 mL) and dissolved completely before chilling the solution to 0° C. Ethyl thioglycolate (0.28 mL, 6.6 mmol) was added dropwise followed by compound S3. The solution was warmed to room temperature while stirring for 16 h. The solution was then heated to 70° C. for 2 h. The solvent was removed under reduced pressure and the resulting crude was dissolved in EtOAc (50 mL), washed with water and brine (50 mL each), and dried over sodium sulfate. The second crude product was concentrated under reduced pressure and purified by flash chromatography (6:1 hexane: EtOAc) to obtain compound S4 (0.85 g, 60% yield).

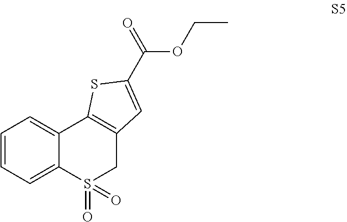 [SMILES: CCOC(=O)c1cc2c(s1)-c1ccccc1S(=O)(=O)C2]

Ethyl 4H-thieno[3,2-c]thiochromene-2-carboxylate 5,5-dioxide (S5). Compound S4 (2.4 g, 8.8 mmol) was dissolved in acetic acid (24 mL) and heated to 60° C. NaBO₃·4H₂O (6.8 g, 44 mmol) was added slowly. The solution was stirred at 60° C. for 5 h. The solution was cooled, and solvent was removed under reduced pressure. The residue was washed with water and dried to obtain compound S5 which was taken to the next step without further purification (1.8 g, 65% yield).

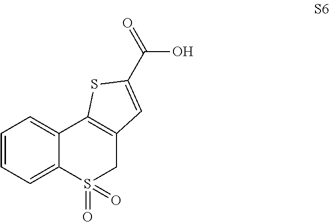 [SMILES: O=C(O)c1cc2c(s1)-c1ccccc1S(=O)(=O)C2]

4H-Thieno [3,2-c]thiochromene-2-carboxylic acid 5,5-dioxide (S6). To a solution of compound S5 (1.2 g, 3.9 mmol) in THF (30 mL) was added water (10 mL) and NaOH (1.2 g, 29 mmol). The resulting solution was heated to 80° C. and stirred for 3 h. THF was removed under reduced pressure and the aqueous solution was acidified with 6 M HCl (7 mL). The precipitated solid was collected by vacuum filtration and air dried to obtain compound S6 (1.1 g, 99% yield).

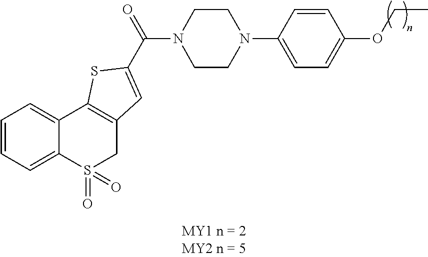

(5,5-Dioxido-4H-thieno[3,2-c]thiochromen-2-yl) (4-(4-propoxyphenyl)piperazin-1-yl) methanone (MY1) & (5,5-dioxido-4H-thieno[3,2-c]thiochromen-2-yl) (4-(4-(hexyloxy)phenyl)piperazin-1-yl) methanone (MY2). MY1 and 2 were synthesized from compound S6 and compounds S1 and S2 respectively by procedure 1 (52% and 86% yield respectively). MY1: ¹H NMR (500 MHZ, CDCl₃) δ 8.05 (dd, J=7.8, 1.3 Hz, 1H), 7.71-7.60 (m, 2H), 7.54 (td, J=7.5, 1.3 Hz, 1H), 7.21 (s, 1H), 6.94-6.88 (m, 2H), 6.88-6.84 (m, 2H), 4.44 (s, 2H), 3.95-3.91 (m, 4H), 3.88 (t, J=6.6 Hz, 2H), 3.18-3.04 (m, 4H), 1.87-1.71 (m, 2H), 1.03 (t, J=7.4 Hz, 3H). ¹³C NMR (126 MHz, CDCl₃) δ 162.16, 154.17, 144.89, 137.41, 136.23, 133.98, 133.62, 130.10, 129.82, 129.27, 127.85, 125.87, 124.29, 119.06, 115.30, 69.93, 51.27, 51.22, 22.67, 10.55. HRMS (ESI) calc. for [M+H]⁺ C₂₅H₂₇N₂O₄S₂ 483.14068, obs. 483.41061. MY2: ¹H NMR (500 MHZ, CDCl₃) δ 8.06 (dd, J=7.8, 1.2 Hz, 1H), 7.66 (ddd, J=18.3, 7.7, 1.3 Hz, 2H), 7.55 (td, J=7.5, 1.4 Hz, 1H), 7.24 (s, 1H), 6.96-6.90 (m, 2H), 6.90-6.85 (m, 2H), 4.46 (s, 2H), 3.94 (td, J=6.7, 5.9, 2.7 Hz, 6H), 3.19-3.09 (m, 4H), 1.84-1.71 (m, 2H), 1.47 (ddd, J=8.2, 5.3, 2.7 Hz, 2H), 1.35 (ddd, J=7.2, 4.6, 3.3 Hz, 4H), 0.96-0.87 (m, 3H). ¹³C NMR (126 MHZ, CDCl₃) δ 162.1, 154.2, 144.9, 137.4, 136.2, 134.0, 133.6, 130.1, 130.0, 129.3, 127.9, 125.9, 124.3, 119.0, 115.3, 68.4, 51.2, 51.2, 31.6, 29.3, 25.8, 22.6, 14.1. HRMS (ESI) calc. for [M+H]⁺ C₂₈H₃₃N₂O₄S2 525.18763, obs. 525.18730.

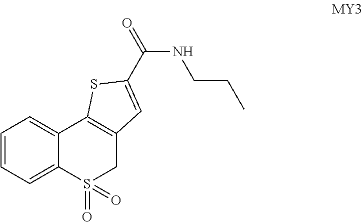 [SMILES: CCCNC(=O)c1cc2c(s1)-c1ccccc1S(=O)(=O)C2]

N-Propyl-4H-thieno[3,2-c]thiochromene-2-carboxamide 5,5-dioxide (MY3). MY3 was synthesized from propylamine and compound S6 by procedure 1 (70% yield). 1H NMR (500 MHz, DMSO) δ 8.72 (t, J=5.7 Hz, 1H), 7.96 (dd, J=7.6, 1.1 Hz, 1H), 7.83-7.78 (m, 2H), 7.77 (s, 1H), 7.65 (ddd, J=8.5, 6.8, 2.0 Hz, 1H), 4.94 (s, 2H), 3.27-3.19 (m, 2H), 1.54 (q, J=7.2 Hz, 2H), 0.90 (t, J=7.4 Hz, 3H). ¹³C NMR (126 MHz, DMSO) δ 160.7, 140.6, 135.9, 134.7, 134.2, 130.8, 130.3, 129.9, 129.7, 126.5, 123.8, 50.9, 41.5, 22.8, 11.9. HRMS (ESI) calc. for [M+H]⁺ C₁₅H₂₆NO₃S₂ 322.05661, obs. 322.05640.

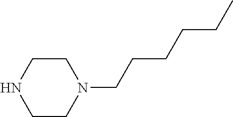

1-hexylpiperazine (S7). A solution of piperazine (1.2 g, 14 mmol) and 1-bromohexane (0.50 mL, 3.6 mmol) in EtOH (30 mL) was heated to reflux for 16 h. Solvent was removed under reduced pressure. The residue was re-dissolved in DCM (50 mL) and washed with saturated aqueous sodium bicarbonate and brine (50 mL each). The organic layer was dried over sodium sulfate and concentrated under reduced pressure. Crude material was purified by flash chromatography to give compound S7 (0.51 g, 85% yield).

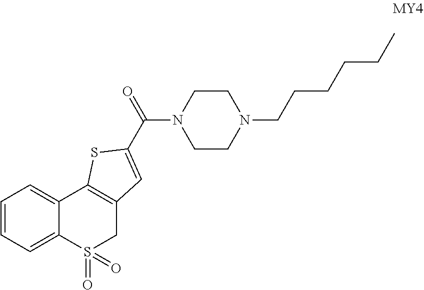 [SMILES: CCCCCCN1CCN(C(=O)c2cc3c(s2)-c2ccccc2S(=O)(=O)C3)CC1]

(5,5-dioxido-4H-thieno[3,2-c]thiochromen-2-yl) (4-hexylpiperazin-1-yl) methanone (MY4). MY4 was synthesized from compounds S6 and S7 by procedure 1 (82% yield). ¹H NMR (500 MHZ, CDCl₃) δ 8.04 (dd, J=7.9, 1.3 Hz, 1H), 7.66 (dd, J=7.4, 1.3 Hz, 1H), 7.62 (dd, J=7.9, 1.3 Hz, 1H), 7.54 (td, J=7.5, 1.3 Hz, 1H), 7.18 (s, 1H), 4.44 (s, 2H), 3.79 (t, J=5.0 Hz, 4H), 2.50 (t, J=4.9 Hz, 4H), 2.44-2.31 (m, 2H), 1.51 (t, J=7.5 Hz, 2H), 1.40-1.21 (m, 6H), 0.96-0.83 (m, 3H). ¹³C NMR (126 MHz, CDCl₃) δ 162.0, 137.6, 136.0, 133.9, 133.6, 130.1, 129.7, 129.2, 127.8, 125.8, 124.2, 58.6, 53.2, 51.2, 31.8, 27.2, 26.7, 22.6, 14.1. HRMS (ESI) calc. for [M+H]⁺ C₂₂H₂₉N₂O₃S₂ 433.16141, obs. 433.16133.

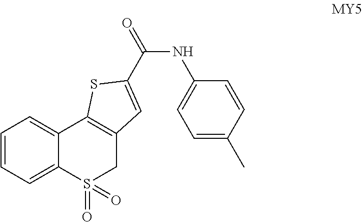 [SMILES: Cc1ccc(NC(=O)c2cc3c(s2)-c2ccccc2S(=O)(=O)C3)cc1]

N-(p-tolyl)-4H-thieno[3,2-c]thiochromene-2-carboxamide 5,5-dioxide (5). MY5 was synthesized from p-toluidine hydrochloride and compound S6 by procedure 1 (91% yield). ¹H NMR (500 MHZ, DMSO) δ 10.41 (s, 1H), 8.03 (s, 1H), 7.98 (dd, J=7.9, 1.2 Hz, 1H), 7.86 (dd, J=7.9, 1.2 Hz, 1H), 7.81 (td, J=7.6, 1.3 Hz, 1H), 7.67 (td, J=7.6, 1.3 Hz, 1H), 7.64-7.60 (m, 2H), 7.18 (d, J=8.2 Hz, 2H), 5.00 (s, 2H), 2.29 (s, 3H). ¹³C NMR (126 MHz, DMSO) δ 159.4, 140.5, 136.9, 136.4, 134.7, 134.3, 133.6, 130.9, 130.7, 130.1, 129.6, 126.6, 123.8, 120.8, 50.9, 21.0. HRMS (ESI) calc. for [M+H]⁺ C₁₉H₁₅NO₃S₂ 370.05661, obs. 370.05646.

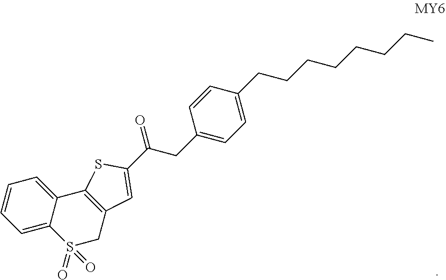 [SMILES: CCCCCCCCc1ccc(CC(=O)c2cc3c(s2)-c2ccccc2S(=O)(=O)C3)cc1]

N-(4-octylphenyl)-4H-thieno[3,2-c]thiochromene-2-carboxamide 5,5-dioxide (MY6). MY6 was synthesized from 4-octylaniline and compound S6 by procedure 1 (85% yield). ¹H NMR (500 MHz, CDCl₃) δ 8.23 (s, 1H), 7.97 (dd, J=7.7, 1.2 Hz, 1H), 7.53-7.45 (m, 2H), 7.45-7.37 (m, 4H), 7.08-7.02 (m, 2H), 4.34 (s, 2H), 2.54 (dd, J=8.8, 6.7 Hz, 2H), 1.56 (d, J=7.2 Hz, 2H), 1.36-1.20 (m, 10H), 0.89 (t, J=6.9 Hz, 3H). ¹³C NMR (126 MHz, CDCl₃) δ 158.8, 140.2, 139.5, 137.9, 134.9, 134.1, 133.0, 130.0, 129.3, 128.8, 128.6, 128.2, 126.1, 124.1, 120.1, 51.2, 35.4, 31.9, 31.6, 29.5, 29.4, 29.3, 22.7, 14.2. HRMS (ESI) calc. for [M+H]⁺ C₂₆H₃₀NO₃S₂ 468.16616, obs. 468.16606.

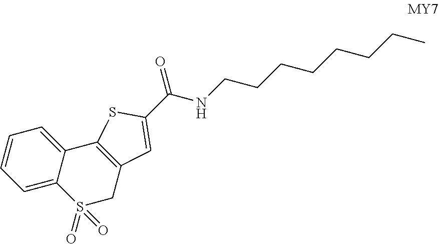 [SMILES: CCCCCCCCNC(=O)c1cc2c(s1)-c1ccccc1S(=O)(=O)C2]

N-octyl-4H-thieno[3,2-c]thiochromene-2-carboxamide 5,5-dioxide (MY7). MY7 was synthesized from octylamine and compound S6 by procedure 1 (77% yield). ¹H NMR (500 MHz, CDCl₃) δ 8.04 (dd, J=7.8, 1.3 Hz, 1H), 7.70-7.61 (m, 2H), 7.54 (td, J=7.5, 1.4 Hz, 1H), 7.34 (s, 1H), 6.09 (dt, J=11.5, 6.0 Hz, 1H), 4.42 (s, 2H), 3.40 (td, J=7.2, 5.8 Hz, 2H), 1.64-1.51 (m, 3H), 1.40-1.19 (m, 12H), 0.88 (t, J=6.8 Hz, 3H). ¹³C NMR (126 MHz, CDCl₃) δ 160.7, 139.6, 137.1, 134.0, 133.7, 130.3, 129.3, 128.4, 128.3, 125.9, 124.3, 51.3, 40.3, 31.8, 29.6, 29.3, 29.2, 27.0, 22.7, 14.1. HRMS (ESI) calc. for [M+H]⁺ C₂₀H₂₆NO₃S₂ 392.13486, obs. 392.13462.

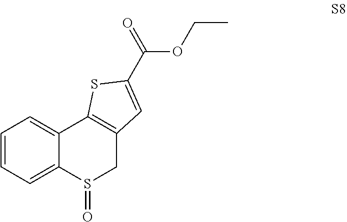 [SMILES: CCOC(=O)c1cc2c(s1)-c1ccccc1S(=O)C2]

Ethyl 4H-thieno[3,2-c]thiochromene-2-carboxylate 5-oxide (S8). To a solution of compound S4 (1.0 g, 3.6 mmol) in DCM (40 mL) was added meta-chloroperoxybenzoic acid (m-CPBA, 0.81 g, 3.6 mmol). The reaction mixture was stirred at room temperature for 1 h then washed twice with saturated aqueous sodium bicarbonate and brine (15 mL each). The organic layer was dried over sodium sulfate and concentrated under reduced pressure. The crude product was purified by flash chromatography (1:1→5:6 hexane: EtOAc) to give compound S8 (78% yield).

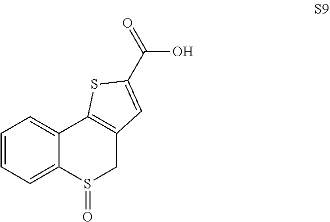 [SMILES: O=C(O)c1cc2c(s1)-c1ccccc1S(=O)C2]

4H-Thieno [3,2-c]thiochromene-2-carboxylic acid 5-oxide (S9). Compound S8 (0.20 g, 0.68 mmol) was dissolved in MeOH (7 mL) and 1 M aqueous NaOH (3 mL) was added. The mixture was stirred at room temperature for 2 h then heated to 60° C. for 1 h. MeOH was removed under reduced pressure and the crude material was diluted with water (5 mL). The solution was acidified with 6 M HCl (1 mL) and the resulting precipitate was collected by vacuum filtration to give compound S9 (0.18 g, 99% yield).

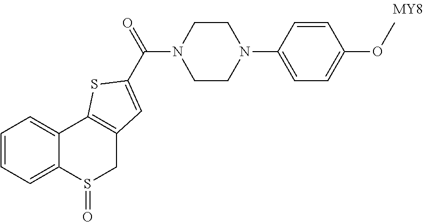 [SMILES: COc1ccc(N2CCN(C(=O)c3cc4c(s3)-c3ccccc3S(=O)C4)CC2)cc1]

(4-(4-Methoxyphenyl)piperazin-1-yl) (5-oxido-4H-thieno[3,2-c]thiochromen-2-yl) methanone (MY8). MY8 was synthesized from 1-(4-methoxyphenyl)piperazine and compound S9 by procedure 1 (43% yield). ¹H NMR (500 MHZ, CDCl₃) § 7.88 (dd, J=7.6, 1.3 Hz, 1H), 7.67-7.59 (m, 2H), 7.52 (td, J=7.4, 1.6 Hz, 1H), 7.28 (s, 1H), 6.97-6.92 (m, 2H), 6.91-6.86 (m, 2H), 4.49 (d, J=15.3 Hz, 1H), 4.00-3.92 (m, 5H), 3.80 (s, 3H), 3.14 (t, J=5.1 Hz, 4H). ¹³C NMR (126 MHz, CDCl₃) δ 162.5, 154.6, 145.1, 137.9, 136.9, 135.3, 133.1, 130.8, 129.4, 129.3, 128.1, 125.6, 125.5, 119.0, 114.6, 55.6, 51.2, 47.4. HRMS (ESI) calc. for [M+H]⁺ C₂₃H₂₃N₂O₃S₂ 439.11446, obs. 439.11415.

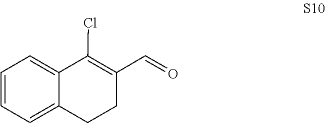 [SMILES: O=CC1=C(Cl)c2ccccc2CC1]

1-chloro-3,4-dihydro-2-naphthaldehyde (S10). POCl₃ (0.67 mL, 7.3 mmol) was added dropwise to DMF (6 mL). The solution was chilled to 0° C. and alpha tetralone (0.91 mL, 6.8 mmol) was added drop wise. The solution was warmed to room temperature then heated to 80° C. for 1.5 h. The solution was cooled to room temperature then poured slowly into 1 M aqueous sodium acetate (50 mL) to quench the reaction. This solution was extracted 3 times with DCM (50 mL each). The organic layers were pooled and dried over sodium sulfate. The solvent was removed under reduced pressure and the resulting product was taken to the next step without further purification.

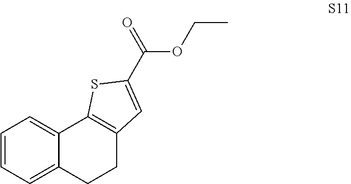 [SMILES: CCOC(=O)c1cc2c(s1)-c1ccccc1CC2]

Ethyl 4,5-dihydronaphtho[1,2-b]thiophene-2-carboxylate (S11). Sodium metal (0.20 g, 8.7 mmol) was slowly added to ethanol (20 mL) and dissolved completely before chilling the solution to 0° C. Ethyl thioglycolate (0.83 mL, 7.5 mmol) was added dropwise followed by compound S10 from the previous reaction. The solution was warmed to room temperature while stirring for 16 h. The solution was then heated to 70° C. for 2 h. The solvent was then removed under reduced pressure and the resulting crude was dissolved in DCM (15 mL), washed with water and brine (15 mL each), and dried over sodium sulfate. The second crude product was concentrated under reduced pressure and purified by flash chromatography (hexane→3:2 hexane: DCM) to give compound S11 (1.0 g, 3.9 mmol, 57% yield over two steps). ¹H NMR (500 MHZ, CDCl₃) § 7.62 (s, 1H), 7.45 (dt, J=6.1, 1.7 Hz, 1H), 7.29-7.22 (m, 3H), 4.39 (q, J=7.1 Hz, 2H), 2.99 (dd, J=8.8, 6.5 Hz, 2H), 2.86 (dd, J=8.6, 6.3 Hz, 2H), 1.42 (t, J=7.2 Hz, 3H). ¹³C NMR (126 MHz, CDCl₃) δ 162.5, 143.1, 137.7, 135.4, 133.6, 130.7, 130.5, 128.3, 128.3, 127.2, 123.6, 61.1, 28.9, 23.8, 14.4.

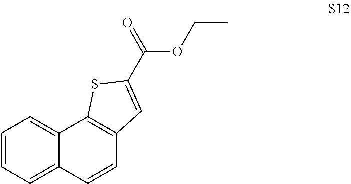 [SMILES: CCOC(=O)c1cc2ccc3ccccc3c2s1]

Ethyl naphtho[1,2-b]thiophene-2-carboxylate (S12). Compound S11 (1.0 g, 3.9 mmol), N-bromosuccinimide (NBS, 0.69 g, 3.9 mmol) and benzoyl peroxide (BPO, 1.0 mg, 0.041 mmol) were dissolved in CCl₄ (30 mL) and heated to reflux for 2 h. Additional NBS and BPO (0.69 g, 3.9 mmol and 1.0 mg, 0.041 mmol respectively) were added at this time and the mixture refluxed again for 2 h. The mixture was cooled and vacuum filtered to remove the solid. The filtrate was concentrated under reduced pressure and the crude material was purified by flash chromatography (hexane→3:17 ethylacetate (EtOAc):hexane) to give compound S12 (0.88 g, 3.4 mmol, 89% yield). ¹H NMR (500 MHZ, CDCl₃) δ 8.21-8.10 (m, 2H), 7.92 (dd, J=6.1, 3.7 Hz, 1H), 7.80 (dd, J=8.7, 2.3 Hz, 1H), 7.74 (dd, J=8.8, 2.2 Hz, 1H), 7.60 (qd, J=7.2, 4.9 Hz, 2H), 4.52-4.40 (m, 2H), 1.48 (t, J=7.2 Hz, 3H). ¹³C NMR (126 MHz, CDCl₃) δ 162.8, 141.2, 136.6, 132.6, 131.8, 131.3, 128.9, 128.7, 127.0, 127.0, 126.2, 123.9, 122.7, 61.6, 14.4.

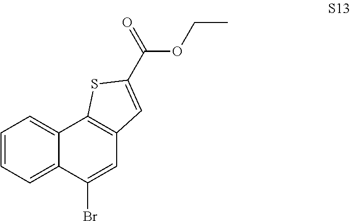 [SMILES: CCOC(=O)c1cc2cc(Br)c3ccccc3c2s1]

Ethyl 5-bromonaphtho[1,2-b]thiophene-2-carboxylate (S13). Compound S12 (0.88 g, 3.4 mmol) was dissolved in the acetic acid (17 mL) and bromine was added (0.2 mL, 3.9 mmol). The solution was heated to reflux for 4 h. After cooling to room temperature, the liquid was poured into water (100 mL) and extracted three times with EtOAc (100 mL each). The organic fractions were pooled, dried over anhydrous sodium sulfate, and concentrated under reduced pressure. The crude product was purified by flash chromatography (hexane→2:3 DCM:hexane) to give compound S13 (0.52 g, 1.6 mmol, 47% yield). ¹H NMR (500 MHZ, CDCl₃) δ 8.37 (dd, J=8.3, 1.4 Hz, 1H), 8.21-8.12 (m, 2H), 8.09 (s, 1H), 7.75-7.65 (m, 2H), 4.47 (q, J=7.1 Hz, 2H), 1.47 (t, J=7.1 Hz, 3H). ¹³C NMR (126 MHz, CDCl₃) δ 162.4, 140.8, 136.7, 133.5, 130.2, 130.0, 129.3, 128.6, 128.1, 128.0, 126.4, 124.3, 120.8, 61.8, 14.4.

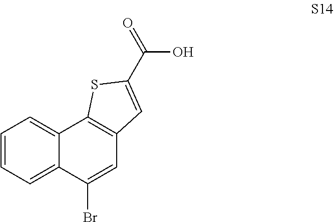 [SMILES: O=C(O)c1cc2cc(Br)c3ccccc3c2s1]

5-Bromonaphtho[1,2-b]thiophene-2-carboxylic acid (S14). Compound S13 (0.52 g, 1.6 mmol) was dissolved in tetrahydrofuran (THF, 7 mL) to which was added a 2 M aqueous NaOH solution (7 mL). The mixture was heated to reflux for 16 h. After cooling, THF was removed under reduced pressure. The remaining aqueous solution was acidified with 6 M HCl (5 mL). The precipitated compound S15 was collected by vacuum filtration and taken to the next step without further purification (0.46 g, 1.5 mmol, 94% yield). 1H NMR (500 MHz, DMSO) § 13.63 (s, 1H), 8.45-8.40 (m, 1H), 8.30-8.23 (m, 2H), 8.23-8.19 (m, 1H), 7.83-7.75 (m, 2H). ¹³C NMR (126 MHz, DMSO) δ 163.5, 140.3, 137.4, 135.2, 131.0, 129.5, 129.2, 129.1, 129.0, 128.3, 127.3, 125.0, 120.2.

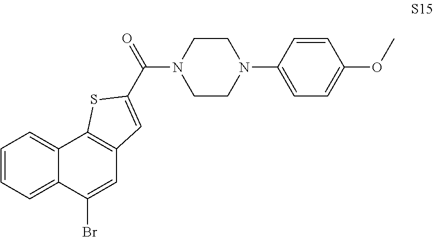 [SMILES: COc1ccc(N2CCN(C(=O)c3cc4cc(Br)c5ccccc5c4s3)CC2)cc1]

(5-Bromonaphtho[1,2-b]thiophen-2-yl) (4-(4-methoxyphenyl)piperazin-1-yl) methanone (S15). Compound S15 was synthesized from compound S14 and 1-(4-methoxyphenyl)piperazine by procedure 1 (47% yield). ¹H NMR (500 MHZ, CDCl₃) δ 8.43-8.33 (m, 1H), 8.16-8.11 (m, 2H), 7.73-7.65 (m, 2H), 7.60 (s, 1H), 6.99-6.94 (m, 2H), 6.91-6.87 (m, 2H), 4.07-3.95 (m, 4H), 3.81 (s, 3H), 3.18 (t, J=5.1 Hz, 4H). ¹³C NMR (126 MHz, CDCl₃) δ 163.3, 154.6, 145.1, 138.4, 136.5, 136.5, 129.6, 129.6, 128.6, 127.9, 127.7, 126.0, 125.6, 124.2, 120.7, 119.1, 114.6, 55.6, 53.5, 51.3.

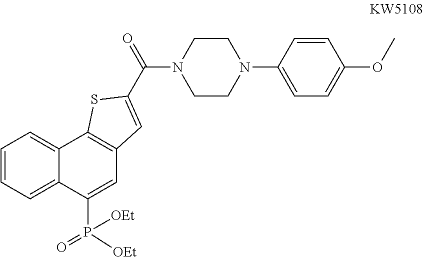 [SMILES: CCOP(=O)(OCC)c1cc2cc(C(=O)N3CCN(c4ccc(OC)cc4)CC3)sc2c2ccccc12]

Diethyl (2-(4-(4-methoxyphenyl)piperazine-1-carbonyl)naphtho[1,2-b]thiophen-5-yl)phosphonate (KW5108). Compound S15 (0.51 g, 1.1 mmol), diethyl phosphonate (0.20 mL, 1.5 mmol), N,N-diisopropylethylamine (DIPEA, 0.36 mL, 2 mmol), Pd(OAc)₂ (11 mg, 0.049 mmol) and triphenyl phosphine (33 mg, 0.13 mmol) were combined in ethanol (44 mL). The mixture was heated to reflux for 16 h. The reaction was cooled, and the solvent was removed under reduced pressure. The crude material was purified by flash chromatography (1:1 EtOAC: hexane, then 3:97 methanol: DCM) to give KW5108 (0.534 g, 0.99 mmol, 93% yield). ¹H NMR (500 MHZ, CDCl₃) δ 8.66 (d, J=17.1 Hz, 1H), 8.62-8.58 (m, 1H), 8.21 (dt, J=7.1, 2.4 Hz, 1H), 7.71 (s, 1H), 7.70-7.66 (m, 2H), 6.99-6.93 (m, 2H), 6.92-6.86 (m, 2H), 4.26 (dp, J=10.2, 7.2 Hz, 2H), 4.12 (ddq, J=10.1, 8.3, 7.1 Hz, 2H), 4.04-3.97 (m, 4H), 3.80 (d, J=0.9 Hz, 3H), 3.18 (t, J=5.0 Hz, 4H), 1.34 (t, J=7.0 Hz, 6H). ¹³C NMR (126 MHz, CDCl₃) δ 163.2, 154.6, 145.1, 143.5, 143.5, 136.7, 134.9, 134.8, 132.0, 131.9, 129.8, 129.7, 129.0, 128.9, 128.0, 128.0, 127.6, 127.5, 126.8, 126.8, 124.4, 124.4, 123.6, 122.2, 119.1, 114.6, 62.4, 62.3, 55.6, 51.3, 16.4, 16.4. ³¹P NMR (202 MHZ, CDCl₃) δ 19.2. HRMS (ESI) calc. for [M+H]⁺ C₂₈H₃₂N₂O₅PS 539.17641, obs. 539.17735.

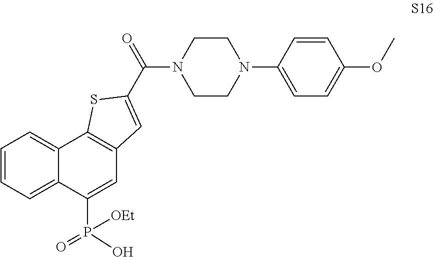 [SMILES: CCOP(=O)(O)c1cc2cc(C(=O)N3CCN(c4ccc(OC)cc4)CC3)sc2c2ccccc12]

Ethyl hydrogen (2-(4-(4-methoxyphenyl)piperazine-1-carbonyl)naphtho[1,2-b]thiophen-5-yl)phosphonate (S16). Compound 9 (0.50 g, 0.92 mmol) and LiBr (0.951 g, 11 mmol) were combined in acetone (9 mL) and heated to reflux for 3 days. Solvent was removed under reduced pressure. The crude material was purified by flash chromatography (DCM→1:9 MeOH:DCM) to give compound S16 (0.168 g, 0.33 mmol, 36% yield). 1H NMR (500 MHz, DMSO) δ 8.64 (dt, J=6.4, 3.5 Hz, 1H), 8.57 (d, J=16.5 Hz, 1H), 8.27 (dt, J=6.0, 2.5 Hz, 1H), 8.12 (s, 1H), 7.73 (dt, J=6.9, 3.5 Hz, 2H), 7.00-6.93 (m, 2H), 6.89-6.83 (m, 2H), 3.99-3.82 (m, 6H), 3.70 (s, 3H), 3.14 (t, J=5.2 Hz, 4H), 1.17 (t, J=7.0 Hz, 3H). ¹³C NMR (126 MHz, DMSO) δ 162.4, 153.9, 145.5, 141.8, 137.3, 135.7, 135.6, 130.8, 130.1, 128.6, 128.5, 128.2, 128.1, 124.7, 118.6, 114.8, 61.3, 55.7, 50.6, 16.8. ³¹P NMR (202 MHz, DMSO) δ 6.3.

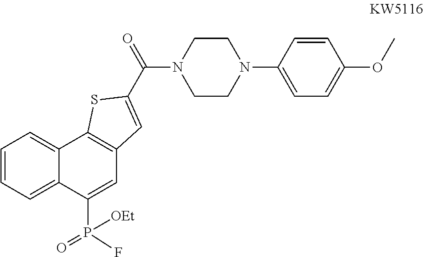 [SMILES: CCOP(=O)(F)c1cc2cc(C(=O)N3CCN(c4ccc(OC)cc4)CC3)sc2c2ccccc12]

Ethyl (2-(4-(4-methoxyphenyl)piperazine-1-carbonyl)naphtho[1,2-b]thiophen-5-yl)phosphonofluoridate (KW5116). Compound S16 (42 mg, 0.082 mmol) was dissolved in DCM (25 mL) to which (diethylamino) sulfur trifluoride (22 μL, 0.17 mmol) was added drop wise. The solution was stirred at room temperature for 3 h. Solvent was removed under reduced pressure. The resulting material was purified by flash chromatography (1:49 methanol: DCM) to give KW5116 (16 mg, 0.031 mmol, 38% yield). ¹H NMR (500 MHZ, CDCl₃) δ 8.61 (d, J=18.5 Hz, 1H), 8.45 (dq, J=7.9, 2.8 Hz, 1H), 8.22 (dt, J=7.2, 2.4 Hz, 1H), 7.74-7.68 (m, 3H), 6.97-6.91 (m, 2H), 6.90-6.85 (m, 2H), 4.50-4.36 (m, 2H), 3.98 (t, J=5.1 Hz, 4H), 3.78 (s, 3H), 3.17 (t, J=5.0 Hz, 4H), 1.45 (t, J=7.1 Hz, 3H). ¹³C NMR (126 MHz, CDCl₃) δ 162.9, 154.6, 145.1, 144.4, 144.4, 137.2, 134.6, 134.5, 131.9, 131.8, 131.8, 129.2, 129.1, 128.9, 128.8, 128.2, 128.0, 127.3, 127.3, 126.6, 124.6, 124.6, 119.1, 114.6, 77.3, 77.0, 76.8, 76.8, 64.4, 64.3, 55.6, 51.3, 29.7, 16.4, 16.4. ¹⁹F NMR (470 MHZ, CDCl₃) δ−60.3, −62.5. ³¹P NMR (202 MHZ, CDCl₃) δ 20.2, 15.1. HRMS (ESI) calc. for [M+H]⁺ C₂₆H₂₇FN₂O₄PS 513.14077, obs. 513.14041.

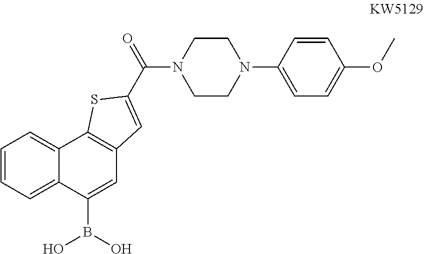 [SMILES: COc1ccc(N2CCN(C(=O)c3cc4cc(B(O)O)c5ccccc5c4s3)CC2)cc1]

(2-(4-(4-Methoxyphenyl)piperazine-1-carbonyl)naphtho[1,2-b]thiophen-5-yl)boronic acid (KW5129). KW5129 was synthesized from compound S15 by procedure 3 (34% yield). KW5129 was converted to the corresponding aryl trifluoroborate salt by treating it with KHF₂ to obtain NMR spectra due the compound having poor solubility in CD₃OD and forming a mixture of dehydrated adducts in other solvents. ¹H NMR (500 MHZ, DMSO) & 8.53 (d, J=8.0 Hz, 1H), 8.03-7.99 (m, 1H), 7.95 (d, J=3.7 Hz, 1H), 7.83 (s, 1H), 7.51-7.41 (m, 1H), 6.97 (d, J=6.6 Hz, 2H), 6.86 (d, J=8.6 Hz, 2H), 3.89 (s, 4H), 3.70 (s, 3H), 3.14 (t, J=5.0 Hz, 4H). ¹³C NMR (126 MHz, DMSO) δ 163.3, 137.2, 136.2, 135.7, 134.8, 131.9, 128.3, 128.0, 125.8, 125.4, 125.4, 125.2, 123.5, 118.6, 114.8, 55.7, 50.7, 46.2. ¹⁹F NMR (470 MHz, DMSO) δ−135.6. ¹¹B NMR (160 MHz, DMSO) δ 3.3. HRMS data was obtained from KW5129 directly. HRMS (ESI) calc. for [M+H]⁺ C₂₄H₂₄N₂O₄BS 447.15499, obs. 447.15580.

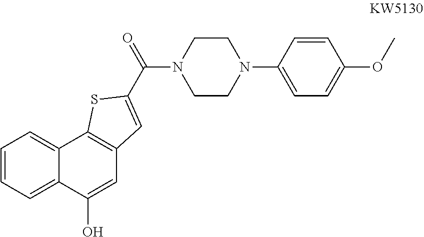 [SMILES: COc1ccc(N2CCN(C(=O)c3cc4cc(O)c5ccccc5c4s3)CC2)cc1]

(5-Hydroxynaphtho[1,2-b]thiophen-2-yl) (4-(4-methoxyphenyl)piperazin-1-yl) methanone (10A). KW5130 was isolated as a side product of the synthesis of KW5129 (9% yield). ¹H NMR (500 MHZ, DMSO) δ 10.34 (s, 1H), 8.27 (dd, J=8.2, 1.4 Hz, 1H), 8.07 (d, J=8.0 Hz, 1H), 7.81 (s, 1H), 7.66 (ddd, J=8.2, 7.0, 1.4 Hz, 1H), 7.60 (ddd, J=8.3, 6.9, 1.3 Hz, 1H), 7.27 (s, 1H), 6.96 (d, J=8.8 Hz, 2H), 6.91-6.80 (m, 2H), 3.87 (t, J=5.0 Hz, 4H), 3.70 (s, 3H), 3.12 (t, J=5.0 Hz, 4H). ¹³C NMR (126 MHz, DMSO) δ 162.9, 153.9, 152.1, 145.5, 137.5, 136.4, 129.3, 128.9, 128.0, 127.1, 126.3, 125.0, 124.0, 124.0, 118.6, 114.8, 103.6, 55.7, 55.4, 50.7. HRMS (ESI) calc. for [M+H]⁺ C₂₄H₂₃N₂O₃S 419.14239, obs. 419.14238.

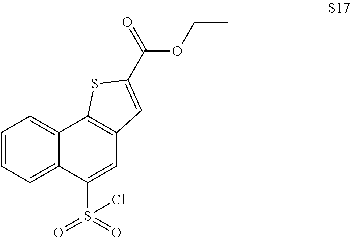 [SMILES: CCOC(=O)c1cc2cc(S(=O)(=O)Cl)c3ccccc3c2s1]

Ethyl 5-(chlorosulfonyl)naphtho[1,2-b]thiophene-2-carboxylate (S17). Chlorosulfonic acid (78 μL, 1.2 mmol) was added to chloroform (6 mL) and chilled to 0° C. In a separate vessel, compound S12 (0.10 g, 0.38 mmol) was dissolved in chloroform (3 mL) and added dropwise to the solution on ice. The solution was warmed gradually to room temperature and stirred for 4 h. The mixture was poured into ice cold water (50 ml) and stirred for 30 min. The water was saturated with NaCl and the precipitate that formed was decanted into a vacuum filter apparatus to collect the solid. This solid was purified by flash chromatography (acetone→1:9 MeOH:acetone) to give compound S17 (30 mg, 0.83 mmol, 22% yield). ¹H NMR (500 MHZ, DMSO) δ 9.01-8.96 (m, 1H), 8.45 (s, 1H), 8.39 (s, 1H), 8.24-8.19 (m, 1H), 7.68-7.65 (m, 2H), 4.39 (q, J=7.1 Hz, 2H), 1.37 (t, J=7.1 Hz, 3H). ¹³C NMR (126 MHz, DMSO) δ 162.3, 143.3, 141.5, 135.6, 133.1, 132.6, 129.7, 128.7, 128.4, 127.6, 127.4, 124.1, 121.8, 62.0, 14.7.

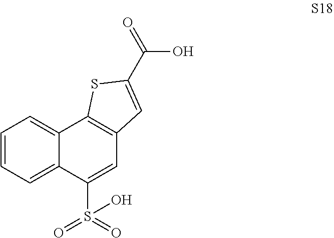 [SMILES: O=C(O)c1cc2cc(S(=O)(=O)O)c3ccccc3c2s1]

5-sulfonaphtho[1,2-b]thiophene-2-carboxylic acid (S18). Compound S17 (0.16 g, 0.44 mmol) was added to a 2M aqueous NaOH solution (8 mL) and stirred and room temperature for 16 h. The solution was acidified with 6 M HCl (5 mL) and saturated with NaCl to precipitate compound S18. The solid was collected by vacuum filtration and taken to the next step without further purification (0.053 g, 0.17 mmol, 39% yield) ¹H NMR (500 MHz, DMSO) δ 9.00-8.95 (m, 1H), 8.44 (s, 1H), 8.31 (s, 1H), 8.22-8.16 (m, 1H), 7.70-7.62 (m, 2H). ¹³C NMR (126 MHz, DMSO) δ 163.8, 143.0, 141.5, 135.7, 134.4, 132.6, 129.6, 128.7, 128.2, 127.6, 127.2, 124.1, 121.9.

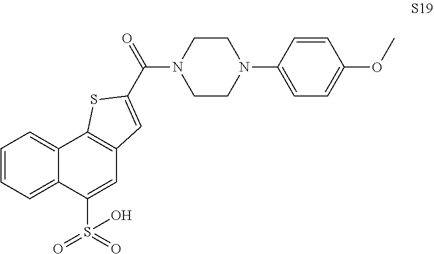 [SMILES: COc1ccc(N2CCN(C(=O)c3cc4cc(S(=O)(=O)O)c5ccccc5c4s3)CC2)cc1]

2-(4-(4-methoxyphenyl)piperazine-1-carbonyl)naphtho[1,2-b]thiophene-5-sulfonic acid (S19). Compound S19 was synthesized from compound S18 and 1-(4-methoxyphenyl)piperazine by procedure 1 (25% yield). ¹H NMR (500 MHZ, CDCl₃) δ 8.88 (d, J=8.5 Hz, 1H), 8.74 (d, J=2.0 Hz, 1H), 8.29 (dd, J=8.1, 1.5 Hz, 1H), 7.85 (ddd, J=8.6, 7.1, 1.4 Hz, 1H), 7.80 (ddd, J=8.1, 7.0, 1.2 Hz, 1H), 7.76 (d, J=2.1 Hz, 1H), 6.96-6.91 (m, 2H), 6.90-6.85 (m, 2H), 3.99 (t, J=5.1 Hz, 4H), 3.78 (s, 3H), 3.17 (t, J=5.1 Hz, 4H). ¹³C NMR (126 MHz, CDCl₃) δ 162.4, 154.7, 146.0, 145.0, 138.6, 138.0, 133.2, 129.4, 129.1, 128.8, 126.7, 126.6, 125.7, 124.8, 124.4, 119.1, 114.6, 55.6, 53.4, 50.9.

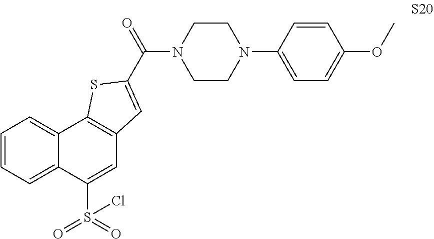 [SMILES: COc1ccc(N2CCN(C(=O)c3cc4cc(S(=O)(=O)Cl)c5ccccc5c4s3)CC2)cc1]

2-(4-(4-Methoxyphenyl)piperazine-1-carbonyl)naphtho[1,2-b]thiophene-5-sulfonyl chloride (S20). Compound S19 (0.24 g, 0.49 mmol) was dissolved in water (3 mL) and 2M aqueous NaOH (270 μL) was added. The solution was stirred at room temperature for 15 min then evaporated to dryness under reduced pressure. POCl₃ (5 mL) was added to the resulting solid and the mixture was stirred at room temperature for 16 h. The solution was concentrated under reduced pressure and the resulting product was taken to the next step without further purification.

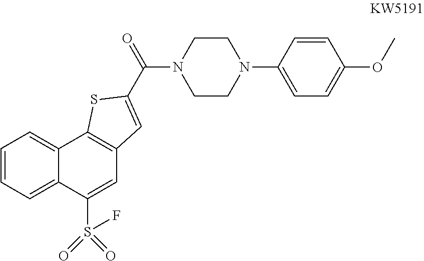 [SMILES: COc1ccc(N2CCN(C(=O)c3cc4cc(S(=O)(=O)F)c5ccccc5c4s3)CC2)cc1]

2-(4-(4-Methoxyphenyl)piperazine-1-carbonyl)naphtho[1,2-b]thiophene-5-sulfonyl fluoride (KW5191). Compound S20 (30 mg, 0.06 mmol) was dissolved in a 1:1 solution of acetonitrile and water (0.6 mL). In a separated vessel, KHF₂ (52 mg, 0.67 mmol) was dissolved in water (0.6 mL) and added to the first solution drop wise. The resulting solution was stirred at room temperature. The progress of the reaction was monitored by LC-MS. When all the starting material had been consumed, the solution was diluted with water (10 mL) and extracted with EtOAc 3 times (10 mL each). The organic fractions were pooled, dried over sodium sulfate, and concentrated under reduced pressure to give KW5191 (11 mg, 0.023 mmol, 38% yield). ¹H NMR (500 MHZ, CDCl₃) δ 8.74 (d, J=1.6 Hz, 1H), 8.62 (dt, J=8.1, 2.2 Hz, 1H), 8.28 (ddd, J=8.5, 5.3, 1.7 Hz, 1H), 7.88-7.77 (m, 2H), 7.76 (d, J=2.3 Hz, 1H), 6.97-6.91 (m, 2H), 6.90-6.85 (m, 2H), 4.02-3.96 (m, 4H), 3.79 (s, 3H), 3.17 (t, J=5.0 Hz, 4H). ¹³C NMR (126 MHz, CDCl₃) δ 162.4, 154.7, 146.0, 145.0, 138.5, 133.5, 133.2, 129.2, 128.8, 128.3, 126.5, 125.7, 124.8, 119.1, 114.6, 55.6, 51.3. ¹⁹F NMR (470 MHZ, CDCl₃) δ 62.8. HRMS (ESI) calc. for [M+H]⁺ C₂₄H₂₂N₂O₄S₂F 485.09995, obs. 485.09997.

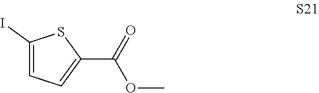

Methyl 5-iodo-2-thiophenecarboxylate (S21). Methyl-2-thiophenecarboxylate (0.18 g, 1.3 mmol), [bis(trifluoroacetoxy) iodo] benzene (0.32 g, 0.73 mmol), and iodine (0.17 g, 0.67 mmol) were dissolved in CCl₄ (1.8 mL) and stirred at room temperature for 2 h. The solvent was removed under reduced pressure. The crude product was purified by flash chromatography (hexane→1:3 DCM:hexane) to give compound S21 (0.20 g, 0.76 mmol, 58% yield). ¹H NMR (500 MHz, CDCl₃) δ 7.45 (d, J=3.9 Hz, 1H), 7.28 (d, J=3.9 Hz, 1H), 3.89 (s, 3H). ¹³C NMR (126 MHz, CDCl₃) δ 161.3, 139.2, 137.8, 134.5, 82.8, 52.3.

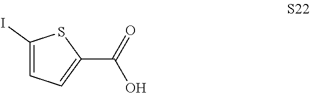

5-Iodo-2-thiophenecarboxylic acid (S22). LiOH (0.152 g, 3.6 mmol) was dissolved in water (5.7 mL) to which was added a solution of compound S21 (0.20 g, 0.76 mmol) in THF (0.76 mL). The mixture was stirred at room temperature for 4 h. The solution was acidified with 6 M HCl to pH 4 and then extracted with EtO Ac three times (10 mL each). The organic fractions were pooled, dried over sodium sulfate, and concentrated under reduced pressure to give compound S22, which was taken to the next step without further purification (0.131 g, 0.52 mmol, 68% yield). ¹H NMR (500 MHZ, DMSO) § 13.29 (s, 1H), 7.42 (d, J=3.8 Hz, 1H), 7.40 (d, J=3.8 Hz, 1H). ¹³C NMR (126 MHz, DMSO) δ 162.1, 140.6, 138.5, 135.1, 86.3.

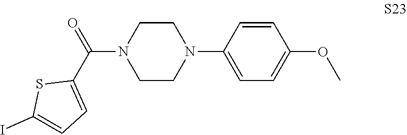 [SMILES: COc1ccc(N2CCN(C(=O)c3ccc(I)s3)CC2)cc1]

(5-Iodothiophen-2-yl) (4-(4-methoxyphenyl)piperazin-1-yl) methanone (S23). Compound S23 was synthesized from 1-(4-methoxyphenyl)piperazine and compound S22 by procedure 1 (60% yield). ¹H NMR (500 MHZ, CDCl₃) δ 7.21 (d, J=3.8 Hz, 1H), 6.99 (d, J=3.8 Hz, 1H), 6.93-6.88 (m, 2H), 6.87-6.83 (m, 2H), 3.91-3.84 (m, 4H), 3.77 (s, 3H), 3.13-3.05 (m, 4H). ¹³C NMR (126 MHz, CDCl₃) δ 162.2, 154.5, 145.1, 143.0, 136.7, 130.5, 119.0, 114.6, 55.6, 51.2, 45.5.

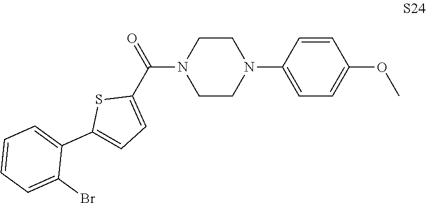 [SMILES: COc1ccc(N2CCN(C(=O)c3ccc(-c4ccccc4Br)s3)CC2)cc1]

(5-(2-Bromophenyl) thiophen-2-yl) (4-(4-methoxyphenyl)piperazin-1-yl) methanone (S24). Compound S24 was synthesized from compound S23 by procedure 2 (92% yield). ¹H NMR (500 MHZ, CDCl₃) δ 7.69 (dd, J=8.1, 1.2 Hz, 1H), 7.49 (dd, J=7.7, 1.7 Hz, 1H), 7.35 (td, J=7.5, 1.2 Hz, 1H), 7.33 (s, 1H), 7.24 (d, J=3.8 Hz, 1H), 7.22 (ddd, J=8.0, 7.5, 1.8 Hz, 1H), 6.95-6.90 (m, 2H), 6.88-6.83 (m, 2H), 3.95 (dd, J=6.0, 4.1 Hz, 4H), 3.78 (s, 3H), 3.17-3.08 (m, 4H). ¹³C NMR (126 MHZ, CDCl₃) δ 163.3, 154.5, 145.2, 145.1, 137.2, 134.3, 133.9, 131.9, 129.7, 129.0, 127.6, 127.4, 122.7, 119.0, 114.6, 55.6, 51.3, 45.1.

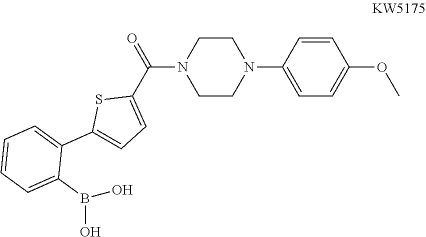 [SMILES: COc1ccc(N2CCN(C(=O)c3ccc(-c4ccccc4B(O)O)s3)CC2)cc1]

(2-(5-(4-(4-methoxyphenyl)piperazine-1-carbonyl) thiophen-2-yl)phenyl)boronic acid (KW5175). KW5175 was synthesized from compound S23 by procedure 3 (45% yield). ¹H NMR (500 MHZ, MeOD) δ 7.58 (dt, J=7.6, 1.0 Hz, 1H), 7.48-7.38 (m, 4H), 7.14 (d, J=3.8 Hz, 1H), 7.01-6.96 (m, 2H), 6.89-6.84 (m, 2H), 3.98-3.89 (m, 4H), 3.75 (s, 3H), 3.17-3.08 (m, 4H). ¹³C NMR (126 MHz, MeOD) δ 163.7, 154.7, 149.5, 145.2, 136.1, 135.9, 132.0, 130.3, 129.1, 127.8, 127.7, 124.2, 118.8, 114.1, 54.5, 51.0, 48.5. ¹¹B NMR (160 MHz, MeOD) δ 31.2. HRMS (ESI) calc. for [M+H]⁺ C₂₂H₂₄N₂O₄BS 423.1550 obs. 423.1150.

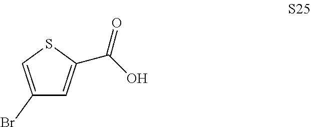

4-bromo-2-thiophenecarboxylic acid (S25). LiOH (0.48 g, 11 mmol) was dissolved in water (18 mL) to which was added a solution of methyl 4-bromo-2-thiophenecarboxylate (0.55 g, 2.5 mmol) in THF (2.5 mL). The mixture was stirred at room temperature for 4 h. The liquid was acidified to pH 4 with 6 M HCl and extracted three times with EtOAc (20 mL each). The organic fractions were pooled, dried over sodium sulfate, and concentrated under reduced pressure to give compound S25 which was taken to the next step without further purification (0.44 g, 2.1 mmol, 84% yield). ¹H NMR (500 MHZ, DMSO) δ 13.49 (s, 1H), 8.01 (s, 1H), 7.69 (s, 1H). ¹³C NMR (126 MHz, DMSO) δ 162.3, 136.6, 135.1, 131.3, 110.0.

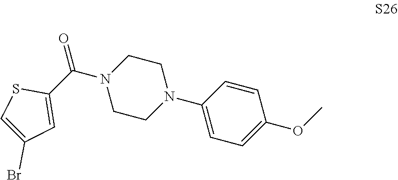 [SMILES: COc1ccc(N2CCN(C(=O)c3cc(Br)cs3)CC2)cc1]

(4-Bromothiophen-2-yl) (4-(4-methoxyphenyl)piperazin-1-yl) methanone (S26). Compound S26 was synthesized from compound S25 and 1-(4-methoxyphenyl)piperazine by procedure 1 (73% yield). ¹H NMR (500 MHZ, CDCl₃) δ 7.37 (s, 1H), 7.21 (s, 1H), 6.94-6.89 (m, 2H), 6.88-6.83 (m, 2H), 3.91-3.85 (m, 4H), 3.77 (s, 3H), 3.13-3.07 (m, 4H). ¹³C NMR (126 MHz, CDCl₃) δ 162.0, 154.5, 145.1, 138.3, 130.9, 126.2, 119.0, 114.6, 109.4, 55.6. 51.2. 46.0.

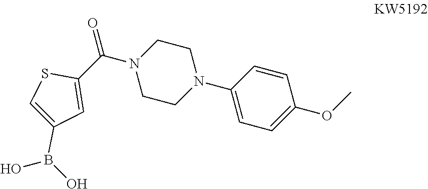 [SMILES: COc1ccc(N2CCN(C(=O)c3cc(B(O)O)cs3)CC2)cc1]

(5-(4-(4-Methoxyphenyl)piperazine-1-carbonyl) thiophen-3-yl)boronic acid (KW5192). KW5192 was synthesized by procedure 3 from compound S25 (59% yield). 1H NMR (500 MHz, MeOD) δ 8.05 (s, 1H), 7.64 (s, 1H), 7.01-6.91 (m, 2H), 6.90-6.81 (m, 2H), 3.89 (t, J=5.1 Hz, 4H), 3.74 (s, 3H), 3.08 (t, J=5.1 Hz, 4H). ¹³C NMR (126 MHZ, MeOD) δ 164.2, 154.7, 145.1, 137.7, 136.1, 134.1, 118.8, 114.1, 54.5, 51.0, 48.5. ¹¹B NMR (160 MHz, MeOD) δ 26.3. HRMS (ESI) calc. for [M+H]⁺ C₁₆H₂₀N₂O₄BS 347.1237, obs. 347.1235.

### Example 4

This example describes use of examples of compounds of the present disclosure as APT1 and/or APT2 inhibitors.

The IC₅₀ value for various compounds is shown in Table 2. The IC₅₀ values were determined by the methods described in Example 2.

TABLE 2

IC₅₀ (μM)

| Compound | APT1 | APT2 | |
|---|---|---|---|
| ML348 | ++ | − | |
| ML349 | − | ++ | |
| APT-MY1 | − | ++ | |
| APT-MY2 | − | + | |
| APT-MY3 | − | − | |
| APT-MY4 | − | + | |
| APT-MY5 | − | − | |
| APT-MY6 | − | − | |
| APT-MY7 | − | − | |
| APT-MY8 | − | + | |
| APT-MY9 | − | + | |
| APT-MY10 | − | − | |
| APT-MY11 | + | + | |
| | | (++ with 30 min | |
| | | preincubation) | |
| APT-MY12 | + | + | |
| KW05129 | ++ | +++ | |
| KW05175 | * | + | |
| KW05191 | * | + | |
| KW05192 | * | + | |
| KW06034 | ++ | ++ | |
| KW05126pp | − | + | |
| KW05151 | + | − | |
| KW05153 | − | − | |
| KW05169 | ++ | − | |
| KW05177 | − | + | |
| KW05179 | + | ++ | |
| *Not measured. | | | |
| IC₅₀ < 0.100 μ | | | : +++ ; |
| 0.100 μM < IC₅₀ < 1 μ | | | : ++ ; |
| 1 μM < IC₅₀ < 40 μ | | | : +; |

40 μM < IC₅₀: −

### Example 5

This example describes use of examples of compounds of the present disclosure as APT1 and/or APT2 inhibitors.

The IC₅₀ value for various compounds is shown in Table 3. The IC₅₀ values were determined by the methods described in Example 2.

TABLE 3

| Compound | R₁ | R₂ | R₃ | APT1 | APT2 | |
|---|---|---|---|---|---|---|
| KW05200 | H | H | CF₃ | − | ++ | |
| KW05201 | H | Cl | OMe | − | ++ | |
| KW05202 | H | C1 | CF₃ | − | + | |
| KW05203 | H | OMe | OMe | − | ++ | |
| KW05204 | Cl | H | CF₃ | − | ++ | |
| KW05205 | OMe | H | OMe | − | ++ | |
| KW05206 | OMe | H | CF₃ | − | ++ | |
| KW05207 | H | H | NH(CH₃) | − | + | |
| KW05208 | H | H | NMe₂ | − | + | |
| KW05209 | F | H | CF₃ | − | − | |
| KW05210 | H | H | CHF2 | − | − | |
| KW05211 | H | H | C(CH₃)₃ | − | ++ | |
| KW05212 | H | H | N(CH₃)₂ | − | + | |
| KW05213 | OH | H | CF₃ | − | ++ | |
| KW05214 | H | OH | CF₃ | − | − | |
| KW05215 | NH(CH₃) | H | OMe | − | − | |
| KW05216 | N(CH₃)₂ | H | OMe | − | − | |
| KW05217 | H | H | F | − | − | |
| KW05218 | H | H | CN | − | − | |
| KW05219 | H | H | NO₂ | − | − | |
| KW05220 | F | H | OMe | − | ++ | |
| KW05221 | H | F | OMe | − | ++ | |
| KW05222 | CN | H | OMe | − | ++ | |
| KW05223 | H | CN | OMe | − | ++ | |
| KW05224 | H | CN | CF₃ | − | − | |
| KW05225 | CN | H | CF₃ | − | ++ | |
| IC₅₀ < 0.100 μ | | | | | | : +++; |
| 0.100 μM < IC₅₀ < 1 μ | | | | | | : ++; |
| 1 μM < IC₅₀ < 40 μ | | | | | | : +; |

40 μM < IC₅₀: −

The R₁, R₂, and R₃ designations are based on the following structure:

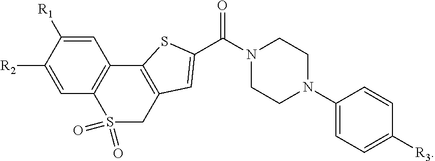

### Example 6

This example describes synthesis and characterization of examples of compounds of the present disclosure.

Method for KW05200. FIG. 15 shows a reaction scheme for KW05200. Step 1: To a solution of compound 0_1 (3.28 g, 20.00 mmol) in DMF (30 mL) were slowly added phosphorus oxychloride (1.86 mL, 20.00 mmol) on ice-bath. The resulting mixture was stirred for 0.5 h and then stirred at 80° C. for 6 h. LCMS showed that the compound 0_1 was consumed completely, and the desired product was detected. The reaction was quenched by water (20 mL) and stirred at room temperature for 30 min, and then the mixture solution was extracted with ethyl acetate (100 mL×3). The organic layers were combined, washed with brine and concentrated in vacuo to afford crude compound 0_2 (3.00 g, 14.29 mmol, 71%) as a brown solid which was used for next step without further purification. MS m/z (ESI):=211.1 [M+H]⁺.

Step 2: To a solution of crude compound 0_2 (3.00 g, 14.29 mmol) in CH3CN (30 mL) were slowly added ethyl 2-mercaptoacetate (1.71 g, 14.29 mmol) and K₂CO₃ (5.91 g, 42.87 mmol) on ice-bath, the resulting mixture was stirred at 0° C. for 0.5 h and at 60° C. for 4 h. LCMS showed that the compound 0_2 was consumed completely, and the desired product was formed. The reaction was diluted with water (120 mL) and extracted with ethyl acetate (150 mL). The organic layer was separated, washed with brine and concentrated in vacuo. The residue was purified using silica gel column chromatography (eluting with Petroleum ether:Ethyl acetate=30:1) to afford compound 0_3 (3.10 g, 11.23 mmol, 78%) as yellow solid. MS m/z (ESI):=276.8 [M+H]⁺.

Step 3: To a solution of compound 0_3 (3.10 g, 11.23 mmol) in DCM (30 mL) was slowly added 3-chloroperbenzoic acid (3.86 g, 22.46 mmol) on ice-bath, the resulting mixture was stirred at room temperature for 3.5 h. LCMS showed that the compound 0_3 was consumed completely, and the desired product was detected. The reaction was quenched with saturated aqueous sodium sulfite, diluted with water (150 mL) and extracted with ethyl acetate (150 mL×2). The organic layers were separated, combined and washed with brine and concentrated in vacuo. The residue was purified by silica gel column chromatography (eluted with Petroleum ether:Ethyl acetate=3:1) to give compound 0_4 (2.00 g, 6.49 mmol, 58%) as a yellow solid. MS m/z (ESI):=638.8 [2M+Na]+.

Step 4: To a solution of compound 0_4 (2.00 g, 6.49 mmol) in THF (10 mL)-MeOH (5 mL)-H₂O (5 mL) were slowly added lithium hydroxide monohydrate (545.0 mg, 12.98 mmol) on ice-bath, and the resulting mixture was stirred at room temperature for 0.5 h. LCMS showed that the compound 0_4 was consumed completely, and the desired product was detected. The reaction was diluted with water (150 mL) and washed with ethyl acetate (150 mL×2). The water layer was adjusted to pH 2 by 2N HCl. The mixture solution was diluted with water (60 mL) and extracted with ethyl acetate (50 mL×3). The organic layer were combined, washed with brine and concentrated to afford compound 0_5 (1.70 g, 6.07 mmol, 93%) as a white solid. MS m/z (ESI):=582.7 [2M+Na]+.

Step 5: To a solution of compound 0_5 (140.15 mg, 0.50 mmol) in DCM (3 mL) were added 1-(4-(trifluoromethyl)phenyl)piperazine (165.00 mg, 0.50 mmol), T₃P (50% in ethyl acetate, 471.2 mg, 0.38 mL, 1.50 mmol) and DIEA (0.26 mL, 1.50 mmol). The resulting mixture was stirred at room temperature for 5 h. TLC (DCM:MeOH=20:1) showed that most of compound 0_5 was consumed, and a new major spot was formed. The reaction was diluted with water (60 mL) and extracted with DCM (100 mL×3). The organic layers were combined, washed with brine and concentrated in vacuo. The residue was purified using silica gel column chromatography (DCM:MeOH=50:1˜20:1) to give compound KW05200 (17.48 mg, 0.09 mmol, 7.10%) as a yellow solid. MS m/z (ESI):=492.9 [M+H]⁺; ¹H NMR (400 MHZ, DMSO-d6) δ7.98 (d, J=7.8 Hz, 1H), 7.83-7.78 (m, 2H), 7.66 (ddd, J=8.3, 6.3, 2.3 Hz, 1H), 7.59-7.53 (m, 3H), 7.09 (d, J=8.6 Hz, 2H), 4.91 (s, 2H), 3.85 (t, J=5.3 Hz, 4H), 3.45 (dd, J=6.5, 4.0 Hz, 4H).

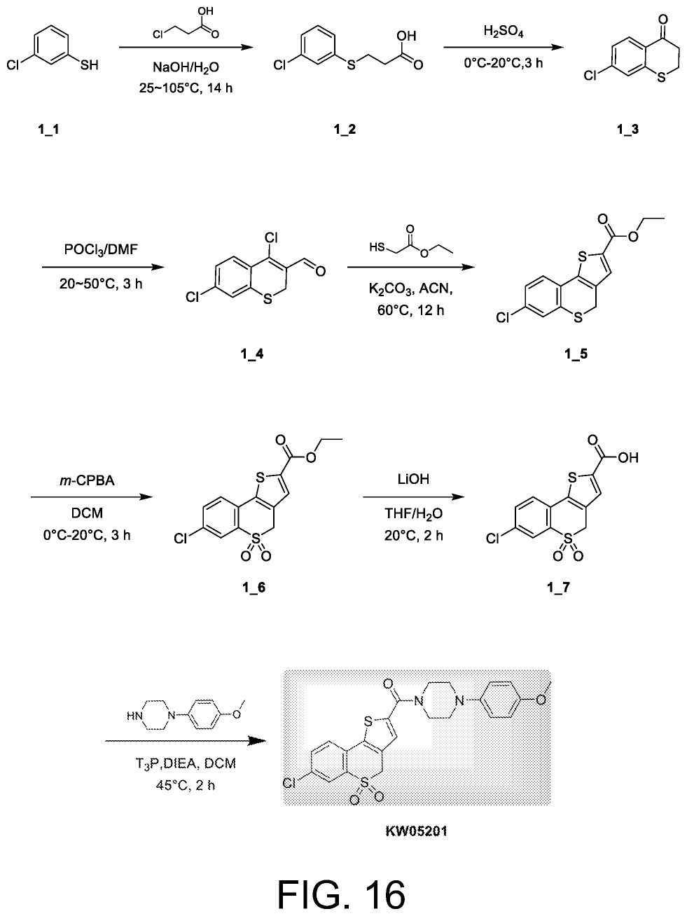

Method for KW05201. FIG. 16 shows a reaction scheme for KW05201. Step 1: To a mixture of NaOH (1 mol/L, 50.0 mL, 50.0 mmol) and Na₂CO₃ (1 mol/L, 50.0 mL, 50.0 mmol) solution was added a solution of compound 1_1 (12.0 mL, 104 mmol) in EtOH (60 mL) under N₂. The mixture was stirred at 25° C. for 5 minutes. Then a solution of 3-chloropropanoic acid (9.31 mL, 109 mmol) in H₂O (40.0 mL) was added dropwise to the reaction mixture under N₂. The resulting mixture was stirred at 25° C. for 2 h and then at 105° C. for 12 h. TLC (Petroleum ether:Ethyl acetate=3:1) indicated starting material was consumed and one major spot was formed. The mixture was concentrated under reduced pressure to give a residue. The residue was adjusted the pH=4 with 2 N HCl, diluted with water (150 mL) and extracted with ethyl acetate (200 mL×2). The combined organic layers were washed with brine (150 mL), dried and concentrated to give crude compound 1_2 (17.7 g, 81.7 mmol, 78.8%) as a white solid.

Step 2: Compound 1_2 (1.75 g, 8.08 mmol) was added to concentrated H₂SO₄ (4 mL) slowly at 0° C. The resulting mixture was stirred at 25° C. for 3 h. TLC (Petroleum ether:Ethyl acetate=5:1) indicated starting material was consumed and one major spot was formed. The reaction mixture was poured to ice water slowly and extracted with ethyl acetate (150 mL). The organic layer was washed with water and bine, dried with anhydrous Na₂SO₄, filtered and concentrated. The residue was purified by chromatography (SiO₂, Petroleum ether:Ethyl acetate=0:1) to give compound 1_3 (930 mg, 4.68 mmol, 57.96%) as a white solid.

Step 3: To a mixture of compound 1_3 (10.3 g, 51.8 mmol) in DMF (100 mL) was added POCl₃ (7.22 mL, 77.7 mmol) at 20° C. The resulting mixture was stirred at 50° C. for 3 h. TLC (Petroleum ether:Ethyl acetate=5:1) indicated starting material was consumed and one major spot was formed. The reaction was cooled to 0° C. and quenched with 30 mL of ice water. The mixture was filtered. The filter cake was washed with water (100 mL), then dried in vacuum to give compound 1_4 (12.1 g, 49.4 mmol, 95.3%) as a yellow solid.

Step 4: To a mixture of compound 1_4 (1 g, 4.08 mmol) and K₂CO₃ (0.85 g, 6.12 mmol) in acetonitrile (10.0 mL) was added ethyl sulfanylacetate (0.67 mL, 6.12 mmol). The resulting mixture was stirred at 60° C. for 12 h. TLC (Petroleum ether:Ethyl acetate=5:1) indicated starting material was consumed and one major spot was formed. The reaction was quenched by water (100 mL) and extracted with ethyl acetate (120 mL). The organic layer was dried and concentrated to give crude compound 1_5 (1.10 g, 3.54 mmol, 86.8%) as a yellow solid which was used for next step without further purification.

Step 5: To a solution of compound 1_5 (1.00 g, 3.22 mmol) in DCM (15.0 mL) was added m-CPBA (1.11 g, 6.43 mmol) at 0° C. The resulting mixture was stirred at 20° C. for 3 h. TLC (Petroleum ether:Ethyl acetate=2:1) indicated starting material was consumed and one major spot was formed. The reaction was quenched by saturated aqueous sodium sulfite solution (50.0 mL) and extracted with DCM (120 mL). The combined organic layers were dried and concentrated to give a residue. The residue was purified by chromatography (SiO₂, Petroleum ether:Ethyl acetate=50:1˜30/1) to give compound 1_6 (140 mg, 0.41 mmol, 12.7%) as a yellow solid.

Step 6: To a solution of 1_6 (140 mg, 0.41 mmol) in THF (2.00 mL) and H₂O (2.00 mL) was added LiOH·H₂O (51.4 mg, 1.23 mmol). The resulting mixture was stirred at 20° C. for 2 h. TLC (Petroleum ether:Ethyl acetate=3:1) showed starting material was consumed and one major spot was formed. The reaction solution was adjusted to pH=4 with saturated citric acid solution, diluted with water (30.0 mL) and extracted with ethyl acetate (50.0 mL×2). The combined organic layers were separated, washed with brine (30.0 mL), dried and concentrated to give crude compound 1_7 (120 mg, 0.38 mmol, 93.4%) as a yellow solid which was used for next step without further purification.

Step 7: To a solution of compound 1_7 (60 mg, 0.19 mmol), 1-(4-methoxyphenyl)piperazine (52.3 mg, 0.23 mmol) and DIEA (0.09 mL, 0.57 mmol) in ethyl acetate (2.00 mL) was added T₃P (50% in ethyl acetate, 182 mg, 146.8 μL, 0.57 mmol). The resulting mixture was stirred at 45° C. for 2 h. LCMS showed the reaction was completed. The mixture was quenched by water (20.0 mL) and extracted with DCM (30.0 mL). The combined organic layers were dried and concentrated to give a residue. The residue was triturated with ethyl acetate (1.00 mL) to give KW05201 (32.6 mg, 0.06 mmol, 33.0%) as a yellow solid. MS m/z (ESI):=489.1 [M+H]⁺. ¹H NMR (400 MHZ, DMSO-d6) δ=7.95 (s, 1H), 7.87-7.83 (m, 2H), 7.55 (s, 1H), 6.96-6.92 (m, 2H), 6.88-6.83 (m, 2H), 4.96 (s, 2H), 3.81 (br s, 4H), 3.69 (s, 3H), 3.12-3.06 (m, 4H).

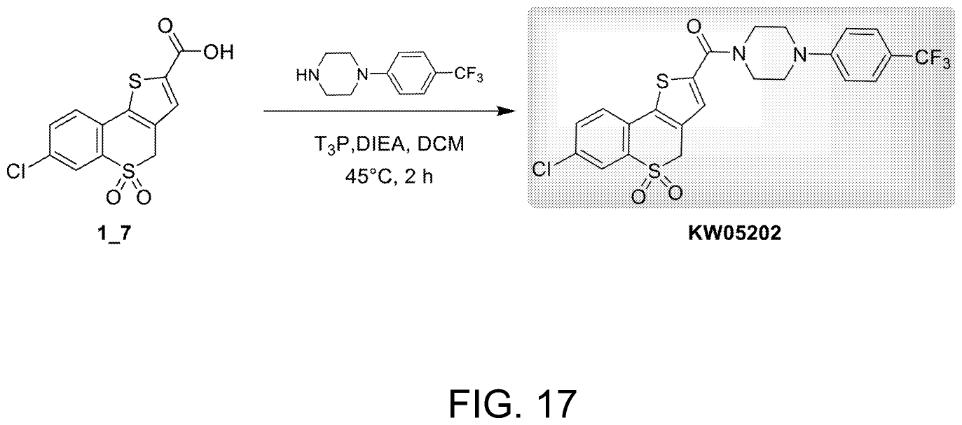

Method for KW05202. FIG. 17 shows a reaction scheme for KW05202. Step 1: To a solution of compound 1_7 (60.0 mg, 0.19 mmol), 1-(4-trifluoromethylphenyl) piperazine (52.3 mg, 0.23 mmol) and DIEA (0.09 mL, 0.57 mmol) in ethyl acetate (2.0 mL) was added T₃P (50% in ethyl acetate, 182 mg, 146.8 μL, 0.57 mmol). The resulting mixture was stirred at 45° C. for 2 h. The desired product mass was detected on LCMS. The mixture was quenched by water (20.0 mL) and extracted with DCM (30.0 mL). The combined organic layers were dried and concentrated to give a residue. The residue was triturated with ethyl acetate (1.00 mL) to give compound KW05202 (14.5 mg, 0.03 mmol, 13.6%) as a yellow solid. MS m/z (ESI):=527.1, 529.1 [M+H]⁺. ¹H NMR (400 MHZ, DMSO-d₆): δ=7.96 (s, 1H), 7.86 (s, 2H), 7.58-7.52 (m, 3H), 7.08 (br d, J=8.8 Hz, 2H), 4.96 (s, 2H), 3.84 (br s, 4H), 3.47-3.41 (m, 4H).

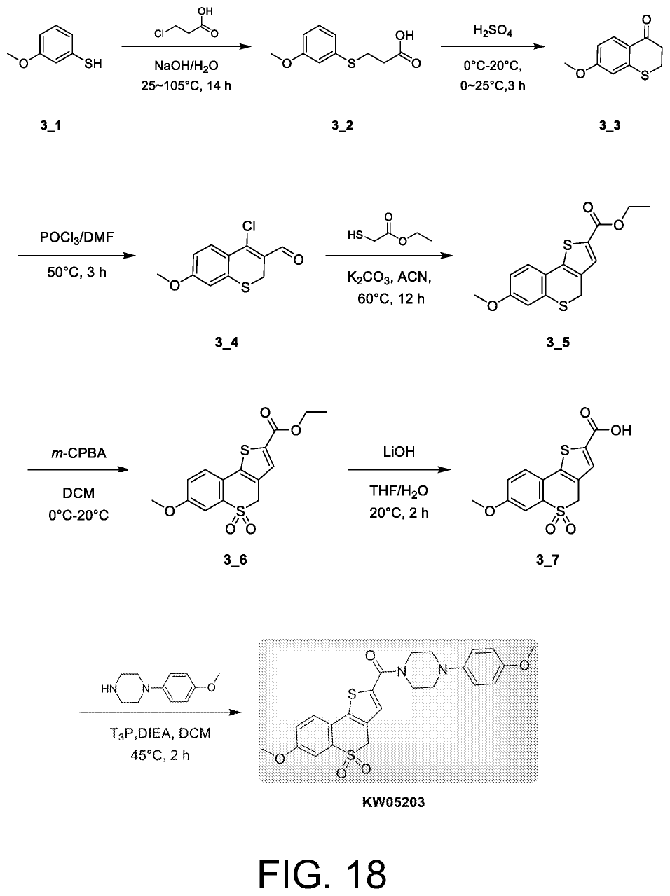

Method for KW05203. FIG. 18 shows a reaction scheme for KW05203. Step 1:

To a solution of compound 3_1 (4.42 mL, 35.7 mmol) in EtOH (20.0 mL) under N₂, a mixture of NaOH solution (1M, 17.1 mL, 17.1 mmol) and Na₂CO₃ solution (1M, 17.1 mL, 17.1 mmol) was added. The resulting mixture was stirred at 25° C. for 5 minutes. Then a solution of 3-chloropropanoic acid (3.20 mL, 37.4 mmol) in H₂O (13.0 mL) was added dropwise to the reaction mixture under N₂. The resulting mixture was stirred at 25° C. for 2 h and then at 105° C. for another 12 h. TLC (Petroleum ether:Ethyl acetate=3:1) indicated starting material was consumed and one major spot was formed. The mixture was concentrated to give a residue. The residue was adjusted to pH=4 with 2N HCl, diluted with water (150 mL) and extracted with ethyl acetate (200 mL×2). The combined organic layers were separated, washed with brine (150 mL), dried and concentrated to give crude 3_2 (7.20 g, 33.9 mmol, 95.1%) as a white solid.

Step 2: Concentrated H₂SO₄ (16.0 mL) was added to compound 3_2 (7.20 g, 33.2 mmol) at 0° C. The resulting mixture was stirred at 25° C. for 3 h. TLC (Petroleum ether: Ethyl acetate=5:1) indicated starting material was consumed and one major spot was formed. The reaction mixture was poured to ice water slowly and extracted with ethyl acetate (150 mL). The organic layer was washed with water and bine, dried by Na₂SO₄, filtered and concentrated. The residue was purified by chromatography (SiO₂, Petroleum ether:Ethyl acetate=0:1) to give compound 3_3 (3.40 g, 17.5 mmol, 51.6%) as a white solid.

Step 3: To a mixture of compound 3_3 (3.40 g, 17.5 mmol) in DMF (10.0 mL) at 0° C. was added POCl₃ (2.44 mL, 26.3 mmol). The resulting mixture was stirred at 50° C. for 3 h. TLC (Petroleum ether:Ethyl acetate=5:1) indicated starting material was consumed and one major spot was formed. The reaction mixture was cooled to 0° C. and quenched by addition of 20 mL of ice water. The mixture was filtered. The filter cake was washed with 50 mL of water and then dried in vacuum to give compound 3_4 (2.90 g, 12.1 mmol, 68.8%) as a yellow solid.

Step 4: To a mixture of compound 3_4 (320 mg, 1.31 mmol) and K₂CO₃ (276 mg, 1.99 mmol) in acetonitrile (5.00 mL) was added ethyl sulfanylacetate (0.22 mL, 1.99 mmol). The resulting mixture was stirred at 60° C. for 12 h. TLC (Petroleum ether:Ethyl acetate=5:1) indicated starting material was consumed and one major spot was formed. The reaction was quenched by water (50.0 mL) and extracted with ethyl acetate (60.0 mL). The combined organic layers were dried and concentrated to give crude compound 3_5 (220 mg, 0.72 mmol, 54.0%) as a yellow solid which was used for next step without further purification.

Step 5: To a solution of compound 3_5 (220 mg, 0.71 mmol) in DCM (3.00 mL) at 0° C. was added m-CPBA (248.5 mg, 1.44 mmol). The resulting mixture was stirred at 20° C. for 3 h. TLC (Petroleum ether:Ethyl acetate=2:1) indicated starting material was consumed and one major spot was formed. The reaction was quenched by addition of saturated sodium sulfite solution (50.0 mL) and extracted with DCM (120 mL). The combined organic layers were dried and concentrated to give a residue. The residue was purified by chromatography (SiO₂, Petroleum ether:Ethyl acetate=0:1) to give compound 3_6 (100 mg, 0.30 mmol, 41.16%) as a yellow solid.

Step 6: To a solution of compound 3_6 (100 mg, 0.29 mmol) in THF (1.00 mL) and H₂O (1.00 mL), was added LiOH·H₂O (21.3 mg, 0.89 mmol). The resulting mixture was stirred at 20° C. for 2 h. TLC (Petroleum ether:Ethyl acetate=2:1) showed starting material was consumed and one major spot was formed. The reaction solution was adjusted to pH=4 with saturated citric acid solution, diluted with water (20.0 mL) and extracted with ethyl acetate (30.0 mL×2). The combined organic layers were separated, washed with brine (20.0 mL), dried and concentrated to give crude compound 3_7 (70.0 mg, 0.23 mmol, 76.3%) as a yellow solid which was used for next step without further purification.

Step 7: To a solution of compound 3_7 (61.9 mg, 0.27 mmol), 1-(4-methoxyphenyl)piperazine (62.9 mg, 0.33 mmol) and DIEA (0.11 mL, 0.68 mmol) in DCM was added T₃P (50% in ethyl acetate, 215 mg, 173.4 μL, 0.68 mmol). The resulting mixture was stirred at 45° C. for 2 h. LCMS showed starting material was consumed and desired product was formed. The mixture was quenched by water (20.0 mL) and extracted with DCM (30.0 mL). The combined organic layers were dried and concentrated to give a residue. The residue was triturated with ethyl acetate (1.0 mL) to afford compound KW05203 (32.9 mg, 0.06 mmol, 28.1%) as a yellow solid. MS m/z (ESI):=485.1 [M+H]⁺. ¹H NMR (400 MHZ, DMSO-d₆) δ=7.75 (d, J=8.8 Hz, 1H), 7.52 (s, 1H), 7.41 (d, J=2.8 Hz, 1H), 7.37-7.31 (s, 1H), 6.97-6.91 (m, 2H), 6.87-6.81 (m, 2H), 4.85 (s, 2H), 3.91 (s, 3H), 3.82 (br s, 4H), 3.69 (s, 3H), 3.06-3.13 (m, 4H).

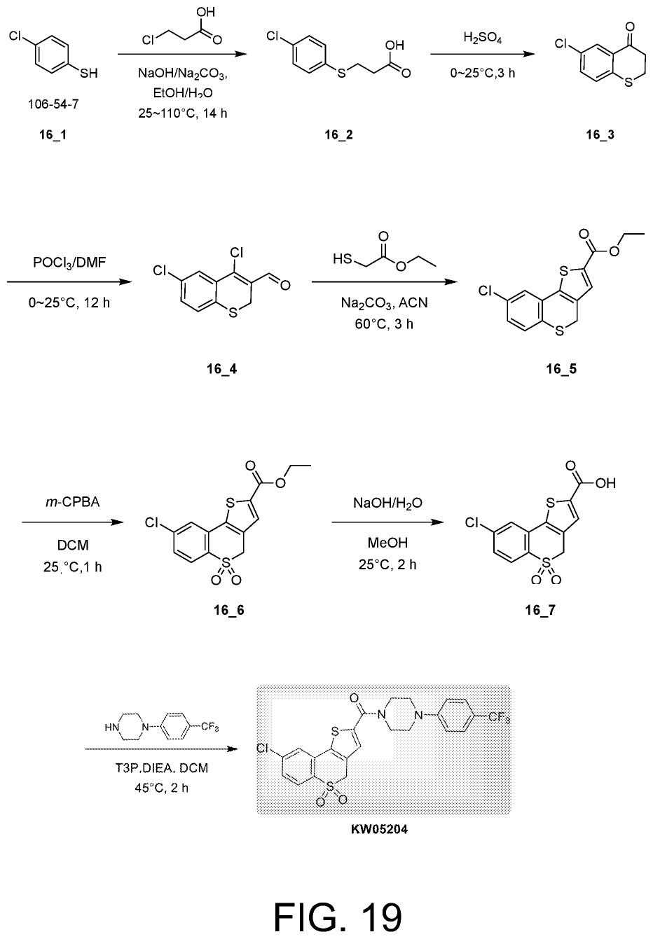

Method for KW05204. FIG. 19 shows a reaction scheme for KW05204. Step 1: To a solution of compound 4_1 (1.22 mL, 10 mmol) in EtOH (6 mL) was added a mixture of NaOH solution (2 M, 5 mL, 5.00 mmol) and Na₂CO₃ solution (2M, 5 mL, 5.00 mmol), followed by 3-chloropropanoic acid (0.90 mL, 10.50 mmol) in H₂O (4 mL). The resulting mixture was stirred at 25° C. for 2 h and at 110° C. for 12 h. TLC (Petroleum ether:Ethyl acetate=4:1) indicated compound 4_1 was consumed completely, and one major spot was formed. The mixture was cooled to 0° C., then 2N HCl was added to adjust the pH to 2. The mixture was extracted with DCM (90 mL). The organic layer was dried and concentrated to give crude compound 4_2 (1.85 g, 8.54 mmol, 85.4%) as light brown solid which was used for the next step without further purification. ¹H NMR (400 MHZ, DMSO-d₆) δ 12.34 (br, 1H), 7.30-7.44 (m, 4H), 3.14 (t, J=7.0 Hz, 2H), 2.51-2.56 (m, 2H).

Step 2: Compound 4_2 (1.8 g, 8.31 mmol) was added to concentrated H₂SO₄ (5 mL, 1.62 mmol) at 0° C. slowly, then the mixture was stirred at 25° C. for 3 h. TLC (Petroleum ether:Ethyl acetate=10:1) indicated one major spot was formed. The mixture was poured into ice and extracted with DCM (60 mL). The combined organic layers were dried and concentrated to give a residue. The residue was purified by chromatography (SiO₂, Petroleum ether:Ethyl acetate=1:0 to 10:1) to give compound 4_3 (1450 mg, 7.30 mmol, 87.86%) as yellow solid. ¹H NMR (400 MHZ, DMSO-d₆) δ 7.88 (d, J=2.4 Hz, 1H), 7.53 (dd, J=8.5, 2.5 Hz, 1H), 7.42 (d, J=8.4 Hz, 1H), 3.33-3.36 (m, 2H), 2.87-2.96 (m, 2H)

Step 3: To a solution of compound 4_2 (840 mg, 4.23 mmol) in DMF (9 mL) at 0° C. was added POCl₃ (0.59 mL, 6.34 mmol). The resulting mixture was stirred at 25° C. for 12 h. TLC (Petroleum ether:Ethyl acetate=20:1) indicated one major spot was formed. The mixture was diluted with H₂O (15 mL) and extracted with ethyl acetate (75 mL). The combined organic layers were dried and concentrated to give a residue. The residue was purified by chromatography (SiO₂, Ethyl acetate: Petroleum ether=0:1 to 1:20) to give compound 4_3 (800 mg, 3.26 mmol, 77.19%) as yellow oil. ¹H NMR (400 MHZ, DMSO-d₆) § 10.21 (s, 1H), 7.89 (dd, J=1.8, 0.7 Hz, 1H), 7.43- 7.66 (m, 2H), 3.75 (s, 2H).

Step 4: To a mixture of compound 4_3 (800 mg, 3.26 mmol) and ethyl sulfanylacetate (0.43 mL, 3.92 mmol) in acetonitrile (10 mL) was added K₂CO₃ (676.6 mg, 4.90 mmol). The resulting mixture was stirred at 60° C. for 3 h. TLC (Petroleum ether:Ethyl acetate=10/1) indicated compound 4_3 was consumed completely, and one major spot was formed. The mixture was diluted with H₂O (20 mL) and extracted with ethyl acetate (75 mL).

The combined organic layers were dried and concentrated to give a residue. The residue was purified by chromatography (SiO₂, Petroleum ether:Ethyl acetate=20:1 to 8:1) to give compound 4_4 (800 mg, 2.57 mmol, 78.86%) as yellow solid. ¹H NMR (400 MHZ, DMSO-d₆) δ 7.73 (s, 1H), 7.62 (d, J=2.2 Hz, 1H), 7.44 (d, J=8.4 Hz, 1H), 7.32 (dd, J=8.4, 2.3 Hz, 1H), 4.27-4.32 (m, 2H), 4.09 (s, 2H), 1.29-1.33 (m, 3H)

Step 5: To a solution of compound 4_5 (800 mg, 2.57 mmol) in DCM (10 mL) was added m-CPBA (0.88 g, 5.15 mmol). The resulting mixture was stirred at 25° C. for 1 h. TLC (Petroleum ether:Ethyl acetate=4:1) indicated compound 4_5 was consumed completely, and two major spots were formed. The reaction was quenched by saturated aqueous Na₂SO₃ solution and extracted with DCM (60 mL). The combined organic layers were dried and concentrated to give a residue. The residue was purified by chromatography (SiO₂, Petroleum ether:Ethyl acetate=20:1 to 1:1) to give crude compound 4_6 (580 mg, 1.69 mmol, 65.73%) as yellow solid which was used for next step without further purification.

Step 6: To a solution of compound 4_6 (150 mg, 0.44 mmol) in MeOH (2 mL) and H₂O (0.5 mL) was added LiOH·H₂O (55 mg, 1.31 mmol). The resulting mixture was stirred at 25° C. for 2 h. TLC (Petroleum ether:Ethyl acetate=4:1) indicated starting material was consumed completely and one major spot was formed. The mixture was concentrated to give a residue, then 2N HCl was added to adjust pH to 2-3. The mixture was extracted with ethyl acetate (60 mL). The combined organic layers were dried and concentrated to give crude compound 4_7 (135 mg, 0.43 mmol, 98.02%) as a light brown solid which was used for the next step without further purification. ¹H NMR (400 MHZ, DMSO-d₆) δ 7.93-8.02 (m, 2H), 7.78 (s, 1H), 7.68-7.75 (m, 1H), 4.97 (s, 2H).

Step 6: To a mixture of compound 4_7 (90 mg, 0.29 mmol) and 1-(4-(trifluoromethyl)phenyl)piperazine (85.2 mg, 0.37 mmol) in DCM (2 mL) was added T₃P (50% in ethyl acetate, 363.9 mg, 293.5 μL, 1.14 mmol) and DIEA (0.19 mL, 1.14 mmol). The resulting mixture was stirred at 45° C. for 2 h. TLC (Petroleum ether:Ethyl acetate=1:1) indicated one major spot was formed. The mixture was diluted with H₂O (10 mL) and extracted with DCM (60 mL). The combined organic layers were dried and concentrated to give a residue. The residue was purified by chromatography (SiO₂, Petroleum ether:Ethyl acetate=20:1 to 0:1) to give a crude product, which was triturated with DCM (2 mL) at 25° C. for 10 min to give compound KW05204 (62.46 mg, 0.12 mmol, 41.45%) as yellow solid. MS m/z (ESI):=527.0 [M+H]⁺. ¹H NMR (400 MHZ, DMSO-d₆) δ 7.97 (d, J=8.4 Hz, 1H), 7.92 (d, J=1.9 Hz, 1H), 7.70 (dd, J=8.3, 1.9 Hz, 1H), 7.53-7.58 (m, 3H), 7.08 (d, J=8.8 Hz, 2H), 4.94 (s, 2H), 3.84 (br, 4H), 3.41-3.47 (m, 4H).

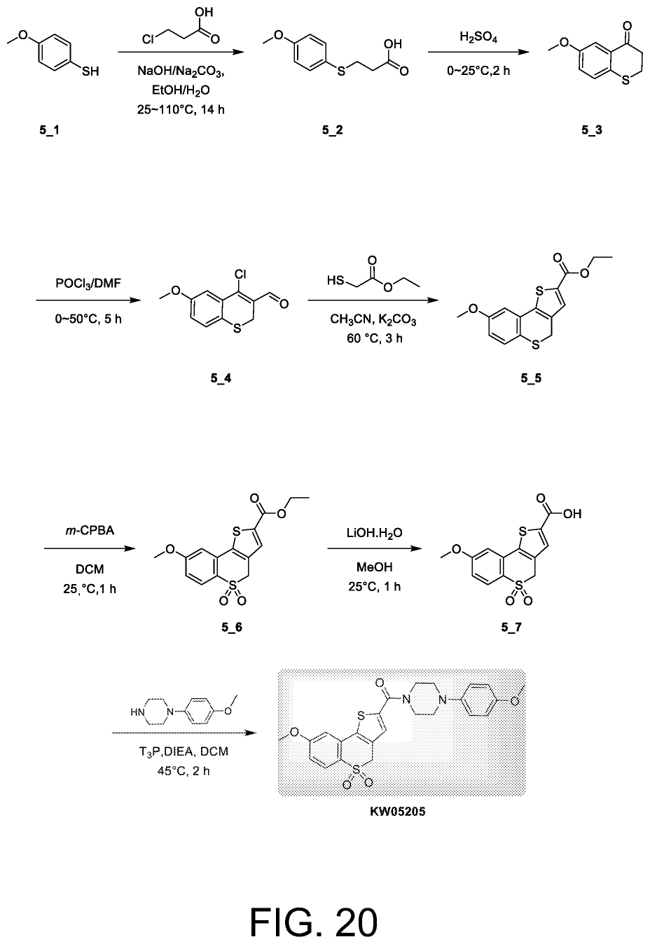

Method for KW05205. FIG. 20 shows a reaction scheme for KW05205. Step 1: To a mixture of NaOH solution (2M, 5 mL, 5.00 mmol) and Na₂CO₃ solution (2M, 5 mL, 5.00 mmol), was added a solution of compound 5_1 (1.23 mL, 10 mmol) in EtOH (6 mL) and then a solution of 3-chloropropanoic acid (0.90 mL, 10.50 mmol) in H₂O (4 mL). The resulting mixture was stirred at 25° C. for 2 h and then at 110° C. for 12 h. TLC (Petroleum ether:Ethyl acetate=4:1) indicated one major spot was formed. The mixture was cooled to 0° C., then 2N HCl was added to adjust pH to 2. The mixture was extracted with DCM (30 mL×3). The combined organic layers were dried and concentrated to give a residue. The residue was purified by chromatography (SiO₂, Ethyl acetate: Petroleum ether=0:1 to 1:10) to give compound 5_2 (1.5 g, 7.07 mmol, 70.7%) as an off-white solid. ¹H NMR (400 MHZ, DMSO-d₆) δ 12.28 (s, 1H), 7.34 (d, J=8.8 Hz, 2H), 6.87-6.97 (m, 2H), 3.75 (s, 3H), 2.99 (t, J=7.1 Hz, 2H), 2.44 (t, J=7.1 Hz, 2H).

Step 2: Compound 5_2 (1.40 g, 6.60 mmol) was added to concentrated H₂SO₄ (5 mL) at 0° C. slowly. The resulting mixture was stirred at 25° C. for 2 h. TLC (Petroleum ether:Ethyl acetate=10:1) indicated two new spots were formed. The mixture was poured to ice, then the mixture was extracted with DCM (75 mL×2). The combined organic layers were dried and concentrated to give a residue. The residue was purified by chromatography (SiO₂, Petroleum ether:Ethyl acetate=20:1 to 5:1) to give compound 5_3 (540 mg, 2.78 mmol, 42.15%) as a yellow oil. ¹H NMR (400 MHZ, DMSO-d₆) δ 7.47 (d, J=2.8 Hz, 1H), 7.29 (d, J=8.5 Hz, 1H), 7.12 (dd, J=8.7, 3.0 Hz, 1H), 3.77 (s, 3H), 3.25-3.30 (m, 2H), 2.86-2.91 (m, 2H).

Step 3: To a solution of compound 5_3 (540 mg, 2.78 mmol) in DMF (5 mL) at 0° C. was added POCl₃ (0.39 mL, 4.17 mmol). The resulting mixture was stirred at 50° C. for 5 h. TLC (Petroleum ether:Ethyl acetate=5:1) indicated compound 5_3 was consumed completely, and one major spot was formed. The mixture was diluted with H₂O (15 mL) and extracted with ethyl acetate (60 mL×2). The combined organic layers were dried and concentrated to give a residue. The residue was purified by chromatography (SiO₂, Ethyl acetate: Petroleum ether=0:1 to 1:10) to give compound 5_4 (530 mg, 2.20 mmol, 79.20%) as yellow solid. ¹H NMR (400 MHZ, DMSO-d₆) δ 10.22 (s, 1H), 7.35-7.45 (m, 2H), 7.09 (dd, J=8.5, 2.7 Hz, 1H), 3.82 (s, 3H), 3.68 (s, 2H).

Step 4: To a solution of compound 5_4 (550 mg, 2.28 mmol) in acetonitrile (6 mL) was added ethyl sulfanylacetate (0.30 mL, 2.74 mmol) and K₂CO₃ (473.70 mg, 3.43 mmol). The resulting mixture was stirred at 60° C. for 3 h. TLC (Petroleum ether:Ethyl acetate=5:1) indicated compound 5_4 was consumed completely, and one major spot was formed. The mixture was diluted with H₂O (15 mL) and extracted with ethyl acetate (60 mL). The combined organic layers were dried and concentrated to give a residue. The residue was purified by chromatography (SiO₂, ethyl acetate: Petroleum ether=0:1 to 1:8) to give compound 5_5 (500 mg, 1.63 mmol, 71.42%) as a yellow solid. ¹H NMR (400 MHZ, DMSO-d₆) δ 7.73 (s, 1H), 7.33 (d, J=8.7 Hz, 1H), 7.10 (d, J=2.6 Hz, 1H), 6.90 (dd, J=8.7, 2.6 Hz, 1H), 4.27-4.33 (m, 2H), 4.02 (s, 2H), 3.80 (s, 3H), 1.29-1.33 (m, 3H).

Step 5: To a solution of compound 5_5 (490 mg, 1.60 mmol) in DCM (5 mL) was added m-CPBA (552 mg, 3.20 mmol). The resulting mixture was stirred at 25° C. for 1 h. TLC (Petroleum ether:Ethyl acetate=2:1) indicated several new spots were formed. The mixture was quenched by 10% aqueous Na₂SO₃ solution (10 mL) and the mixture was stirred at 25° C. for 10 min. Then the mixture was extracted with ethyl acetate (25 mL×3). The combined organic layers were dried and concentrated to give a residue. The residue was purified by chromatography (SiO₂, Petroleum ether:Ethyl acetate=20:1 to 0:1) to give compound 5_6 (310 mg, 0.92 mmol, 57.28%) as a light-yellow solid. ¹H NMR (400 MHZ, DMSO-d₆) δ 7.90 (d, J=8.7 Hz, 1H), 7.85 (s, 1H), 7.31 (d, J=2.4 Hz, 1H), 7.21 (dd, J=8.7, 2.4 Hz, 1H), 4.87 (s, 2H), 4.34 (q, J=7.2 Hz, 2H), 3.93 (s, 3H), 1.32 (t, J=7.1 Hz, 3H)

Step 6: To a mixture of compound 5_6 (320 mg, 0.95 mmol) in MeOH (4 mL) and H₂O (1 mL) was added LiOH·H₂O (68 mg, 2.84 mmol). The resulting mixture was stirred at 25° C. for 1 h. TLC (Petroleum ether:Ethyl acetate=2:1) indicated compound 5_6 was consumed completely, and one major spot was formed. The solvent was removed under reduced pressure, then 2N HCl was added to adjust the pH to 2-3. The mixture was extracted with ethyl acetate (25 mL×3) and the combined organic layers were dried and concentrated to give crude compound 5_7 (240 mg, 0.77 mmol, 81.78%) as a light-brown solid which was used for next step without further purification. ¹H NMR (400 MHZ, DMSO-d₆) δ 7.89 (d, J=8.7 Hz, 1H), 7.77 (s, 1H), 7.28 (d, J=2.4 Hz, 1H), 7.20 (dd, J=8.7, 2.4 Hz, 1H), 4.86 (s, 2H), 3.93 (s, 3H).

Step 6: To a mixture of compound 5_7 (70 mg, 0.23 mmol) and 1-(4-methoxyphenyl)piperazine (61.91 mg, 0.27 mmol) in DCM (1.5 mL) was added T₃P (50% in ethyl acetate, 215.30 mg, 0.68 mmol) and DIEA (0.15 mL, 0.90 mmol). The resulting mixture was stirred at 45° C. for 2 h. TLC (Petroleum ether:Ethyl acetate=1:1) indicated one major spot was formed. The mixture was diluted with H₂O (10 mL) and extracted with DCM (15 mL×3). The combined organic layers were dried and concentrated to give a residue. The residue was purified by prep-TLC (SiO₂, Petroleum ether:ethyl acetate=1:1) to give compound KW05205 (36 mg, 0.07 mmol, 31.01%) as a light-yellow solid. MS m/z (ESI):=485.1 [M+H]⁺. ¹H NMR (400 MHZ, DMSO-d₆) δ 7.89 (d, J=8.7 Hz, 1H), 7.54 (s, 1H), 7.23 (d, J=2.4 Hz, 1H), 7.18 (dd, J=8.7, 2.4 Hz, 1H), 6.91-6.99 (m, 2H), 6.81-6.89 (m, 2H), 4.82 (s, 2H), 3.92 (s, 3H), 3.82 (br, 4H), 3.69 (s, 3H), 3.04-3.15 (m, 4H).

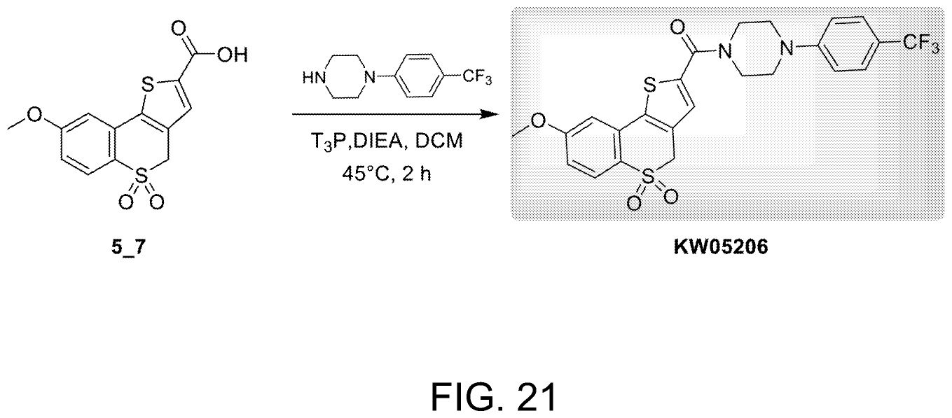

Method for KW05206. FIG. 21 shows a reaction scheme for KW05206. Step 1: To a mixture of compound 5_7 (80 mg, 0.26 mmol) and 1-(4-(trifluoromethyl)phenyl) piperazine (71.4 mg, 0.31 mmol) in DCM (1.5 mL) was added T₃P (50% in ethyl acetate, 246.06 mg, 0.77 mmol) and DIEA (0.13 mL, 0.77 mmol). The resulting mixture was stirred at 45° C. for 2 h. TLC (Petroleum ether:Ethyl acetate=1:1) indicated a new major spot was formed. The mixture was diluted with H₂O (10 mL) and extracted with ethyl acetate (20 mL×3). The combined organic layers were dried and concentrated to give a residue. The residue was triturated with ethyl acetate (5 mL) at 25° C. for 15 min to give compound KW05206 (56.93 mg, 0.10 mmol, 40.35%) as a light-yellow solid. MS m/z (ESI):=522.9 [M+H]⁺. ¹H NMR (400 MHz, DMSO-d₆) δ 7.89 (d, J=8.8 Hz, 1H), 7.52-7.58 (m, 3H), 7.24 (d, J=2.4 Hz, 1H), 7.18 (dd, J=8.7, 2.4 Hz, 1H), 7.08 (d, J=8.8 Hz, 2H), 4.83 (s, 2H), 3.93 (s, 3H), 3.85 (br, 4H), 3.39-3.49 (m, 4H).

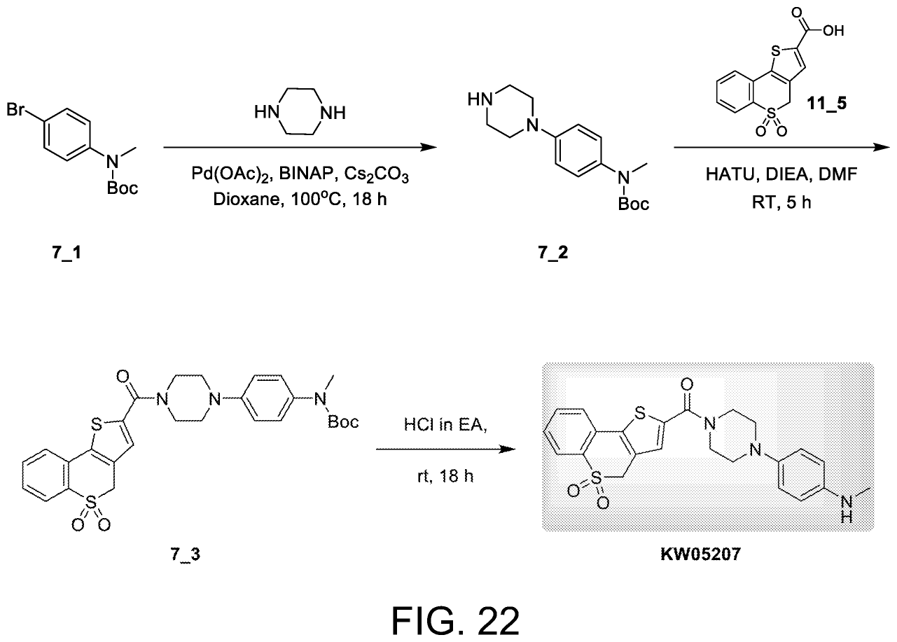

Method for KW05207. FIG. 22 shows a reaction scheme for KW05207. Step 1: To a solution of compound 7_1 (2.86 g, 9.99 mmol) in dioxane (50 mL) were added piperazine (1.17 mL, 14.99 mmol), Cs₂CO₃ (9.77 g, 29.98 mmol), Pd(OAc)₂ (0.22 g, 1.00 mmol) and BINAP (0.62 g, 1.00 mmol). The resulting mixture was stirred at 100° C. under nitrogen for 18 h. TLC (DCM:MeOH=20:1) and LCMS showed that the compound 7_1 was consumed completely, and one major new spot was formed. The reaction was diluted with water (200 mL) and extracted with ethyl acetate (200 mL×2). The organic layer was separated, combined, washed with brine and concentrated in vacuo. The residue was purified using silica gel column chromatography (DCM:MeOH=20:1) to afford compound 7_2 (1.10 g, 3.40 mmol, 34.00%) as a brown oil. MS m/z (ESI):=292.1 [M+H]⁺.

Step 2: To a solution of compound 7_2 (291.39 mg, 1.00 mmol) in DMF (5 mL) were added compound 11_5 (280.33 mg, 1.00 mmol), HATU (456.28 mg, 1.20 mmol), and DIEA (0.50 mL, 3.00 mmol). The resulting mixture was stirred at room temperature for 5 h under nitrogen. LCMS showed that the compound 7_2 was consumed completely, and the desired product was detected. The reaction mixture was diluted with water (100 mL) and extracted with ethyl acetate (100 mL×2). The organic layer was separated, combined, washed with brine and concentrated in vacuo. The residue was purified using silica gel column chromatography (eluted with Petroleum ether:Ethyl acetate=1:1) to afford compound 7_3 (350 mg, 0.60 mmol, 60.05%) as a yellow solid. MS m/z (ESI):=554.1 [M+H]⁺.

Step 3: To a solution of compound 7_3 (120.55 mg, 0.22 mmol) in ethyl acetate (1 mL) was added HCl (1.0 mL, 0.88 mmol). The resulting mixture was stirred at room temperature for 18 h. LCMS showed that the compound 7_3 was consumed completely, and the desired product was detected. The reaction mixture was concentrated in vacuo, triturated by ethyl acetate (3 mL). Then the mixture was filtered and dried to afford compound KW05207 (98.23 mg, 0.21 mmol, 98.03%) as a white solid. MS m/z (ESI):=454.1 [M+H]⁺. ¹H NMR (400 MHZ, DMSO-d₆) δ 7.98-7.96 (m, 1H), 7.82-7.77 (m, 2H), 7.67-7.63 (m, 1H), 7.58 (s, 1H), 7.35 (d, J=8.3 Hz, 2H), 7.17 (d, J=8.5 Hz, 2H), 4.90 (s, 2H), 3.88 (d, J=6.3 Hz, 4H), 3.36 (t, J=5.2 Hz, 4H), 2.83 (s, 3H).

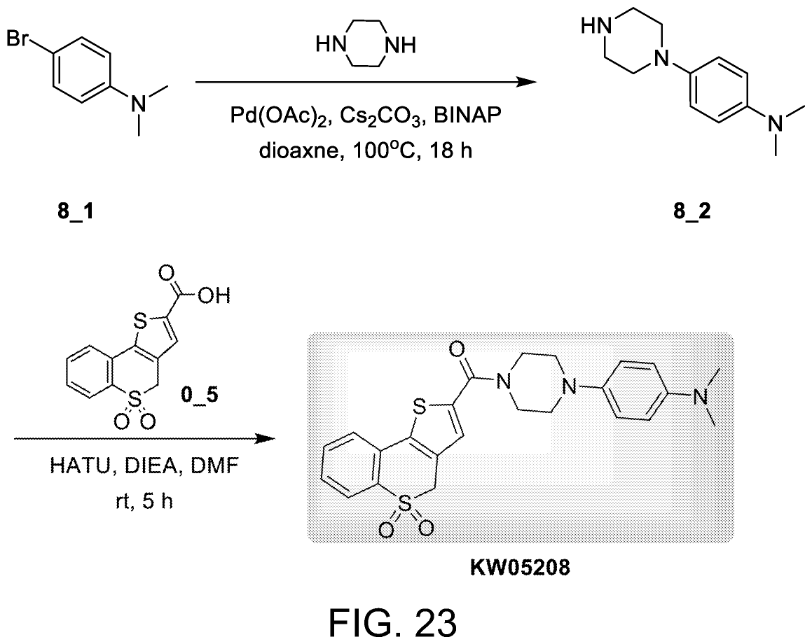

Method for KW05208. FIG. 23 shows a reaction scheme for KW05208. Step 1: To a solution of compound 8_1 (2.00 g, 10.00 mmol) in dioxane (40 mL) were added piperazine (1.17 mL, 14.99 mmol), Cs₂CO₃ (9.77 g, 29.99 mmol), Pd(OAc)₂ (0.22 g, 1.00 mmol) and BINAP (0.62 g, 1.00 mmol). The resulting mixture was stirred at 100° C. for 18 h under nitrogen. LCMS showed that the starting material was consumed completely, and the desired product was detected. The reaction mixture was filtered with diatomite and washed with ethyl acetate (100 mL). The solution was diluted with water (100 mL) and extracted with ethyl acetate (100 mL×2). The organic layer was separated, combined, washed with brine and concentrated in vacuo. The residue was purified using silica gel column chromatography (eluted with DCM: MeOH=20:1) to give compound 8_2 (1.20 g, 5.55 mmol, 55.55%) as a brown solid. MS m/z (ESI):=206.1 [M+H]⁺.

Step 2: To a solution of compound 8_2 (205.31 mg, 1.00 mmol) in DMF (5 mL) were added compound 0_5 (280.33 mg, 1.00 mmol), HATU (456.28 mg, 1.20 mmol), and DIEA (0.50 mL, 3.00 mmol). The resulting mixture was stirred at room temperature for 5 h under nitrogen. LCMS showed that the compound 8_2 was consumed completely, and the desired product was detected. The reaction was poured into water (100 mL) and extracted with ethyl acetate (100 mL×2). The organic layer was separated, combined, washed with brine and concentrated in vacuo. The residue was purified using silica gel column chromatography (eluted with Petroleum ether:Ethyl acetate=1:1) to afford compound KW05208 (71.75 mg, 0.15 mmol, 15.06%) as a green solid. MS m/z (ESI):=468.1 [M+H]⁺ ¹H NMR (400 MHz, DMSO-d₆) δ 7.97 (d, J=7.7 Hz, 1H), 7.82-7.77 (m, 2H), 7.67-7.63 (m, 1H), 7.54 (s, 1H), 6.89 (d, J=8.7 Hz, 2H), 6.71 (d, J=8.4 Hz, 2H), 4.90 (s, 2H), 3.81 (t, J=5.0 Hz, 4H), 3.05 (t, J=5.1 Hz, 4H), 2.80 (s, 6H).

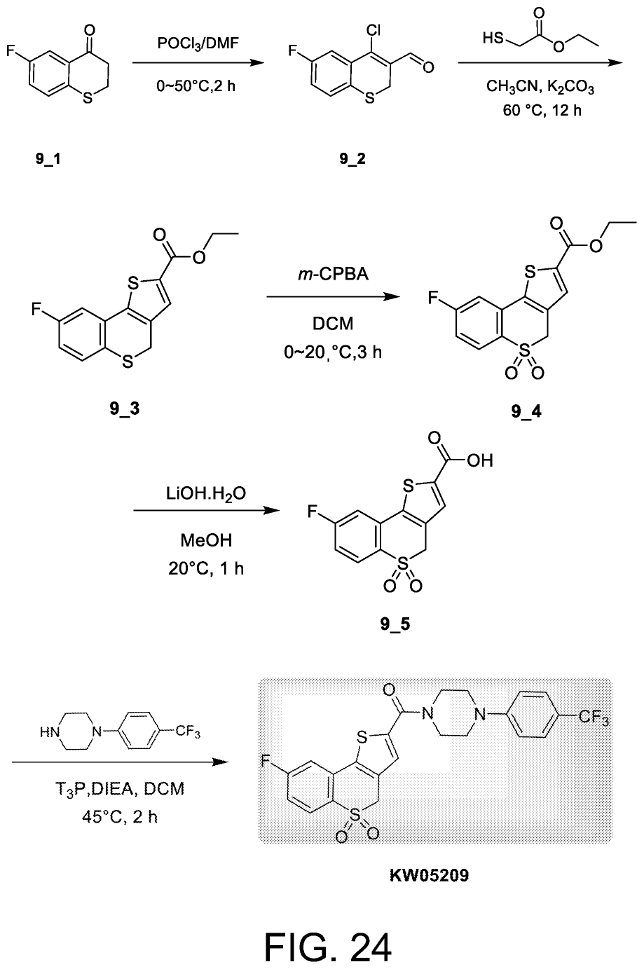

Method for KW05209. FIG. 24 shows a reaction scheme for KW05209. Step 1: To a solution of compound 9_1 (3.00 g, 16 mmol) in DMF (45.0 mL) at 0° C. was added POCl₃ (2.29 mL, 24.7 mmol). The resulting mixture was stirred at 50° C. for 2 h. TLC (Petroleum ether:Ethyl acetate=5:1) indicated starting material was consumed and one major spot was formed. The reaction mixture was cooled to 0° C. and then quenched by addition of ice water (20 mL). The mixture was filtered, and the filter cake was washed with water (80.0 mL) and dried in vacuum to give compound 9_2 (1.50 g, 6.56 mmol, 39.8%) as a yellow solid.

Step 2: To a solution of compound 9_2 (1.00 g, 4.37 mmol) and K₂CO₃ (0.91 g, 6.56 mmol) in acetonitrile (10.0 mL) was added ethyl sulfanylacetate (0.72 mL, 6.56 mmol). The resulting mixture was stirred at 60° C. for 12 h. TLC (Petroleum ether:Ethyl acetate=5:1) indicated starting material was consumed and one major spot was formed. The reaction was quenched by water (90 mL) and extracted with ethyl acetate (50 mL×2). The combined organic layers were dried and concentrated to give crude compound 9_3 (1.00 g, 3.40 mmol, 77.7%) as a yellow solid which was used for the next step without further purification.

Step 3: To a solution of compound 9_3 (1.00 g, 3.22 mmol) in DCM (15.0 mL) at 0° C. was added m-CPBA (1.17 g, 6.79 mmol). The resulting mixture was stirred at 20° C. for 3 h. TLC (Petroleum ether:Ethyl acetate=2:1) indicated starting material was consumed and one major spot was formed. The reaction was quenched by saturated aqueous sodium sulfite solution (50.0 mL) and extracted with DCM (60 mL×2). The combined organic layers were dried and concentrated to give a residue. The residue was purified by chromatography (SiO₂, Petroleum ether:Ethyl acetate=0:1 to 0:1) to give compound 9_4 (270 mg, 0.83 mmol, 24.4%) as a yellow solid.

Step 4: To a solution of compound 9_4 (270 mg, 0.79 mmol) in THF (3.0 mL) and H₂O (3.0 mL) was added LiOH·H₂O (59 mg, 2.48 mmol). The resulting mixture was stirred at 20° C. for 2 h. TLC (Petroleum ether:Ethyl acetate=3:1) showed starting material was consumed and one major spot was formed. The reaction solution was treated with saturated citric acid solution to adjust the pH to 4, diluted with water (30.0 mL) and extracted with ethyl acetate (50 mL×2). The combined organic layers were separated, washed with brine (30 mL), dried and concentrated to give crude compound 9_5 (180 mg, 0.60 mmol, 72.9%) as a yellow solid which was used for the next step without further purification.

Step 5: To a solution of compound 9_5 (60.0 mg, 0.19 mmol), 1-(4-trifluoromethylphenyl)piperazine (52.3 mg, 0.23 mmol) in ethyl acetate (2.0 mL) was added T₃P (50% in ethyl acetate, 320 mg, 1.01 mmol) and DIEA (0.17 mL, 1.01 mmol). The resulting mixture was stirred at 45° C. for 2 h. LCMS showed the reaction was completed. The reaction was quenched by water (20.0 mL) and extracted with DCM (15 mL×2). The combined organic layers were dried and concentrated to give a residue. The residue was triturated with ethyl acetate (1.0 mL) to give compound KW05209 (49.8 mg, 0.09 mmol, 28.1%) as a white solid. ¹H NMR (400 MHZ, DMSO-d₆) δ=8.04 (dd, J=8.8, 5.6 Hz, 1H), 7.76 (dd, J=9.6, 2.4 Hz, 1H), 7.58-7.53 (m, 3H), 7.49 (td, J=8.4, 2.4 Hz, 1H), 7.08 (d, J=8.8 Hz, 2H), 4.93 (s, 2H), 3.84 (br s, 4H), 3.46-3.41 (m, 4H).

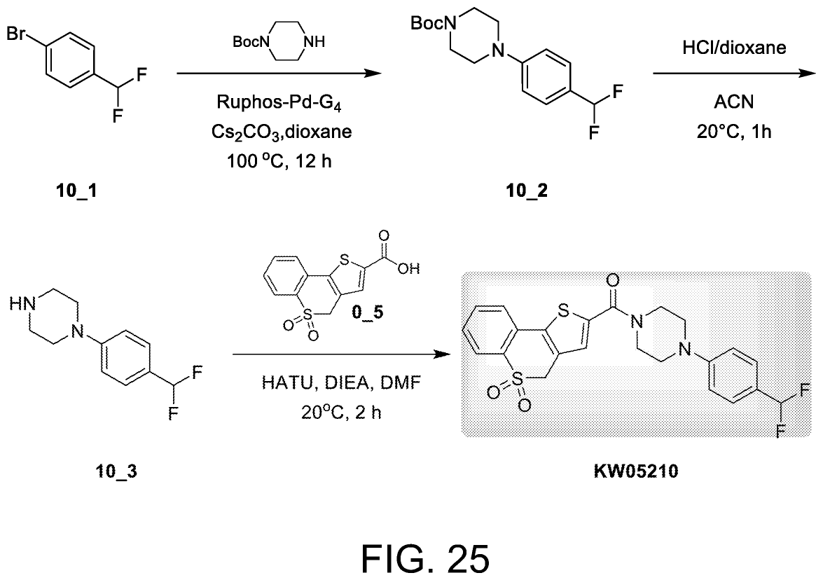

Method for KW05210. FIG. 25 shows a reaction scheme for KW05210. Step 1: To a solution of compound 10_1 (100 mg, 0.48 mmol) and Boc-piperazine (108 mg, 0.58 mmol) in dioxane (7 mL), were added Ruphos-Pd-G4 (41.1 mg, 0.05 mmol) and Cs₂CO₃ (472 mg, 1.45 mmol). The resulting mixture was stirred at 100° C. for 12 h. LC-MS showed the reaction was completed. The reaction was quenched by water (20.0 mL) and extracted with ethyl acetate (10 mL×2). The combined organic layers were washed with brine (20.0 mL), dried and concentrated to give a residue. The residue was purified by chromatography (SiO₂, DCM: MeOH=1:0 to 2:1) to give compound 10_2 (130 mg, 0.61 mmol, 31.7%) as a yellow solid. MS m/z (ESI):=313.0 [M+H] *.

Step 2: To a solution of 10_2 (110 mg, 0.35 mmol) in ACN (3.00 mL) was added HCl/dioxane (4M, 3.00 mL). The resulting mixture was stirred at 20° C. for 1 h. LC-MS showed the reaction was completed. The mixture was concentrated to give crude compound 10_3 (100 mg) as a white solid which was used for next step without purification. MS m/z (ESI):=213.1 [M+H]⁺.

Step 3: To a solution of crude compound 10_3 (83.3 mg, 0.39 mmol) in DMF (2.0 mL), were added DIEA (0.15 mL, 1.07 mmol) and HATU (163.5 mg, 0.43 mmol). The resulting mixture was stirred at 20° C. for 2 h. TLC (Petroleum ether:Ethyl acetate=1:1) indicated starting material was consumed and one main spot formed. The reaction was quenched by water (20.0 mL) and extracted with DCM (10 m×2L). The combined organic layers were washed with brine, dried with anhydrous Na₂SO₄ and then concentrated to give a residue. The residue was triturated with ethyl acetate (1.0 mL) to give compound KW05210 (47.2 mg, 0.10 mmol, 26.9%) as a green solid. MS m/z (ESI):=475.1 [M+H]⁺; ¹H NMR (400 MHZ, DMSO-d₆) δ=7.97 (br d, J=8.0 Hz, 1H), 7.86-7.73 (m, 2H), 7.65 (br t, J=6.4 Hz, 1H), 7.56 (s, 1H), 7.43 (br d, J=8.0 Hz, 2H), 7.05 (br d, J=8.0 Hz, 2H), 4.90 (s, 2H), 3.84 (br s, 4H), 3.37 (br s, 4H).

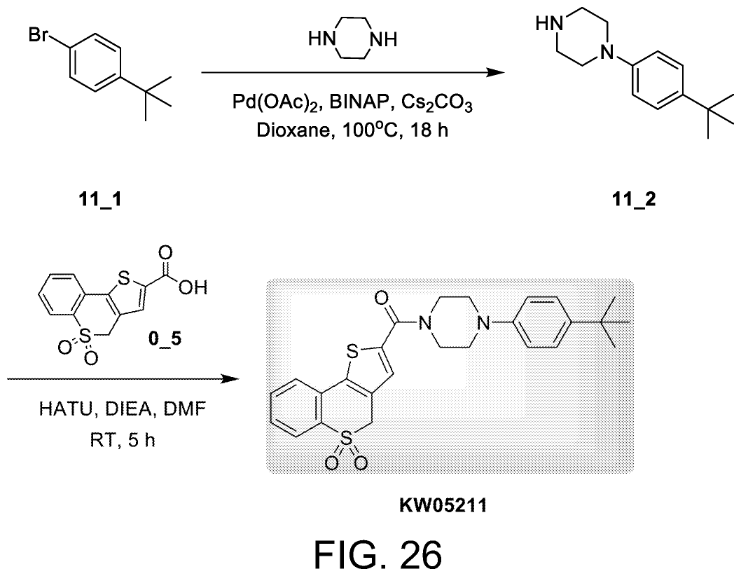

Method for KW05211. FIG. 26 shows a reaction scheme for KW05211. Step 1: To a solution of compound 11_1 (1.70 g, 7.98 mmol) in dioxane (50 mL) were added piperazine (1.03 g, 11.97 mmol), Cs₂CO₃ (7.80 g, 23.93 mmol), Pd(OAc)₂ (0.18 g, 0.80 mmol) and BINAP (0.50 g, 0.80 mmol). The resulting mixture was stirred at 100° C. for 18 h under nitrogen. LC-MS showed that the compound 11_1 was consumed, and the desired product was formed. The reaction mixture was filtered with diatomite, washed with ethyl acetate (100 mL). The solution was washed with water (100 mL×2) and brine (100 mL), dried and concentrated in vacuo. The residue was purified using silica gel column chromatography (DCM:MeOH=20:1) to afford compound 11_2 (0.8 g, 3.48 mmol, 43.64%) as a brown solid. MS m/z (ESI):=219.1 [M+H]⁺

Step 2: To a solution of compound 11_2 (109.17 mg, 0.50 mmol) in DMF (3 mL) were added compound 0_5 (140.16 mg, 0.50 mmol), HATU (228.14 mg, 0.60 mmol), and DIEA (0.25 mL, 1.50 mmol). The resulting mixture was stirred at room temperature for 5 h under nitrogen. LCMS showed that the compound 11_2 was consumed, and the product was detected. The reaction mixture was diluted with water (50 mL) and extracted with ethyl acetate (100 mL×3). The combined organic layers were washed with brine and concentrated in vacuo. The residue was purified using silica gel column chromatography (eluted with Petroleum ether:Ethyl acetate=1:1) to give compound KW05211 (44.81 mg, 0.09 mmol, 18.01%) as a yellow solid. MS m/z (ESI):=481.1 [M+H]⁺ ¹H NMR (500 MHZ, DMSO-d₆) δ 7.98-7.96 (m, 1H), 7.82-7.77 (m, 2H), 7.66-7.63 (m, 1H), 7.55 (s, 1H), 7.28-7.24 (m, 2H), 6.92-6.90 (m, 2H), 4.90 (s, 2H), 3.82 (t, J=5.1 Hz, 4H), 3.19 (t, J=5.2 Hz, 4H), 1.24 (s, 9H).

Method for KW05212. FIG. 27 shows a reaction scheme for KW05212. Step 1: To a mixture of compound 0_5 (100 mg, 0.36 mmol) in DCM (1 mL) was added N,N′-dimethyl-4-(piperazin-1-yl) aniline (88.3 mg, 0.43 mmol), T₃P (50% in ethyl acetate, 905 mg, 1.43 mmol) and DIEA (0.24 mL, 1.43 mmol). The resulting mixture was stirred at 45° C. for 1.5 h. TLC (DCM:MeOH=10:1) indicated compound 0_5 was consumed, and one major spot was formed. The reaction mixture was filtered and concentrated to give a residue. The residue was triturated with MeOH (1 mL) at 25° C. to give compound KW05212 (30.91 mg, 0.06 mmol, 17.87%) as yellow solid. MS m/z (ESI):=468.1 [M+H]⁺.

¹H NMR (400 MHZ, DMSO-d₆) δ 7.95-7.99 (m, 1H), 7.76-7.82 (m, 2H), 7.62-7.68 (m, 1H), 7.53-7.56 (m, 1H), 6.86-6.91 (m, 2H), 6.68-6.73 (m, 2H), 4.87-4.92 (m, 2H), 3.76-3.86 (m, 4H), 3.01-3.09 (m, 4H), 2.78-2.81 (m, 6H).

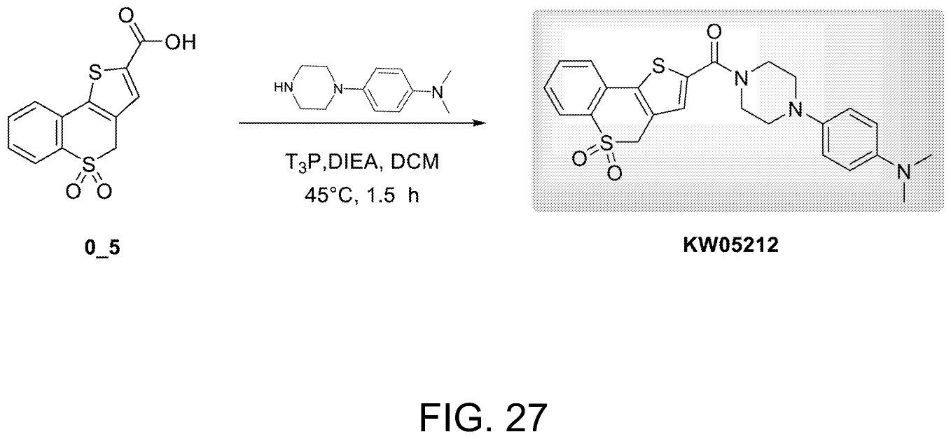

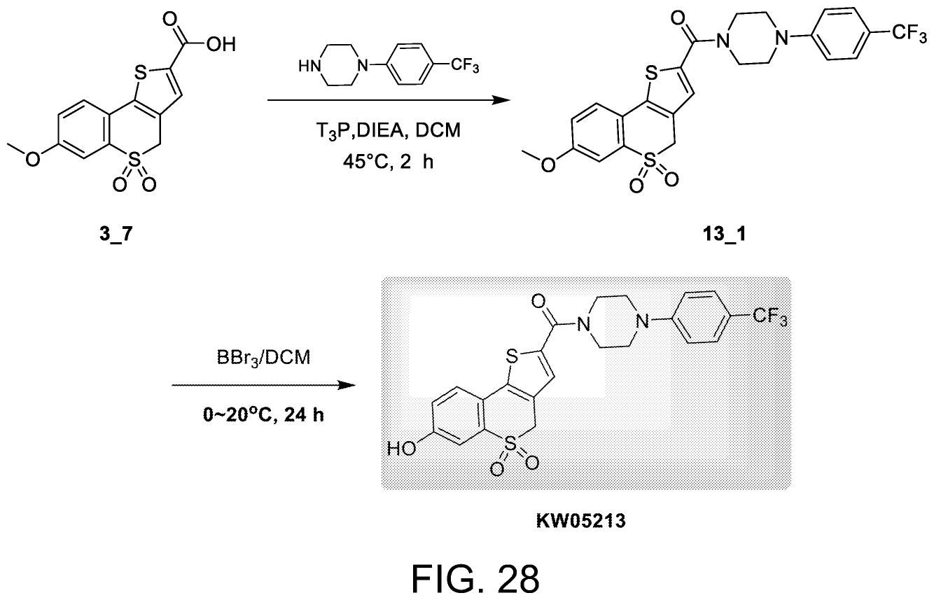

Method for KW05213. FIG. 28 shows a reaction scheme for KW05213. Step 1: To a mixture of compound 3_7 (260 mg, 0.84 mmol) and 1-[4-(trifluoromethyl)phenyl] piperazine (0.19 mL, 1.01 mmol), were added DIEA (0.42 mL, 2.51 mmol) and T₃P (50% in ethyl acetate, 800 mg, 2.51 mmol). The resulting mixture was stirred at 45° C. for 2 h. LC-MS showed the reaction was completed. The reaction was quenched by water (20.0 mL) and extracted with DCM (10 mL×3). The combined organic layers were dried and concentrated to give a residue. The residue was triturated with ethyl acetate (1.0 mL) to give compound 13_1 (150 mg, 0.29 mmol, 34.2%) as a yellow solid.

Step 2: To a solution of compound 13_1 (100 mg, 0.19 mmol) in DCM (5.0 mL) at 0° C. was added tribromoborane (0.18 mL, 1.91 mmol). The resulting mixture was stirred at 20° C. for 24 h. TLC (DCM:MeOH=10:1) showed 50% starting material was remained and 50% desired product was found. The reaction was quenched by water (30.0 mL) and extracted with DCM (10 mL×3). The combined organic layers were dried and concentrated to give a residue. The residue was purified by prep-TLC (DCM:MeOH=10:1) to give compound KW05213 (12.20 mg, 0.02 mmol, 11.9%) as a yellow solid. MS m/z (ESI): =509.0 [M+H]⁺; ¹H NMR (400 MHZ, DMSO-d₆) δ=10.95-10.50 (m, 1H), 7.64 (d, J=8.4 Hz, 1H), 7.57-7.50 (m, 3H), 7.30 (d, J=2.4 Hz, 1H), 7.14 (dd, J=8.8, 2.8 Hz, 1H), 7.08 (d, J=8.8 Hz, 2H), 4.81 (s, 2H), 3.84 (br s, 4H), 3.40-3.45 (m, 4H).

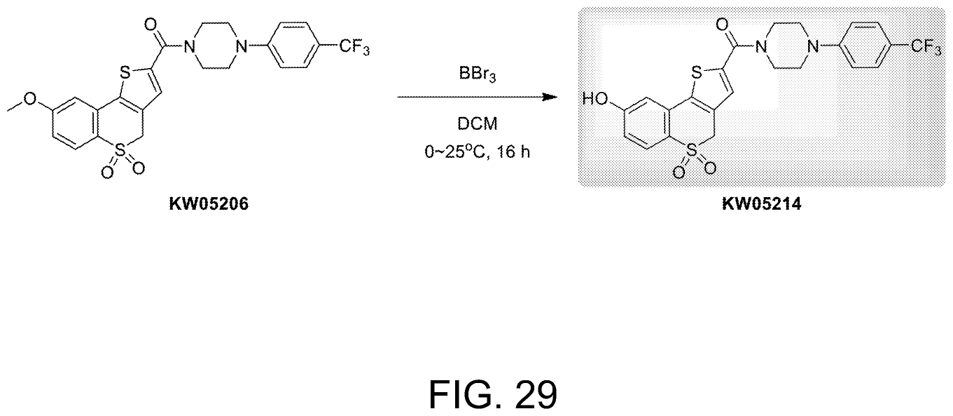

Method for KW05214. FIG. 29 shows a reaction scheme for KW05214. Step 1: To a mixture of compound KW05206 (150 mg, 0.29 mmol) in DCM (3 mL) at 0° C. was added BBr₃ (0.54 mL, 5.74 mmol). The resulting mixture was stirred at 25° C. for 16 h. The reaction mixture was poured into H₂O (15 mL) slowly, then the mixture was extracted with DCM (20 mL×3). The combined organic layers were dried and concentrated to give a residue. The residue was purified by pre-HPLC (ACN/H₂O, 0.1% FA) to give compound KW05214 (27 mg, 0.05 mmol, 18.48%) as an off-white solid. MS m/z (ESI):=509.1 [M+1]⁺; ¹H NMR (400 MHZ, DMSO-d₆) δ 10.80 (s, 1H), 7.79 (d, J=8.5 Hz, 1H), 7.54 (t, J=4.4 Hz, 3H), 7.02-7.15 (m, 3H), 6.98 (dd, J=8.6, 2.3 Hz, 1H), 4.78 (s, 2H), 3.84 (br, 4H), 3.37-3.53 (m, 4H).

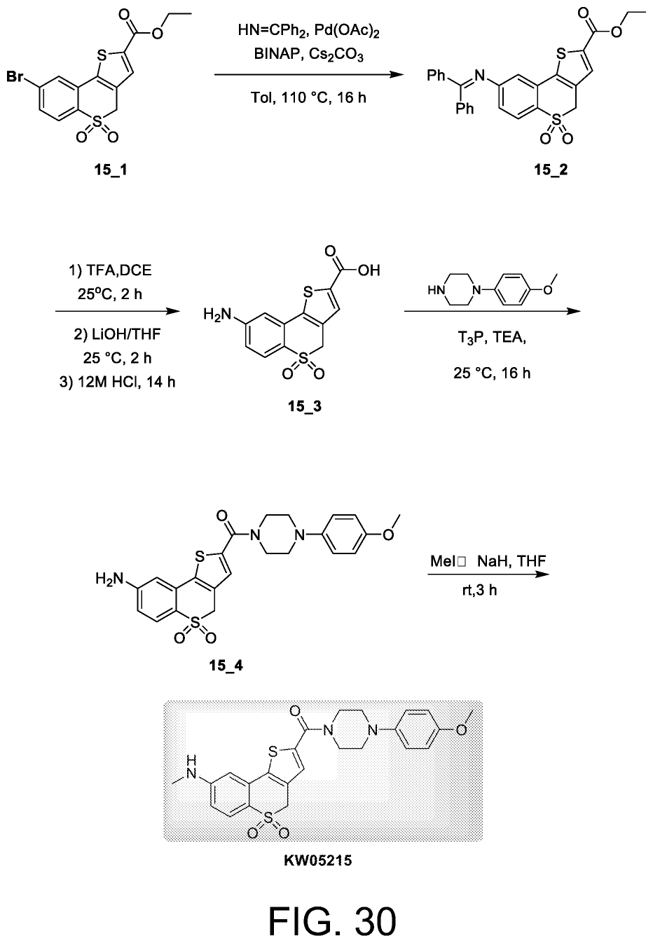

Method for KW05215. FIG. 30 shows a reaction scheme for KW05215. Step 1: To a solution of diphenylmethanimine (1169.00 mg, 6.45 mmol), BINAP (160.65 mg, 0.26 mmol), Cs₂CO₃ (420.31 mg, 1.29 mmol) and compound 15_1 (500 mg, 1.29 mmol) in toluene (15 mL) was added Pd(OAc)₂ (29.19 mg, 0.13 mmol). The resulting mixture was stirred at 110° C. for 16 h under N₂. TLC (Petroleum ether:Ethyl acetate=5:1) indicated starting material was consumed and one major spot was formed. The reaction mixture was cooled down to room temperature and filtered. The filtrate was diluted with H₂O (10 mL) and extracted with ethyl acetate (20 mL×3). The combined organic layers were washed with H₂O (20 mL) and brine (20 mL), dried and concentrated. The residue was purified by chromatography (SiO₂, Petroleum ether:Ethyl acetate=2:1˜0:1) to give compound 15_2 (200 mg, 0.41 mmol, 31.8%) as a yellow solid. MS m/z (ESI):=488.1 [M+H]⁺.

Step 2: To a solution of compound 15_2 (120 mg, 0.39 mmol) in DCE (5 mL) was added TFA (1 mL). The resulting mixture was stirred at 25° C. for 2 h. To the mixture were added H₂O (2 mL), THF (8 mL) and LiOH·H₂O (51.61 mg, 1.23 mmol). The resulting mixture was stirred at 25° C. for 2 h. Then 12N HCl (0.31 mL, 3.69 mmol) was added and the mixture was stirred at 25° C. for another 14 h. TLC (DCM:MeOH=10:1) indicated starting material was consumed and one major spot was formed. The reaction mixture was neutralized with saturated NaHCO₃ aqueous solution to adjust pH to 7 and extracted with ethyl acetate (20 mL×3). The combined organic layers were washed with H₂O (20 mL) and brine (20 mL), concentrated under reduced pressure to give crude compound 15_3 (100 mg, 0.31 mmol, 75.39%) as a brown oil which was used for next step directly. MS m/z (ESI):=296.0 [M+H]⁺.

Step 3: To a solution of crude compound 15_3 (150 mg, 0.51 mmol) in DCM (5 mL) were added 1-(4-methoxyphenyl)piperazine (0.09 mL, 0.51 mmol), T₃P (50% in ethyl acetate, 384.4 mg, 0.52 mmol) and TEA (0.21 mL, 1.53 mmol). The resulting mixture was stirred at room temperature for 18 h. LC-MS indicated starting material was consumed and 15_4 was formed. The mixture was diluted with water and extracted with ethyl acetate. The combined organic layers were concentrated under reduced pressure. The crude product was purified by pre-HPLC (ACN:H₂O, 0.1% FA) and freeze dried to give compound 15_4 (22.19 mg, 0.05 mmol, 13.87% yield) as a light-yellow solid. MS m/z (ESI):=470.5 [M+H]⁺. ¹H NMR (500 MHZ, DMSO-d₆) δ:7.57 (d, J=8.5 Hz, 1H), 7.50 (s, 1H), 6.91-6.96 (m, 2H), 6.80-6.88 (m, 3H), 6.68 (dd, J=8.6, 2.1 Hz, 1H), 6.26 (s, 2H), 4.67 (s, 2H), 3.82 (br s, 4H), 3.69 (s, 3H), 3.03-3.16 (m, 4H).

Step 4: To a solution of compound 15_4 (50 mg, 0.11 mmol) in THF (1 mL) was added NaH (8.52 mg, 0.21 mmol, 60% purity) at 0° C. MeI (22.67 mg, 0.16 mmol) was added at 25° C. The resulting mixture was stirred at 25° C. for 3 h. LC-MS indicated starting material was consumed and KW05215 was formed. The reaction was quenched with water (1 mL) and dissolved in DMF (3 mL). The crude product was purified by Prep-HPLC (ACN: H₂O, 0.1% FA) and freeze dried to give KW05215 (4.33 mg, 0.01 mmol, 8.32% yield) as a light-yellow solid. MS m/z (ESI):=484.3 [M+H]⁺; ¹H NMR (500 MHZ, DMSO-d₆) δ:7.58 (d, J=8.5 Hz, 1H), 7.52-7.56 (m, 1H), 6.92-6.96 (m, J=9.1 Hz, 2H), 6.83-6.87 (m, 2H), 6.78-6.82 (m, 1H), 6.68 (dd, J=8.6, 2.1 Hz, 1H), 6.27 (s, 2H), 4.55-4.63 (m, 1H), 3.82 (br s, 4H), 3.69 (s, 3H), 3.02-3.17 (m, 4H), 1.52 (d, J=7.1 Hz, 3H).

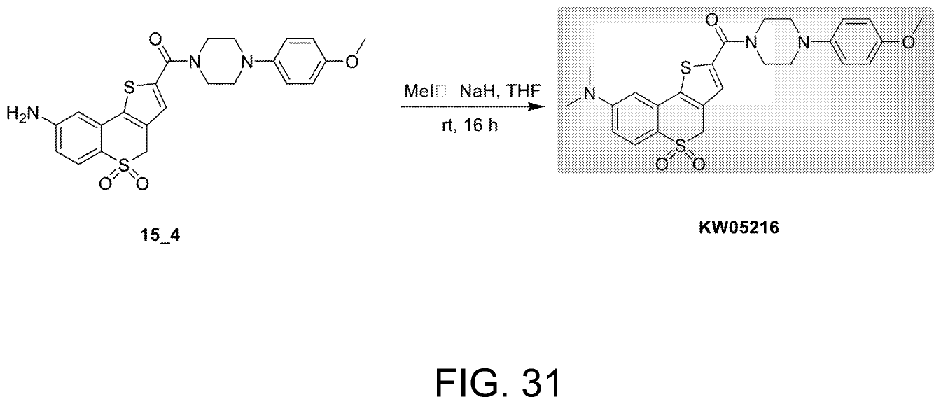

Method for KW05216. FIG. 31 shows a reaction scheme for KW05216. Step 1: To a solution of compound 15_4 (150 mg, 0.32 mmol) in THF (5 mL) was added NaH (63.89 mg, 1.60 mmol) at 0° C. MeI (136.02 mg, 0.96 mmol) was added at 25° C. The mixture was stirred at 25° C. for 16 h. LCMS indicated starting material was consumed and KW05216 was formed. The mixture was quenched with water (1 mL) and dissolved in DMF (3 mL). Then the mixture was purified by Prep-HPLC (ACN/H2O, 0.1% FA) directly and freeze dried to give compound KW05216 (4.90 mg, 0.01 mmol, 8.32% yield) as light brown solid. MS m/z (ESI):=498.5 [M+H]⁺; ¹H NMR (500 MHz, DMSO-d₆) δ: 7.49-7.62 (m, 2H), 6.92 (br d, J=9.1 Hz, 2H), 6.83 (br d, J=9.1 Hz, 2H), 6.75-6.79 (m, 1H), 6.66 (dd, J=8.6, 1.9 Hz, 1H), 6.26 (s, 2H), 3.80 (br s, 4H), 3.67 (s, 4H), 3.08 (br s, 4H), 1.56 (s, 6H).

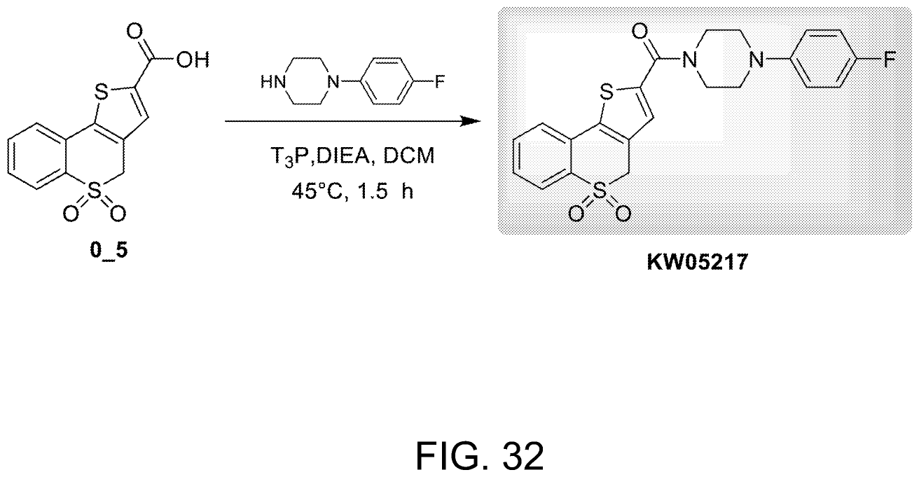

Method for KW05217. FIG. 32 shows a reaction scheme for KW05217. Synthetic method for KW05217 was same as for KW05212: (134.29 mg, 0.30 mmol, 84.30% yield,) as white solid. MS m/z (ESI):=443.1 [M+H]⁺; ¹H NMR (400 MHZ, DMSO-d₆) δ 7.97 (d, J=7.6 Hz, 1H), 7.76-7.82 (m, 2H), 7.65 (ddd, J=8.0, 5.8, 2.6 Hz, 1H), 7.55 (s, 1H), 7.05-7.12 (m, 2H), 6.96-7.02 (m, 2H), 4.90 (s, 2H), 3.78-3.87 (m, 4H), 3.14-3.22 (m, 4H).

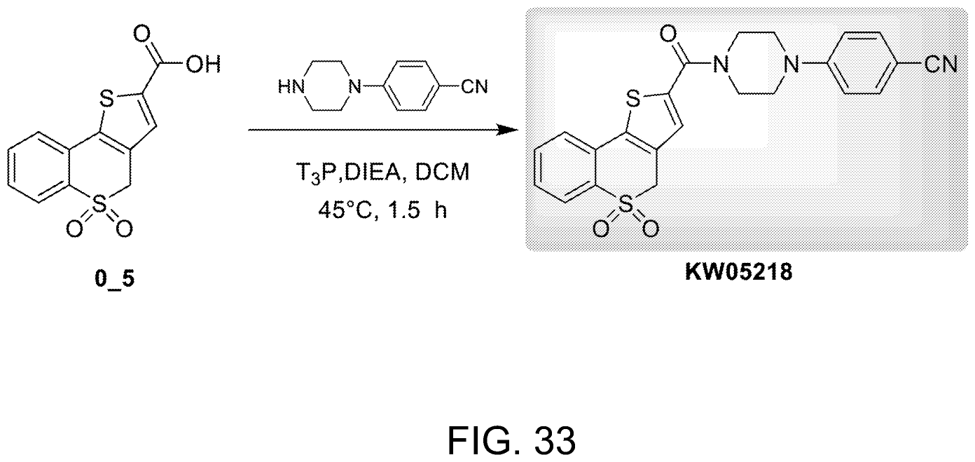

Method for KW05218. FIG. 33 shows a reaction scheme for KW05218. Synthetic method for KW05218 was same as for KW05212: (31.48 mg, 0.06 mmol, 18.05% yield) as light-yellow solid. MS m/z (ESI):=450.1 [M+H]⁺; ¹H NMR (400 MHZ, DMSO-d₆) δ 7.97 (d, J=7.5 Hz, 1H), 7.76-7.83 (m, 2H), 7.60-7.68 (m, 3H), 7.57 (s, 1H), 7.03 (d, J=9.0 Hz, 2H), 4.90 (s, 2H), 3.84 (br, 4H), 3.46-3.55 (m, 4H).

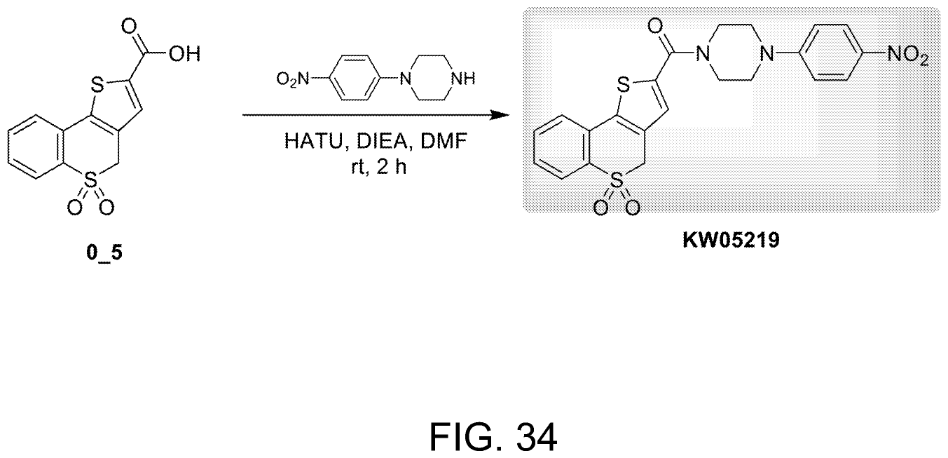

Method for KW05219. FIG. 34 shows a reaction scheme for KW05219. Step 1: To a solution of compound 0_5 (100 mg, 0.36 mmol) in DMF (10 mL) were added 1-(4-nitrophenyl)piperazine (0.06 mL, 0.36 mmol), HATU (162.76 mg, 0.43 mmol), and DIEA (0.18 mL, 1.07 mmol). The resulting mixture was stirred at room temperature for 2 h. LCMS showed that most of compound 0_5 was consumed, and the desired product was formed. The reaction mixture was filtered and washed with ethyl acetate (200 mL). The filter cake was collected and dried to afford compound KW05219 (122 mg, 0.26 mmol, 72.04%) as yellow solid. MS m/z (ESI):=470.1 [M+H]⁺; ¹H NMR (400 MHZ, DMSO-d₆) δ 8.10 (d, J=9.3 Hz, 2H), 7.97 (d, J=8.0 Hz, 1H), 7.77-7.83 (m, 2H), 7.63-7.68 (m, 1H), 7.58 (s, 1H), 7.02 (d, J=9.4 Hz, 2H), 4.91 (s, 2H), 3.79-3.96 (m, 4H), 3.66 (br d, J=5.4 Hz, 4H).

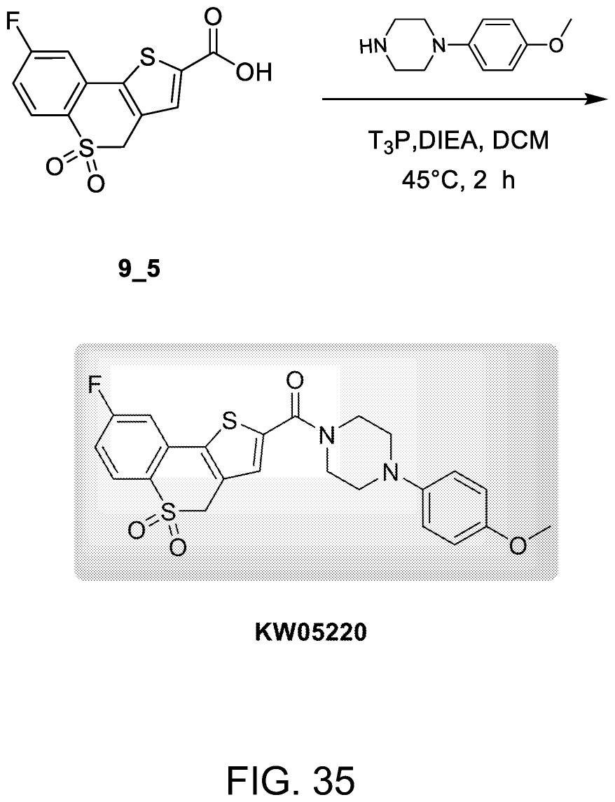

Method for KW05220. FIG. 35 shows a reaction scheme for KW05220. Step 1: To a solution of compound 9_5 (40.0 mg, 0.13 mmol) and 1-(4-methoxyphenyl)piperazine (36.8 mg, 0.16 mmol) in DCM (1.5 mL) was added T₃P (50% in ethyl acetate, 128 mg, 0.40 mmol) and DIEA (0.07 mL, 0.40 mmol). The resulting mixture was stirred at 45° C. for 2 h. TLC (Petroleum ether:Ethyl acetate=1:1) showed starting material was consumed and one new main spot was formed. The reaction was quenched by water (20.0 mL) and extracted with DCM (10 mL×2). The combined organic layers were dried and concentrated to give a residue. The residue was triturated with ethyl acetate (1.0 mL) to give compound KW05220 (10.0 mg, 0.02 mmol, 15.1%) as a green solid. MS m/z (ESI):=473.0 [M+H]⁺.

¹H NMR (400 MHZ, DMSO-d₆) δ=8.04 (dd, J=8.8, 5.4 Hz, 1H), 7.76 (dd, J=9.2, 2.0 Hz, 1H), 7.55 (s, 1H), 7.48 (td, J=8.8, 2.4 Hz, 1H), 6.99-6.90 (m, 2H), 6.85 (br d, J=9.2 Hz, 2H), 4.92 (s, 2H), 3.82 (br s, 4H), 3.69 (s, 3H), 3.10 (br d, J=4.4 Hz, 4H).

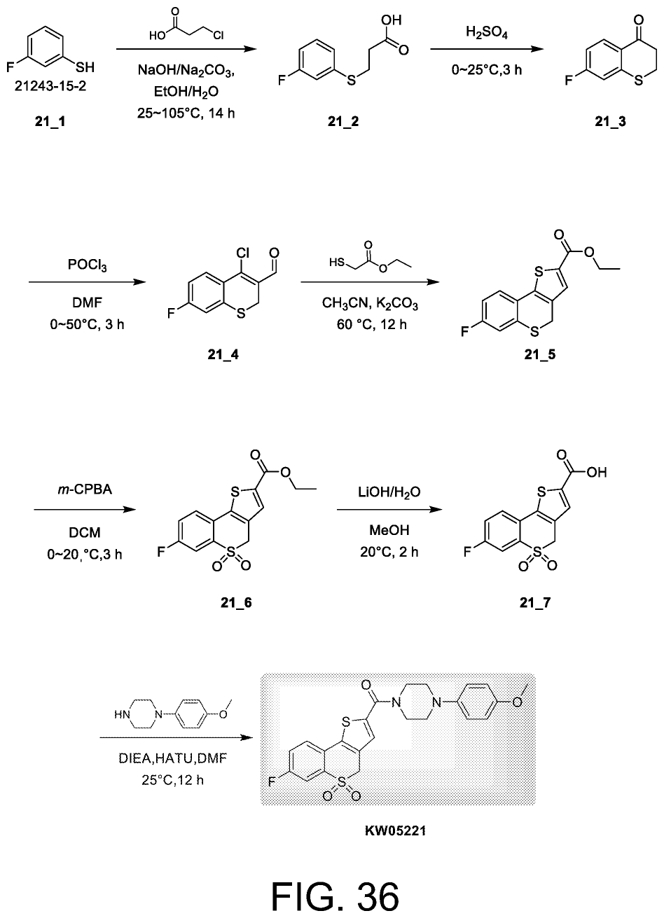

Method for KW05221. FIG. 36 shows a reaction scheme for KW05221. Step 1: To a mixture of NaOH solution (1M, 18.7 mL, 18.7 mmol) and Na₂CO₃ solution (1M, 18.7 mL, 18.7 mmol) was added a solution of compound 21_1 (5.00 g, 39.0 mmol) in EtOH (20.0 mL) under N₂. The resulting mixture was stirred at 25° C. for 5 minutes. Then a solution of 3-chloropropanoic acid (3.50 mL, 41.0 mmol) in H₂O (13.0 mL) was added dropwise. The resulting mixture was stirred at 25° C. for 2 h and then at 105° C. for another 12 h. TLC (Petroleum ether:Ethyl acetate=3:1) indicated starting material was consumed and one major spot was formed. The mixture was concentrated to give a residue. The residue was treated with 2N HCl to adjust pH to 4 and then diluted with water (150 mL) and extracted with ethyl acetate (200 mL×2). The combined organic layers were washed with brine (150 mL), dried and concentrated to give compound 21_2 (5.00 g, 25.0 mmol, 64.0%) as a white solid.

Step 2: Concentrated H₂SO₄ (25.0 mL) was added slowly to compound 21_2 (5.00 g, 25.0 mmol) at 0° C. The resulting mixture was stirred at 25° C. for 3 h. TLC (Petroleum ether:Ethyl acetate=5:1) indicated starting material was consumed and one major spot was formed. The reaction mixture was poured into ice water slowly and extracted with ethyl acetate (50 mL×3). The combined organic layers were washed with water and bine, dried with anhydrous Na₂SO₄, filtered and concentrated to give crude 21_3 (2.10 g, 11.5 mmol, 46.2%) as white solid which was used for next step without further purification.

Step 3: To a mixture of 21_3 (2.10 g, 11.5 mmol) in DMF (30.0 mL) at 0° C. was added POCl₃ (1.61 mL, 17.3 mmol). The resulting mixture was stirred at 50° C. for 3 h. TLC (Petroleum ether:Ethyl acetate=5:1) indicated starting material was consumed and one major spot was formed. The reaction mixture was cooled to 0° C. and then quenched by ice water (20 mL). The mixture was filtered, and the filter cake was washed with water (50 mL) and dried in vacuum to give compound 21_4 (2.10 g, 9.18 mmol, 79.7%) as a yellow solid.

Step 4: To a mixture of compound 21_4 (2.50 g, 10.2 mmol) and K₂CO₃ (2.54 g, 18.4 mmol) in acetonitrile (15.0 mL) was added ethyl sulfanylacetate (1.52 mL, 13.8 mmol). The resulting mixture was stirred at 60° C. for 12 h. TLC (Petroleum ether:Ethyl acetate=5:1) indicated starting material was consumed and one major spot was formed. The mixture was filtered and concentrated to give a residue. The residue was triturated with petroleum ether (6.0 mL) to give crude compound 21_5 (1.80 g, 6.11 mmol, 66.6%) as a yellow solid which was used for the next step without further purification.

Step 5: To a solution of compound 21_5 (1.80 g, 6.11 mmol) in DCM (30.0 mL) at 0° C. was added m-CPBA (2.11 g, 12.2 mmol). The resulting mixture was stirred at 20° C. for 3 h. TLC (Petroleum ether:Ethyl acetate=2:1) indicated starting material was consumed and one major spot was formed. The reaction mixture was quenched by saturated aqueous sodium sulfite solution (50.0 mL) and extracted with DCM (40 mL×3). The combined organic layers were dried and concentrated to give a residue. The residue was triturated with ethyl acetate (5.0 mL) to give compound 21_6 (450 mg, 1.38 mmol, 22.6%) as a yellow solid.

Step 6: To a solution of compound 21_6 (270 mg, 0.79 mmol) in THF (6.00 mL) and H₂O (6.00 mL) was added LiOH·H₂O (59.4 mg, 2.48 mmol). The resulting mixture was stirred at 20° C. for 2 h. TLC (Petroleum ether:Ethyl acetate=3:1) showed starting material was consumed and one major spot was formed. The reaction mixture was treated with saturated citric acid solution to adjust pH to 4, diluted with water (30.0 mL) and extracted with ethyl acetate (50.0 mL×2). The combined organic layers were washed with brine (30.0 mL), dried and concentrated to give crude compound 21_7 (120 mg, 0.40 mmol, 48.6%) as a yellow solid which was used for next step without further purification.

Step 7: To a solution of compound 21_7 (100 mg, 0.34 mmol), 1-(4-methoxyphenyl)piperazine (92.0 mg, 0.40 mmol) and DIEA (0.28 mL, 1.68 mmol) in DMF (3.00 mL) was added HATU (19 mg, 0.50 mmol). The resulting mixture was stirred at 25° C. for 12 h. LCMS showed starting material was consumed and desired product was formed. The reaction was quenched by water (100 mL) and extracted with ethyl acetate (100 mL). The combined organic layers were dried and concentrated to give a residue. The residue was triturated with ethyl acetate (5.00 mL) to give compound KW05221 (23.3 mg, 0.05 mmol, 14.6%) as a yellow solid. MS m/z (ESI):=473.1 [M+H]⁺; ¹H NMR (400 MHZ, DMSO-d₆) δ=7.90 (dd, J=8.8, 4.8 Hz, 1H), 7.83 (dd, J=8.0, 2.8 Hz, 1H), 7.67 (td, J=8.8, 2.8 Hz, 1H), 7.54 (s, 1H), 6.97-6.91 (m, 2H), 6.87-6.83 (m, 2H), 4.94 (s, 2H), 3.82 (br s, 4H), 3.69 (s, 3H), 3.13-3.06 (m, 4H).

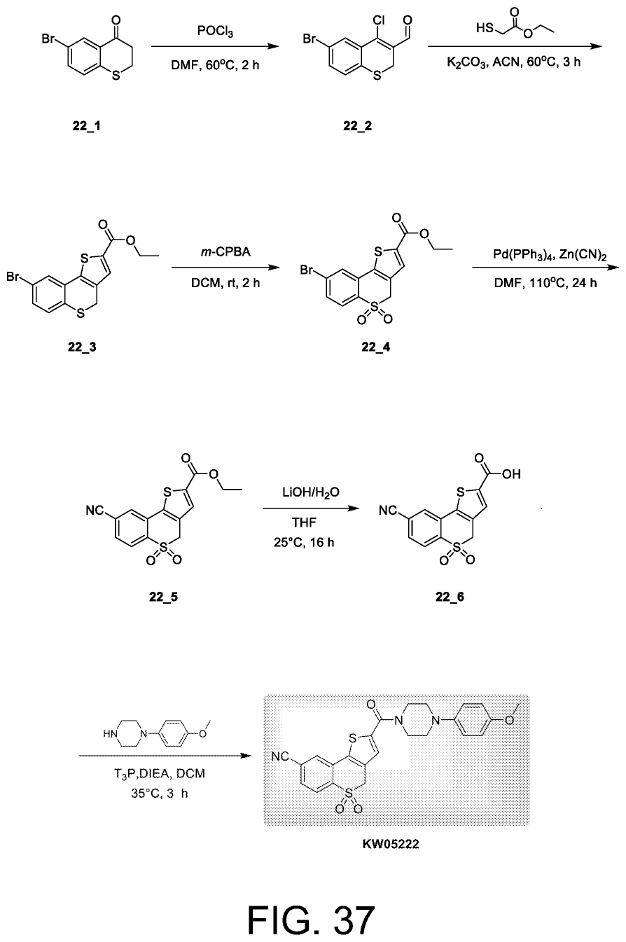

Method for KW05222. FIG. 37 shows a reaction scheme for KW05222. Step 1: To a solution of compound 22_1 (4.50 g, 18.51 mmol) in DMF (40 mL) was added POCl₃ (1.72 mL, 18.51 mmol) dropwise and the resulting mixture was stirred at room temperature for 30 min. The reaction mixture was stirred at 60° C. for 2 h. TLC (Petroleum ether:Ethyl acetate=10:1) showed that the compound 22_1 was consumed completely, and a major new spot was formed. The reaction was quenched with water (50 mL) and extracted with DCM (100 mL×2). The combined organic layers were washed with brine, dried and concentrated in vacuo. The residue was purified using silica gel column chromatography (eluted with Petroleum ether:Ethyl acetate 10:1) to afford compound 22_2 (4.50 g, 14.76 mmol, 79.76%) as a yellow solid. MS m/z (ESI):=288.9 [M+H]⁺

Step 2: To a solution of 22_2 (1.16 g, 4.01 mmol) in CH3CN (15 mL) were added ethyl sulfanylacetate (0.53 mL, 4.81 mmol) and K₂CO₃ (1.11 g, 8.01 mmol). The resulting mixture was stirred at 60° C. for 3 h. TLC (Petroleum ether:Ethyl acetate=5:1) showed that compound 22_2 was consumed completely, and a new spot was formed. The reaction mixture was concentrated in vacuo to give a residue. The residue was dissolved in H₂O (50 mL) and DCM (200 mL). The organic layer was separated, washed with brine, dried and concentrated in vacuo. The residue was diluted with petroleum ether (50 mL), filtered, and collected the filter cake. The filter cake was dried to afford compound 22_3 (1.00 g, 2.67 mmol, 66.75%) as a yellow solid. MS m/z (ESI):=354.5 [M+H]⁺.

Step 3: To a solution of 22_3 (2.50 g, 7.04 mmol) in DCM (100 mL) was added 3-chlorobenzene-1-carboperoxoic acid (2.43 g, 14.07 mmol) and the resulting mixture was stirred at room temperature for 2 h. TLC (Petroleum ether:Ethyl acetate=4:1) showed that most of compound 22_3 was consumed, and a new spot was formed. The reaction mixture was diluted with DCM (200 mL) and quenched by saturated aqueous NaHCO₃ solution (100 mL). The resulting mixture was stirred for 1 h. The organic layer was separated, washed with brine, dried with anhydrous Na₂SO₄ and concentrated in vacuo. The residue was purified using silica gel column chromatography (eluted with Petroleum ether: Ethyl acetate=5:1) to give compound 22_4 (2 g, 4.91 mmol, 69.72%) as a yellow solid. MS m/z (ESI):=386.9 [M+H]⁺.

Step 4: To a mixture of compound 22_4 (250 mg, 0.65 mmol) and Zn (CN) 2 (227.41 mg, 1.94 mmol) in DMF (3 mL) was added Pd(PPh₃)₄ (89.52 mg, 0.08 mmol). The resulting mixture was degassed with N₂ flow and stirred at 110° C. for 24 h. TLC (Petroleum ether:Ethyl acetate=3:1) indicated compound 22_4 was consumed completely, and one major spot was formed. The reaction mixture was diluted with H₂O (15 mL) and extracted with ethyl acetate (45 mL). The combined organic layers were dried and concentrated to give a residue. The residue was triturated with the mixture solution of petroleum ether/ethyl acetate (1:1, 5 mL) at 25° C. for 10 min to give compound 22_5 (125 mg, 0.37 mmol, 48.4%) as a yellow solid. ¹H NMR (400 MHZ, DMSO-d₆) δ 8.47 (s, 1H), 8.10-8.17 (m, 2H), 7.87 (s, 1H), 5.06 (s, 2H), 4.35 (q, J=7.1 Hz, 2H), 1.33 (t, J=7.1 Hz, 3H). ¹³C NMR (400 MHZ, DMSO-d₆) δ 161.19, 137.80, 136.91, 135.20, 134.21, 134.01, 132.31, 130.86, 130.58, 125.03, 117.56, 117.23, 62.19, 50.49, 14.59.

Step 5: To a mixture of compound 22_5 (270 mg, 0.81 mmol) in MeOH (2 mL), THF (2 mL) and H₂O (0.5 mL) was added LiOH·H₂O (78.5 mg, 1.87 mmol). The resulting mixture was stirred at 25° C. for 16 h. TLC (Petroleum ether:Ethyl acetate=3:1) indicated compound 22_5 was consumed completely, and one major spot was formed. The reaction was treated with 2N HCl to adjust the pH to 2-3, then the mixture was diluted with H₂O (5 mL) and extracted with ethyl acetate (15 mL×3). The combined organic layers were dried and concentrated to give crude compound 22_6 (80 mg, 0.26 mmol, 69.88%) as a yellow solid which was used for next step without further purification.

Step 6: To a mixture of compound 22_6 (20 mg, 0.07 mmol) and 1-(4-methoxyphenyl)piperazine (44.95 mg, 0.20 mmol) in ethyl acetate (0.5 mL) was added T₃P (50% in ethyl acetate, 312.64 mg, 0.49 mmol) and DIEA (0.11 mL, 0.66 mmol). The resulting mixture was stirred at 35° C. for 3 h. TLC (Petroleum ether:Ethyl acetate=1:1) indicated several new spots were formed. The reaction mixture was diluted with H₂O (15 mL) and extracted with DCM (15 mL×3). The combined organic layers were dried and concentrated to give a residue. The residue was purified by prep-TLC (SiO₂, DCM:EtOAc=2:1) to give compound KW05222 (25.80 mg, 0.05 mmol, 32.51%) as a yellow solid. MS m/z (ESI):=480.5 [M+1]⁺; ¹H NMR (400 MHZ, DMSO-d₆) § 8.38 (s, 1H), 8.10-8.16 (m, 1H), 8.04-8.09 (m, 1H), 7.56 (s, 1H), 6.90-6.97 (m, 2H), 6.82-6.87 (m, 2H), 5.01 (s, 2H), 3.82 (br, 4H), 3.69 (s, 3H), 3.07-3.13 (m, 4H).

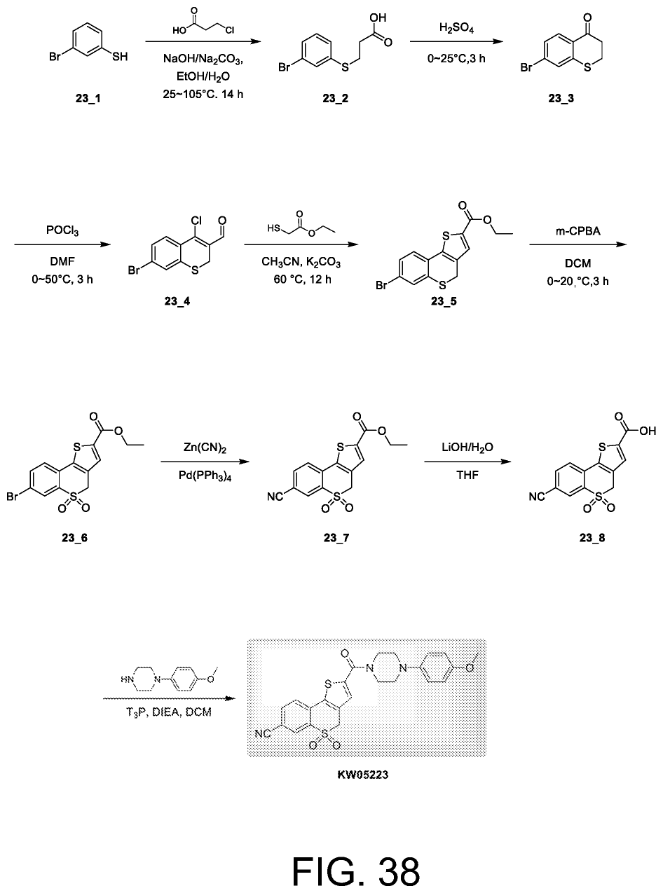

Method for KW05223. FIG. 38 shows a reaction scheme for KW05223 Step 1: To a solution of compound 23_1 (2.32 mL, 20 mmol) in EtOH (12 mL) were added NaOH solution (10.00 mL, 10.00 mmol), Na₂CO₃ solution (10 mL, 10.00 mmol) and 3-chloropropanoic acid (1.88 mL, 22.0 mmol) in H₂O (8 mL). The resulting mixture was stirred at 25° C. for 1 h and then at 110° C. for 12 h. TLC (Petroleum ether:Ethyl acetate=5:1) indicated compound 23_1 was consumed completely, and one major spot was formed. The solvent was removed and 2N HCl was added to adjust the pH to 2-3. The mixture was extracted with DCM (40 mL×3). The combined organic layers were dried and concentrated to give a residue. The residue was triturated with petroleum ether/Ethyl acetate (5:1, 50 mL) at 25° C. for 30 min to give compound 23_2 (3750 mg, 14.36 mmol, 71.80%) as a white solid. ¹H NMR (CDCl₃) δ 7.51 (t, J=1.8 Hz, 1H), 7.33-7.38 (m, 1H), 7.30-7.28 (m, 1H), 7.14-7.22 (m, 1H), 3.19 (t, J=7.3 Hz, 2H), 2.71 (t, J=7.3 Hz, 2H).

Step 2: Compound 23_2 (2750 mg, 10.53 mmol) was added slowly to concentrated H₂SO₄ (7.5 mL) at 0° C. The resulting mixture was stirred at 25° C. for 12 h. The reaction was quenched with ice water and extracted with ethyl acetate (40 mL×3). The combined organic layers were dried and concentrated to give a residue. The residue was purified by chromatography (SiO₂, Petroleum ether:ethyl acetate=1:0 to 10:1) to give compound 23_3 (280 mg, 0.32 mmol, 6.21%) as a yellow oil. ¹H NMR (400 MHZ, CDCl₃) δ 7.96 (d, J=8.5 Hz, 1H), 7.47 (d, J=1.9 Hz, 1H), 7.31 (dd, J=8.5, 1.9 Hz, 1H), 3.23-3.30 (m, 2H), 2.95-3.02 (m, 2H).

Step 3: To a solution of compound 23_3 (1500 mg, 6.17 mmol) in DMF (15 mL) at 0° C. was added POCl₃ (1.15 mL, 12.34 mmol). The resulting mixture was stirred at 50° C. for 4 h. TLC (Petroleum ether:Ethyl acetate=10:1) indicated compound 23_3 was consumed completely, and one major spot was formed. The reaction mixture was diluted with H₂O (30 mL) and extracted with ethyl acetate (120 mL). The combined organic layers were dried and concentrated to give a residue. The residue was triturated with the mixture of petroleum ether/ethyl acetate (8:1, 20 mL) at 25° C. for 10 min to give compound 23_4 (250 mg, 1.06 mmol, 62.72%) as a yellow solid. ¹H NMR (400 MHZ, CDCl₃) δ 10.31 (s, 1H), 7.77 (d, J=8.6 Hz, 1H), 7.54 (d, J=1.9 Hz, 1H), 7.40 (dd, J=8.6, 1.9 Hz, 1H), 3.71 (s, 2H).

Step 4: To a mixture of compound 23_4 (250 mg, 1.06 mmol) and ethyl sulfanylacetate (1.07 mL, 9.67 mmol) in acetonitrile (20 mL) was added K₂CO₃ (1336.3 mg, 9.67 mmol). The resulting mixture was stirred at 60° C. for 12 h. TLC (Petroleum ether:Ethyl acetate=10:1) indicated compound 23_4 was consumed completely, and one major spot was formed. The reaction mixture was diluted with H₂O (30 mL) and extracted with DCM (40 mL×3). The combined organic layers were dried and concentrated to give a residue. The residue was triturated with the mixture of petroleum ether/ethyl acetate (6:1, 50 mL) at 25° C. for 15 min to give compound 23_5 (1200 mg, 3.38 mmol, 69.86%) as a light-yellow solid. 1H NMR (400 MHZ, CDCl₃) δ7.57 (s, 1H), 7.52 (d, J=1.9 Hz, 1H), 7.28-7.36 (m, 2H), 4.37 (q, J=7.1 Hz, 2H), 3.94 (s, 2H), 1.39 (t, J=7.1 Hz, 3H).

Step 5: To a solution of compound 23_5 (600 mg, 1.69 mmol) in DCM (10 mL) at 0° C. was added m-CPBA (583.3 mg, 3.38 mmol) slowly. The resulting mixture was stirred at 25° C. for 0.5 h. TLC (Petroleum ether:Ethyl acetate=4:1) indicated compound 23 5 was consumed completely, and one major spot was formed. The reaction was quenched with saturated Na₂SO₃ solution (20 mL) and stirred at 25° C. for 0.5 h. The mixture was extracted with DCM (20 mL×3) and the combined organic layers were dried and concentrated to give a residue. The residue was triturated with ethyl acetate (20 mL) at 25° C. for 15 min to give compound 23_6 (600 mg, 1.39 mmol, 82.56%) as a white solid. ¹H NMR (400 MHZ, CDCl₃) δ 8.18 (d, J=2.0 Hz, 1H), 7.80 (dd, J=8.4, 2.0 Hz, 1H), 7.69 (s, 1H), 7.53 (d, J=8.4 Hz, 1H), 4.45 (s, 2H), 4.40 (q, J=7.1 Hz, 2H), 1.41 (t, J=7.1 Hz, 3H).

Step 6: To a mixture of compound 23_6 (500 mg, 1.29 mmol) and Zn (CN) 2 (379.01 mg, 3.23 mmol) in DMF (8 mL) was added Pd(PPh₃)₄ (149.20 mg, 0.13 mmol). The resulting mixture was degassed with N₂ flow and then stirred at 110° C. for 24 h. TLC (Petroleum ether:Ethyl acetate=3:1) indicated compound 23_6 was consumed completely, and one major spot was formed. The reaction mixture was diluted with H₂O (15 mL) and extracted with ethyl acetate (20 mL×3). The combined organic layers were dried and concentrated to give a residue. The residue was triturated with ethyl acetate (5 mL) at 25° C. for 15 min to give compound 23_7 (270 mg, 0.81 mmol, 62.73%) as a light-yellow solid. ¹³C NMR (400 MHZ, DMSO-d₆) δ 161.11, 138.16, 137.09, 135.35, 135.31, 134.94, 133.72, 133.34, 128.18, 127.79, 117.65, 112.62, 62.26, 50.68, 14.58.

Step 7: To a mixture of compound 23_7 (135 mg, 0.40 mmol) in MeOH (2 mL), THF (2 mL) and H₂O (0.5 mL) was added LiOH·H₂O (84.8 mg, 2.02 mmol). The resulting mixture was stirred at 25° C. for 1 h. TLC (Petroleum ether:Ethyl acetate=3:1) indicated compound 23_7 was consumed completely, and one major spot was formed. The mixture was treated with 2N HCl to adjust pH to 2-3, then diluted with H₂O (5 mL) and extracted with ethyl acetate (15 mL×3). The combined organic layers were dried and concentrated to give crude compound 23_8 (90 mg, 0.29 mmol, 72.8%) as yellow solid which was used for next step without further purification.

Step 8: To a mixture of compound 23_7 (20 mg, 0.07 mmol) and 1-(4-methoxyphenyl)piperazine (17.98 mg, 0.08 mmol) in ethyl acetate (0.5 mL) were added T₃P (50% in ethyl acetate, 125.05 mg, 0.20 mmol) and DIEA (0.04 mL, 0.26 mmol). The resulting mixture was stirred at 25° C. for 12 h. TLC (Petroleum ether:Ethyl acetate=1:1) indicated several new spots were formed. The reaction mixture was filtered and washed with ethyl acetate (3 mL) to give the crude product. The crude product was triturated with ethyl acetate (3 mL) at 25° C. for 15 min to give compound KW05223 (10.31 mg, 0.02 mmol, 30.45%) as a light-brown solid. MS m/z (ESI):=480.2 [M+1]⁺; ¹H NMR (400 MHZ, DMSO-d₆) § 8.43 (d, J=1.6 Hz, 1H), 8.23 (dd, J=8.2, 1.7 Hz, 1H), 8.00 (d, J=8.1 Hz, 1H), 7.58 (s, 1H), 6.92-6.97 (m, 2H), 6.82-6.87 (m, 2H), 5.02 (s, 2H), 3.81 (br, 4H), 3.69 (s, 3H), 3.07-3.12 (m, 4H).

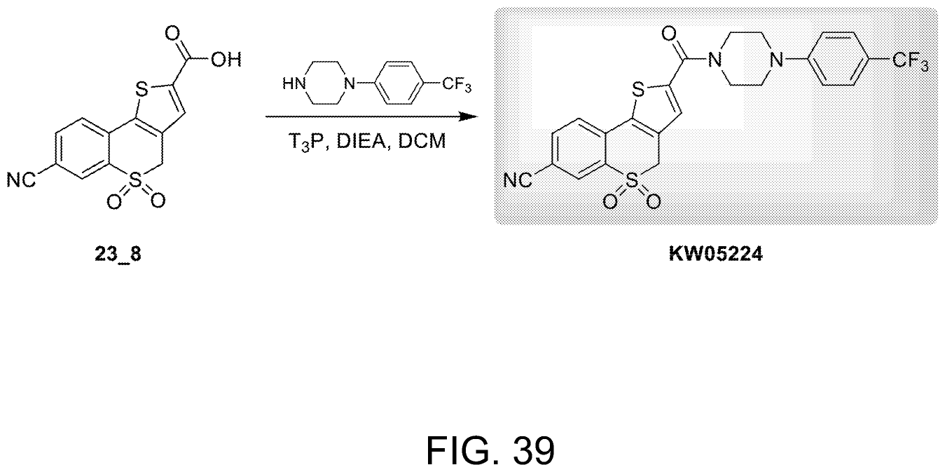

Method for KW05224. FIG. 39 shows a reaction scheme for KW05224. Step 1: To a mixture of compound 23_8 (50 mg, 0.16 mmol) and 1-(4-(trifluoromethyl)phenyl) piperazine (0.04 mL, 0.20 mmol) in DCM (1.2 mL) was added T₃P (50% in ethyl acetate, 208.42, mg, 0.33 mmol) and DIEA (0.05 mL, 0.33 mmol). The resulting mixture was stirred at 35° C. for 12 h. TLC (Petroleum ether:Ethyl acetate=1:1) indicated several new spots were formed. The reaction mixture was filtered and washed with ethyl acetate (3 mL) to give the crude product. The crude product was purified by pre-HPLC (CAN: H₂O, 0.1% FA) to give compound KW05224 (14.20 mg, 0.03 mmol, 16.42%) as a yellow solid. MS m/z (ESI):=518.0 [M+1]⁺; ¹H NMR (400 MHz, DMSO-d₆) δ 8.39 (d, J=1.1 Hz, 1H), 8.11-8.17 (m, 1H), 8.06-8.10 (m, 1H), 7.52-7.59 (m, 3H), 7.09 (d, J=8.8 Hz, 2H), 5.02 (s, 2H), 3.84 (br, 4H), 3.39-3.50 (m, 4H).

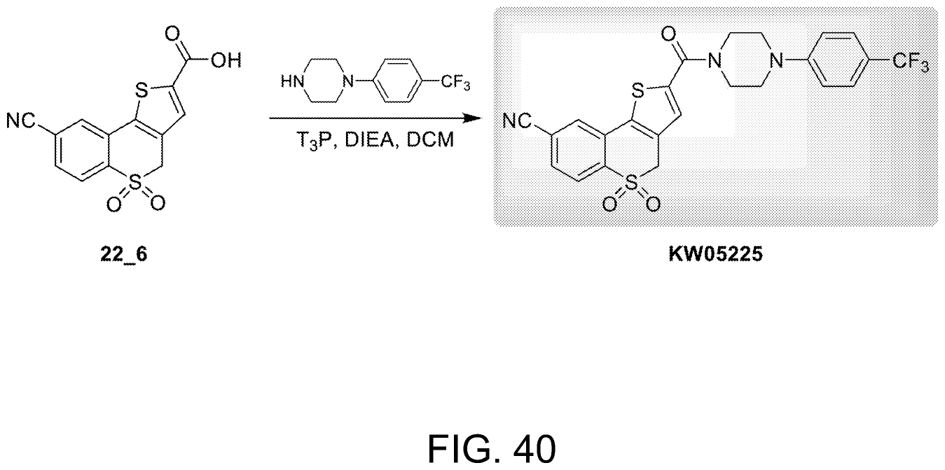

Method for KW05225. FIG. 40 shows a reaction scheme for KW05225. Step 1: To a mixture of compound 22_6 (30 mg, 0.10 mmol) and 1-(4-(trifluoromethyl)phenyl) piperazine (27.6 mg, 0.12 mmol) in DCM (1 mL) were added T₃P (50% in ethyl acetate, 125.05 mg, 0.20 mmol) and DIEA (0.03 mL, 0.20 mmol). The resulting mixture was stirred at 35° C. for 12 h. TLC (Petroleum ether:Ethyl acetate=1/1) indicated several new spots were formed. The reaction mixture was filtered and washed with ethyl acetate (3 mL) to give a crude product. The crude product was purified by prep-HPLC (ACN/H₂O, 0.1% FA) to give compound KW05225 (7.13 mg, 0.01 mmol, 13.98%) as a light-yellow solid. MS m/z (ESI):=518.0 [M+1]⁺; ¹H NMR (400 MHZ, DMSO-d₆) δ 8.43 (d, J=1.5 Hz, 1H), 8.23 (dd, J=8.2, 1.6 Hz, 1H), 8.01 (d, J=8.3 Hz, 1H), 7.60 (s, 1H), 7.55 (d, J=8.8 Hz, 2H), 7.08 (d, J=8.8 Hz, 2H), 5.03 (s, 2H), 3.83 (br, 4H), 3.40-3.48 (m, 4H).

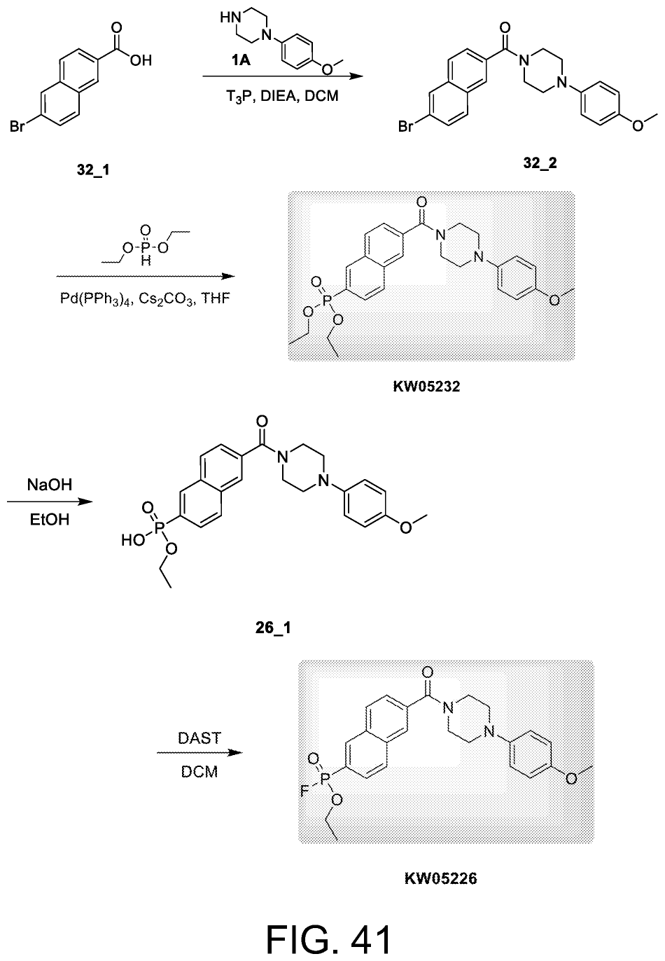

Methods for KW05226 and KW05232. FIG. 41 shows a reaction scheme for each of KW05232 (Steps 1 and 2) and KW-5226 (Steps 3 and 4). Step 1: To a mixture of compound 32_1 (1.00 g, 3.98 mmol) and 1-(4-methoxyphenyl)piperazine (1.37 g, 5.97 mmol) in DCM (20 mL) were added T₃P (50% in ethyl acetate, 5.07 g, 7.97 mmol) and DIEA (3.30 mL, 19.91 mmol). The resulting mixture was stirred at 25° C. for 12 h. TLC (Petroleum ether:Ethyl acetate=1:1) indicated several new spots were formed. The mixture was diluted with H₂O (10 mL) and extracted with ethyl acetate (20 mL×2). The combined organic layers were dried and concentrated to give a residue. The residue was triturated with mixture of petroleum ether/ethyl acetate (2:1, 3 mL) at 25° C. to give compound 32_2 (1.6 g, 3.76 mmol, 94.45%) as a yellow solid. MS m/z (ESI):=426.1 [M+2]⁺. ¹H NMR (400 MHZ, DMSO-d₆) & 8.29 (d, J=1.8 Hz, 1H), 8.07 (s, 1H), 8.01 (s, 1H), 7.99 (d, J=1.5 Hz, 1H), 7.72 (dd, J=8.8, 2.0 Hz, 1H), 7.61 (dd, J=8.4, 1.6 Hz, 1H), 6.90-6.96 (m, 2H), 6.81-6.87 (m, 2H), 3.74-3.94 (m, 2H), 3.69 (s, 3H), 3.46-3.62 (m, 2H), 2.97-3.16 (m, 4H).

Step 2: To a mixture of compound 32_2 (100 mg, 0.22 mmol) and diethyl phosphonate (519.50 mg, 3.76 mmol) in THF (10 mL) were added Pd(PPh₃)₄ (217.35 mg, 0.19 mmol) and Cs₂CO₃ (919.25 mg, 2.82 mmol). The resulting mixture was stirred at 120° C. for 20 min under microwave. TLC (Petroleum ether:Ethyl acetate=0:1) indicated several new spots were formed. The mixture was filtered and washed with ethyl acetate (60 mL). The solution was concentrated to give a residue. The residue was purified by chromatography (SiO₂, Petroleum ether:Ethyl acetate=20:1 to 0:1) to give compound KW05232 (80 mg, 0.15 mmol, 71.21%) as a light-yellow oil. MS m/z (ESI):=483.8 [M+1]⁺; ¹H NMR (400 MHz, DMSO-d₆) δ 8.46 (d, J=15.3 Hz, 1H), 8.22 (d, J=8.4 Hz, 1H), 8.16 (dd, J=8.4, 3.8 Hz, 1H), 8.11 (s, 1H), 7.77 (t, J=9.6 Hz, 1H), 7.65 (d, J=8.3 Hz, 1H), 6.89-6.97 (m, 2H), 6.79-6.88 (m, 2H), 4.00-4.15 (m, 4H), 3.82 (br, 2H), 3.68 (s, 3H), 3.50 (br, 2H), 2.97-3.19 (m, 4H), 1.25 (t, J=7.1 Hz, 7H). ³¹PNMR (400 MHZ, DMSO-d₆) δ:18.5.

Step 3: To a mixture of compound KW05232 (150 mg, 0.31 mmol) in EtOH (1 mL) was added NaOH solution (2M, 1 mL, 2.00 mmol). The resulting mixture was stirred at 60° C. for 2 h. LCMS showed the reaction was completed. The solvent was removed and 2N HCl was added to adjust the pH to 2˜3. The mixture was diluted with H₂O (5 mL) and extracted with ethyl acetate (15 m×3L). The combined organic layers were dried and concentrated to give a residue. The residue was purified by prep-HPLC (ACN/H2O, 0.1% FA) to give compound 26_1 (6.32 mg, 0.01 mmol, 4.41%) as a white solid. MS m/z (ESI):=455.1 [M+1]^{+ 1}H NMR (400 MHZ, DMSO-d₆) δ 8.38 (d, J=14.6 Hz, 1H), 8.16 (d, J=8.3 Hz, 1H), 8.05-8.13 (m, 2H), 7.76 (t, J=9.7 Hz, 1H), 7.61 (dd, J=8.4, 0.9 Hz, 1H), 6.90-6.96 (m, 2H), 6.80-6.88 (m, 2H), 3.86-3.96 (m, 2H), 3.68 (s, 3H), 3.50 (d, J=5.1 Hz, 4H), 2.99-3.15 (m, 4H), 1.18 (t, J=7.1 Hz, 3H).

Step 4: To a mixture of compound 26_1 (350 mg, 0.77 mmol) in DCM (5 mL) at 0° C. was added DAST (0.20 mL, 1.54 mmol). The resulting mixture was stirred at 25° C. for 12 h. TLC (Petroleum ether:Ethyl acetate=1:1) indicated several new spots were formed. The mixture was diluted with H₂O (15 mL) and extracted with ethyl acetate (20 mL×3). The combined organic layers were dried and concentrated to give a residue. The residue was purified by pre-TLC (SiO₂, Petroleum: Ethyl acetate=1:1) to give the crude product, which was purified by pre-HPLC (ACN:H₂O, 0.1% FA) to afford compound KW05226 (18.03 mg, 0.04 mmol, 5.13%) as a white solid. MS m/z (ESI):=457.2 [M+1] *; ¹H NMR (400 MHZ, DMSO-d₆) δ ¹H NMR (DMSO-d₆) δ:8.63 (d, J=16.7 Hz, 1H), 8.28 (d, J=8.5 Hz, 1H), 8.24 (dd, J=8.4, 4.6 Hz, 1H), 8.16 (s, 1H), 7.88-7.83 (m, 1H), 7.70 (dd, J=8.5, 1.6 Hz, 1H), 6.90-6.96 (m, 2H), 6.81-6.88 (m, 2H), 4.26-4.41 (m, 2H), 3.83 (br, 2H), 3.69 (s, 3H), 3.44-3.55 (m, 2H), 2.99-3.19 (m, 4H), 1.36 (t, J=7.1 Hz, 3H).

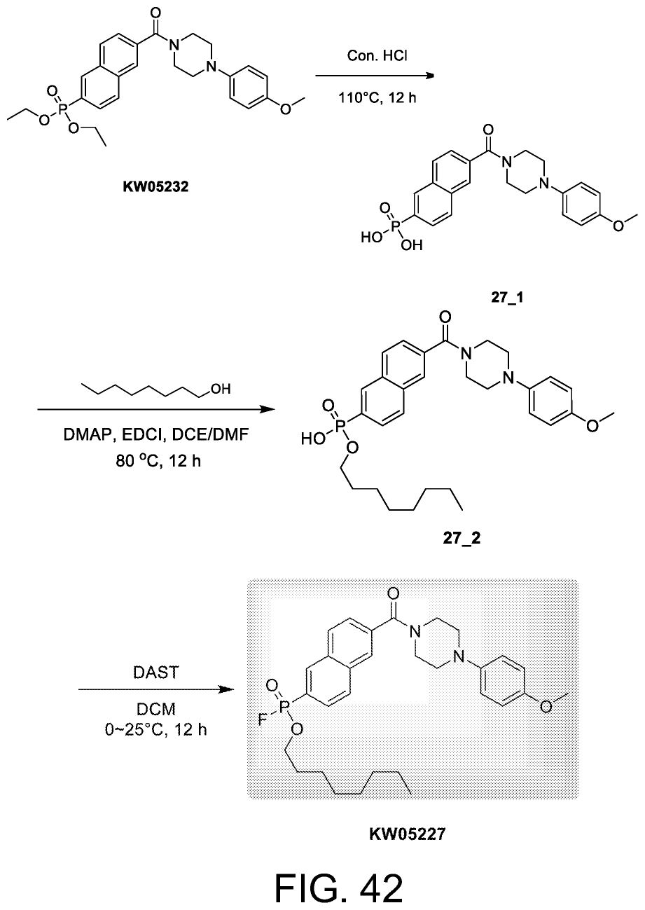

Method for KW05227. FIG. 42 shows a reaction scheme for KW05227. Step 1: A mixture of KW052321 (600 mg, 1.24 mmol) in conc. HCl (4 mL) was stirred at 110° C. for 12 h. TLC (Petroleum ether:Ethyl acetate=1:1) indicated several new spots were formed. The mixture was diluted with H₂O (30 mL) and extracted with ethyl acetate (60 mL). Water was removed through lyophilization to give compound 27_1 (300 mg, 0.70 mmol, 56.58%) as a light brown solid. ¹H NMR (400 MHZ, DMSO-d₆) δ 9.42 (br, 2H), 8.63 (s, 1H), 8.35 (d, J=14.9 Hz, 1H), 8.10-8.24 (m, 2H), 8.02 (dd, J=8.5, 1.5 Hz, 1H), 7.82-7.77 (m, 1H), 7.07 (d, J=8.9 Hz, 2H), 6.88-6.91 (m, 2H), 3.71 (s, 3H), 3.25-3.35 (m, 8H).

Step 2: To a mixture of DMAP (8.60 mg, 0.07 mmol) in octan-1-ol (0.11 mL, 0.70 mmol) at 0° C., were added compound 27_1 (300 mg, 0.70 mmol) in DMF (1 mL)-DCE (1.8 mL) under N₂ and then EDCI (202.31 mg, 1.06 mmol). The resulting mixture was stirred at 80° C. for 12 h. TLC (Petroleum ether:Ethyl acetate=0:1) indicated several new spots were formed. The mixture was diluted with H₂O (10 mL) and treated with 2N HCl (2 N) to adjust pH to 3-4. The mixture was extracted with ethyl acetate (30 mL×3). The combined organic layers were dried and concentrated to give crude compound 27_2 (200 mg, 0.07 mmol, 10.56%) as a brown oil which was used for next step without further purification.

Step 3: To a mixture of compound 27_2 (220 mg, 0.41 mmol) in DCM (1 mL) at 0° C. was added DAST (0.11 mL, 0.82 mmol). The resulting mixture was stirred at 25° C. for 12 h. The mixture was diluted with H₂O (10 mL) and extracted with ethyl acetate (20 mL×3). The combined organic layers were dried and concentrated to give a residue. The residue was purified by pre-HPLC (ACN/H₂O, 0.1% FA) to give compound KW05227 (4.68 mg, 0.01 mmol, 2.08%) as a yellow solid. MS m/z (ESI):=541.2 [M+1]⁺; ¹H NMR (400 MHz, DMSO-d₆) δ:8.63 (d, J=16.8 Hz, 1H), 8.21-8.32 (m, 2H), 8.16 (s, 1H), 7.87-7.82 (m, 1H), 7.70 (dd, J=8.4, 1.4 Hz, 1H), 6.89-6.97 (m, 2H), 6.79-6.87 (m, 2H), 4.23-4.32 (m, 2H), 3.84 (br, 2H), 3.68 (s, 3H), 3.50 (br, 2H), 2.95-3.20 (m, 4H), 1.65-1.75 (m, 2H), 1.29-1.39 (m, 2H), 1.17-1.26 (m, 8H), 0.82 (t, J=6.9 Hz, 3H). ¹⁹FNMR (400 MHZ, DMSO-d₆) δ: −64.5, −62.0. ³¹PNMR (400 MHZ, DMSO-d₆) δ: 20.0, 24.5.

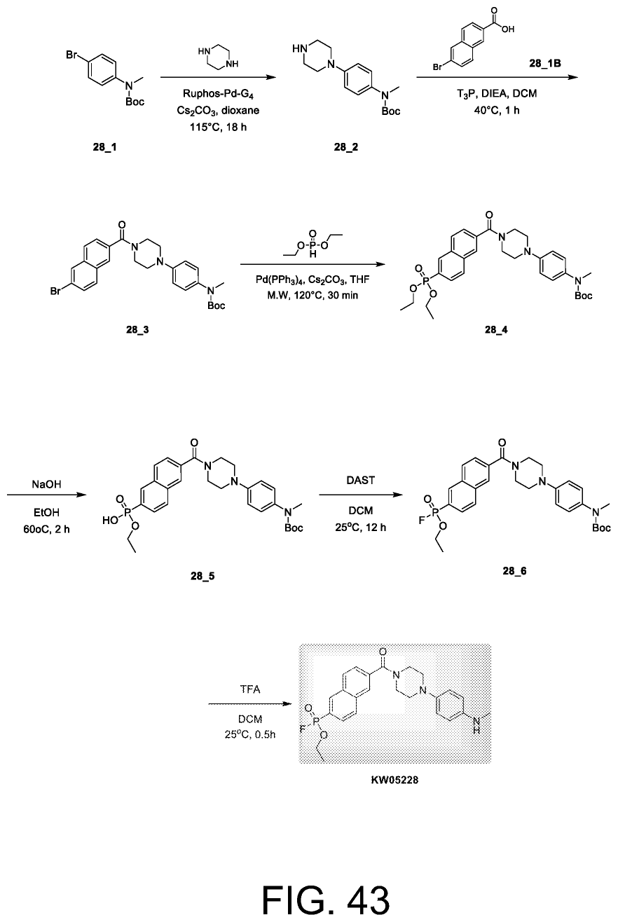

Method for KW05228. FIG. 43 shows a reaction scheme for KW05228. Step 1: To a mixture of compound 28_1 (1000 mg, 3.49 mmol) and piperazine (0.41 mL, 5.24 mmol) in dioxane (15 mL) were added Ruphos Pd G4 (297.64 mg, 0.35 mmol) and Cs₂CO₃ (3415.66 mg, 10.48 mmol). The resulting mixture was stirred at 115° C. for 18 h. LCMS showed starting material was consumed and product was formed. The mixture was filtered and washed with ethyl acetate (50 mL). The combined solution was dried and concentrated to give a residue. The residue was purified by chromatography (SiO₂, Petroleum ether:Ethyl acetate=20:1 to DCM: MeOH=10:1) to give compound 28_2 (530 mg, 1.82 mmol, 52.05%) as a yellow oil. MS m/z (ESI):=292.2 [M+1]⁺. ¹H NMR (400 MHZ, CDCl₃) δ 7.12 (d, J=8.5 Hz, 2H), 6.88 (d, J=8.9 Hz, 2H), 3.22 (s, 3H), 3.13-3.20 (m, 4H), 3.04-3.11 (m, 4H), 1.44 (s, 9H).

Step 2: To a mixture of compound 28_2 (500 mg, 1.99 mmol) and 6-bromo-2-naphthoic acid (695.84 mg, 2.39 mmol) in DCM (12 mL) were added T₃P (50% in ethyl acetate, 1.27 g, 3.98 mmol) and DIEA (0.66 mL, 3.98 mmol). The resulting mixture was stirred at 40° C. for 1 h. TLC (Petroleum ether:Ethyl acetate=0:1) indicated several new spots were formed. The mixture was diluted with H₂O (10 mL) and extracted with DCM (20 mL×3). The combined organic layers were dried and concentrated to give a residue. The residue was purified by chromatography (SiO₂, Petroleum ether:Ethyl acetate=20:1˜2:1) to give compound 28_3 (730 mg, 1.39 mmol, 69.95%) as a white solid. MS m/z (ESI):=468.0 [M-56]^{+ 1}H NMR (400 MHZ, DMSO-d₆) δ 8.28 (d, J=1.8 Hz, 1H), 8.05 (s, 1H), 7.98 (dd, J=8.7, 3.3 Hz, 2H), 7.70 (dd, J=8.8, 2.0 Hz, 1H), 7.59 (dd, J=8.5, 1.6 Hz, 1H), 7.09 (d, J=8.9 Hz, 2H), 6.90 (d, J=9.0 Hz, 2H), 3.68-3.92 (m, 2H), 3.41-3.65 (m, 2H), 3.11-3.30 (m, 4H), 3.09 (s, 3H), 1.35 (s, 9H).

Step 3: To a mixture of compound 28_3 (420 mg, 0.80 mmol) and diethyl phosphonate (165.89 mg, 1.20 mmol) in THF (7 mL) were added Pd(PPh₃)₄ (92.54 mg, 0.08 mmol) and Cs₂CO₃ (391.39 mg, 1.20 mmol). The resulting mixture was stirred at 120° C. for 30 min under microwave. TLC (Petroleum ether:Ethyl acetate=0:1) indicated several new spots were formed. The mixture was diluted with H₂O (20 mL) and extracted with ethyl acetate (25 mL×3). The combined organic layers were dried and concentrated to give a residue. The residue was purified by chromatography (SiO₂, Petroleum ether:Ethyl acetate=20:1˜0:1) to give compound 28_4 (340 mg, 0.58 mmol, 72.99%) as a yellow oil. MS m/z (ESI):=526.2 [M-56]^{+ 1}H NMR (400 MHZ, DMSO-d₆) δ 8.47 (d, J=15.3 Hz, 1H), 7.93-8.05 (m, 3H), 7.79-7.89 (m, 1H), 7.61 (dd, J=8.4, 1.4 Hz, 1H), 7.17 (d, J=8.3 Hz, 2H), 6.86-7.06 (m, 2H), 4.12-4.29 (m, 4H), 3.23 (s, 3H), 1.45 (s, 9H), 1.36 (t, J=7.1 Hz, 6H).

Step 4: To a solution of compound 28_4 (700 mg, 1.20 mmol) in EtOH (4 mL) was added NaOH solution (2M, 4 mL, 8.00 mmol). The resulting mixture was stirred at 60° C. for 2 h. TLC (Petroleum ether:Ethyl acetate=0:1) indicated compound 28_4 was consumed completely, and one major spot was formed. The EtOH solvent was removed and 2N HCl was added to adjust the pH to 2-3. The mixture was extracted with ethyl acetate (40 mL×3) and the combined organic layer was dried and concentrated to give crude compound 28_5 (80 mg, 0.17 mmol, 96.63%) as a yellow oil which was used for next step directly.

Step 5: To a mixture of compound 28_5 (620 mg, 1.12 mmol) in DCM (5 mL) at 0° C. was added DAST (0.30 mL, 2.24 mmol). The resulting mixture was stirred at 25° C. for 12 h. LCMS showed starting material was consumed and product was formed. The mixture was diluted with H₂O (5 mL) and extracted with ethyl acetate (10 mL×3). The combined organic layers were dried and concentrated to give a residue. The residue was purified by prep-HPLC (ACN:H₂O, 0.1% FA) to give compound 28_6 (150 mg, 0.26 mmol, 23.14%) as a light-yellow oil. MS m/z (ESI):=500.5 [M-56]⁺.

Step 6: To a solution of compound 28_6 (50 mg, 0.09 mmol) in DCM (0.5 mL) was added TFA (0.12 mL, 1.61 mmol). The resulting mixture was stirred at 25° C. for 0.5 h. TLC (Petroleum ether:Ethyl acetate=4:1) indicated one major spot was formed. The mixture was concentrated to give a residue, then NaHCO₃ solution was added to adjust the pH to 7-8. The mixture was extracted with ethyl acetate (15 mL×3) and the combined organic layers were dried and concentrated to give a residue. The residue was purified by chromatography (SiO₂, MeOH:DCM=0:1 to 1:10) to give compound KW05228 (22.41 mg, 0.05 mmol, 53.25%) as a yellow-brown solid. MS m/z (ESI):=456.0 [M+1]⁺. ¹H NMR (400 MHZ, DMSO-d₆) δ 8.63 (d, J=16.8 Hz, 1H), 8.20-8.34 (m, 2H), 8.15 (s, 1H), 7.81-7.91 (m, 1H), 7.70 (dd, J=8.4, 1.1 Hz, 1H), 6.80 (d, J=8.1 Hz, 2H), 6.48 (d, J=8.0 Hz, 2H), 4.28-4.40 (m, 2H), 3.81 (br, 2H), 3.47 (br, 2H), 2.84-3.13 (m, 4H), 2.62 (s, 3H), 1.36 (t, J=7.0 Hz, 3H).

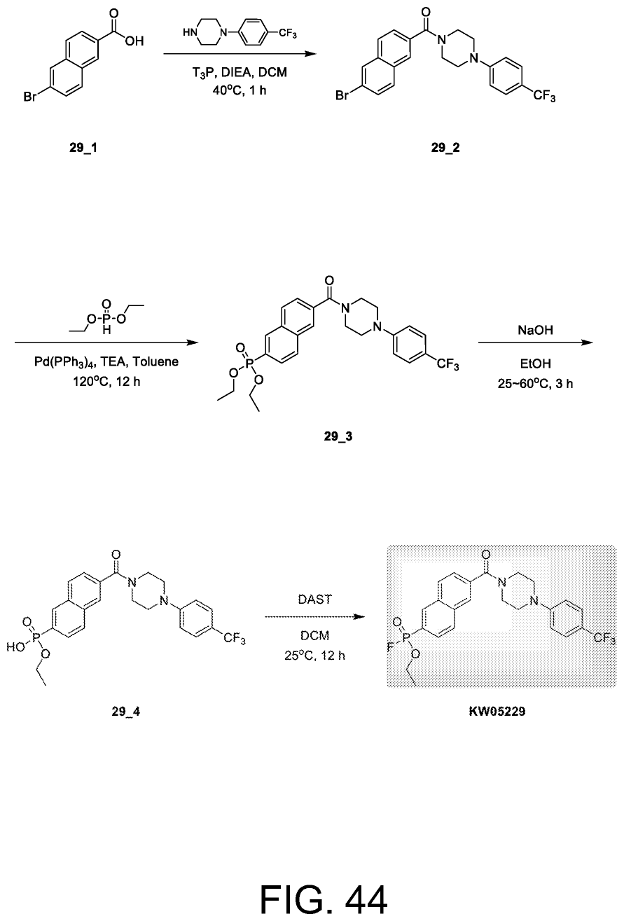

Method for KW05229. FIG. 44 shows a reaction scheme for KW05229. Step 1: To a mixture of compound 29_1 (500 mg, 1.99 mmol) and 1-(4-(trifluoromethyl)phenyl) piperazine (0.50 mL, 2.59 mmol) in DCM (12 mL) were added T₃P (50% in ethyl acetate, 1.27 g, 3.98 mmol) and DIEA (0.66 mL, 3.98 mmol). The resulting mixture was stirred at 40° C. for 1 h. TLC (Petroleum ether:Ethyl acetate=1:1) indicated several new spots were formed. The mixture was diluted with H₂O (15 mL) and extracted with DCM (20 mL×3). The combined organic layers were dried and concentrated to give a residue. The residue was purified by chromatography (SiO₂, Petroleum ether:ethyl acetate=20:1 to 2:1) to give compound 29_2 (550 mg, 1.19 mmol, 59.61%) as a white solid. MS m/z (ESI):=465.0 [M+2]⁺. ¹H NMR (400 MHZ, DMSO-d₆) δ8.29 (s, 1H), 8.30 (d, J=9.0 Hz, 1H), 8.10 (d, J=8.2 Hz, 1H), 8.08 (s, 1H), 8.00 (dd, J=8.6, 5.8 Hz, 2H), 7.67-7.77 (m, 1H), 7.62 (dd, J=8.5, 1.6 Hz, 1H), 7.53 (d, J=8.8 Hz, 2H), 7.08 (d, J=8.8 Hz, 2H), 3.81-3.33 (m, 8H).

Step 2: To a mixture of compound 29_2 (570 mg, 1.23 mmol) and diethyl phosphonate (254.86 mg, 1.85 mmol) in toluene (7 mL) were added Pd(PPh₃)₄ (213.26 mg, 0.18 mmol) and TEA (0.26 mL, 1.85 mmol). The resulting mixture was stirred at 120° C. for 12 h. TLC (Petroleum ether:Ethyl acetate=0:1) indicated several new spots were formed. The mixture was diluted with H₂O (15 mL) and extracted with ethyl acetate (25 mL×3). The combined organic layers were dried and concentrated to give a residue. The residue was purified by chromatography (SiO₂, Petroleum ether:ethyl acetate=20:1 to 0:1) to give compound 29_3 (110 mg, 0.21 mmol, 17.18%) as a yellow oil. MS m/z (ESI):=521.2 [M+1]⁺

Step 3: A mixture of compound 29_3 (482.52 mg, 1 mmol) in aqueous NaOH solution (2M, 0.5 mL, 1.00 mmol) and EtOH (1 mL) was stirred at 25° C. for 1 h. TLC (Petroleum ether:Ethyl acetate=1:1) indicated compound 29_3 was remained, and one new spot was formed. Then the mixture was stirred at 60° C. for another 2 h. TLC (Petroleum ether:Ethyl acetate=0:1) indicated that compound 29_3 was consumed, and one major spot was formed. The EtOH solvent was removed and 2N HCl was added to adjust pH to 2-3. The mixture was extracted with DCM (25 mL×3) and the combined organic layers were dried and concentrated to give crude compound 29_4 (90 mg, 0.11 mmol, 57.08%) as a light-yellow solid which was used for next step without further purification.

Step 4: To a mixture of compound 29_4 (170 mg, 0.35 mmol) in DCM (2 mL) at 0° C. was added DAST (0.09 mL, 0.69 mmol). The resulting mixture was stirred at 25° C. for 12 h. TLC (Petroleum ether:Ethyl acetate=0:1) indicated several new spots were formed. The mixture was diluted with H₂O (10 mL) and extracted with ethyl acetate (15 mL×3). The combined organic layers were dried and concentrated to give a residue. The residue was purified by pre-HPLC (ACN/H₂O, 0.1% FA) to give compound KW05229 (16 mg, 0.03 mmol, 9.37%) as a white solid. MS m/z (ESI):=495.2 [M+1]⁺; ¹H NMR (400 MHz, DMSO-d₆) δ 8.64 (d, J=16.8 Hz, 1H), 8.21-8.34 (m, 2H), 8.18 (s, 1H), 7.86 (dd, J=11.1, 9.1 Hz, 1H), 7.72 (d, J=8.3 Hz, 1H), 7.53 (d, J=8.6 Hz, 2H), 7.08 (d, J=8.6 Hz, 2H), 4.28-4.41 (m, 2H), 3.83 (br, 2H), 3.34-3.68 (m, 6H), 1.36 (t, J=7.0 Hz, 3H).

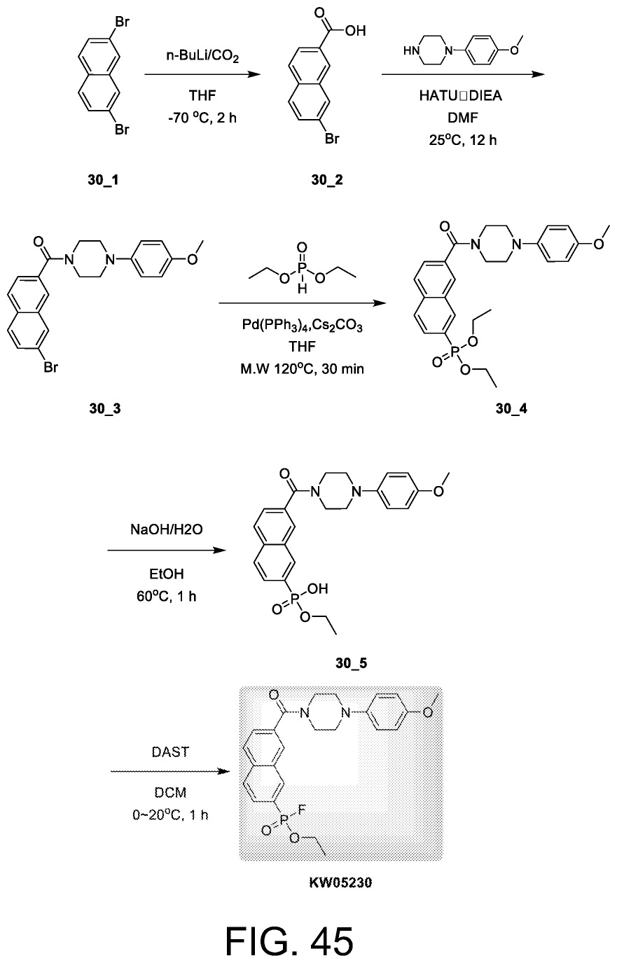

Method for KW05230. FIG. 45 shows a reaction scheme for KW05230. Step 1: In a 100 mL round-bottomed flask, compound 30_1 (2.00 g, 6.99 mmol) was dissolved in THF (28.0 mL). The solution was cooled to −70° C. and n-BuLi (3.08 mL, 7.69 mmol) was added dropwise. The reaction mixture was stirred at −70° C. for 15 min and then CO₂ (gas) was bubbled through the reaction mixture. After 15 minutes, the cold bath was removed, and the reaction mixture was allowed to warm to room temperature over 1.5 h with continued CO₂ bubbling. The mixture was stirred at 20° C. for 12 h. TLC (Petroleum ether:Ethyl acetate=10:1) indicated starting material was consumed and one major spot formed. The reaction mixture was quenched with water (5 mL) and acidified with 1 N HCl until pH=2 and then extracted with ethyl acetate (30 mL×3). The combined organic layers were washed with brine (30.0 mL), dried over anhydrous sodium sulfate, filtered and concentrated to give compound 30_2 (350 mg, 1.39 mmol, 19.9%) as a white solid. ¹H NMR (400 MHZ, DMSO-d₆) δ=14.37-12.01 (m, 1H), 8.60 (br s, 1H), 8.42 (br s, 1H), 8.08-7.92 (m, 3H), 7.76 (br d, J=8.4 Hz, 1H).

Step 2: To a solution of compound 30_2 (350 mg, 1.39 mmol), 1-(4-methoxyphenyl)piperazine (383 mg, 1.67 mmol) and DIEA (0.69 mL, 4.18 mmol) in DMF (7.00 mL) was added HATU (795 mg, 2.09 mmol). The resulting mixture was stirred at 25° C. for 12 h. LCMS showed the starting material was consumed and product was formed. The mixture was quenched by water (100 mL) and extracted with ethyl acetate (30 mL×3). The combined organic layers were dried and concentrated to give a residue. The residue was triturated with ethyl acetate (2.0 mL) to give compound 30_3 (400 mg, 0.94 mmol, 67.5%) as yellow solid. MS m/z (ESI):=425.1, 427.1 [M+1]⁺.

Step 3: To a mixture of compound 30_3 (400 mg, 0.94 mmol) and diethyl phosphonate (260 mg, 1.88 mmol) in THF (8.00 mL) were added Cs₂CO₃ (460 mg, 1.41 mmol) and Pd(PPh₃)₄ (109 mg, 0.09 mmol). The resulting mixture was stirred at 120° C. in microwave for 30 minutes. TLC (Petroleum ether:Ethyl acetate=5:1) indicated 30% starting material was remained and one new spot was formed. The reaction was quenched by water (50.0 mL) and extracted with ethyl acetate (30 mL×3). The combined organic layers were dried and concentrated to give a residue. The residue was purified by chromatography (SiO₂, Petroleum ether:Ethyl acetate=1:1 to 0:1) to give compound 30_4 (320 mg, 0.66 mmol, 70.5%) as a yellow solid. MS m/z (ESI):=483.1 [M+1]⁺.

Step 4: To a solution of compound 30_4 (320 mg, 0.66 mmol) in EtOH (2.0 mL) was added aqueous NaOH solution (2M, 2.00 mL, 4.00 mmol). The resulting mixture was stirred at 60° C. for 1 h. TLC (Petroleum ether:Ethyl acetate-3:1) indicated starting material was consumed and one major spot formed. The reaction solution was concentrated to give a residue. The residue was treated with saturated citric acid solution to adjust the pH to 4, diluted with water (30.0 mL) and extracted with ethyl acetate (60.0 mL×2). The combined organic layers were washed with brine (20.0 mL), dried and concentrated to give crude compound 30_5 (150 mg, 0.33 mmol, 49.8%) as a yellow solid and used for next step without further purification.

Step 5: To a solution of compound 30_5 (160 mg, 0.35 mmol) in DCM (5.00 mL) at 0° C. was added DAST (0.07 mL, 0.53 mmol). The resulting mixture was stirred at 20° C. for 1 h. LCMS showed the reaction was completed. The reaction was quenched by water (30.0 mL) and extracted with DCM (10 mL×3). The combined organic layers were dried and concentrated to give a residue. The residue was purified by prep-HPLC (ACN:H₂O=10:90˜95; 0.1% FA) to give compound KW05230 (2.86 mg, 0.01 mmol, 1.78%) as a yellow solid. MS m/z (ESI):=457.1 [M+1]⁺; ¹H NMR (400 MHZ, DMSO-d₆) δ=8.68 (d, J=16.4 Hz, 1H), 8.30 (s, 1H), 8.21 (dd, J=8.4, 4.4 Hz, 1H), 8.15 (d, J=8.4 Hz, 1H), 7.86 (ddd, J=12.0, 8.4, 1.2 Hz, 1H), 7.76 (dd, J=8.4, 1.2 Hz, 1H), 6.97-6.87 (m, 2H); ³¹P NMR (400 MHZ, DMSO-d₆) δ=13.5, 20.0; ¹⁹F NMR (400 MHZ, DMSO-d₆) δ=−61, −64.5.

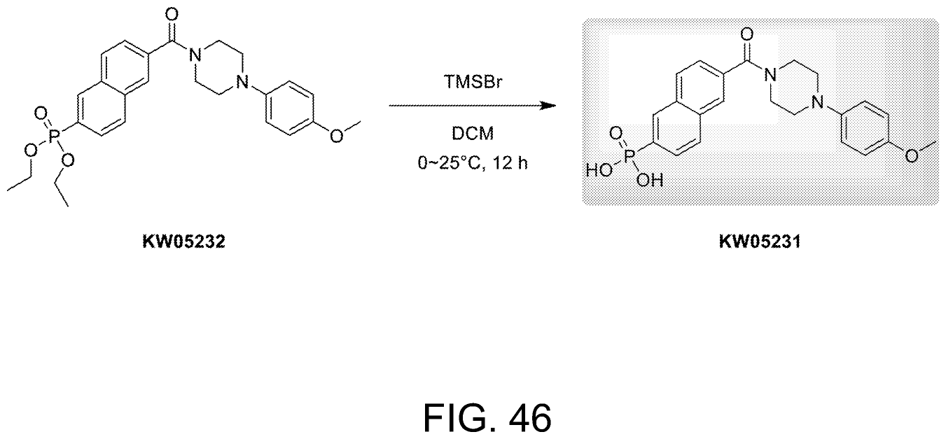

Method for KW05231. FIG. 46 shows a reaction scheme for KW05231. Step 1: To a solution of compound KW05232 (100 mg, 0.21 mmol) in DCM (1 mL) at 0° C. was added TMSBr (0.16 mL, 1.24 mmol). The resulting mixture was stirred at 25° C. for 12 h. TLC (Petroleum ether:Ethyl acetate=0:1) indicated compound KW05232 was consumed completely, and one major spot was formed. The reaction mixture was concentrated to give a residue. The residue was triturated with ethyl acetate (5 mL) at 25° C. for 15 min to give compound KW05231 (16.05 mg, 0.04 mmol, 17.89%) as an off-white solid. MS m/z (ESI):=427.1 [M+1]⁺; ¹H NMR (400 MHZ, DMSO-d₆) δ 8.34 (d, J=15.1 Hz, 1H), 8.15 (d, J=8.4 Hz, 1H), 8.04-8.11 (m, 2H), 7.73-7.83 (m, 1H), 7.62 (d, J=8.4 Hz, 1H), 6.98-7.18 (m, 2H), 6.83-6.96 (m, 2H), 3.79-3.90 (m, 4H), 3.71 (s, 3H), 3.15-3.26 (m, 4H). 31P NMR (400 MHz, DMSO-d₆) δ:28.0.

### Example 7

This example describes use of examples of compounds of the present disclosure as APT1 and/or APT2 inhibitors.

The IC₅₀ value for various compounds is shown in Table 3. The IC₅₀ values were determined by the methods described in Example 2.

TABLE 4

| Compound | APT1 | APT2 | |
|---|---|---|---|
| KW05226 | +++ | +++ | |
| KW05227 | +++ | +++ | |
| KW05228 | ++ | ++ | |
| KW05229 | +++ | +++ | |
| KW05230 | +++ | +++ | |
| KW05231 | − | − | |
| KW05232 | − | − | |
| IC₅₀ < 0.100 μ | | | : +++; |
| 0.100 μM < IC₅₀ < 1 μ | | | : ++; |
| 1 μM < IC₅₀ < 40 μ | | | : +; |

40 μM < IC₅₀: −

The structure of the compounds referenced in Table 4 follow (in order from top to bottom):

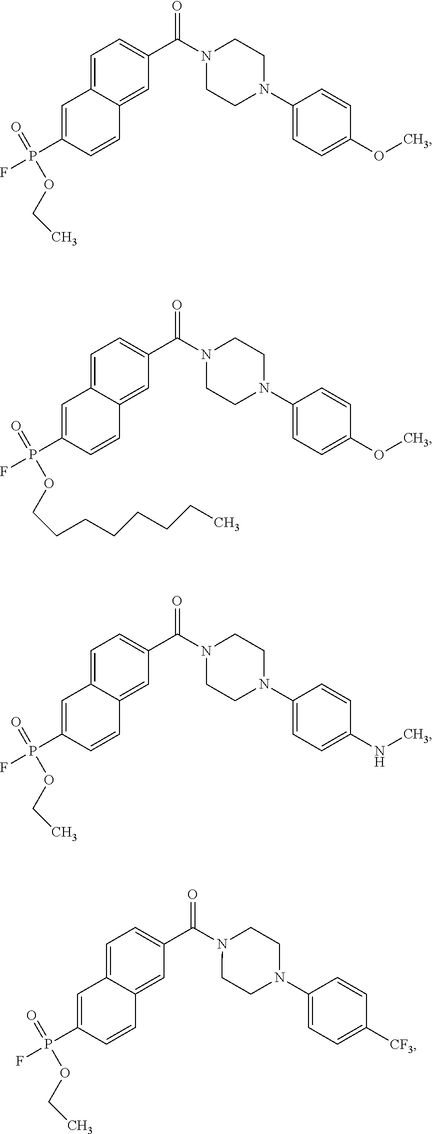 [SMILES: CCOP(=O)(F)c1ccc2cc(C(=O)N3CCN(c4ccc(OC)cc4)CC3)ccc2c1 ; CCCCCCCCOP(=O)(F)c1ccc2cc(C(=O)N3CCN(c4ccc(OC)cc4)CC3)ccc2c1 ; CCOP(=O)(F)c1ccc2cc(C(=O)N3CCN(c4ccc(NC)cc4)CC3)ccc2c1 ; CCOP(=O)(F)c1ccc2cc(C(=O)N3CCN(c4ccc(C(F)(F)F)cc4)CC3)ccc2c1]

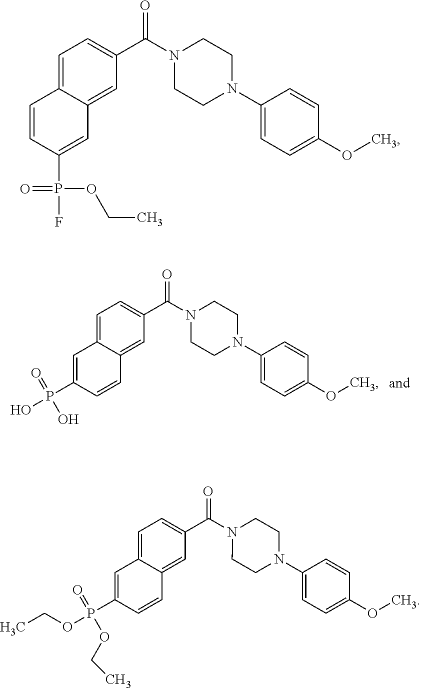 [SMILES: CCOP(=O)(F)c1ccc2ccc(C(=O)N3CCN(c4ccc(OC)cc4)CC3)cc2c1 ; COc1ccc(N2CCN(C(=O)c3ccc4cc(P(=O)(O)O)ccc4c3)CC2)cc1 ; "and" ; CCOP(=O)(OCC)c1ccc2cc(C(=O)N3CCN(c4ccc(OC)cc4)CC3)ccc2c1]

Although the present disclosure has been described with respect to one or more particular examples, it will be understood that other examples of the present disclosure may be made without departing from the scope of the present disclosure.

1. A compound having the following structure: 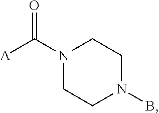
wherein the A group is an aryl group or heterocyclic group, or a substituted derivative or analog thereof,
and
the B group is chosen from alkyl groups, phenyl groups, alkoxylphenyl groups, N-aryl alkylamide groups, N-alkyl alkylamide groups, and substituted derivatives and analogs thereof,
or a pharmaceutically acceptable salt, a salt, a partial salt, a solvate, a polymorph, or a prodrug thereof, or
a stereoisomer or a mixture of stereoisomers, an isotopic variant, or tautomer thereof,
with the proviso the compound does not have the following structure:

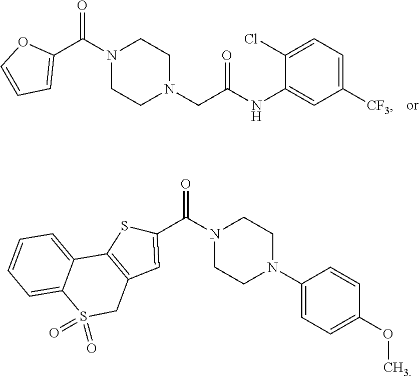 [SMILES: O=C(CN1CCN(C(=O)c2ccco2)CC1)Nc1cc(C(F)(F)F)ccc1Cl ; COc1ccc(N2CCN(C(=O)c3cc4c(s3)-c3ccccc3S(=O)(=O)C4)CC2)cc1]

2. The compound of claim 1, wherein the A group is chosen from furanyl groups, thienyl groups, naphtho thiophen-2-yl groups, oxido/dioxido thieno thiochromeneyl groups, naphthalenyl groups, phenyl groups, and substituted derivatives and analogs thereof.

3. The compound of claim 1, wherein the A group comprises one or more 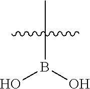 [SMILES: *B(O)O]
group(s) or ester(s) or cyclic esters(s) thereof, one or more 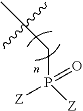
group(s), wherein n is 0, 1, or 2, and each Z is independently chosen from —OH group, halide groups, and alkoxyl groups, one or more 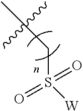
group(s), wherein n is 0, 1, or 2, and W is a halide group, or any combination thereof.

4. The compound of claim 3, wherein the A group is chosen from 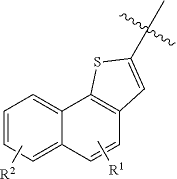
and positional isomers thereof,
wherein R¹ and R² are independently chosen from —H group, —OH group, halide groups, alkyl groups, alkoxyl groups, aryl groups, halogenated hydrocarbon groups, acyl groups, alkylamino groups, cyano group, nitro group, thioether groups, a 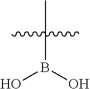 [SMILES: *B(O)O]
group and esters thereof and cyclic esters thereof, 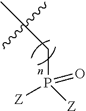
groups, wherein n is 0, 1, or 2, and each Z is independently chosen from —OH group, halide groups, and alkoxyl groups, and 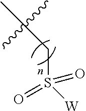
groups, wherein n is 0, 1, or 2, and W is a halide group.

5. The compound of claim 1, wherein the A group is chosen from 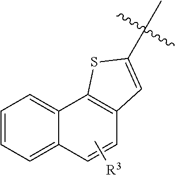
and positional isomers thereof, wherein R³ is chosen from —H group, —OH group, halide groups, alkyl groups, alkoxyl groups, aryl groups, halogenated hydrocarbon groups, acyl groups, alkylamino groups, cyano group, nitro group, thioether groups, 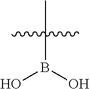 [SMILES: *B(O)O]
group and esters thereof and cyclic esters thereof, 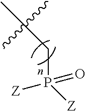
groups, wherein n is 0, 1, or 2, and each Z is independently chosen from —OH group, halide groups, and alkoxyl groups, and 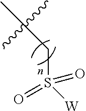
groups, wherein n is 0, 1, or 2, and W is a halide group.

6. The compound of claim 1, wherein the A group has the following structure: 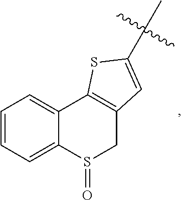 [SMILES: *c1cc2c(s1)-c1ccccc1S(=O)C2]
or a positional isomer thereof.

7. The compound of claim 1, wherein the A group is chosen from 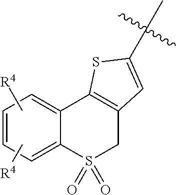
and positional isomers thereof,
wherein each R⁴ is independently chosen from —H group, —OH group, halide groups, alkyl groups, alkoxyl groups, aryl groups, halogenated hydrocarbon groups, acyl groups, alkylamino groups, cyano group, nitro group, thioether groups, 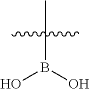 [SMILES: *B(O)O]
group and esters thereof and cyclic esters thereof, 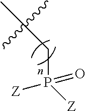
groups, wherein n is 0, 1, or 2, and each Z is independently chosen from —OH group, halide groups, and alkoxyl groups, and 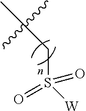
groups, wherein n is 0, 1, or 2, and W is a halide group.

8. The compound of claim 7, wherein the A group has the following structure: 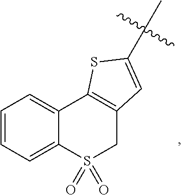 [SMILES: *c1cc2c(s1)-c1ccccc1S(=O)(=O)C2]
or a positional isomer thereof.

9. The compound of claim 1, wherein the A group has the following structure: 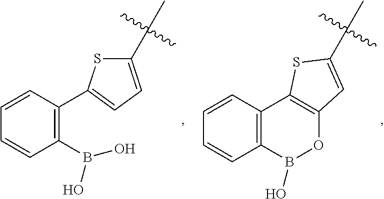 [SMILES: *c1ccc(-c2ccccc2B(O)O)s1 ; *c1cc2c(s1)-c1ccccc1B(O)O2]
or a positional isomer thereof.

10. The compound of claim 1, wherein the A group has the following structure: 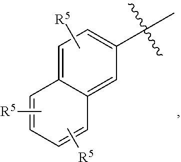
or a positional isomer thereof,
wherein each R⁵ is independently chosen from —H group, —OH group, halide groups, alkyl groups, alkoxyl groups, aryl groups, halogenated hydrocarbon groups, acyl groups, alkylamino groups, cyano group, nitro group, thioether groups, a 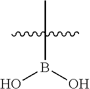 [SMILES: *B(O)O]
group and esters thereof and cyclic esters thereof, 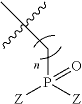
groups, wherein n is 0, 1, or 2, and each Z is independently chosen from —OH group, —OH group, halide groups, and alkoxyl groups, and 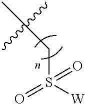
groups, wherein n is 0, 1, or 2, and W is a halide group.

11. The compound of claim 1, wherein the B group is chosen from 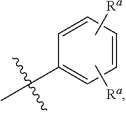
wherein each R^{a} is independently chosen from —H group, —OH group, halide groups, alkyl groups, alkoxyl groups, halogenated hydrocarbon groups, acyl groups, alkylamino groups, cyano group, nitro group, thioether groups, 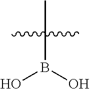 [SMILES: *B(O)O]
group and esters thereof and cyclic esters thereof, 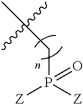
groups, wherein n is 0, 1, or 2, and each Z is independently chosen from —OH group, halide groups, and alkoxyl groups, and 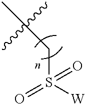
groups, wherein n is 0, 1, or 2, and W is a halide group.

12. The compound of claim 1, wherein the B group is chosen from 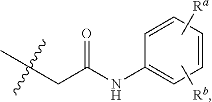
wherein each R^{b} is independently chosen from —H group, —OH group, halide groups, alkyl groups, alkoxyl groups, halogenated hydrocarbon groups, acyl groups, alkylamino groups, cyano group, nitro group, thioether groups, 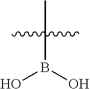 [SMILES: *B(O)O]
group and esters thereof and cyclic esters thereof, 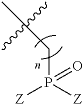
groups, wherein n is 0, 1, or 2, and each Z is independently chosen from —OH group, halide groups, and alkoxyl groups, and 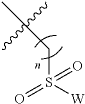
groups, wherein n is 0, 1, or 2, and W is a halide group.

13. The compound of claim 12, wherein the B group has the following structure:

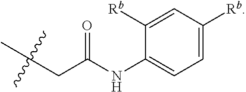

14. The compound according to claim 1, wherein the compound has the following structure: 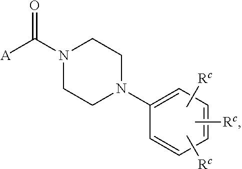
and positional isomers thereof, wherein each R^{c} is independently chosen from —H group, —OH group, halide groups, alkyl groups, alkoxyl groups, halogenated hydrocarbon groups, acyl groups, alkylamino groups, cyano group, nitro group, thioether groups, 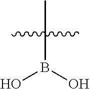 [SMILES: *B(O)O]
group and esters thereof and cyclic esters thereof, 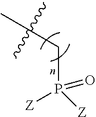
groups, wherein n is 0, 1, or 2, and each Z is independently chosen from —OH group, halide groups, and alkoxyl groups, and 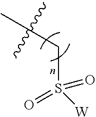
groups, wherein n is 0, 1, or 2, and W is a halide group.

15. The compound of claim 14, wherein the compound has the following structure:

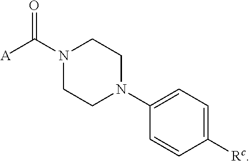

16. The compound of claim 1, wherein the compound is chosen from compounds having the following structure: 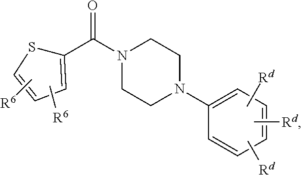
or a positional isomer thereof,
each R^{d} is independently chosen from —H group, —OH group, halide groups, alkyl groups, alkoxyl groups, halogenated hydrocarbon groups, acyl groups, alkylamino groups, cyano group, nitro group, and thioether groups, and each R⁶ is independently chosen from —H group, —OH group, halide groups, alkyl groups, alkoxyl groups, halogenated hydrocarbon groups, acyl groups, alkylamino groups, cyano group, nitro group, thioether groups, 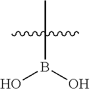 [SMILES: *B(O)O]
group and esters thereof and cyclic esters thereof, 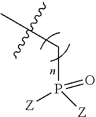
groups, wherein n is 0, 1, or 2, and each Z is independently chosen from —OH group, halide groups, and alkoxyl groups, and 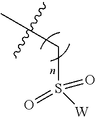
groups, wherein n is 0, 1, or 2, and W is a halide group.

17. The compound of claim 16, wherein only one R⁶ is a 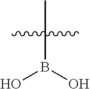 [SMILES: *B(O)O]
group or an ester thereof or a cyclic ester thereof, a 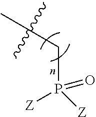
group, wherein n is 0, 1, or 2, and each Z is independently chosen from —OH group, halide groups, and alkoxyl groups, or a 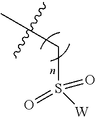
group, wherein n is 0, 1, or 2, and W is a halide group.

18. The compound of claim 16, wherein the compound has the following structure:

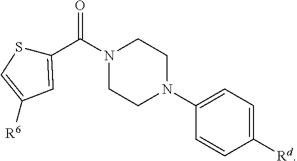

19. The compound of claim 1, wherein the compound is chosen from compounds having the following structure: 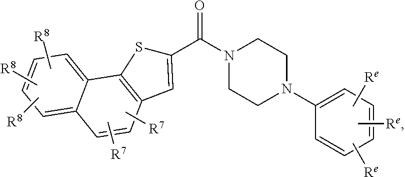
or a positional isomer thereof,
wherein each R^{e} is independently chosen from —H group, —OH group, halide groups, alkyl groups, alkoxyl groups, halogenated hydrocarbon groups, acyl groups, alkylamino groups, cyano group, nitro group, and thioether groups, and
each R⁸ is independently chosen from —H group, —OH group, halide groups, alkyl groups, alkoxyl groups, halogenated hydrocarbon groups, acyl groups, alkylamino groups, cyano group, nitro group, thioether groups, 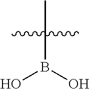 [SMILES: *B(O)O]
group and esters thereof and cyclic esters thereof, 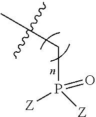
groups, wherein n is 0, 1, or 2, and each Z is independently chosen from —OH group, halide groups, and alkoxyl groups, and 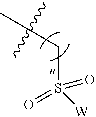
groups, wherein n is 0, 1, or 2, and W is a halide group.

20. The compound of claim 19, wherein only one R⁷ and/or only one R⁸ are present and R⁷ and/or R⁸ is/are independently a 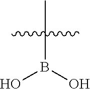 [SMILES: *B(O)O]
group or an ester thereof or a cyclic ester thereof, a 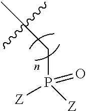
group, wherein n is 0, 1, or 2, and each Z is independently chosen from —OH group, halide groups, and alkoxyl groups, or a 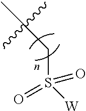
group, wherein n is 0, 1, or 2, and W is a halide group.

21. The compound of claim 19, wherein the compound has the following structure:

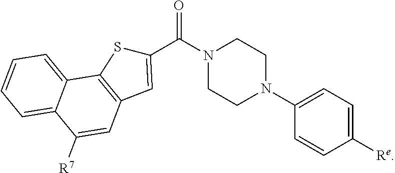

22. The compound of claim 1, wherein the compound is chosen from compounds having the following structure: 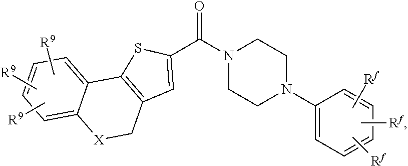
or a positional isomer thereof,
wherein each R^{f} is independently chosen from —H group, —OH group, halide groups, alkyl groups, alkoxyl groups, halogenated hydrocarbon groups, acyl groups, alkylamino groups, cyano group, nitro group, and thioether groups, and
each R⁹ is independently chosen from —H group, —OH group, halide groups, alkyl groups, alkoxyl groups, halogenated hydrocarbon groups, acyl groups, alkylamino groups, cyano group, nitro group, and thioether groups, and
X is —S(O)— or —S(O)₂—.

23. The compound of claim 22, wherein the compound has the following structure:

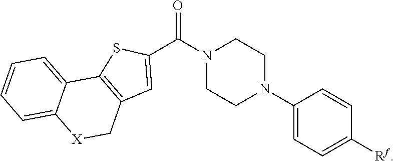

24. The compound of claim 22, wherein the compound has the following structure: 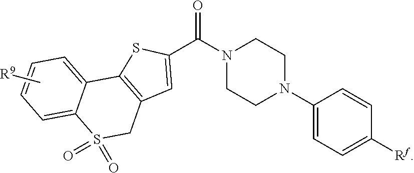
or a positional isomer thereof,
wherein R^{f} is chosen from —H group, halide groups, alkyl groups, alkoxyl groups, alkylamino groups, cyano group, and nitro group, and
R⁹ is chosen from —H group, halide groups, alkoxyl groups, alkylamino groups, cyano group, and nitro group.

25. The compound of claim 1, wherein the compound has chosen from compounds having the following structure: 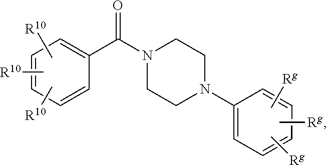
or a positional isomer thereof, wherein each R^{g} is independently chosen from —H group, —OH group, halide groups, alkyl groups, alkoxyl groups, halogenated hydrocarbon groups, acyl groups, alkylamino groups, cyano group, nitro group, thioether groups, 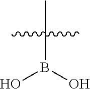 [SMILES: *B(O)O]
group and esters thereof and cyclic esters thereof, 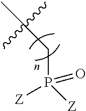
groups, wherein n is 0, 1, or 2, and each Z is independently chosen from —OH group, halide groups, and alkoxyl groups, and 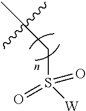
groups,
wherein n is 0, 1, or 2, and W is a halide group, and
groups,
each R¹⁰ is independently chosen from chosen from —H group, —OH group, halide groups, alkyl groups, alkoxyl groups, halogenated hydrocarbon groups, acyl groups, alkylamino groups, cyano group, nitro group, thioether groups, 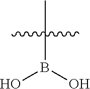 [SMILES: *B(O)O]
group and esters thereof and cyclic esters thereof, 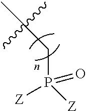
groups, wherein n is 0, 1, or 2, and each Z is independently chosen from —OH group, halide groups, and alkoxyl groups, and 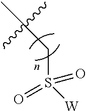
groups, wherein n is 0, 1, or 2, and W is a halide group.

26. The compound of claim 25, wherein only one R^{g} and/or only one R¹⁰ group is/are independently a 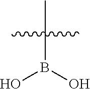 [SMILES: *B(O)O]
group or an ester thereof or a cyclic ester thereof, a 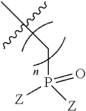
group, wherein n is 0, 1, or 2, and each Z is independently chosen from —OH group, halide groups, and alkoxyl groups, or a 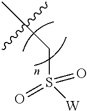
group, wherein n is 0, 1, or 2, and W is a halide group.

27. The compound of claim 25, wherein the compound has the following structure:

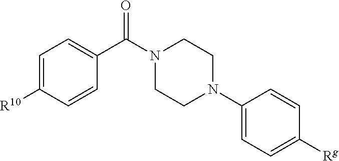

28. The compound of claim 1, wherein the compound has the following structure: 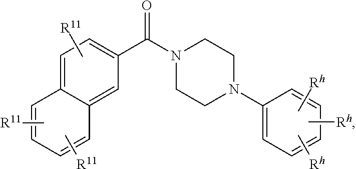
or a positional isomer thereof, wherein each R^{h} is independently chosen from —H group, —OH group, halide groups, alkyl groups, alkoxyl groups, halogenated hydrocarbon groups, acyl groups, alkylamino groups, cyano group, nitro group, thioether groups, 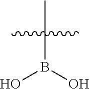 [SMILES: *B(O)O]
group and esters thereof and cyclic esters thereof, 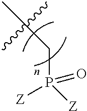
groups, wherein n is 0, 1, or 2, and each Z is independently chosen from —OH group, halide groups, and alkoxyl groups, and 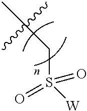
groups,
wherein n is 0, 1, or 2, and W is a halide group, and
each R¹¹ is independently chosen from chosen from —H group, —OH group, halide groups, alkyl groups, alkoxyl groups, halogenated hydrocarbon groups, acyl groups, alkylamino groups, cyano group, nitro group, thioether groups, 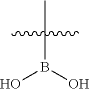 [SMILES: *B(O)O]
group and esters thereof and cyclic esters thereof, 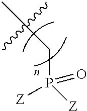
groups, wherein n is 0, 1, or 2, and each Z is independently chosen from —OH group, halide groups, and alkoxyl groups, and 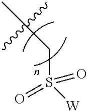
groups, wherein n is 0, 1, or 2, and W is a halide group.

29. The compound of claim 28, wherein only one R^{h} and/or only one R¹¹ group is/are present and R^{g} and/or R¹¹ is/are independently a 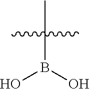 [SMILES: *B(O)O]
group or an ester thereof or a cyclic ester thereof, a 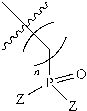
group, wherein n is 0, 1, or 2, and each Z is independently chosen from —OH group, halide groups, and alkoxyl groups, or a 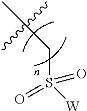
group, wherein n is 0, 1, or 2, and W is a halide group.

30. The compound of claim 28, wherein the compound has the following structure: 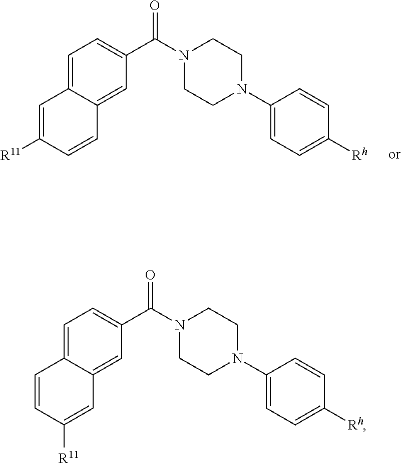
wherein R^{h} is chosen from alkyl groups, alkoxyl groups, and alkyl amino groups, and R¹¹ is a 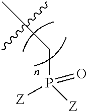
group, wherein n is 0, 1, or 2 and each Z is independently chosen from —OH group, halide groups, and alkoxyl groups.

31. The compound of claim 1, wherein the compound is APT-MY1, APT-MY2, APT-MY4, APT-MY8, APT-MY9, APT-MY11, APT-MY12, KW05129, KW05175, KW05191, KW05192, KW06034, KW0526pp, KW05151, KW05153, KW05169, KW05177, KW05179, KW05200, KW05201, KW05202, KW05203, KW05204, KW05205, KW05206, KW05207, KW05208, KW05209, KW05210, KW05211, KW05212, KW05213, KW05214, KW05215, KW05216, KW05217, KW05218, KW05219, KW05220, KW05221, KW05222, KW05223, KW05224, KW05225, KW05226, KW05227, KW05228, KW05229, KW05230, KW05231, KW05232, KW05130, KW05108, or KW05116.

32. The compound of claim 1, wherein the compound has the following structure:

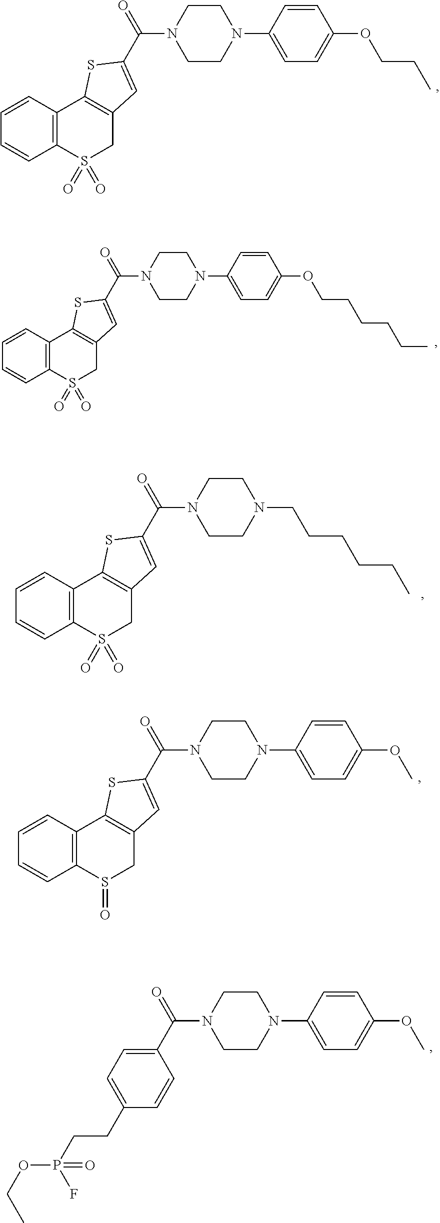 [SMILES: CCCOc1ccc(N2CCN(C(=O)c3cc4c(s3)-c3ccccc3S(=O)(=O)C4)CC2)cc1 ; CCCCCCOc1ccc(N2CCN(C(=O)c3cc4c(s3)-c3ccccc3S(=O)(=O)C4)CC2)cc1 ; CCCCCCN1CCN(C(=O)c2cc3c(s2)-c2ccccc2S(=O)(=O)C3)CC1 ; COc1ccc(N2CCN(C(=O)c3cc4c(s3)-c3ccccc3S(=O)C4)CC2)cc1 ; CCOP(=O)(F)CCc1ccc(C(=O)N2CCN(c3ccc(OC)cc3)CC2)cc1]

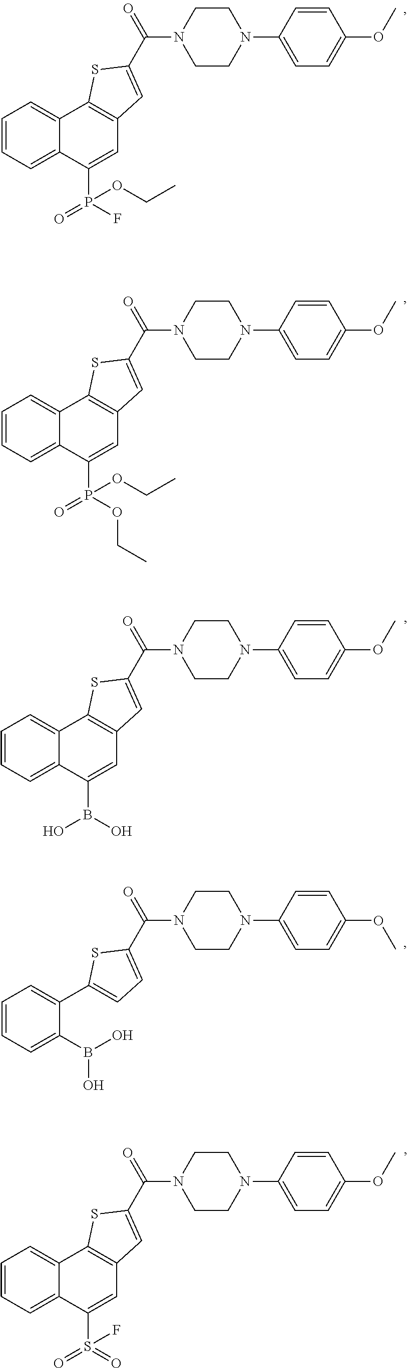 [SMILES: CCOP(=O)(F)c1cc2cc(C(=O)N3CCN(c4ccc(OC)cc4)CC3)sc2c2ccccc12 ; CCOP(=O)(OCC)c1cc2cc(C(=O)N3CCN(c4ccc(OC)cc4)CC3)sc2c2ccccc12 ; COc1ccc(N2CCN(C(=O)c3cc4cc(B(O)O)c5ccccc5c4s3)CC2)cc1 ; COc1ccc(N2CCN(C(=O)c3ccc(-c4ccccc4B(O)O)s3)CC2)cc1 ; COc1ccc(N2CCN(C(=O)c3cc4cc(S(=O)(=O)F)c5ccccc5c4s3)CC2)cc1]

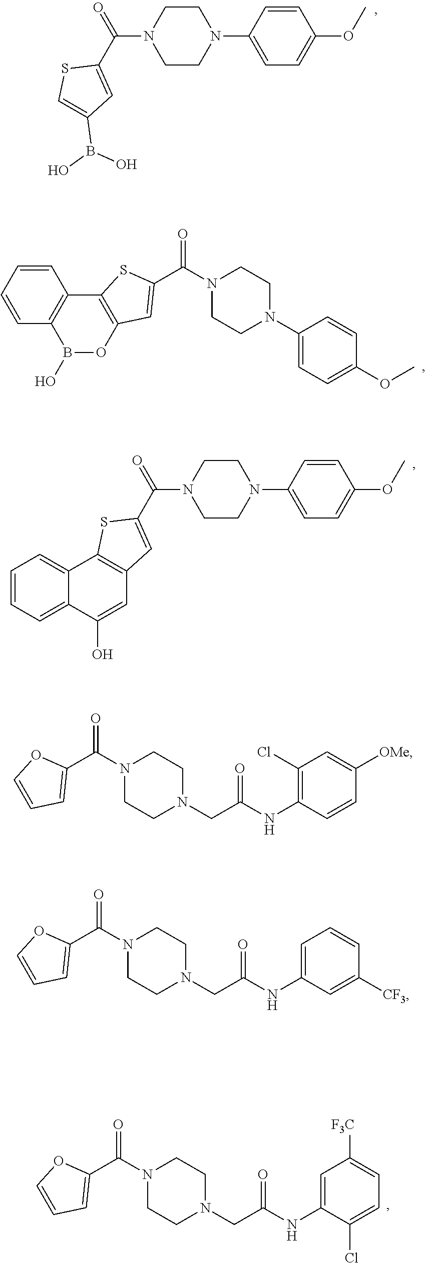 [SMILES: COc1ccc(N2CCN(C(=O)c3cc(B(O)O)cs3)CC2)cc1 ; COc1ccc(N2CCN(C(=O)c3cc4c(s3)-c3ccccc3B(O)O4)CC2)cc1 ; COc1ccc(N2CCN(C(=O)c3cc4cc(O)c5ccccc5c4s3)CC2)cc1 ; COc1ccc(NC(=O)CN2CCN(C(=O)c3ccco3)CC2)c(Cl)c1 ; O=C(CN1CCN(C(=O)c2ccco2)CC1)Nc1cccc(C(F)(F)F)c1 ; O=C(CN1CCN(C(=O)c2ccco2)CC1)Nc1cc(C(F)(F)F)ccc1Cl]

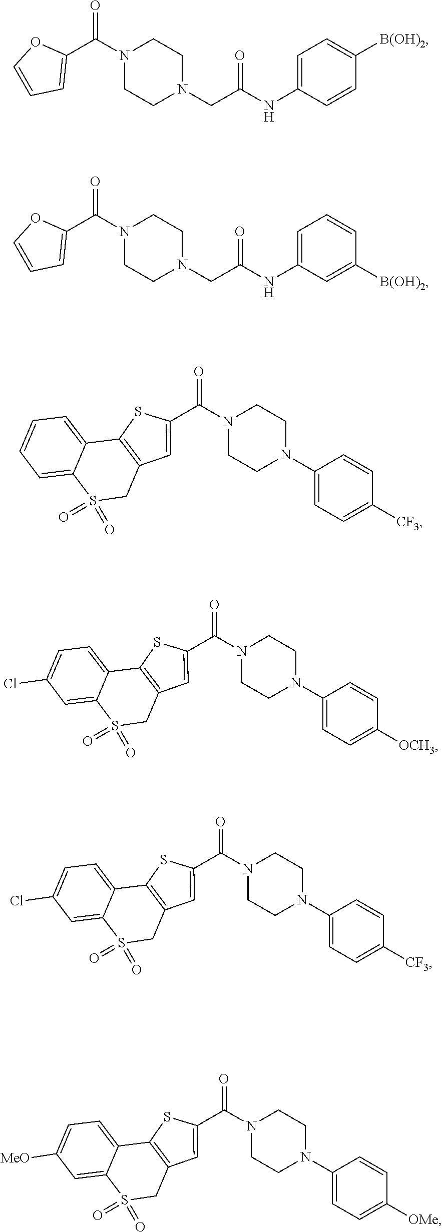 [SMILES: O=C(CN1CCN(C(=O)c2ccco2)CC1)Nc1ccc(B(O)O)cc1 ; O=C(CN1CCN(C(=O)c2ccco2)CC1)Nc1cccc(B(O)O)c1 ; O=C(c1cc2c(s1)-c1ccccc1S(=O)(=O)C2)N1CCN(c2ccc(C(F)(F)F)cc2)CC1 ; COc1ccc(N2CCN(C(=O)c3cc4c(s3)-c3ccc(Cl)cc3S(=O)(=O)C4)CC2)cc1 ; O=C(c1cc2c(s1)-c1ccc(Cl)cc1S(=O)(=O)C2)N1CCN(c2ccc(C(F)(F)F)cc2)CC1 ; COc1ccc(N2CCN(C(=O)c3cc4c(s3)-c3ccc(OC)cc3S(=O)(=O)C4)CC2)cc1]

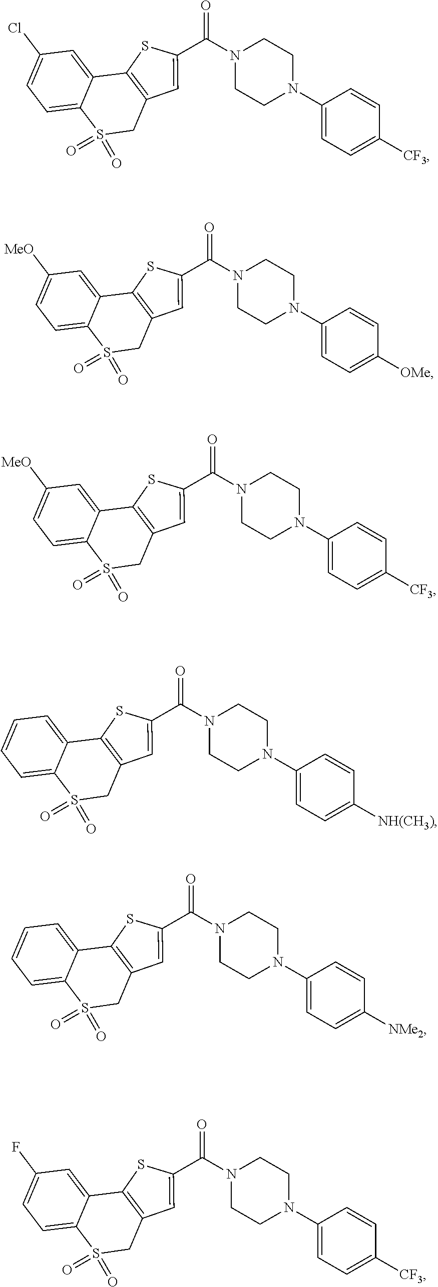

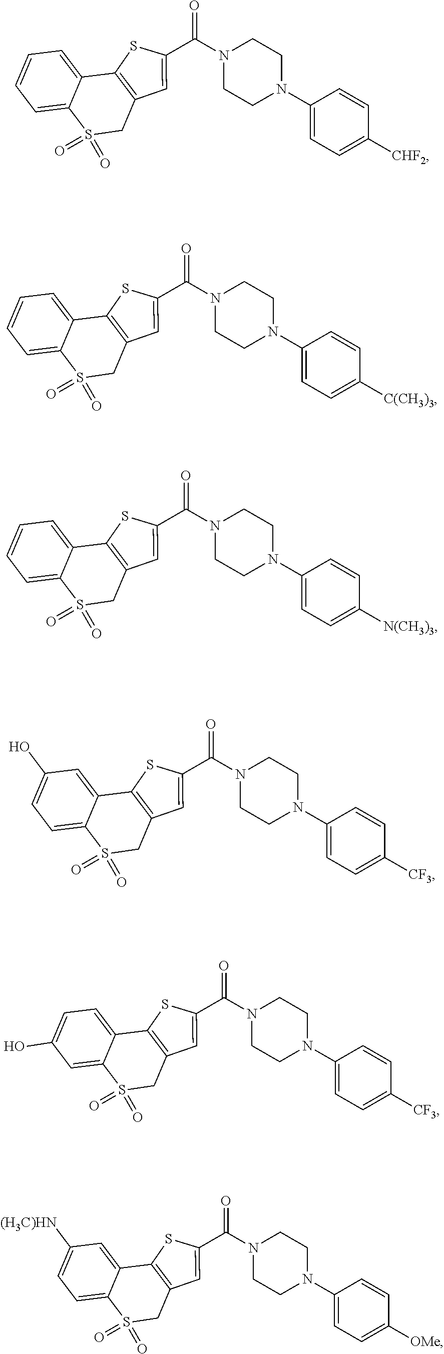

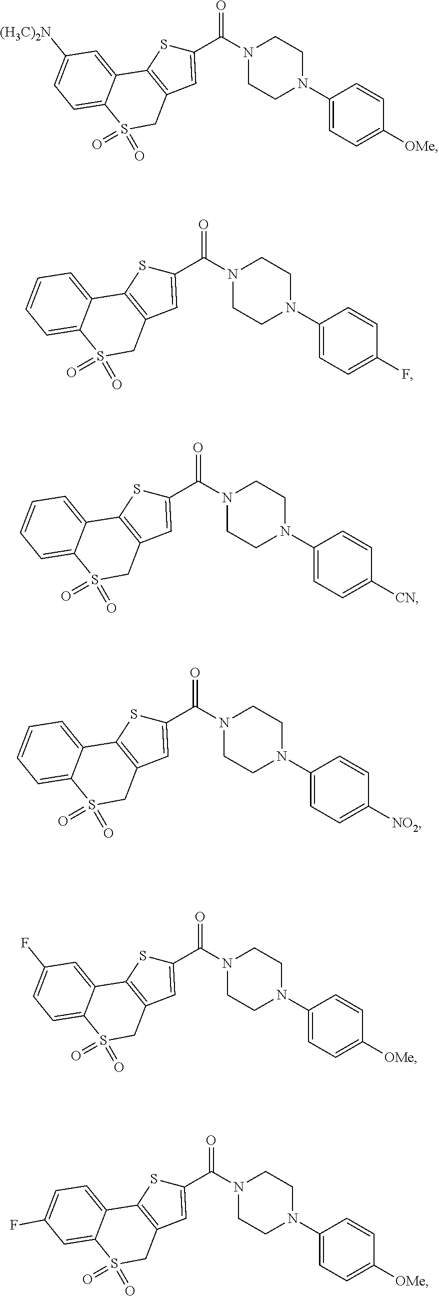

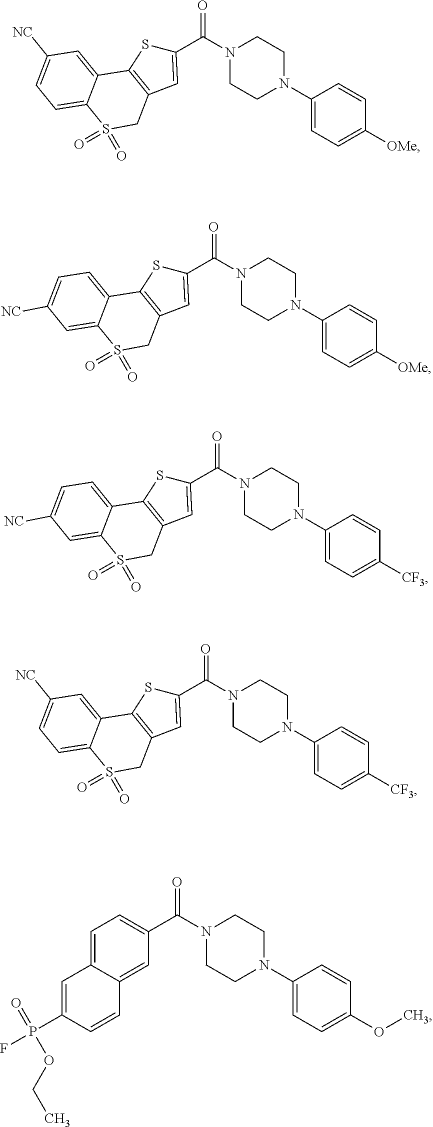 [SMILES: COc1ccc(N2CCN(C(=O)c3cc4c(s3)-c3cc(C#N)ccc3S(=O)(=O)C4)CC2)cc1 ; COc1ccc(N2CCN(C(=O)c3cc4c(s3)-c3ccc(C#N)cc3S(=O)(=O)C4)CC2)cc1 ; N#Cc1ccc2c(c1)S(=O)(=O)Cc1cc(C(=O)N3CCN(c4ccc(C(F)(F)F)cc4)CC3)sc1-2 ; N#Cc1ccc2c(c1)-c1sc(C(=O)N3CCN(c4ccc(C(F)(F)F)cc4)CC3)cc1CS2(=O)=O ; CCOP(=O)(F)c1ccc2cc(C(=O)N3CCN(c4ccc(OC)cc4)CC3)ccc2c1]

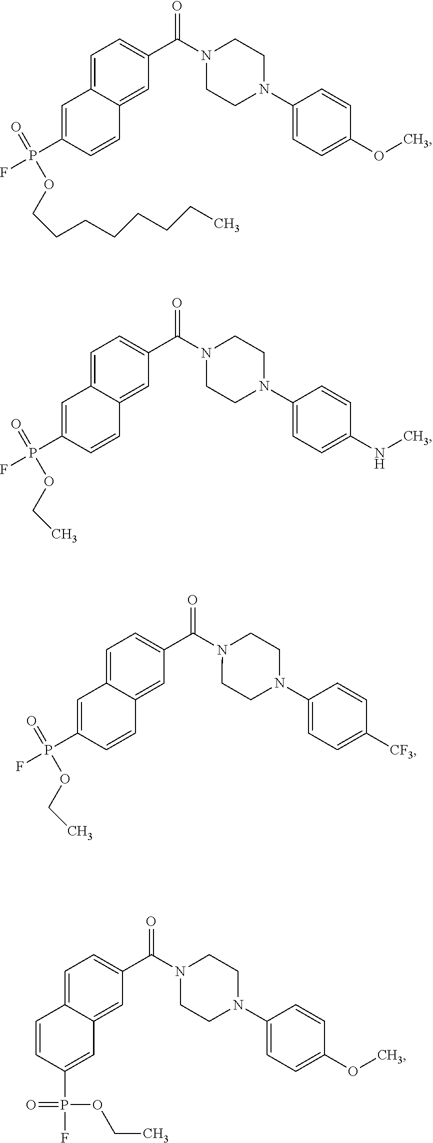 [SMILES: CCCCCCCCOP(=O)(F)c1ccc2cc(C(=O)N3CCN(c4ccc(OC)cc4)CC3)ccc2c1 ; CCOP(=O)(F)c1ccc2cc(C(=O)N3CCN(c4ccc(NC)cc4)CC3)ccc2c1 ; CCOP(=O)(F)c1ccc2cc(C(=O)N3CCN(c4ccc(C(F)(F)F)cc4)CC3)ccc2c1 ; CCOP(=O)(F)c1ccc2ccc(C(=O)N3CCN(c4ccc(OC)cc4)CC3)cc2c1]

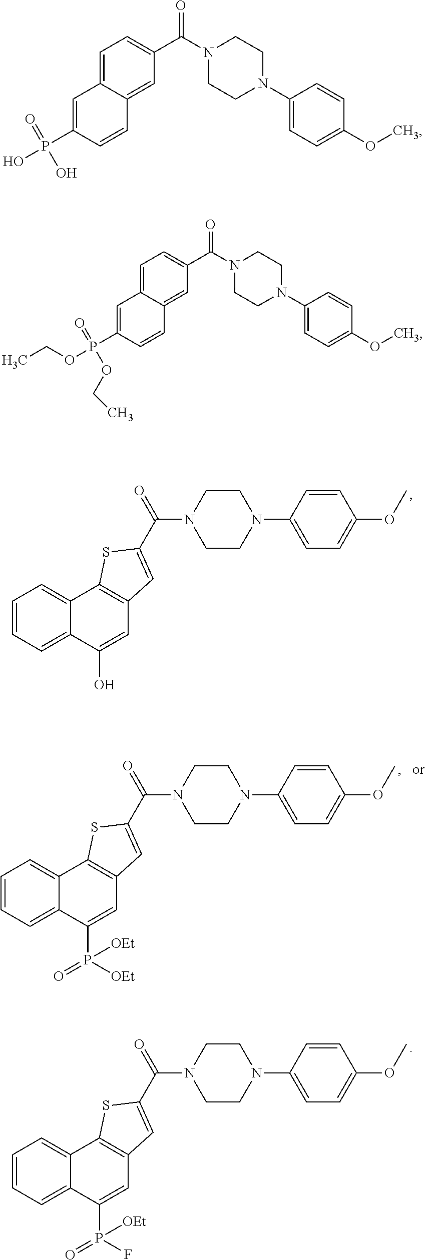 [SMILES: COc1ccc(N2CCN(C(=O)c3ccc4cc(P(=O)(O)O)ccc4c3)CC2)cc1 ; CCOP(=O)(OCC)c1ccc2cc(C(=O)N3CCN(c4ccc(OC)cc4)CC3)ccc2c1 ; COc1ccc(N2CCN(C(=O)c3cc4cc(O)c5ccccc5c4s3)CC2)cc1 ; CCOP(=O)(OCC)c1cc2cc(C(=O)N3CCN(c4ccc(OC)cc4)CC3)sc2c2ccccc12 ; CCOP(=O)(F)c1cc2cc(C(=O)N3CCN(c4ccc(OC)cc4)CC3)sc2c2ccccc12]

33. A pharmaceutical composition comprising one or compound(s) of claim 1.

34. The pharmaceutical composition of claim 33, wherein the pharmaceutical composition further comprises one or more pharmaceutically acceptable excipient(s).

35. A method of at least partially or completely inhibiting Acyl Protein Thioesterase 1 (APT1) and/or Acyl Protein Thioesterase 2 (APT2), the method comprising:
administering to a subject an effective amount of one or more compound(s) of claim 1,
wherein the APT1 and/or the APT2 in the subject is at least partially or completely inhibited.

36. The method of claim 35, wherein the APT1 is at least partially or completely inhibited and the APT2 is not at least partially or completely inhibited, or the APT2 is at least partially or completely inhibited and the APT1 is not at least partially or completely inhibited.

37. A method for treating a disorder characterized by accelerated differentiation of T helper 17 (Th17) cells, the method comprising:
administering to subject diagnosed with or suffering from the disorder an effective amount of one or more inhibitor(s) of an enzyme or enzymes that regulates the S-palmitoylation of STAT3.

38. The method of claim 37, wherein the enzyme is Acyl Protein Thioesterase (APT1) and/or Acyl Protein Thioesterase (APT2).

39. The method of claim 37, wherein the inhibitor(s) is/are a compound or compounds of claim 1.

40. The method of claim 39, wherein the inhibitor(s), individually, are a non-covalent inhibitor or non-covalent inhibitors and/or a covalent inhibitor or covalent inhibitors of APT1 and/or APT2.

41. The method of claim 37, wherein the disorder is an autoimmune disorder, a neurodegenerative disorder, an inflammatory disorder, or an immune-mediated disease.

42. The method of claim 41, wherein the autoimmune disorder is inflammatory bowel disease, multiple sclerosis, rheumatoid arthritis, lupus, graft versus host disease, type I diabetes, psoriasis, atopic dermatitis, eczema, alopecia areata, myeloproliferative neoplasms, dermatomyositis, or Guillain-Barre Syndrome; the neurodegenerative disorder is Parkinson's disease, Alzheimer's disease, or Huntington's disease; or the immune mediated disease is lymphoma or leukemia.

43. A method of treating a subject diagnosed with or is in need of treatment for an autoimmune disorder, a neurodegenerative disorder, or an inflammatory disorder, the method comprising
administering to a subject an effective amount of one or more compound(s) of claim 1,
wherein one or more symptom(s) and/or indication(s) of the subject is at least partially alleviated.

44. The method of claim 43, wherein the subject has APT1 and/or APT2 and the APT1 and/or the APT2 is at least partially or completely inhibited.
